(19) United States
(12) Patent Application Publication
(10) Pub. No.: US 2026/0240970 A1
(43) Pub. Date: Aug. 20, 2026

(54) NEOANTIGEN IMMUNOTHERAPIES

(71) Applicant: IOGENETICS, LLC, Sun Prairie, WI (US)

(72) Inventors: Jane Homan, Sun Prairie, WI (US); Robert D. Bremel, Sun Prairie, WI (US)

(21) Appl. No.: 19/539,385

(22) Filed: Feb. 13, 2026

Related U.S. Application Data
(63) Continuation of application No. 16/898,924, filed on Jun. 11, 2020, now Pat. No. 12,569,546.
(60) Provisional application No. 62/859,962, filed on Jun. 11, 2019.
(60) Provisional application No. 62/983,197, filed on Feb. 28, 2020.

(51) Int. Cl. A61K 39/00; A61K 40/11; A61K 40/42; A61K 45/06

(52) U.S. Cl. CPC A61K 39/0011; A61K 39/001104; A61K 39/001106; A61K 39/001151; A61K 39/001162; A61K 39/001163; A61K 40/11; A61K 40/4201; A61K 40/4204; A61K 40/4205; A61K 2039/54; A61K 45/06; A61K 2239/47; A61K 2239/55

(57) ABSTRACT

This invention provides a method for maximizing the immune response to mutated tumor specific proteins, either by means of stimulation of dendritic cells or T cells in vitro followed by administration of these cells to a patient, or by means of administration of a neoantigen vaccine in which de novo peptides, or their encoding nucleic acids, have been designed to ensure an appropriate level of binding affinity to a particular cancer patient's MHC alleles. This invention further provides for modulating the immune response in an immunopathology other than cancer.

### CROSS-REFERENCE TO RELATED APPLICATIONS

The present application is a continuation of U.S. patent application Ser. No. 16/898,924, filed Jun. 11, 2020, which claims the benefit of U.S. Prov. Appl. 62/983,197, filed Feb. 28, 2020, and U.S. Prov. Appl. 62/859,962, filed Jun. 11, 2019, each of which are incorporated by reference herein in their entireties.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 939,311 Byte ASCII (Text) file named “IOGEN_37855_304_SequenceListing.xml” created on Feb. 13, 2026.

### FIELD OF THE INVENTION

This invention provides a method for maximizing the immune response to mutated tumor specific proteins, either by means of stimulation of dendritic cells or T cells in vitro followed by administration of these cells to a patient, or by means of administration of a neoantigen vaccine in which de novo peptides, or their encoding nucleic acids, have been designed to ensure an appropriate level of binding affinity to a particular cancer patient's MHC alleles. In addition it provides for enhancing B cell responses to tumors with exposed B cell epitopes. A further application of the present invention is to provide for the design of peptides to modulate the T cell immune response in immunopathologies other than solid tumors.

### BACKGROUND OF THE INVENTION

Immunology is based on self-non-self discrimination. Most pathogens contain molecular signatures that can be recognized by the host and trigger immune responses. Unlike pathogens, these molecular signatures are not generally expressed by tumor cells, making them more difficult to be distinguished from normal cells. However, T cells can recognize tumor antigens expressed by tumor cells. A class of tumor antigens, named tumor-associated antigens, is expressed in some normal tissues at low levels but is over-expressed in malignant cells. Many of the tumor-associated antigens have been identified as the targets of tumor-reactive T cells, isolated from tumor infiltrating lymphocytes (TILs), from draining lymph nodes or from peripheral blood. However, expression of these antigens in normal cells can trigger central and peripheral tolerance mechanisms that lead to the selection of T cells with low-affinity T cell receptors (TCR). Conversely, attempts to target tumor-associated antigens with high-affinity TCRs can lead to severe toxicities due to normal tissue destruction.

Another class of tumor antigens is tumor-specific neoantigens, which arise via mutations that alter amino acid coding sequences (non-synonymous somatic mutations). Some of these mutated peptides can be expressed, processed and presented on the cell surface, and subsequently recognized by T cells. Because normal tissues do not possess these somatic mutations, neoantigen-specific T cells are not subject to central and peripheral tolerance, and also lack the ability to induce normal tissue destruction. As a result, neoantigens are targets for T cell-based cancer immunotherapy.

In some instances tumor mutations may change the B cell epitopes in a tumor protein and create new epitope targets for antibody mediated therapy. Furthermore, changes in T cell neoantigens may alter T cell help to B cell epitopes.

In immunopathologies other than solid tumors, including but not limited to autoimmunity, allergies and inflammation, an excessive immune response by T cells may drive the pathology. In such a situation the provision of a very high affinity MHC binding peptide may allow dampening of the T cell response by causing specific clones to become exhausted and anergic. As this is a clonal specific intervention, the design of peptides which can bring about such modulation may be specific to the individual subject.

### SUMMARY OF THE INVENTION

This invention provides a method for maximizing the immune response to mutated tumor specific proteins, either by means of stimulation of dendritic cells or T cells in vitro followed by administration of these cells to a patient, or by means of administration of a neoantigen vaccine in which de novo peptides, or their encoding nucleic acids, have been designed to ensure an appropriate level of binding affinity to a particular cancer patient's MHC alleles. In addition, it provides for enhancing B cell responses to tumors with exposed B cell epitopes.

In some preferred embodiments, the present invention provides methods for treating cancer in a subject comprising designing a group of one or more tumor-specific T-cell stimulating peptides, or nucleic acids encoding T cell stimulating peptides, which have a desired predicted binding affinity for the MHC alleles of the subject, comprising the following steps: obtaining a biopsy of the subject's tumor; sequencing proteins in said biopsy and identifying the mutated amino acids in said proteins and the peptide comprising each said mutated amino acids; determining T cell exposed motifs which comprise mutated amino acids in each of the proteins; determining the predicted binding affinity to the subject's MHC alleles of peptides which comprises each said T cell exposed motif, or a subset thereof; generating an array of alternative peptides not present in the tumor, wherein each peptide in the array comprises the amino acids of one of said T cell exposed motifs, and in which the amino acids not within the T cell exposed motif are substituted to change the predicted MHC binding affinity; selecting a group of one or more selected peptides from said array of alternative peptides which have a desired predicted binding affinity for one or more of the subject's MHC alleles; and synthesizing said group of one or more selected peptides, or nucleic acids encoding the selected peptides. In some preferred embodiments, the group of one or more selected peptides, or nucleic acids encoding the peptides, stimulate a tumor-specific T cell response in said subject upon administration. In some preferred embodiments, the group of one or more selected peptides, or nucleic acids encoding the peptides, are administered to said subject to stimulate a tumor-specific T cell response.

In some preferred embodiments, the MHC alleles are MHC type I and said T cell response is a CD8+ response. In some preferred embodiments, the MHC alleles are MHC type II and said T cell response is a CD4+ response. In some preferred embodiments, the selected peptides are 9 or 10 amino acids long. In some preferred embodiments, the selected peptides are 13-20 amino acids long.

In some preferred embodiments, the group of one or more selected peptides comprises at least 5 unique peptides not present in the proteins sequenced in the tumor. In some preferred embodiments, the group of one or more selected peptides comprises at least 20 unique peptides not present in the proteins sequenced in the tumor. In some preferred embodiments, the group of one or more selected peptides comprises at least 60 peptides not present in the proteins sequenced in the tumor.

In some preferred embodiments, the group of one or more selected peptides comprises more than 5 different T cell exposed motifs identified in the tumor. In some preferred embodiments, the group of one or more selected peptides comprises more than 10 different T cell exposed motifs identified in the tumor. In some preferred embodiments, the group of one or more selected peptides comprises more than 50 distinct T cell exposed motifs identified in the tumor. In some preferred embodiments, the group of one or more selected peptides comprises peptides each of which binds to one of at least 2 MHC alleles carried by said subject. In some preferred embodiments, the group of one or more selected peptides comprises peptides each of which binds to one of at least 4 MHC alleles carried by said subject.

In some preferred embodiments, the desired predicted binding affinity exceeds 85% of the binding affinity of all peptides in the tumor protein that comprises the mutated amino acid. In some preferred embodiments, the desired predicted binding affinity exceeds 95% of the binding affinity of all peptides in the tumor protein that comprises the mutated amino acid. In some preferred embodiments, the desired predicted binding affinity exceeds 99% of the binding affinity of all peptides in the tumor protein that comprises the mutated amino acid.

In some preferred embodiments, the desired predicted binding affinity is less than 20 nanomolar. In some preferred embodiments, the desired predicted binding affinity is less than 50 nanomolar. In some preferred embodiments, the desired predicted binding affinity is less than 100 nanomolar. In some preferred embodiments, the desired predicted binding affinity is less than 500 nanomolar.

In some preferred embodiments, the group of one or more selected peptides includes only peptides which are soluble in a desired solvent.

In some preferred embodiments, the proteins in the subject's biopsy comprise mutations that are unique to that subject. In some preferred embodiments, the group of one or more selected peptides, or nucleic acids encoding the peptides are unique to the subject. In some preferred embodiments, the proteins in the subject's biopsy comprise mutations that are found in a multiplicity of cancers affecting a multiplicity of subjects. In some preferred embodiments, the group of one or more selected peptides, or nucleic acids encoding the peptides are applicable to multiple subjects of shared MHC alleles.

In some preferred embodiments, the mutated amino acids comprise a substituted amino acid. In some preferred embodiments, the mutated amino acids comprise the product of insertion or deletion of one or more amino acids. In some preferred embodiments, the mutated amino acids comprise a new sequence that is the product of an in-frame nucleotide mutation. In some preferred embodiments, the mutated amino acids comprise a new sequence that is the product of a fusion of two gene. In some preferred embodiments, the protein sequencing is derived from a whole genome sequence. In some preferred embodiments, the MHC alleles of said subject are also determined from the whole genome sequence. In some preferred embodiments, the HLA alleles are determined by comparison of the sequence of chromosome 6 with a HLA sequence database.

In some preferred embodiments, each of said one or more selected peptides are linked by a linker to a fusion partner. In some preferred embodiments, the a multiplicity of said one or more selected peptides are linked by a linker to a fusion partner. In some preferred embodiments, the fusion partner is selected from the group consisting of a multimer of hydrophobic amino acids, or an unnatural hydrophobic amino acid, and a lipid core peptide system. In some preferred embodiments, the fusion partner facilitates nanoparticle formation. In some preferred embodiments, the fusion partner is selected from the group consisting of an immunoglobulin, Fc portion of an immunoglobulin, and fragment of an immunoglobulin. In some preferred embodiments, the linker is a cleavable linker. In some preferred embodiments, the linker is selected from the group consisting of linkers comprising one or more lysines, linkers comprising one or more arginines, and a cathepsin cleavable linker.

In some preferred embodiments, the group of one or more selected peptides, or nucleic acids encoding the peptides, are prescribed for an identified individual patient. In some preferred embodiments, the group of one or more selected peptides, or nucleic acids encoding the peptides, are formulated by a compounding pharmacy.

In some preferred embodiments, the peptides are selected from the group consisting of SEQ ID NO: 1-244 and combinations thereof.

In some preferred embodiments, where the peptides comprise a deletion, the deletion is the deletion in EGFRviii. In some preferred embodiments, the array of alternative peptides spans the deletion of exons 2-7 in EGFRviii. In some preferred embodiments, the peptides comprise the T cell exposed motifs from the group EEKKG (SEQ ID NO: 252), EKKGN (SEQ ID NO: 246), KKGNY (SEQ ID NO: 245), KGNYV (SEQ ID NO: 250), GNYVV (SEQ ID NO: 247). In some preferred embodiments, the array of alternative peptides comprise any of the peptides of SEQ 245-284. In other embodiments said dendritic cells are contacted with autologous T cells from the subject or donor T cells, and the T cells, or clonal populations arising from them, are then subsequently administered to the subject. In yet other preferred embodiments, the selected peptide and the MHC which binds it is engineered into a T cell and said T cell grown to provide an expanded clone which is subsequently administered to a subject.

In some preferred embodiments, the group of one or more selected peptides is provided to stimulate T cells in vitro which are subsequently administered to a subject. In some preferred embodiments, the group of one or more selected peptides, or the nucleic acids that encode them, is provided to contact dendritic cells in vitro, and the dendritic cells are subsequently administered to a subject.

In some preferred embodiments, the group of one or more selected peptides is administered to a subject as a vaccine.

In some preferred embodiments, the peptides in said group of one or more selected peptides are each encoded in nucleic acid which is administered to a subject as a vaccine. In some preferred embodiments, the nucleic acid is RNA. In some preferred embodiments, the nucleic acid is DNA.

In some preferred embodiments, the foregoing methods further comprise down-selecting the group of tumor-specific T-cell stimulating peptides on an allele-specific basis to remove those which have low probability of being accessible to T cell targeting in the subject, comprising: evaluating the predicted binding affinity to each of the subject's MHC alleles of the peptide which comprises each said T cell exposed motif in the mutated protein; determining if said predicted binding affinity is in the lower 50% of binding affinity for that MHC allele relative to predicted binding of other peptides in the same protein; and removing from the group of one or more selected peptides those peptides with low probability of being accessible to T cell targeting for that specific allele-T cell exposed motif combination.

In some preferred embodiments, the present invention provides a diagnostic test comprising peptides identified according to the foregoing methods.

In some preferred embodiments, the present invention provides a vaccination regimen comprising administering a group of peptides, or nucleic acids encoding the same peptides, or fusions selected according to the methods described above to a subject with cancer. In some preferred embodiments, the group of peptides, or nucleic acids encoding the same peptides, is divided into subgroups and each subgroup administered at a different timepoint. In some preferred embodiments, the subgroups of peptides, or nucleic acids encoding the same peptides, are selected so that each subgroup comprises peptides which collectively binds to a multiplicity of different MHC alleles, and include a multiplicity of different T cell exposed motif targets. In some preferred embodiments, the peptides included in said subgroups of peptides, or nucleic acids encoding the same peptides, are prioritized according to the frequency classification in the human proteome of the T cell exposed motif which each peptide comprises. In some preferred embodiments, the vaccination is accompanied by administration of an immunotherapy intervention. In some preferred embodiments, the immunotherapy intervention is a checkpoint inhibitor immunotherapeutic. In some preferred embodiments, the vaccination is followed by administration of an immunotherapy intervention. In some preferred embodiments, the immunotherapy intervention is a checkpoint inhibitor immunotherapeutic. In some preferred embodiments, the vaccination by each subgroup of peptides is followed by administration of an immunotherapy intervention. In some preferred embodiments, the immunotherapy intervention is a checkpoint inhibitor immunotherapeutic.

In some preferred embodiments, the present invention provides vaccines for administration to a subject with cancer comprising a group of peptides, or nucleic acids encoding the same peptides, or fusions selected according to the methods described above. In some preferred embodiments, the group of peptides or nucleic acids encoding the same peptides, is selected to stimulate T cells that target mutations unique to the particular subject. In some preferred embodiments, the group of peptides or nucleic acids encoding the same peptides, is selected to stimulate T cells that target mutations shared among a multiplicity of cancers. In some preferred embodiments, the group of peptides or nucleic acids encoding the same peptides, comprises both peptides selected to stimulate T cells that target mutations unique to the particular subject and those selected to stimulate T cells that target mutations shared among a multiplicity of cancers. In some preferred embodiments, the vaccine is administered to a subject parenterally. In some preferred embodiments, the vaccine is administered to a subject intradermally. In some preferred embodiments, the vaccine is administered by microneedle array. In some preferred embodiments, the vaccine comprises an adjuvant. In some preferred embodiments, the vaccine is accompanied by the application of a local pro-inflammatory agent. In some preferred embodiments, the vaccine also comprises peptides which occur naturally in the tumor protein. In some preferred embodiments, the vaccine also comprises one or more peptides which comprise a B cell epitope.

In some preferred embodiments, the present invention provides arrays of peptides comprising peptides selected by the methods described above to have a desired MHC binding affinity to stimulate T cells targeting mutated T cell exposed motifs shared by more than one cancer. In some preferred embodiments, the array of peptides includes peptides which are designed to stimulate T cells in multiple individuals carrying MHC of one or more specific HLA alleles. In some preferred embodiments, the desired binding affinity of each peptide is less than 20 nanomolar. In some preferred embodiments, the desired binding affinity of each peptide is less than 50 nanomolar. In some preferred embodiments, the desired binding affinity of each peptide is less than 100 nanomolar. In some preferred embodiments, the desired binding affinity of each peptide is less than 500 nanomolar. In some preferred embodiments, the mutated T cell exposed motifs are shared by 3 or more cancer types. In some preferred embodiments, the mutated T cell exposed motifs are shared by cancers affecting 3 or more tissue types. In some preferred embodiments, the mutated T cell exposed motifs are drawn from 5 or more proteins. In some preferred embodiments, the mutated T cell exposed motifs are drawn from 10 or more proteins. In some preferred embodiments, the array comprises any of the peptides of SEQ 1-244. In some preferred embodiments, where the peptides comprise a deletion, the deletion is the deletion in EGFRviii. In some preferred embodiments, the array of alternative peptides spans the deletion of exons 2-7 in EGFRviii. In some preferred embodiments, the peptides comprise the T cell exposed motifs from the group EEKKG (SEQ ID NO: 252), EKKGN (SEQ ID NO: 246), KKGNY (SEQ ID NO: 245), KGNYV (SEQ ID NO: 250), GNYVV (SEQ ID NO: 247). In some preferred embodiments, the array of alternative peptides comprise any of the peptides of SEQ 245-284. In some preferred embodiments, the array also comprises peptides which occur naturally in the tumor protein. In some preferred embodiments, the array also comprises one or more peptides which comprise a B cell epitope.

In some preferred embodiments, the present invention provides methods for designing a group of one or more of tumor-specific T-cell stimulating peptides for a particular subject with cancer, and identifying potential adverse targets of the T cells in the self-proteome of that subject, comprising: obtaining a biopsy of the subject's tumor; sequencing proteins in said biopsy and identifying the mutated amino acids in said proteins from said tumor; determining the T cell exposed motifs which comprise mutated amino acids in one or more proteins and which are selected as potential neoantigen targets; identifying those proteins in the normal human proteome which carry the same T cell exposed motifs; determining the predicted binding affinity of the subject's MHC alleles for the peptide which carries each T cell exposed motif in a protein of the normal human proteome; based on its MHC binding affinity, determining the probability that a T cell exposed motif would be presented and exposed to T cells in its natural context in the normal human proteome in this subject; listing the human proteome proteins which share T cell exposed motifs with said potential neoantigen targets and wherein the T cell exposed motif in the normal human proteome protein is are predicted to be exposed to T cells in the particular subject; and identifying those proteins in said listing which are a potential source of adverse effects. In some preferred embodiments, the subject's MHC alleles are MHC I. In some preferred embodiments, the subject's MHC alleles are MHC II. In some preferred embodiments, the predicted binding affinity of the subject's MHC alleles for the peptide which carries each T cell exposed motif in a protein of the normal human proteome is above 100 nm. In some preferred embodiments, the predicted binding affinity of the subject's MHC alleles for the peptide which carries each T cell exposed motif in a protein of the normal human proteome is in the highest 15% of peptides in that protein. In some preferred embodiments, the methods further comprise providing said listing to an oncologist to conduct a risk-benefit analysis of the use of said neoantigens in said subject.

In some preferred embodiments, the present invention provides methods for treating an immunopathology in a subject, comprising designing a group of one or more T-cell epitope peptides, or nucleic acids encoding T cell epitope peptides, which have a desired predicted binding affinity for MHC alleles of the subject, comprising the following steps: identifying a protein of interest comprising an epitope of interest that is causing the immunopathological T cell response; obtaining the sequence for said protein of interest and identifying the peptide comprising the epitope of interest; determining T cell exposed motifs in said epitope of interest; determining the predicted binding affinity to the subject's MHC alleles of peptides which comprise each said T cell exposed motif, or a subset thereof; generating an array of alternative peptides not present in the natural protein sequence, wherein each peptide in the array comprises the amino acids of one of said T cell exposed motifs, and in which one or more of the amino acids not within the T cell exposed motif are substituted to change the predicted MHC binding affinity; selecting a group of one or more selected peptides from said array of alternative peptides which have a desired predicted binding affinity for one or more of the subject's MHC alleles; synthesizing said group of one or more selected peptides, or nucleic acids encoding the selected peptides; and administering said group of one or more selected peptides, or nucleic acids encoding the selected peptides, to the subject.

In some preferred embodiments, the MHC alleles are MHC type I and said T cell response is a CD8+ response. In some preferred embodiments, the MHC alleles are MHC type II and said T cell response is a CD4+ response. In some preferred embodiments, the selected peptides are 9 or 10 amino acids long. In some preferred embodiments, the selected peptides are 13-20 amino acids long. In some preferred embodiments, the group of one or more selected peptides comprises at least 3 unique peptides not present in the original protein of interest in the subject. In some preferred embodiments, the group of one or more selected peptides comprises more than one different T cell exposed motifs. In some preferred embodiments, the group of one or more selected peptides comprises peptides each of which binds to more than one MHC alleles carried by said subject.

In some preferred embodiments, the desired predicted binding affinity exceeds 99% of the binding affinity of all peptides in the protein of interest that comprises the T cell epitope of interest. In some preferred embodiments, the desired predicted binding affinity is less than 500 nanomolar. In some preferred embodiments, the desired predicted binding affinity is less than 100 nanomolar. In some preferred embodiments, the desired predicted binding affinity is less than 50 nanomolar. In some preferred embodiments, the desired predicted binding affinity is less than 20 nanomolar.

In some preferred embodiments, the group of one or more selected peptides includes only peptides which are soluble in a desired solvent. In some preferred embodiments, the group of one or more selected peptides, or nucleic acids encoding the peptides are unique to the subject. In some preferred embodiments, the group of one or more selected peptides, or nucleic acids encoding the peptides are applicable to multiple subjects of shared MHC alleles.

In some preferred embodiments, each of said one or more selected peptides are linked by a linker to a fusion partner. In some preferred embodiments, the fusion partner is selected from the group consisting of a multimer of hydrophobic amino acids, or an unnatural hydrophobic amino acid, and a lipid core peptide system. In some preferred embodiments, the fusion partner facilitates nanoparticle formation. In some preferred embodiments, the fusion partner is selected from the group consisting of an immunoglobulin, Fc portion of an immunoglobulin and a fragment of an immunoglobulin. In some preferred embodiments, the linker is a cleavable linker.

In some preferred embodiments, the group of one or more selected peptides, or nucleic acids encoding the peptides, are prescribed for an identified individual patient. In some preferred embodiments, the group of one or more selected peptides, or nucleic acids encoding the peptides, are formulated by a compounding pharmacy.

In some preferred embodiments, the selected peptides are administered to the subject parenterally. In some preferred embodiments, the selected peptides are administered to the subject intradermally. In some preferred embodiments, the selected peptides are administered to the subject orally. In some preferred embodiments, the selected peptides are administered to the subject by microneedle array. In some preferred embodiments, the subject is afflicted by an allergy. In some preferred embodiments, the subject is afflicted by an autoimmune disease. In some preferred embodiments, the immunopathology arises as an adverse immune response to a biopharmaceutical protein.

In some preferred embodiments, the selected peptides comprise one or more of the peptides in Table 27 or nucleic acids encoding these peptides. In some preferred embodiments, the selected peptides comprise one or more of the peptides in Table 28 or nucleic acids encoding these peptides.

### DESCRIPTION OF THE FIGURES

FIG. 1: Predicted binding affinity with mutant amino acid in T cell exposed motif I positions of wild type (wt) and mutant homologs of 7 tumor specific proteins. Predicted affinity (Y-axis=LN IC₅₀) for 4 MHC I alleles of wild type vs multiple different mutant TCEM I for 7 different proteins commonly mutated in different cancers. The dashed line is at 500 nM, a value commonly used to predict T cell responses. The boxplot is a Tukey outlier type where the box represents the 25 and 75 percentile and the whiskers correspond to 1.5× interquartile range. The yellow shaded area comprises peptides with the highest affinity and for any of the alleles corresponds to approximately 1% of the total TCEM and are all outliers. Overall, the MHC I binding affinity of the peptides containing the TCEM is very low; a median of 10 implies a value of about 22 μM (micromolar), more than 40× lower than the 500 nM (nanomolar) that is the consensus T cell stimulatory level. In addition, there is no statistical difference between the wt and mutant TCEM-containing peptides as is shown graphically by the boxplots and the datapoint scatter.

FIG. 2: Predicted binding affinity with mutant amino acid in groove exposed motif (GEM I) positions of wild type and multiple different mutant homologs of 7 tumor specific proteins. Predicted affinity (Y-axis=LN IC₅₀) for 4 MHC I alleles of wt vs mutant TCEM I for 7 different proteins commonly mutated in different cancers. The dashed line is at 500 nM, a value commonly used to predict T cell responses. The boxplot is a Tukey outlier type where the box represents the 25 and 75 percentile and the whiskers correspond to 1.5× interquartile range. The yellow shaded area comprises peptides with the highest affinity and for any of the alleles corresponds to approximately 1% of the total TCEM and are all outliers. Overall, the MHC I binding affinity of the peptides containing the TCEM is very low; a median of 10 implies a value of about 22 μM, more than 40× lower than the 500 nM that is the consensus T cell stimulatory level. In addition, there is no statistical difference between the wt and mutant TCEM-containing peptides as is shown graphically by the boxplots and the datapoint scatter.

FIG. 3: Distribution histograms of TCEM I frequency for the 37,622 different TCEM peptides mutants (top panel) and wt motifs (bottom panel) in seven proteins of interest as listed in FIGS. 1 and 2. The base frequency of the TCEM in the proteome was log 2 basis. This frequency was standardized to a zero mean unit variance distribution with a Johnson SI distribution function. The wt distribution shows that the mean is shifted slightly negative from zero mean of the full proteome but the standard deviation is very nearly 1.0 (unit variance). Thus, the it is inferred that the wt TCEM frequency is a relatively random selection from the proteome unit variance distribution. The histogram bar at the far left of the top panel is a coded frequency for TCEM completely absent from the human proteome. This pattern of TCEM generation by mutation shows the stochastic mutation process inserts amino acids into protein sequences that are either much more rare or in many cases (14% overall), completely absent in normal protein sequences in the proteome.

FIG. 4: Paired comparison of the human proteome TCEM I frequency of wild type and 37,621 mutated peptides of 5 different proteins commonly mutated in different cancers. The base frequency of the TCEM in the proteome was log 2 basis. This frequency was standardized with a Johnson SI distribution function and thus the units of both the X-axis and Y-axis are standard deviations. The graphs are the paired differences (wt-mut) (Y-axis) by the paired means (wt+mut)/2 (X-axis). The paired t-test results are for all three alternative hypotheses. The matched responses for each protein is a simple version of repeated measures analysis. The frequency of the wt TCEM in the proteome is about 1 standard deviation greater overall than the mutants. Thus, a mutational event that inserts a new amino acid in the TCEM consistently produces TCEM that are much more rare as compared to the wt TCEM

FIG. 5: The location of the dominant mutations in five proteins in which mutations are shared across multiple cancers. Although the proteins are subject to mutation at many locations there are some amino acid positions that are clearly more susceptible than others.

FIG. 6: Binding affinity of native peptides comprising exposed TCEM mutant compared to peptides generated by simulation. Note the Y axis is centered at zero (the mean) for the natural peptides whereas simulated peptides figures only show those below zero because peptides with binding affinities lower than the mean are deemed to be not useful and selected against in the simulation process. Numbers at bottom indicate the number of available peptides from which to select.

FIG. 7: Few MHC I alleles bind naturally at each of the five unique TCEM positions in the EGFRviii variant. The figure highlights those binding at better than 1 SD units below the mean for the protein (approx. 500 nm). TCEM amino acid motif is shown below each figure.

FIG. 8: Shows an example of determination of a subject's HLA alleles from a chromosome 6 BAM slice. MHC I ABC alleles and DRB1 showing sequential hits matching the IMGT database. Alleles shown in boxes are the clear highest matches for this individual. Figure shows 2 digits of HLA for space; four digit resolution was determined.

FIG. 9A-B: Distribution of tumor mutations in protein topological domains. A: Relative fraction of proteins of four different topological types in two different cancers. Compared to the distribution in the human proteome (red, hg19 including all isoforms). The data is combined from 30 cases each of GBM (blue) and LUSC (yellow). B: Distribution of mutations in protein domains for a) all mutated proteins b) oncogenes and c) tumor suppressors in the intracellular (i), membrane (m) extracellular (o) and secreted (sp) domains relative to the length of each domain. The Y axis indicates the domain length as C—N(C-terminal minus N-terminal) positions of the amino acid within the protein molecules.

FIG. 10: Shows the creation of new higher probability B cell epitopes in one LUSC case example. Mutation positions in the 104 mutated proteins in this case were centered at zero. The Y axis shows the difference in the probability that a mutant vs a wildtype peptide 9mer centered at each position comprises part of a B cell epitope (in standard deviation units). The highest probability B cell epitopes are colored blue. Hence the graphic shows that for some proteins the mutation created new prominent B cell epitopes, whereas in other proteins there is a reduction in B cell epitope probability.

FIG. 11: Comparative predicted binding of the mutated region for one of the pairs of HLA A alleles in 60 cases. The plots combines all the data for one of the two A alleles for 60 cases (30 GBM plus 30 LUSC) and compares the binding of the native peptide to the mutated peptide with the mutant amino acid at the substituted position. All binding predictions have been standardized within protein to a zero mean unit variance distribution. The regression line is forced to a have an intercept of zero and a slope of one and essentially represents a null hypothesis that there is no difference in the binding between the wild type and mutated peptides. Squares are the oncogene mutants and triangles the tumor suppressor mutants on the background of all passenger mutants. Amino acid side chains in pocket positions 1,2,3 and 9, the GEM, are in combination bind to the side chains of the peptide in the pocket and are effectively responsible for the binding affinity. The amino acid side chains of pocket position 4,5,6,7,8, the TCEM, protrude from the surface of the histotope and interact with the T cell receptor.

FIG. 12A-B: In 30 cases of GBM mutations in tumor proteins create rarer TCEM I motifs. A. Plot of mutant (y axis) compared to wild type (x axis) TCEM I motif frequency compared to the frequency of the motif in the human proteome. Negative numbers are less common in the proteome and values of −3 are absent completely from the proteome. Motifs are colored according to frequency with darker indicative of rarer motifs. B. The regression line is forced to a line with an intercept of zero and slope of one between the mutant and wild type sequences (i.e. =null hypothesis). The residuals all fall outside the low confidence limit on the quantile plot indicating a consistent difference between the mutant and wild type with the mutant carrying less common motifs.

### DEFINITIONS

As used herein, the term “genome” refers to the genetic material (e.g., chromosomes) of an organism or a host cell.

As used herein, the term “proteome” refers to the entire set of proteins expressed by a genome, cell, tissue or organism. A “partial proteome” refers to a subset the entire set of proteins expressed by a genome, cell, tissue or organism. Examples of “partial proteomes” include, but are not limited to, transmembrane proteins, secreted proteins, and proteins with a membrane motif. Human proteome refers to all the proteins comprised in a human being. Multiple such sets of proteins have been sequenced and are accessible at the InterPro international repository (www.ebi.ac.uk/interpro). Human proteome is also understood to include those proteins and antigens thereof which may be over-expressed in certain pathologies, or expressed in a different isoforms in certain pathologies. Hence, as used herein, tumor associated antigens are considered part of the human proteome. “Proteome” may also be used to describe a large compilation or collection of proteins, such as all the proteins in an immunoglobulin collection or a T cell receptor repertoire, or the proteins which comprise a collection such as the allergome, such that the collection is a proteome which may be subject to analysis. All the proteins in a bacteria or other microorganism are considered its proteome.

As used herein, the terms “protein,” “polypeptide,” and “peptide” refer to a molecule comprising amino acids joined via peptide bonds. In general “peptide” is used to refer to a sequence of 40 or less amino acids and “polypeptide” is used to refer to a sequence of greater than 40 amino acids.

As used herein, the term, “synthetic polypeptide,” “synthetic peptide” and “synthetic protein” refer to peptides, polypeptides, and proteins that are produced by a recombinant process (i.e., expression of exogenous nucleic acid encoding the peptide, polypeptide or protein in an organism, host cell, or cell-free system) or by chemical synthesis.

As used herein, the term “protein of interest” refers to a protein encoded by a nucleic acid of interest. It may be applied to any protein to which further analysis is applied or the properties of which are tested or examined. Similarly, as used herein, “target protein” may be used to describe a protein of interest that is subject to further analysis.

As used herein “peptidase” refers to an enzyme which cleaves a protein or peptide. The term peptidase may be used interchangeably with protease, proteinases, oligopeptidases, and proteolytic enzymes. Peptidases may be endopeptidases (endoproteases), or exopeptidases (exoproteases). The term peptidase would also include the proteasome which is a complex organelle containing different subunits each having a different type of characteristic scissile bond cleavage specificity. Similarly the term peptidase inhibitor may be used interchangeably with protease inhibitor or inhibitor of any of the other alternate terms for peptidase.

As used herein, the term “exopeptidase” refers to a peptidase that requires a free N-terminal amino group, C-terminal carboxyl group or both, and hydrolyses a bond not more than three residues from the terminus. The exopeptidases are further divided into aminopeptidases, carboxypeptidases, dipeptidyl-peptidases, peptidyl-dipeptidases, tripeptidyl-peptidases and dipeptidases.

As used herein, the term “endopeptidase” refers to a peptidase that hydrolyses internal, alpha-peptide bonds in a polypeptide chain, tending to act away from the N-terminus or C-terminus. Examples of endopeptidases are chymotrypsin, pepsin, papain and cathepsins. A very few endopeptidases act a fixed distance from one terminus of the substrate, an example being mitochondrial intermediate peptidase. Some endopeptidases act only on substrates smaller than proteins, and these are termed oligopeptidases. An example of an oligopeptidase is thimet oligopeptidase. Endopeptidases initiate the digestion of food proteins, generating new N- and C-termini that are substrates for the exopeptidases that complete the process. Endopeptidases also process proteins by limited proteolysis. Examples are the removal of signal peptides from secreted proteins (e.g. signal peptidase I) and the maturation of precursor proteins (e.g. enteropeptidase, furin). In the nomenclature of the Nomenclature Committee of the International Union of Biochemistry and Molecular Biology (NC-IUBMB) endopeptidases are allocated to sub-subclasses EC 3.4.21, EC 3.4.22, EC 3.4.23, EC 3.4.24 and EC 3.4.25 for serine-, cysteine-, aspartic-, metallo- and threonine-type endopeptidases, respectively. Endopeptidases of particular interest are the cathepsins, and especially cathepsin B, L and S known to be active in antigen presenting cells.

As used herein, the term “immunogen” refers to a molecule which stimulates a response from the adaptive immune system, which may include responses drawn from the group comprising an antibody response, a cytotoxic T cell response, a T helper response, and a T cell memory. An immunogen may stimulate an upregulation of the immune response with a resultant inflammatory response, or may result in down regulation or immunosuppression. Thus the T-cell response may be a T regulatory response. An immunogen also may stimulate a B-cell response and lead to an increase in antibody titer. Another term used herein to describe a molecule or combination of molecules which stimulate an immune response is “antigen”.

As used herein, the term “native” (or wild type) when used in reference to a protein refers to proteins encoded by the genome of a cell, tissue, or organism, other than one manipulated to produce synthetic proteins.

As used herein the term “epitope” refers to a peptide sequence which elicits an immune response, from either T cells or B cells or antibody

As used herein, the term “B-cell epitope” refers to a polypeptide sequence that is recognized and bound by a B-cell receptor. A B-cell epitope may be a linear peptide or may comprise several discontinuous sequences which together are folded to form a structural epitope. Such component sequences which together make up a B-cell epitope are referred to herein as B-cell epitope sequences. Hence, a B-cell epitope may comprise one or more B-cell epitope sequences. Hence, a B cell epitope may comprise one or more B-cell epitope sequences. A linear B-cell epitope may comprise as few as 2-4 amino acids or more amino acids.

“B cell core peptides” or “core pentamer” when used herein refers to the central 5 amino acid peptide in a predicted B cell epitope sequence. Said B cell epitope may be evaluated by predicting the binding of across a series of 9-mer windows, the core pentamer then is the central pentamer of the 9-mer window

As used herein, the term “predicted B-cell epitope” refers to a polypeptide sequence that is predicted to bind to a B-cell receptor by a computer program, for example, as described in PCT PCT US2011/029192, PCT US2012/055038, US2014/014523, and PCT US2015/039969, each of which is incorporated herein by reference, and in addition by Bepipred (Larsen, et al., Immunome Research 2:2, 2006) and others as referenced by Larsen et al (ibid) (Hopp T et al PNAS 78:3824-3828, 1981; Parker J et al, Biochem. 25:5425-5432, 1986). A predicted B-cell epitope may refer to the identification of B-cell epitope sequences forming part of a structural B-cell epitope or to a complete B-cell epitope.

As used herein, the term “T-cell epitope” refers to a polypeptide sequence which when bound to a major histocompatibility protein molecule provides a configuration recognized by a T-cell receptor. Typically, T-cell epitopes are presented bound to a MHC molecule on the surface of an antigen-presenting cell.

As used herein, the term “predicted T-cell epitope” refers to a polypeptide sequence that is predicted to bind to a major histocompatibility protein molecule by the neural network algorithms described herein, by other computerized methods, or as determined experimentally. As used herein, the term “major histocompatibility complex (MHC)” refers to the MHC Class I and MHC Class II genes and the proteins encoded thereby. Molecules of the MHC bind small peptides and present them on the surface of cells for recognition by T-cell receptor-bearing T-cells. The MHC is both polygenic (there are several MHC class I and MHC class II genes) and polyallelic or polymorphic (there are multiple alleles of each gene). The terms MHC-I, MHC-II, MHC-1 and MHC-2 are variously used herein to indicate these classes of molecules. Included are both classical and nonclassical MHC molecules. An MHC molecule is made up of multiple chains (alpha and beta chains) which associate to form a molecule. The MHC molecule contains a cleft or groove which forms a binding site for peptides. Peptides bound in the cleft or groove may then be presented to T-cell receptors. The term “MHC binding region” refers to the groove region of the MHC molecule where peptide binding occurs.

As used herein, a “MHC II binding groove” refers to the structure of an MHC molecule that binds to a peptide. The peptide that binds to the MHC II binding groove may be from about 11 amino acids to about 23 amino acids in length, but typically comprises a 15-mer. The amino acid positions in the peptide that binds to the groove are numbered based on a central core of 9 amino acids numbered 1-9, and positions outside the 9 amino acid core numbered as negative (N terminal) or positive (C terminal). Hence, in a 15mer the amino acid binding positions are numbered from −3 to +3 or as follows: −3, −2, −1, 1, 2, 3, 4, 5, 6, 7, 8, 9, +1, +2, +3.

As used herein, the term “haplotype” refers to the HLA alleles found on one chromosome and the proteins encoded thereby. Haplotype may also refer to the allele present at any one locus within the MHC. Each class of MHC—Is represented by several loci: e.g., HLA-A (Human Leukocyte Antigen-A), HLA-B, HLA-C, HLA-E, HLA-F, HLA-G, HLA-H, HLA-J, HLA-K, HLA-L, HLA-P and HLA-V for class I and HLA-DRA, HLA-DRB1-9, HLA-, HLA-DQA1, HLA-DQB1, HLA-DPA1, HLA-DPB1, HLA-DMA, HLA-DMB, HLA-DOA, and HLA-DOB for class II. The terms “HLA allele” and “MHC allele” are used interchangeably herein. HLA alleles are listed at hla.alleles.org/nomenclature/naming.html, which is incorporated herein by reference.

The MHCs exhibit extreme polymorphism: within the human population there are, at each genetic locus, a great number of haplotypes comprising distinct alleles—the IMGT/HLA database release (February 2010) lists 948 class I and 633 class II molecules, many of which are represented at high frequency (>1%). MHC alleles may differ by as many as 30-aa substitutions. Different polymorphic MHC alleles, of both class I and class II, have different peptide specificities: each allele encodes proteins that bind peptides exhibiting particular sequence patterns.

The naming of new HLA genes and allele sequences and their quality control is the responsibility of the WHO Nomenclature Committee for Factors of the HLA System, which first met in 1968, and laid down the criteria for successive meetings. This committee meets regularly to discuss issues of nomenclature and has published 19 major reports documenting firstly the HLA antigens and more recently the genes and alleles. The standardization of HLA antigenic specifications has been controlled by the exchange of typing reagents and cells in the International Histocompatibility Workshops. The IMGT/HLA Database collects both new and confirmatory sequences, which are then expertly analyzed and curated before been named by the Nomenclature Committee. The resulting sequences are then included in the tools and files made available from both the IMGT/HLA Database and at hla.alleles.org.

Each HLA allele name has a unique number corresponding to up to four sets of digits separated by colons. See e.g., hla.alleles.org/nomenclature/naming.html which provides a description of standard HLA nomenclature and Marsh et al., Nomenclature for Factors of the HLA System, 2010 Tissue Antigens 2010 75:291-455. HLA-DRB1*13:01 and HLA-DRB1*13:01:01:02 are examples of standard HLA nomenclature. The length of the allele designation is dependent on the sequence of the allele and that of its nearest relative. All alleles receive at least a four digit name, which corresponds to the first two sets of digits, longer names are only assigned when necessary.

The digits before the first colon describe the type, which often corresponds to the serological antigen carried by an allele, The next set of digits are used to list the subtypes, numbers being assigned in the order in which DNA sequences have been determined. Alleles whose numbers differ in the two sets of digits must differ in one or more nucleotide substitutions that change the amino acid sequence of the encoded protein. Alleles that differ only by synonymous nucleotide substitutions (also called silent or non-coding substitutions) within the coding sequence are distinguished by the use of the third set of digits. Alleles that only differ by sequence polymorphisms in the introns or in the 5′ or 3′ untranslated regions that flank the exons and introns are distinguished by the use of the fourth set of digits. In addition to the unique allele number there are additional optional suffixes that may be added to an allele to indicate its expression status. Alleles that have been shown not to be expressed, ‘Null’ alleles have been given the suffix ‘N’. Those alleles which have been shown to be alternatively expressed may have the suffix ‘L’, ‘S’, ‘C’, ‘A’ or ‘Q’. The suffix ‘L’ is used to indicate an allele which has been shown to have ‘Low’ cell surface expression when compared to normal levels. The ‘S’ suffix is used to denote an allele specifying a protein which is expressed as a soluble ‘Secreted’ molecule but is not present on the cell surface. A ‘C’ suffix to indicate an allele product which is present in the ‘Cytoplasm’ but not on the cell surface. An ‘A’ suffix to indicate ‘Aberrant’ expression where there is some doubt as to whether a protein is expressed. A ‘Q’ suffix when the expression of an allele is ‘Questionable’ given that the mutation seen in the allele has previously been shown to affect normal expression levels.

In some instances, the HLA designations used herein may differ from the standard HLA nomenclature just described due to limitations in entering characters in the databases described herein. As an example, DRB1_0104, DRB1*0104, and DRB1-0104 are equivalent to the standard nomenclature of DRB1*01:04. In most instances, the asterisk is replaced with an underscore or dash and the semicolon between the two digit sets is omitted.

As used herein, the term “polypeptide sequence that binds to at least one major histocompatibility complex (MHC) binding region” refers to a polypeptide sequence that is recognized and bound by one or more particular MHC binding regions as predicted by the neural network algorithms described herein or as determined experimentally.

As used herein the terms “canonical” and “non-canonical” are used to refer to the orientation of an amino acid sequence. Canonical refers to an amino acid sequence presented or read in the N terminal to C terminal order; non-canonical is used to describe an amino acid sequence presented in the inverted or C terminal to N terminal order.

As used herein, the term “allergen” refers to an antigenic substance capable of producing immediate hypersensitivity and includes both synthetic as well as natural immunostimulant peptides and proteins. Allergen includes but is not limited to any protein or peptide catalogued in the Structural Database of Allergenic Proteins database http://fermi.utmb.edu/SDAP/index.html

As used herein, the term “transmembrane protein” refers to proteins that span a biological membrane. There are two basic types of transmembrane proteins. Alpha-helical proteins are present in the inner membranes of bacterial cells or the plasma membrane of eukaryotes, and sometimes in the outer membranes. Beta-barrel proteins are found only in outer membranes of Gram-negative bacteria, cell wall of Gram-positive bacteria, and outer membranes of mitochondria and chloroplasts.

As used herein, the term “affinity” refers to a measure of the strength of binding between two members of a binding pair, for example, an antibody and an epitope and an epitope and a MHC-I or II haplotype. K_{d} is the dissociation constant and has units of molarity. The affinity constant is the inverse of the dissociation constant. An affinity constant is sometimes used as a generic term to describe this chemical entity. It is a direct measure of the energy of binding. The natural logarithm of K is linearly related to the Gibbs free energy of binding through the equation ΔG₀=-RT LN(K) where R=gas constant and temperature is in degrees Kelvin. Affinity may be determined experimentally, for example by surface plasmon resonance (SPR) using commercially available Biacore SPR units (GE Healthcare) or in silico by methods such as those described herein in detail. Affinity may also be expressed as the ic50 or inhibitory concentration 50, that concentration at which 50% of the peptide is displaced. Likewise In (ic50) refers to the natural log of the ic50.

The term “K_{off}”, as used herein, is intended to refer to the off rate constant, for example, for dissociation of an antibody from the antibody/antigen complex, or for dissociation of an epitope from an MHC haplotype.

The term “K_{d}”, as used herein, is intended to refer to the dissociation constant (the reciprocal of the affinity constant “K_{d}”), for example, for a particular antibody-antigen interaction or interaction between an epitope and an MHC haplotype.

As used herein, the terms “strong binder” and “strong binding” and “High binder” and “high binding” or “high affinity” refer to a binding pair or describe a binding pair that have an affinity of greater than 2×10⁷ M⁻¹ (equivalent to a dissociation constant of 50 nM Kd)

As used herein, the term “moderate binder” and “moderate binding” and “moderate affinity” refer to a binding pair or describe a binding pair that have an affinity of from 2×10⁷ M⁻¹ to 2×10⁶ M⁻¹.

As used herein, the terms “weak binder” and “weak binding” and “low affinity” refer to a binding pair or describe a binding pair that have an affinity of less than 2×10⁶ M⁻¹ (equivalent to a dissociation constant of 500 nM Kd)

Binding affinity may also be expressed by the standard deviation from the mean binding found in the peptides making up a protein. Hence a binding affinity may be expressed as “−1σ” or <−1σ, where this refers to a binding affinity of 1 or more standard deviations below the mean. A common mathematical transformation used in statistical analysis is a process called standardization wherein the distribution is transformed from its standard units to standard deviation units where the distribution has a mean of zero and a variance (and standard deviation) of 1. Because each protein comprises unique distributions for the different MHC alleles standardization of the affinity data to zero mean and unit variance provides a numerical scale where different alleles and different proteins can be compared. Analysis of a wide range of experimental results suggest that a criterion of standard deviation units can be used to discriminate between potential immunological responses and non-responses. An affinity of 1 standard deviation below the mean was found to be a useful threshold in this regard and thus approximately 15% (16.2% to be exact) of the peptides found in any protein will fall into this category.

The terms “specific binding” or “specifically binding” when used in reference to the interaction of an antibody and a protein or peptide or an epitope and an MHC haplotype means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the term “antigen binding protein” refers to proteins that bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc.

“Adjuvant” as used herein encompasses various adjuvants that are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

As used herein “immunoglobulin” means the distinct antibody molecule secreted by a clonal line of B cells; hence when the term “100 immunoglobulins” is used it conveys the distinct products of 100 different B-cell clones and their lineages.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “support vector machine” refers to a set of related supervised learning methods used for classification and regression. Given a set of training examples, each marked as belonging to one of two categories, an SVM training algorithm builds a model that predicts whether a new example falls into one category or the other.

As used herein, the term “classifier” when used in relation to statistical processes refers to processes such as neural nets and support vector machines.

As used herein “neural net”, which is used interchangeably with “neural network” and sometimes abbreviated as NN, refers to various configurations of classifiers used in machine learning, including multilayered perceptrons with one or more hidden layer, support vector machines and dynamic Bayesian networks. These methods share in common the ability to be trained, the quality of their training evaluated, and their ability to make either categorical classifications of non numeric data or to generate equations for predictions of continuous numbers in a regression mode. Perceptron as used herein is a classifier which maps its input x to an output value which is a function of x, or a graphical representation thereof.

As used herein, the term “principal component analysis”, or as abbreviated “PCA”, refers to a mathematical process which reduces the dimensionality of a set of data (Wold, S., Sjorstrom, M., and Eriksson, L., Chemometrics and Intelligent Laboratory Systems 2001. 58:109-130; Multivariate and Megavariate Data Analysis Basic Principles and Applications (Parts I & II) by L. Eriksson, E. Johansson, N. Kettaneh-Wold, and J. Trygg, 2006 2^{nd} Edit. Umetrics Academy). Derivation of principal components is a linear transformation that locates directions of maximum variance in the original input data, and rotates the data along these axes. For n original variables, n principal components are formed as follows: The first principal component is the linear combination of the standardized original variables that has the greatest possible variance. Each subsequent principal component is the linear combination of the standardized original variables that has the greatest possible variance and is uncorrelated with all previously defined components. Further, the principal components are scale-independent in that they can be developed from different types of measurements. The application of PCA generates numerical coefficients (descriptors). The coefficients are effectively proxy variables whose numerical values are seen to be related to underlying physical properties of the molecules. A description of the application of PCA to generate descriptors of amino acids and by combination thereof peptides is provided in PCT US2011/029192 incorporated herein by reference in its entirety. Unlike neural nets PCA do not have any predictive capability. PCA is deductive not inductive.

As used herein, the term “vector” when used in relation to a computer algorithm or the present invention, refers to the mathematical properties of the amino acid sequence.

As used herein, the term “vector,” when used in relation to recombinant DNA technology, refers to any genetic element, such as a plasmid, phage, transposon, cosmid, chromosome, retrovirus, virion, etc., which is capable of replication when associated with the proper control elements and which can transfer gene sequences between cells. Thus, the term includes cloning and expression vehicles, as well as viral vectors. “Viral vector” as used herein includes but is not limited to adenoviral vectors, adeno-associated viral vectors, lentiviral vectors, retroviral vectors, poliovirus vectors, measles virus vectors, flavivirus vectors, poxvirus vectors, and other viral vectors which may be used to deliver a peptide or nucleic acid sequence to a host cell.

As used herein, the term “host cell” refers to any eukaryotic cell (e.g., mammalian cells, avian cells, amphibian cells, plant cells, fish cells, insect cells, yeast cells), and bacteria cells, and the like, whether located in vitro or in vivo (e.g., in a transgenic organism).

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acids are nucleic acids present in a form or setting that is different from that in which they are found in nature. In contrast, non-isolated nucleic acids are nucleic acids such as DNA and RNA that are found in the state in which they exist in nature.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

A “subject” is an animal such as vertebrate, preferably a mammal such as a human, a bird, or a fish. Mammals are understood to include, but are not limited to, murines, simians, humans, bovines, ovines, cervids, equines, porcines, canines, felines etc.).

An “effective amount” is an amount sufficient to effect beneficial or desired results. An effective amount can be administered in one or more administrations,

As used herein, the term “purified” or “to purify” refers to the removal of undesired components from a sample. As used herein, the term “substantially purified” refers to molecules, either nucleic or amino acid sequences, that are removed from their natural environment, isolated or separated, and are at least 60% free, preferably 75% free, and most preferably 90% free from other components with which they are naturally associated. An “isolated polynucleotide” is therefore a substantially purified polynucleotide.

As used herein “Complementarity Determining Regions” (CDRs) are those parts of the immunoglobulin variable chains which determine how these molecules bind to their specific antigen. Each immunoglobulin variable region typically comprises three CDRs and these are the most highly variable regions of the molecule. T cell receptors also comprise similar CDRs and the term CDR may be applied to T cell receptors.

As used herein, the term “motif” refers to a characteristic sequence of amino acids forming a distinctive pattern.

The term “Groove Exposed Motif” (GEM) as used herein refers to a subset of amino acids within a peptide that binds to an MHC molecule; the GEM comprises those amino acids which are turned inward towards the groove formed by the MHC molecule and which play a significant role in determining the binding affinity. In the case of human MHC-I the GEM amino acids are typically (1,2,3,9). In the case of MHC-II molecules two formats of GEM are most common comprising amino acids (−3,2, −1,1,4,6,9, +1, +2, +3) and (−3,2,1,2,4,6,9,+1,+2,+3) based on a 15-mer peptide with a central core of 9 amino acids numbered 1-9 and positions outside the core numbered as negative (N terminal) or positive (C terminal).

“Immunoglobulin germline” is used herein to refer to the variable region sequences encoded in the inherited germline genes and which have not yet undergone any somatic hypermutation. Each individual carries and expresses multiple copies of germline genes for the variable regions of heavy and light chains. These undergo somatic hypermutation during affinity maturation. Information on the germline sequences of immunoglobulins is collated and referenced by www.imgt.org [1]. “Germline family” as used herein refers to the 7 main gene groups, catalogued at IMGT, which share similarity in their sequences and which are further subdivided into subfamilies.

“Affinity maturation” is the molecular evolution that occurs during somatic hypermutation during which unique variable region sequences generated that are the best at targeting and neutralizing and antigen become clonally expanded and dominate the responding cell populations.

“Germline motif”′ as used herein describes the amino acid subsets that are found in germline immunoglobulins. Germline motifs comprise both GEM and TCEM motifs found in the variable regions of immunoglobulins which have not yet undergone somatic hypermutation.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Illustrative examples of immunopathologies include, but are not limited to, B-cell lymphoma, T-cell lymphomas, Systemic Lupus Erythematosus (SLE), allergies, hypersensitivities, immunodeficiency syndromes, radiation exposure or chronic fatigue syndrome.

“pMHC” Is used to describe a complex of a peptide bound to an MHC molecule. In many instances a peptide bound to an MHC-I will be a 9-mer or 10-mer however other sizes of 7-11 amino acids may be thus bound. Similarly MHC-II molecules may form pMHC complexes with peptides of 15 amino acids or with peptides of other sizes from 11-23 amino acids. The term pMHC is thus understood to include any short peptide bound to a corresponding MHC.

“Somatic hypermutation” (SHM), as used herein refers to the process by which variability in the immunoglobulin variable region is generated during the proliferation of individual B-cells responding to an immune stimulus. SHM occurs in the complementarity determining regions.

“T-cell exposed motif” (also where abbreviated TCEM), as used herein, refers to the sub set of amino acids in a peptide bound in a MHC molecule which are directed outwards and exposed to a T-cell binding to the pMHC complex. A T-cell binds to a complex molecular space-shape made up of the outer surface MHC of the particular HLA allele and the exposed amino acids of the peptide bound within the MHC. Hence any T-cell recognizes a space shape or receptor which is specific to the combination of HLA and peptide. The amino acids which comprise the TCEM in an MHC-I binding peptide typically comprise positions 4, 5, 6, 7, 8 of a 9-mer. The amino acids which comprise the TCEM in an MHC-II binding peptide typically comprise 2, 3, 5, 7, 8 or −1, 3, 5, 7, 8 based on a 15-mer peptide with a central core of 9 amino acids numbered 1-9 and positions outside the core numbered as negative (N terminal) or positive (C terminal). As indicated under pMHC, the peptide bound to a MHC may be of other lengths and thus the numbering system here is considered a non-exclusive example of the instances of 9-mer and 15 mer peptides.

As used herein “histotope” refers to the outward facing surface of the MHC molecules which surrounds the T cell exposed motif and in combination with the T cell exposed motif serves as the binding surface for the T cell receptor.

As used herein the T cell receptor refers to the molecules exposed on the surface of a T cell which engage the histotope of the MHC and the T cell exposed motif of a peptide bound in said MHC. The T cell receptor comprises two protein chains, known as the alpha and beta chain in 95% of human T cells and as the delta and gamma chains in the remaining 5% of human T cells. Each chain comprises a variable region and a constant region. Each variable region comprises three complementarity determining regions or CDRs

“Regulatory T-cell” or “Treg” as used herein, refers to a T-cell which has an immunosuppressive or down-regulatory function. Regulatory T-cells were formerly known as suppressor T-cells. Regulatory T-cells come in many forms but typically are characterized by expression CD4+, CD25, and Foxp3. Tregs are involved in shutting down immune responses after they have successfully eliminated invading organisms, and also in preventing immune responses to self-antigens or autoimmunity.

“uTOPE™ analysis” as used herein refers to the computer assisted processes for predicting binding of peptides to MHC and predicting cathepsin cleavage, described in PCT US2011/029192, PCT US2012/055038, and US2014/01452, each of which is incorporated herein by reference in its entirety.

“Framework region” as used herein refers to the amino acid sequences within an immunoglobulin variable region which do not undergo somatic hypermutation.

“Isotype” as used herein refers to the related proteins of particular gene family. Immunoglobulin isotype refers to the distinct forms of heavy and light chains in the immunoglobulins. In heavy chains there are five heavy chain isotypes (alpha, delta, gamma, epsilon, and mu, leading to the formation of IgA, IgD, IgG, IgE and IgM respectively) and light chains have two isotypes (kappa and lambda). Isotype when applied to immunoglobulins herein is used interchangeably with immunoglobulin “class”.

“Isoform” as used herein refers to different forms of a protein which differ in a small number of amino acids. The isoform may be a full length protein (i.e., by reference to a reference wild-type protein or isoform) or a modified form of a partial protein, i.e., be shorter in length than a reference wild-type protein or isoform.

“Class switch recombination” (CSR) as used herein refers to the change from one isotype of immunoglobulin to another in an activated B cell, wherein the constant region associated with a specific variable region is changed, typically from IgM to IgG or other isotypes.

“Immunostimulation” as used herein refers to the signaling that leads to activation of an immune response, whether said immune response is characterized by a recruitment of cells or the release of cytokines which lead to suppression of the immune response. Thus, immunostimulation refers to both upregulation or down regulation.

“Up-regulation” as used herein refers to an immunostimulation which leads to cytokine release and cell recruitment tending to eliminate a non self or exogenous epitope. Such responses include recruitment of T cells, including effectors such as cytotoxic T cells, and inflammation. In an adverse reaction upregulation may be directed to a self-epitope.

“Down regulation” as used herein refers to an immunostimulation which leads to cytokine release that tends to dampen or eliminate a cell response. In some instances such elimination may include apoptosis of the responding T cells.

“Frequency class” or “frequency classification” as used herein is used to describe logarithmic based bins or subsets of amino acid motifs or cells. When applied to the counts of TCEM motifs found in a given dataset of peptides a logarithmic (log base 2) frequency categorization scheme was developed to describe the distribution of motifs in a dataset. As the cellular interactions between T-cells and antigen presenting cells displaying the motifs in MHC molecules on their surfaces are the ultimate result of the molecular interactions, using a log base 2 system implies that each adjacent frequency class would double or halve the cellular interactions with that motif. Thus, using such a frequency categorization scheme makes it possible to characterize subtle differences in motif usage as well as providing a comprehensible way of visualizing the cellular interaction dynamics with the different motifs. Hence a Frequency Class 2, or FC 2 means 1 in 4, a Frequency class 10 or FC 10 means 1 in 210 or 1 in 1024. In other embodiments the frequency classification of the TCEM motif in the reference dataset is described by the quantile score of the TCEM in the reference dataset. Quantile scores are used, but is not limited to, applications where the reference dataset is the human proteome or a microbial proteome. “Frequency class” or “frequency classification” may also be applied to cellular clonotypic frequency where it refers to subgroups or bins defined by logarithmic based groupings, whether log base 2 or another selected log base.

A “rare TCEM” as used herein is one which is completely missing in the human proteome or present in up to only five instances in the human proteome.

“IGHV” as used herein is an abbreviation for immunoglobulin heavy chain variable regions.

“IGLV” as used herein is an abbreviation for immunoglobulin light chain variable regions “Adverse immune response” as used herein may refer to (a) the induction of immunosuppression when the appropriate response is an active immune response to eliminate a pathogen or tumor or (b) the induction of an upregulated active immune response to a self-antigen or (c) an excessive up-regulation unbalanced by any suppression, as may occur for instance in an allergic response.

“Clonotype” as used herein refers to the cell lineage arising from one unique cell. In the particular case of a B cell clonotype it refers to a clonal population of B cells that produces a unique sequence of IGV. The number of B cells that express that sequence varies from singletons to thousands in the repertoire of an individual. In the case of a T cell it refers to a cell lineage which expresses a particular TCR. A clonotype of cancer cells all arise from one cell and carry a particular mutation or mutations or the derivates thereof. The above are examples of clonotypes of cells and should not be considered limiting.

As used herein “epitope mimic” or “TCEM mimic” is used to describe a peptide which has an identical or overlapping TCEM, but may have a different GEM. Such a mimic occurring in one protein may induce an immune response directed towards another protein which carries the same TCEM motif. This may give rise to autoimmunity or inappropriate responses to the second protein.

“Cytokine” as used herein refers to a protein which is active in cell signaling and may include, among other examples, chemokines, interferons, interleukins, lymphokines, granulocyte colony-stimulating factor tumor necrosis factor and programmed death proteins.

As used herein “oncoprotein” means a protein encoded by an oncogene which can cause the transformation of a cell into a tumor cell if introduced into it. Examples of oncoproteins include but are not limited to the early proteins of papillomaviruses, polyomaviruses, adenoviruses and herpesviruses, however oncoproteins are not necessarily of viral origin.

“MHC subunit chain” as used herein refers to the alpha and beta subunits of MHC molecules. A MHC II molecule is made up of an alpha chain which is constant among each of the DR, DP, and DQ variants and a beta chain which varies by allele. The MHC I molecule is made up of a constant beta macroglobulin and a variable MHC A, B or C chain.

As used here in “virome” comprises the viruses present in a human subject, latently chronically or during acute infection, or a sub set thereof made up of viruses of a particular taxonomic group or of the viruses located in a particular tissue or organ.

“Immunoglobulinome” as used herein refers to the total complement of immunoglobulins produced and carried by any one subject.

As used herein “allergome” refers to all proteins which may give rise to allergies. This includes proteins recorded in allergen datasets such as that represented at www.allergome.com, http://www.allergenonline.org/, http://comparedatabase.org/www.allergen.org as well as included in Uniprot, Swiss prot, etc.

As used herein the term “repertoire” is used to describe a collection of molecules or cells making up a functional unit or whole. Thus, as one non limiting example, the entirely of the B cells or T cells in a subject comprise its repertoire of B cells or T cells. The entirety of all immunoglobulins expressed by said B cells are its immunoglobulinome or the repertoire of immunoglobulins. A collection of proteins or cell clonotypes which make up a tissue sample, an individual subject or a microorganism may be referred to as a repertoire.

As used herein “mutated amino acid” refers to the appearance of an amino acid in a protein that is the result of a nucleotide change, a missense mutation, or an insertion or deletion or fusion.

“Splice variant” as used herein refers to different proteins that are expressed from one gene as the result of inclusion or exclusion of particular exons of a gene in the final, processed messenger RNA produced from that gene or that is the result of cutting and re-annealing of RNA or DNA.

“TRAV” as used herein refers to the T cell receptor alpha variable region family or allele subgroups and “TRBV” refers to T cell receptor beta variable region family or allele subgroups as described in IMGT http://imgt.org/IMGTrepertoire/Proteins/index.php #C http://imgt.org/IMGTrepertoire/Proteins/taballeles/human/TRA/TRAV/Hu_TRAVall.html TRAV comprises at least 41 subgroups, with some having sub-subgroups. TRBV comprises at least 30 subgroups. Most combinations of alpha and beta variable region subgroups are encountered. “hTRAV” refers to human TRAV.

As used here in a “receptor bearing cell” is any cell which carries a ligand binding recognition motif on its surface. In some particular instances a receptor bearing cell is a B cell and its surface receptor comprises an immunoglobulin variable region, said immunoglobulin variable region comprising both heavy and light chains which make up said receptor. In other particular instances a receptor bearing cell may be a T cell which bears a receptor made up of both alpha and beta chains or both delta and gamma chains. Other examples of a receptor bearing cell include cells which carry other ligands such as, in one particular non limiting example, a programmed death protein of which there are multiple isoforms.

As used herein the term “bin” refers to a quantitative grouping and a “logarithmic bin” is used to describe a grouping according to the logarithm of the quantity.

As used herein “immunotherapy intervention” is used to describe any deliberate modification of the immune system including but not limited to through the administration of therapeutic drugs or biopharmaceuticals, radiation, T cell therapy, application of engineered T cells, which may include T cells linked to cytotoxic, chemotherapeutic or radiosensitive moieties, checkpoint inhibitor administration, cytokine or recombinant cytokine or cytokine enhancer, including but not limited to a IL-15 agonist, microbiome manipulation, vaccination, B or T cell depletion or ablation, or surgical intervention to remove any immune related tissues.

As used herein “immunomodulatory intervention” refers to any medical or nutritional treatment or prophylaxis administered with the intent of changing the immune response or the balance of immune responsive cells. Such an intervention may be delivered parenterally or orally or via inhalation. Such intervention may include, but is not limited to, a vaccine including both prophylactic and therapeutic vaccines, a biopharmaceutical, which may be from the group comprising an immunoglobulin or part thereof, a T cell stimulator, checkpoint inhibitor, or suppressor, an adjuvant, a cytokine, a cytotoxin, receptor binder, an enhancer of NK (natural killer) cells, an interleukin including but not limited to variants of IL15, superagonists, and a nutritional or dietary supplement. The intervention may also include radiation or chemotherapy to ablate a target group of cells. The impact on the immune response may be to stimulate or to down regulate.

“Checkpoint inhibitor” or “checkpoint blockade” as used herein refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include, but are not limited to, PD-1/PD-L1 and CTLA-4/B7-1/B7-2.

As used herein the “cluster of differentiation” proteins refers to cell surface molecules providing targets for immunophenotyping of cells. The cluster of differentiation is also known as cluster of designation or classification determinant and may be abbreviated as CD. Examples of CD proteins include those listed at https://www.uniprot.org/docs/cdlist

As used herein “microbiome” refers to the constellation of commensal microorganisms found within the human or other host body, inhabiting sites such as the gastrointestine, skin the urogenital tract, the oral cavity, the upper respiratory tract. While most frequently referring to bacteria, the microbiome also may include the viruses in these sites, referred to as the “virome”, or commensal fungi.

As used herein “tumor associated mutations” refers to all nucleotide or amino acid mutations detected in a tumor. In some cases the tumor associated mutations are commonly found within many patients with a particular tumor type. In other cases tumor associated mutations may be unique to a specific patient. In other instances different patients may carry different tumor associated mutations are in the same protein.

“Pattern” as used herein means a characteristic or consistent distribution of data points.

As used herein a “frequency pattern” is a data set that displays the frequency of TCEMs in a repertoire of proteins from a proteome associated with an individual subject as compared to the frequency of those TCEMs in a reference database. Particular TCEMs, or groups of TCEMs, within the subject's repertoire may occur at the same, lower or higher frequencies than the corresponding TCEMs in the reference database. The frequency pattern allows identification and categorization of unique TCEMs and/or patterns of TCEMs (i.e., unique features of unique TCEM features). The term “frequency pattern” as used herein is also used to describe the distribution of cellular clonotypes within a repertoire of cells from an individual subject, as compared to the frequency of the cellular clonotypes in a reference database. Particular clonotypes, or groups of clonotypes, within the subject's repertoire may occur at the same, lower or higher frequencies than the corresponding cellular clonotypes in the reference database. The frequency pattern allows identification and categorization of unique patterns of clonotypes. In some embodiments, a “frequency class” or “frequency classification” is assigned to a TCEM motif or to a cellular clonotype based on its frequency as described elsewhere herein.

As used herein “clonotype” is a line of cells derived from a committed or fully differentiated progenitor. In the case of T cells and somatic cells other than B cells, a clonotype of cells has a common genotype, i.e. comprises a common nucleotide sequence. Clonotypes with different nucleotide sequences may express a protein of identical amino acid sequence as a result of different codon utilization. Hence multiple genotypes may lead to a shared phenotype among such clonotypes. In B cells, somatic mutation results in a differentiated cell line comprising a nucleotide sequence that expresses antibodies of one isotype and variable region sequence; this is a B cell clonotype.

As used herein “clonotypic diversity” refers to the distribution of the total number of cells in a repertoire among all unique clonotypes in a repertoire. Hence, if a repertoire has 1 million cells, but these comprise 400,000 of clonotype 1 and 600,000 of clonotype 2, the repertoire has a low clonotypic diversity. If the 1 million cells are distributed as 10 each of 100,000 unique clonotypes the repertoire has a high clonotypic diversity.

As used herein “many to one” describes a relationship in which one protein or peptide sequence is encoded be many different synonymous nucleotide sequences.

As used herein “presentome” refers to the peptides bound in MHC and presented on the surface of antigen presented cells. Mass spectroscopy detects some but not all peptides which are part of the presentome.

“Neoantigen” as used herein refers to a novel epitope motif or antigen created as the result of introduction of a mutation into an amino acid sequence. Thus, a neoantigen differentiates a wildtype protein from its mutant-bearing tumor protein homolog, when such mutant is presented to T cells or B cells.

“Tumor specific antigen” or “tumor specific epitope” is used herein to designate an epitope or antigen that differentiates a mutated tumor protein from its unmutated wildtype homologue. Thus, a neoantigen is one type of tumor specific antigen.

As used herein “driver” mutations are those which arise very early in tumorogenesis and are causally associated with the early steps of cell dysregulation. Driver mutations are shared by all clonal offspring arising from the initial tumor cells and offer some additional fitness benefit to the clonal line within its microenvironment. In contrast passenger mutations are those somatic mutations which arise during the differentiation of the tumor and which offer no particular benefit of fitness to the cell. Passengers may serve as biomarkers on tumor cells and may enable some immune evasion. Passenger mutations may differ at different time points in its development and among different parts of a tumor or among metastases. “Driver and passenger” are terms largely interchangeable with “trunk and branch” mutations.

“Bespoke peptides” or “bespoke vaccine” as used herein refers to a peptide or neoantigen or a combination of peptides, or nucleic acid encoding peptides, that are tailored or personalized specifically for an individual patient, taking into account that patient's HLA alleles and mutations.

As used herein “TCGA” refers to The Cancer Genome Atlas (https://www.cancer.gov/about-nci/organization/ccg/research/structural-genomics/tcga)

As used herein a “polyhydrophobic amino acid” refers to a short chain of natural amino acids which are hydrophobic. Examples include, but are not limited to, leucines, isoleucines or tryptophans where these are assembled in multimers of 5-15 repeats of any one such amino acid. As a non-limiting example, a poly leucine comprising 8 leucines would be an example of a polyhydrophobic amino acid.

A “lipid core peptide system”, as used herein, refers to subunit vaccine comprising a lipoamino acid (LAA) moiety which allows the stimulation of immune activity. A combination of T cell stimulating epitopes or T and B cell stimulating epitopes are linked to a LAA. Multiple different constructs can be created with of different spatial orientation or LAA lengths (e.g. C12 2-amino-D,L-dodecanoic acid or C16, 2-amino-D,L-hexadecanoic acid). When dissolved in a standard phosphate buffer LCP particles form and the particles facilitate uptake by antigen presenting cells. Different LAA chain lengths lead to different particle sizes.

As used herein, the term “cleavage site octomer” refers to the 8 amino acids located four each side of the bond at which a peptidase cleaves an amino acid sequence. Cleavage site octomer is abbreviated as CSO. “Cathepsin cleavage site octomer” is used herein where the peptidase is a cathepsin.

As used herein “compounding pharmacy” has the meaning defined in sections 503A and 503B of the Federal Food, Drug, and Cosmetic Act

As used herein, a “BAM” file is a compressed binary version of a Sequence Alignment File “SAM” file wherein the all nucleotides are aligned to a reference genome. A “BAM slice” is a subset of the entire genome defined by genome coordinates. The HLA locus is located on Chromosome 6. In one particular instance a BAM slice is defined to contain just the HLA locus.

“Immunopathology” when used herein describes an abnormality of the immune system. An immunopathology may affect B-cells and their lineage causing qualitative or quantitative changes in the production of immunoglobulins. Immunopathologies may alternatively affect T-cells and result in abnormal T-cell responses. Immunopathologies may also affect the antigen presenting cells. Immunopathologies may be the result of neoplasias of the cells of the immune system. Immunopathology is also used to describe diseases mediated by the immune system such as autoimmune diseases. Representative autoimmune diseases include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenic purpura, Autoimmune uveitis, Bullous Pemphigoid, Castleman's disease, Celiac disease, Cogan syndrome, Cold agglutinin disease, Crohns Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis.

“Antigen presenting cell” as used herein refers to cells which are capable of presentation of peptides to T cells bound to MHC molecules. This includes but is not limited to the so called “professional” antigen presenting cells comprising but not limited to dendritic cells, B cells, and macrophages, but also the so called non-professional antigen presenting cells which carry MHC molecules.

### DESCRIPTION OF THE INVENTION

Cancer has been described as a personal disease. This is true at many different levels. First, mutations arise that cause disrupted metabolic pathways resulting in ongoing proliferation, evasion of growth suppressors, cellular replicative immortality, resistance to cell death and dysregulation of cell energetics, with associated angiogenesis and metastasis [2]. Each tumor comprises multiple genomic mutations. Some are silent mutations (synonymous) which do not change amino acid coding; others result in amino acid changes. Each tumor has a unique combination and number of mutated proteins. In many cases mutations are stochastic and thus unique to the individual. However, some proteins are more prone to mutations than others and have particular locations at which such mutations are more likely to occur. An initial mutation (trunk mutation or driver mutation) may be followed by many more mutations, all stochastic (branch or passenger mutations). Thus, the initial genomic aberration is personal, the combination of unique tumor proteins is personal, and various therapeutic interventions may be prescribed based on this pattern. Each cell comprising a mutated protein is then subject to surveillance by the immune system, which may result in elimination of the cancer cell, or its escape through immune evasion or by inducing anergy or immune suppression [3]. As the immune surveillance depends on an individual patient's combination of HLA alleles, this is also personal. And the presence of cognate T cells which can participate in the process of immune surveillance is determined by the individual's prior immune exposure and T cell repertoire. So this too is personal. Our findings show that mutations present in tumor proteins by the time of clinical diagnosis have developed several means of camouflage from immune surveillance and elimination, and that strategies to overcome such camouflage must be employed to achieve effective immunotherapy. The present invention provides such strategies by devising means to expose and present the tumor specific peptides to T cell recognition and effective elimination by T cells and by utilizing the B cell epitopes also exposed.

This invention provides a method for maximizing the immune response to mutated tumor specific proteins, either by means of stimulation of dendritic cells or T cells in vitro followed by administration of these cells to a patient, or by means of administration of a neoantigen vaccine in which de novo peptides, or their encoding nucleic acids, have been designed to ensure an appropriate level of binding affinity to a particular cancer patient's MHC alleles. Neoantigen selection from mutated tumor proteins is often limited by poor binding to a patient's MHC alleles. This invention overcomes this limitation by providing methods to design novel peptides, not found in the tumor protein, which bind a patient's alleles with a desired binding affinity while still retaining the tumor-specific T cell exposed motif needed to stimulate T cells cognate for the tumor mutation. The invention also provides methods to analyze tumor T cell exposed motifs and identify matches in the human proteome which will be presented by the MHC of the particular subject. It thus enables an informed choice of neoantigens based on risk-benefit analysis of off-target binding. The invention provides methods to design personalized neoantigen peptides for a particular patient based on that patient's alleles and unique mutations and to group these peptides into a vaccination regimen. It also provides methods to design an array of peptides suitable for targeting the mutations common to many tumor proteins and cancer types.

Methods for precisely predicting MHC binding, identifying and analyzing T cell exposed motifs and generating peptides with altered binding affinity are provided in the following co-pending applications, all of which are incorporated herein by reference in their entirety: PCT US2011/029192, PCT US2012/055038, US2014/014523, PCT US2015/039969, PCT US2017/021781, US Publ. No. 20130330335, US Publ. No. 20160132631, US Publ. No. 20170039314, US Publ. No 20170161430 and US Publ. No. 20190070255.

The present invention provides a method for maximizing the number of opportunities to mount a cytotoxic T cell attack on a tumor which carries mutated proteins. In one embodiment the invention provides a method for generating a peptide or an array of peptides that carry the same T cell exposed motifs that are found in the tumor specific proteins, but wherein said peptide or peptides in the array are not present in the tumor, but rather are created by substitution of flanking amino acids to optimize the binding affinity of said peptides to the alleles of a particular tumor-bearing subject. Further embodiments of the invention then enable the selection of a group of peptides so created, which when synthesized, are capable of stimulating tumor specific T cells of the tumor-bearing subject. In particular embodiments these peptides may be encoded in nucleic acid sequences, which may be RNA or DNA. In some embodiments the peptides in the array generated are of 9 or 10 amino acids long. In such embodiments the T cell response stimulated is as the result of binding to MHC I molecules and the response by CD8+ T cells. In other embodiments the peptides in the array generated are 15 amino acids long. In such embodiments the T cell response stimulated is as the result of binding to MHC II molecules and the response by CD4+ T cells. In yet other instances the peptides may be longer, up to about 35 amino acids. In yet other embodiments the T cell response stimulated is as the result of both CD8+ and CD4+ responses.

In particular embodiments a single peptide capable of stimulating tumor specific T cells of the tumor-bearing subject may be selected. In other instances, up to 5 peptides may be selected. In another desired embodiment a group of selected peptides in the array capable of stimulating tumor specific T cells of the tumor-bearing subject comprises at least 5 unique peptides not found in the tumor; in other embodiments the array encompasses at least 20 unique peptides, while in further embodiments the array has more than 60 unique peptides not found in the tumor. Each peptide carries a T cell exposed motif that is shared with the tumor protein at a position that includes the mutated amino acid in the T cell exposed motif. In some embodiments the group of peptides has at least 5 different T cell exposed motifs; in other embodiments the group of selected peptides comprises at least 10 different T cell exposed motifs. In yet other embodiments the group of selected peptides comprises at least 50 different T cell exposed motifs. In some particular embodiments the flanking amino acids of the peptides are selected so each peptide group has peptides collectively predicted to bind to at least 2 different MHC alleles carried by the tumor bearing subject. In other embodiments the flanking amino acids of the peptides are selected so each peptide group has peptides collectively predicted to bind to at least 4 different MHC alleles carried by the tumor bearing subject. In some embodiments a group of peptides created by substitution of the flanking amino acids of one or more T cell exposed motif to optimize binding to MHC allele of an individual subject may be combined in an array with naturally occurring neoepitope peptides.

The signal strength stimulating T cells as the result of presentation of peptides to T cells depends in part on the affinity of the peptide to the MHC. In some cases a very high affinity may be sought; in others a moderately high affinity. It is therefore useful to be able to select peptides of a desired affinity, but which are still present the same T cell exposed motif. In one embodiment of the invention therefore, the invention enables the selection of peptides that bind better than 99% of other peptides in the mutant protein; in other embodiments the invention enables selection of peptides binding better than 95% of other peptides in the mutant protein, while in further instances selection of peptides with a binding affinity of about 85% or better is enabled. Described in a different way, in one embodiment the invention enables selection of peptides which are predicted to bind at concentrations of less than 20 nanomolar, and in other embodiments at less than 50 nanomolar, less than 200 nanomolar or at less than 500 nanomolar concentrations. In some particular embodiments, the peptides in the group of T cell stimulating peptides are selected to include only peptides soluble in aqueous solutions; in yet other embodiments the peptides may be soluble in other solvents, including but not limited to, dimethyl sulphoxide.

The invention addresses both tumor specific mutations which are personal to a specific cancer patient and also those mutations which appear repeatedly in the same protein in cancers of different types in different subjects. In one embodiment, therefore, the invention embodies a method to create a group of peptides, not found in the original mutated protein, which are capable of stimulating T cells specific to the individual tumor-bearing subject and which target the mutations in proteins unique to those in the tumor of that subject. Such a group of peptides is selected to bind to MHC alleles carried by that subject. In yet other embodiments however, the present invention enables selection of a group of peptides that will elicit T cells to respond to mutations that are found in multiple cancers, including cancers arising from different tissues. Such an array of peptides is selected based on the presence of T cell exposed motifs that match those in commonly mutated proteins but also on their binding to any of an extended list of alleles that may be carried by any cancer patient who has a cancer with the common mutation. In one particular embodiment, the sequences of peptides suitable to stimulate T cells targeting common mutations in BRAF, EGFR, ERBB2, PTEN and PIK3CA for individuals carrying any one of 8 MHC I or 4 MHC II alleles are provided.

The T cell stimulating peptides described and selected in this invention may be deployed in several ways. In some embodiments they can be used in vitro to prime dendritic cells which upon administration to the tumor-bearing subject will stimulate T cells. In other embodiments the peptides may be used in vitro to stimulate T cells, whether said T cells are from the tumor bearing subject or from an allele matched donor. The stimulated T cells are then administered to the subject. In preferred embodiments the groups of T cell stimulating peptides designed and selected by the methods of the invention are used as a vaccine administered to the tumor bearing subject. In some embodiments, instead of applying the peptides as a vaccine, nucleic acids encoding the peptides are administered to the subject, wherein said nucleic acids may be RNA or DNA.

The goal of the invention is to provide peptides to stimulate T cells which will target the mutant protein displaying the same T cell exposed motifs. For this to happen the peptides from the mutant protein in the tumor need to be naturally presented at some level by the MHC alleles of the subject. Therefore, another embodiment of the present invention provides for selection of peptides from the initial array which have a sufficient binding affinity to the subject's MHC alleles to allow some presentation. In particular, therefore, the selection of peptides is down-selected to remove targets which are in the lower 50% of probability of presentation by the subject's MHC, i.e. those with less than the mean binding affinity for the protein from which their T cell exposed motif is derived.

Having identified an array of T cell stimulating peptides which are suitable to target the mutated tumor protein in the particular tumor-bearing subject of known MHC alleles, the present invention then embodies the design of a vaccination regimen. In one such embodiment the group of selected peptides is administered at one time. In an alternate embodiment the group of peptides may be divided into multiple subgroups which are administered at different time points. In one embodiment the invention provides for organizing the subgroups to ensure that several T cell exposed motifs are targeted in each subgroup and that the peptides depend on several different alleles for presentation. As motifs which are rare in the human proteome may offer an advantage in stimulating T cells and specifically targeting a tumor, one embodiment provides for prioritizing the peptide subgroup composition according to the frequency classification of the T cell exposed motif that each peptide carries relative to its frequency in the human proteome or human immunoglobulinome. In a preferred embodiment, the rare motifs are included in the early subgroups.

Checkpoint inhibitor drugs prevent or delay the termination of T cell responses. In some embodiments the present invention provides for the administration of a checkpoint inhibitor with the vaccine or, in a preferred embodiment, following a peptide vaccine as described herein, or nucleic acid vaccine encoding peptides. As another embodiment, when the vaccine is administered in multiple subgroups of peptides over time the checkpoint inhibitor may be reapplied after each or some of the subgroups of peptides. Furthermore, there are other immunomodulatory interventions which extend the T cell responses, including but not limited to NK cells, IL-15, and other superagonists. In a further embodiment the present invention provides for the administration of other immunotherapeutic interventions intended to extend or enhance T cell responses with the vaccine or, in a preferred embodiment, following the vaccine.

In embodiments of this invention, a vaccine is provided comprising peptides which carry T cell exposed motifs found in the tumor, but in which flanking amino acids have been interchanged to change the binding of the peptide to optimize to a desired binding to the subject's MHC alleles. In some embodiments said vaccine is delivered to the subject parenterally, in other embodiments delivery is intradermal or transdermal. In the case of transdermal vaccination one preferred embodiment provides for delivery of peptides in a microneedle array. Said microneedle array may be configured to deliver multiple different peptides or nucleotide sequences encoding different peptides in the same array, In some embodiments, vaccination is accompanied by an adjuvant. In some embodiments an adjuvant is incorporated into the solution comprising the neoantigen peptides. When vaccine is delivered transdermally, a particular embodiment is to accompany delivery by a local proinflammatory agent, whether physical, such as, but not limited to, heat, infrared light or friction, or by administration of a proinflammatory drug or cream.

As the present invention identifies T cell stimulating peptides carrying T cell exposed motifs found in multiple cancers and provides suitable binding peptides to deliver such T cell motifs to subjects of different MHC alleles, an embodiment of the invention is to provide an array of peptides, which offer combinations of T cell exposed motifs and binding affinities, for a range of common cancer mutations and for many different alleles. Such an array, in one embodiment, provides peptides with a binding affinity of less than 20 nanomolar, in another less than 50 nanomolar, in another embodiment less than 100 nanomolar and in yet another less than 500 nanomolar concentrations. In yet others the array comprises peptides which individually have binding affinities of between 20 and 500 nanomolar. Said peptide array in one embodiment comprises T cell motifs shared by at least 3 cancers, and in another embodiment comprises T cell exposed motifs carried by cancers affecting more than three tissue types. One embodiment provides an array that encompasses the mutations commonly found in 5 proteins, while in another embodiment the array includes mutations commonly found in 10 proteins that are shared in more than one cancer type. In a particular embodiment, the array includes peptides that include T cell exposed motifs found in the proteins BRAF, EGFR, ERBB2, PTEN and PIK3CA and embodies peptides suitable to administer to individuals carrying any one of 8 MHC I or 4 MHC II particular alleles, in particular embodying sequences for such proteins. In yet other embodiments further peptide arrays are designed to be suitable to administer to individuals with yet other MHC alleles or combinations thereof. In addition to amino acid substitutions found in multiple cancers, there are also insertions and deletions that are common to many cancers, and also gene fusions which generate common junction sites in the resultant protein products. In another embodiment, therefore, the invention provides a method for designing an array of peptides which enable tumor-specific targeting of the junction sites created by insertions, deletions and fusions. In one particular embodiment the invention provides specific peptides which may be used to target EGFRviii, a common oncogenic deletion mutant of epidermal growth factor receptor found in multiple cancers.

In further embodiments a B cell epitope peptide may be administered in conjunction with a T cell stimulating peptide. In some embodiments said B cell epitope may be a separate peptide or alternatively it may be in the same peptide as that designed to stimulate the T cells, or otherwise operably linked via a linker. In some embodiments a modified T cell stimulating peptide is designed to provide stronger T cell help to a B cell epitope through modified binding. Given the polyspecificity of T cell receptor binding, the occurrence of off-target binding of T cells stimulated to respond to a tumor specific mutation is of concern as a source of potential adverse reactions. Therefore, in one embodiment the present invention provides a method to identify potential unintended targets in the human proteome and to determine if such potential collateral targets are of concern for the particular subject according to the MHC alleles said subject carries. The application of this embodiment provides a list of the proteins in the human proteome which may be inadvertently targeted by CD8+ or CD4+ T cells stimulated by the peptide arrays selected for T cell targeting of the tumor and with sufficient binding affinity to MHC alleles of the particular subject to stimulate T cells. In one embodiment said list is flagged to identify proteins of particular concern because they have a critical function or are non-redundant and the list is provided to the oncologist to enable an informed risk benefit analysis.

Determination of the subject's HLA alleles are a necessary prerequisite to designing a peptide of suitable HLA binding affinity for that individual. Therefore, in some embodiments the HLA alleles of the subject are determined from the whole exome sequence which is also used to determine the tumor mutations.

The peptides designed to stimulate an immune response of the subject may be administered as a peptide composition or a nucleic acid composition encoding said peptide or peptides. In yet another embodiment the selected designed peptides may be delivered in a nanoparticular formulation. In some particular embodiments one or more selected designed peptides may be fused to a fusion partner by means of a linker. In some embodiments said linker is cleavable. The fusion partner is selected from the group comprising polyhydrophobic acids or unnatural amino acids or a lipid core system to enhance nanoparticle formation and favor uptake by antigen presenting cells. In some embodiments the fusion partner may also be an immunoglobulin or an immunoglobulin Fc region or other immunoglobulin fragment which facilitates uptake by antigen presenting cells.

The T cell stimulating peptides designed and selected to provide binding for the individual subject MHC alleles and specific to the tumor mutations of that subject are highly personal. In some embodiments, therefore, the particular sequence specification of such peptides are included in a prescription written for that particular patient. In some embodiments the peptides in the prescription may be formulated by a compounding pharmacy.

### Personalized Cancer Vaccines

There is increasing evidence that a variety of T cell immunotherapies can be successful in halting the progression of cancer [4]. Whereas in early days of cancer immunotherapy, the focus was on tumor-associated antigens as targets of both antibodies and T cell based therapies, current focus is now towards proteins comprising specific mutations in cancer cells, so called tumor-specific antigens or tumor neoantigens [5-8]. The fundamental goal in identifying and targeting mutations specific to the tumor is to differentiate normal from tumor tissue and hence eliminate tumor cells while leaving normal cells unharmed. A second current focus, and often combined strategy, is the application of checkpoint inhibitors and other immunomodulatory interventions to unleash T cell responses.

Tumor specific antigens comprise both those common to many cancers, and those which are unique to any single patient and which may change over the life of a tumor. Generally, the higher the mutational load, the more infiltrating T cells and the more inflamed a tumor, the greater probability of a check-point inhibitor leading to a successful T cell driven elimination of the tumor cells. Mutational load tends to differ between cancer types; some such as melanoma and colorectal cancers have a high mutational frequency. Others such as glioblastoma are notoriously low in mutational numbers.

Several recent publications have reported promising, but mixed, results in the development of personalized vaccines for melanoma [9, 10], lung cancer and glioblastoma [12, 13]. These have employed from 1 to 20 different neoantigens. Increasing the number of neoepitopes incorporated in a vaccine allows for a multipronged attack on the tumor using multiple alleles and multiple antigens derived from different proteins. Mutations continue to arise in tumors as they develop, with antigens gained or lost in the process. There may also be heterogeneity of mutations within a tumor and the mutational landscape may not be fully reflected in the sequencing of a biopsy. Hence a high number of cytotoxic “hits” is desirable rather than depending on only one or two antigen targets [8]. A goal of the present invention is to maximize the number of tumor specific epitopes which can be targeted by T cells responding to peptides presented by a particular patient's alleles.

The goal of T cell immunotherapy has been primarily to activate CD8+ cytotoxic T cells which will target tumor cells, but also to stimulate CD4+ T helper cells to enhance CD8+ responses. Stimulation of CD4+ T helper cells may also enhance B cell responses. Selection of peptides for use as neoepitopes has followed several paths. As a starting point, given the diversity of the human genome, it is desirable to compare sequences of proteins in tumor biopsies with a normal tissue from the same patient [14]. However, reference human genomes are frequently used as comparators to determine mutation sites. Practitioners have then used several approaches to select peptides for use, or for encoding in RNA or DNA for administration. In some instances peptides have been selected based on mass spectroscopy [15, 16]; in yet others predictive algorithms, most often NetMHC Pan [17], was used to select peptides [9, 10, 13]. In one instance, both approaches were reported, but in this particular case none of the mutated peptides were detected by mass spectroscopy [12].

Checkpoint inhibitors are not always predictable in their efficacy; despite remarkable benefits to some patients, the percentage of patients who benefit is still low, on average about 20%. There is an effort to define better biomarkers to predict the outcome of checkpoint inhibitor therapy [18-20]. Furthermore, a wide variety of adverse off-target effects have been reported following checkpoint inhibitor treatment [21]. The issue underlying both problems is that checkpoint inhibitors are indiscriminate and will unleash whatever T cells the patient has at the time of administration, whether or not they are targeting the tumor or self-antigens. Combination of neoantigen vaccination with checkpoint inhibitor blockade has been shown to elicit T cells specific of the neoantigens and has been combined with neoantigen vaccines in several of the above referenced studies. Thus, one goal of the present invention is to maximize the number of tumor-targeting T cells which are dis-inhibited by checkpoint inhibitor administration, while also focusing on those T cells which do not target critical self-antigens. This has the potential to greatly increase the efficacy of checkpoint blockade therapy. Other immunomodulatory interventions have been designed to extend T cell responses, including but not limited to NK cells, IL-15, and other superagonists. In a further embodiment the present invention provides for the administration of such other immunotherapeutic interventions intended to extend T cell responses with the vaccine or, in a preferred embodiment, following the vaccine.

Neoepitope vaccines also inevitably give rise to some off-target autoimmunity due to T cell polyspecificity among proteins with shared T cell exposed motifs. The neoepitope vaccine studies cited above have not addressed this potential adverse effect. In a progressing cancer there is necessarily a risk-benefit choice between eliminating the tumor and the effect of the off-target responses. In the present invention we describe how tumor neoantigens can be pre-screened for potential self-protein cross reactivity in a particular patient, based on that patient's alleles. This allows for an informed choice of which neoantigens are beneficial for targeting the tumor while also minimizing adverse effects.

There is therefore a need to facilitate the selection of peptides suitable for use in neoantigen vaccines and to maximize the number and immunogenicity of peptides that are applied. This can then also be used to enhance the benefits of checkpoint inhibitor blockade.

### Common Cancer Associated Driver Mutations

Mutations in cancers include those which are unique to a specific patient. Some are patient specific driver mutations, arising as the root cause of cell dysregulation. Others arise as branch or passenger mutations, which are sequelae to an earlier trunk or driver mutation. Such mutations may continue to evolve throughout the tumor progression. There are also a number of mutations which are found commonly at the same positions in the same proteins, some of which occur repeatedly across many cancer types [23-27]. The Cancer Genome Atlas documents many proteins which are found to share mutations across multiple cancer types (https://www.cancer.gov/about-nci/organization/ccg/research/structural-genomics/tcga). Some of these are simple amino acid substitutions arising from single nucleotide mutations; others involve amino acid duplications. In other cases, the mutations give rise to insertions and deletions (indels) and missense sequences. Where these mutations are shared across many cancers, a set of peptides can be designed for each patient HLA allele which will allow stimulation of T cells to specifically target tumor cells with cytotoxic T cells and/or T helper cells. In Example 7, provided below, therefore, we describe the approach to development of a set of “ready to go” neoantigens which have broad applicability across many cancers and for patients with defined common mutations and known HLA typing. In some embodiments, such “multicancer” neoantigens may be combined with a set of “bespoke” personalized neoantigens. In the case of indels and missense mutations, when these result in an in-frame downstream sequence they provide a target-rich sequence, but every patient is unique and so selection of vaccine peptides for these must be handled as a personalized design effort. In some embodiments consistent indels are found repeatedly in many cancers. In one particular example EGFR (Epidermal growth factor receptor) has two well documented oncogenic deletions, known as EGFRvii and EGFRviii. In EGFRviii, the most common deletion, In EGFRviii exons 2 and 7 are deleted leading to removal of amino acids 6-273 of the mature protein; a glycine is inserted in the bridge and the downstream sequence remains in frame. An effort was made to use a peptide spanning the deletion junction as a vaccine. This peptide, comprising 14 amino acids comprises a B cell epitope and was viewed as a way of inducing antibody dependent cytotoxicity when combined with a linked adjuvant [28]. In Example 8 we provide an approach to increasing the potential number of HLA alleles that could benefit from a peptide spanning the deletion junction in EGFRviii, and hence provide an example of an array of peptides which could be used for T cell stimulation to target this mutated EGFR.

In some preferred embodiments, mutated proteins in biopsy samples are identified by sequencing the genome, proteome or transcriptome of cells from the biopsy. The present invention is not limited to any particular method of obtaining sequences of mutated in a biopsy. A variety of sequencing methods are readily available to those of ordinary skill in the art.

In some preferred embodiments, the present invention utilizes nucleic acid sequencing techniques. The nucleic acid sequences are preferably converted in silico to protein sequences from the identification of mutated amino acids and peptides comprising the mutated amino acids.

In some embodiments, the sequencing is Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), semiconductor sequencing, massive parallel clonal, massive parallel single molecule SBS, massive parallel single molecule real-time, massive parallel single molecule real-time nanopore technology, etc. Morozova and Marra provide a review of some such technologies in Genomics, 92:255 (2008), herein incorporated by reference in its entirety. Those of ordinary skill in the art will recognize that because RNA is less stable in the cell and more prone to nuclease attack experimentally RNA is usually reverse transcribed to DNA before sequencing.

DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, the sequencing is automated sequencing. In some embodiments, the sequencing is parallel sequencing of partitioned amplicons (PCT Publication No: WO2006084132 to Kevin McKernan et al., herein incorporated by reference in its entirety). In some embodiments, the sequencing is DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55:641-658, 2009; MacLean et al., Nature Rev. Microbiol., 7:287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), Life Technologies/Ion Torrent, the Solexa platform commercialized by Illumina, GnuBio, and the Supported Oligonucleotide Ligation and Detection (SOLID) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., and Pacific Biosciences, respectively.

In pyrosequencing (Voelkerding et al., Clinical Chem., 55:641-658, 2009; MacLean et al., Nature Rev. Microbiol., 7:287-296; U.S. Pat. Nos. 6,210,891; 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotiter plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55:641-658, 2009; MacLean et al., Nature Rev. Microbiol., 7:287-296; U.S. Pat. Nos. 6,833,246; 7,115,400; 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 250 nucleotides, with overall output exceeding 1 billion nucleotide pairs per analytical run.

Sequencing nucleic acid molecules using SOLID technology (Voelkerding et al., Clinical Chem., 55:641-658, 2009; MacLean et al., Nature Rev. Microbiol., 7:287-296; U.S. Pat. Nos. 5,912,148; 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLID system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, sequencing is nanopore sequencing (see, e.g., Astier et al., J. Am. Chem. Soc. 2006 Feb. 8; 128 (5): 1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, sequencing is HeliScope by Helicos BioSciences (Voelkerding et al., Clinical Chem., 55:641-658, 2009; MacLean et al., Nature Rev. Microbiol., 7:287-296; U.S. Pat. Nos. 7,169,560; 7,282,337; 7,482,120; 7,501,245; U.S. Pat. Nos. 6,818,395; 6,911,345; 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327 (5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ~99.6% for 50 base reads, with ~100 Mb to 100 Gb generated per run. The read-length is 100-300 base pairs. The accuracy for homopolymer repeats of 5 repeats in length is ~98%. The benefits of ion semiconductor sequencing are rapid sequencing speed and low upfront and operating costs.

In some embodiments, sequencing is the technique developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety. Other emerging single molecule sequencing methods include real-time sequencing by synthesis using a VisiGen platform (Voelkerding et al., Clinical Chem., 55:641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In other preferred embodiments, the present invention utilizes protein sequencing techniques. In some embodiments, proteins my be sequenced by Edman degradation. See, e.g., Edman and Begg (1967). “A protein sequenator”. Eur. J. Biochem. 1(1):80-91; Alterman and Hunziker (2011) Amino Acid Analysis: Methods and Protocols. Humana Press. ISBN 978-1-61779-444-5. In other embodiments, mass spectrometry techniques are utilized to sequence proteins. See, e.g., Shevchenko et al., (2006) “In-gel digestion for mass spectrometric characterization of proteins and proteomes”. Nature Protocols. 1(6):2856-60; Gundry et al., (2009) “Preparation of proteins and peptides for mass spectrometry analysis in a bottom-up proteomics workflow” Current Protocols in Molecular Biology. Chapter 10: Unit10.25.

### Considerations in Selection of Tumor Specific Antigens

### T Cell Exposed Motifs

The goal of stimulating a cytotoxic T cell response to a tumor is to specifically and differentially destroy the tumor cells while leaving normal cells intact. It follows that to drive a T cell response specific to the cancer, the T cell receptor must recognize an epitope unique to the tumor. Thus, the mutated amino acid must be located in the exposed pentameric motif exposed to the T cell receptor. When a mutated amino acid is located in a pocket or groove exposed motif, it may or may not affect binding affinity, but it is hidden from the T cell receptor and cannot elicit tumor-specific T cell responses. In some instances, the natural binding affinity of the mutated peptide and its neighboring peptides in the affected protein may give rise to better binding in positions which do not expose the mutated amino acid. In some cases, so-called neoepitope peptides have been selected which do not, in fact, differentiate tumor and normal T cell exposed motifs [11, 29]. In the present invention we seek to maximize use of the T cell exposed motifs containing mutant amino acids, and hence focus the T cell response on these differentiating epitopes, and likewise subsequent expansion of this response as the result of administration of checkpoint inhibitors.

### Peptide Binding Affinity

Many investigators have considered how to identify peptides in mutated tumor proteins which bind to a patient's MHC alleles. Some have employed mass spectrometry to identify the “presentome” of peptides bound and presented to T cells [15]. However, this has the bias of identifying very high affinity peptides. In some cases, the peptides containing mutant amino acids were never detected by mass spectroscopy [12].

It is not clear that the highest binding peptides are those which will actually generate the best cytotoxic T cell response. Indeed, evidence in other settings suggests that this is not the case and that an intermediate binding affinity may be most effective in stimulating a T cell response and good memory T cells [30]. Low affinity peptides may initiate a CD8+ response but this is not sustained [31]. Furthermore, also drawing on experience in an anti-microbial setting, an active interferon gamma response is also needed to trigger the development of T memory cells [32]. Strength of T cell receptor-pMHC binding may be a factor in determining whether the T cell response to a tumor leads to T cell exhaustion and tolerance [3].

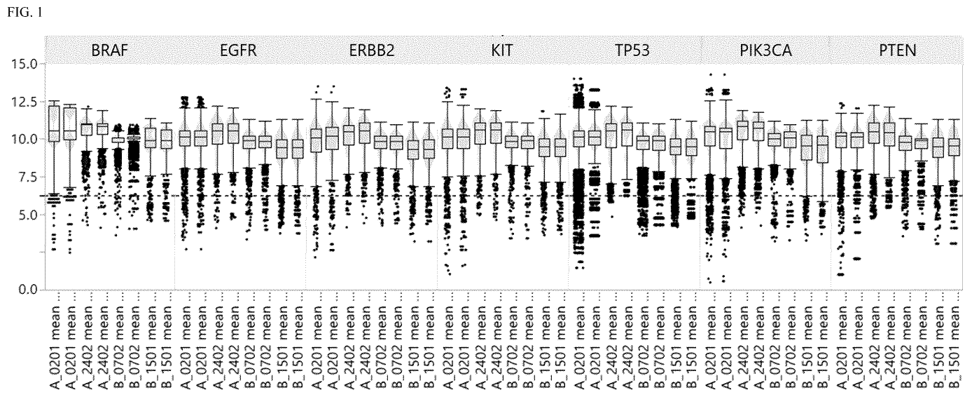

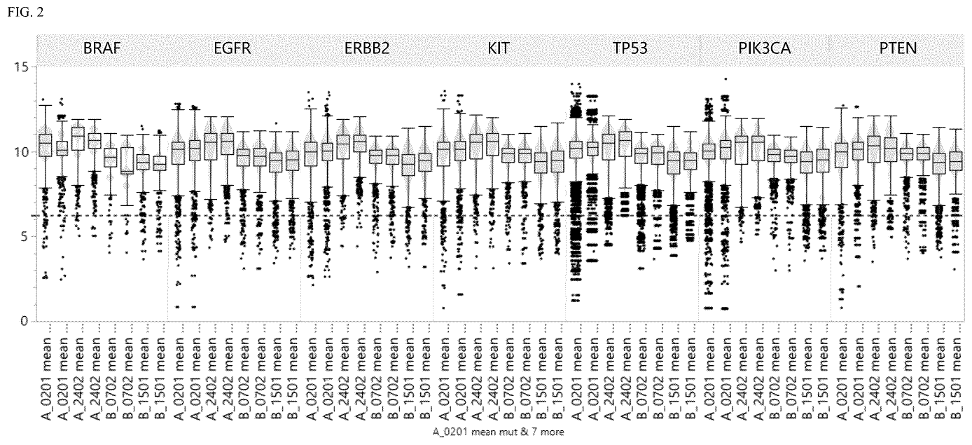

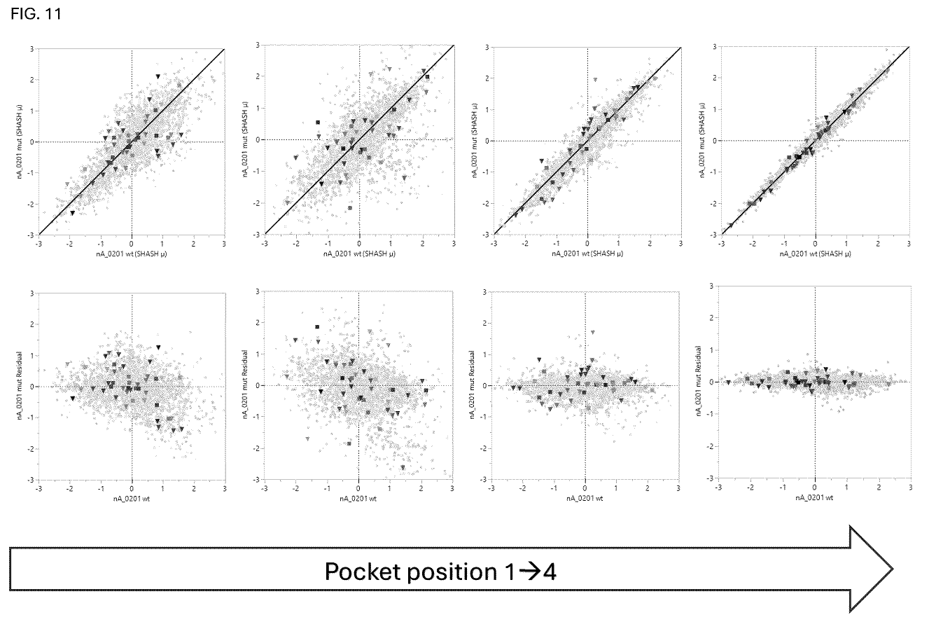

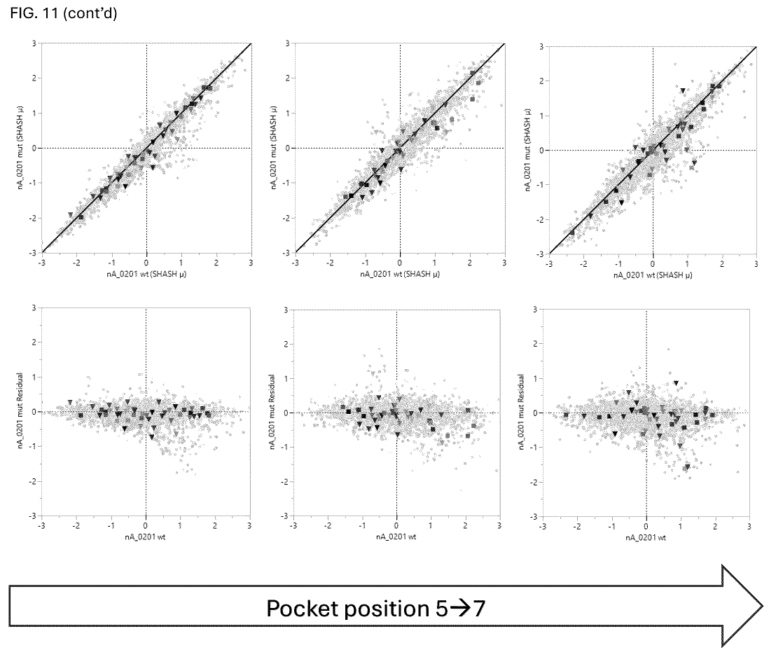

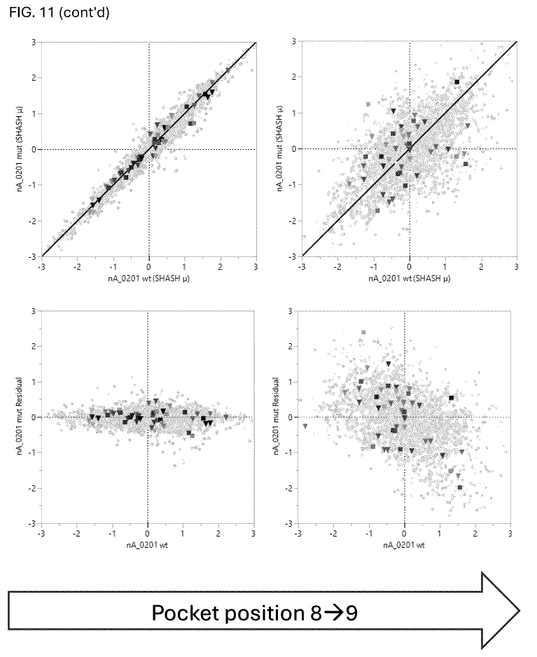

Analysis of the predicted MHC binding of peptides comprising mutations among proteins documented in the TCGA shows no statistical difference in overall predicted binding affinity between mutant and wildtype homolog (FIGS. 1 and 2). However, for TCEM I there is a significant impact when the mutant amino acid lies in positions 2 or 9 of a 9mer (FIG. 11). Overall, based on analysis of proteins with mutations recorded in TCGA, the MHC I binding affinity of the peptides containing the T cell exposed motif which become mutated is very low; about 22 μM, which is more than 40× lower than the 500 nanoM that is the consensus T cell stimulatory level. This indicates that such peptides are overall not highly likely to naturally elicit an effective and sustained cytotoxic T cell response and memory.

In one embodiment, the present invention enables the design of peptides presenting the T cell exposed motif of interest with a range of MHC binding affinities, allowing for selection of very high affinity binders or intermediate binding affinity to the alleles of a particular patient with the goal of stimulating and effective cytotoxic response.

### Frequency Characteristics of Peptides Generated by Mutations in Cancer

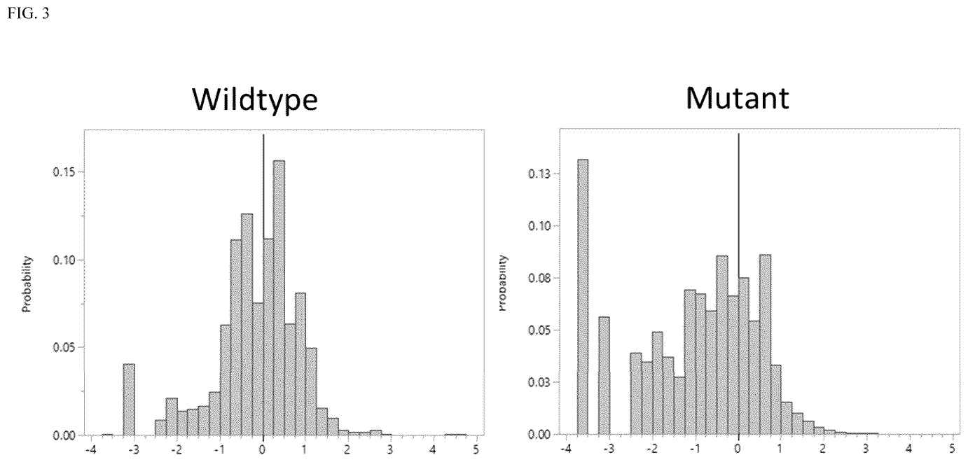

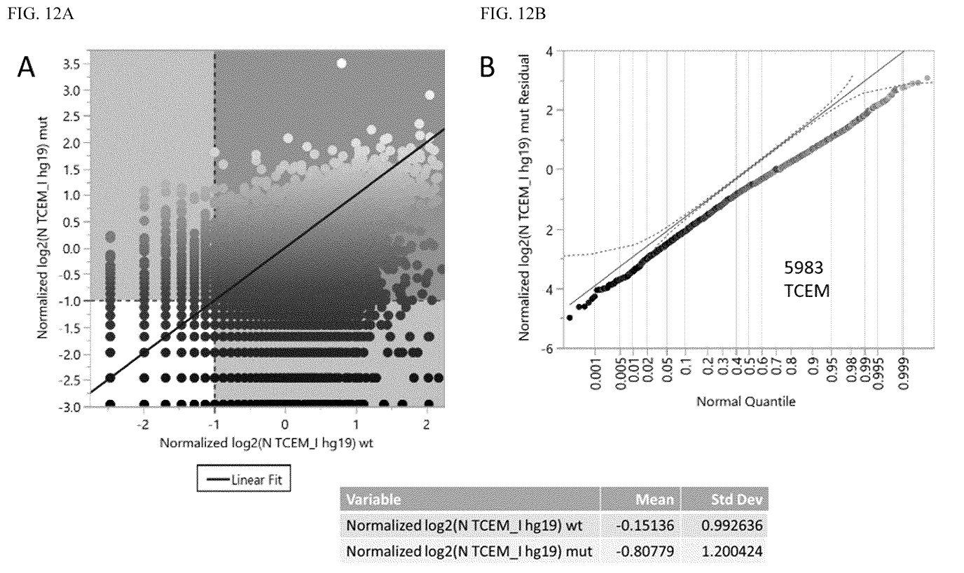

Comparison of the frequency distribution of the T cell exposed motifs in peptides comprising mutations (for TCEM I cognate for MHC I molecules), among those documented in the TCGA, reveals that those comprising mutated amino acids are motifs that occur less commonly in the human proteome than their wildtype homologues (FIGS. 3 and 12). Overall the mutant peptides are biased towards those that are rare or even completely absent in the human proteome; the comparator here being all T cell exposed motif in all peptides of all isoforms of human proteins, approximately 88,000 proteins. The mutational event that inserts a new amino acid in the T cell exposed motif consistently produces T cell exposed motif that are much more rare as compared to the wildtype T cell exposed motif.

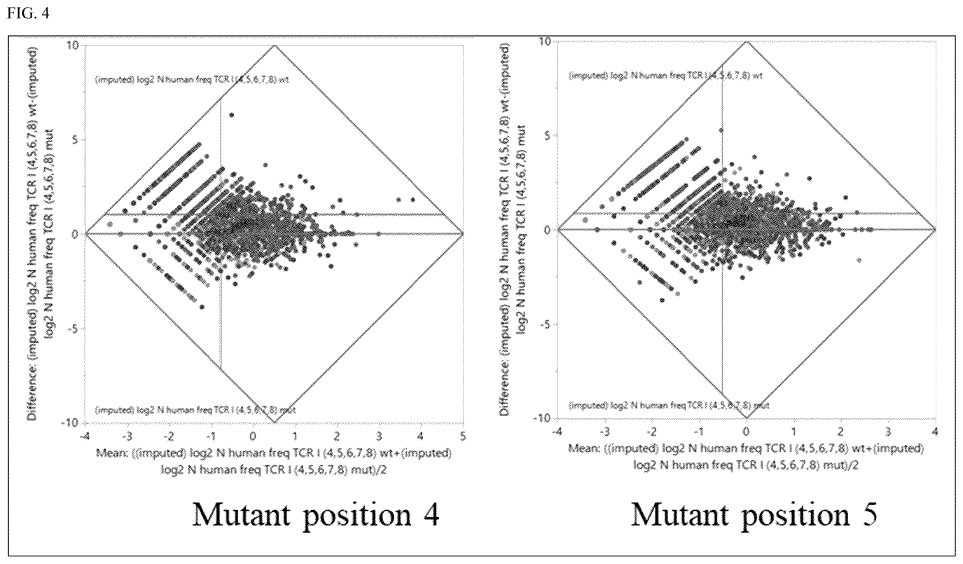

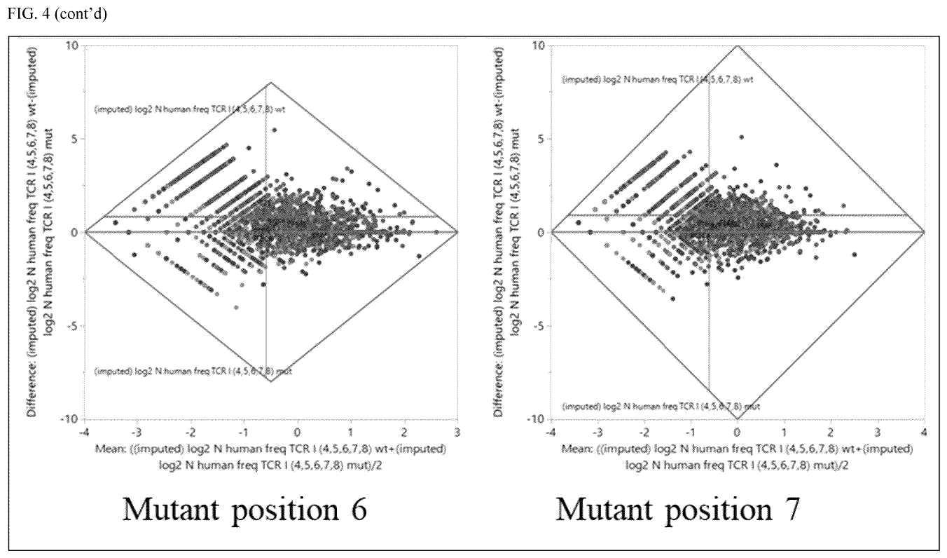

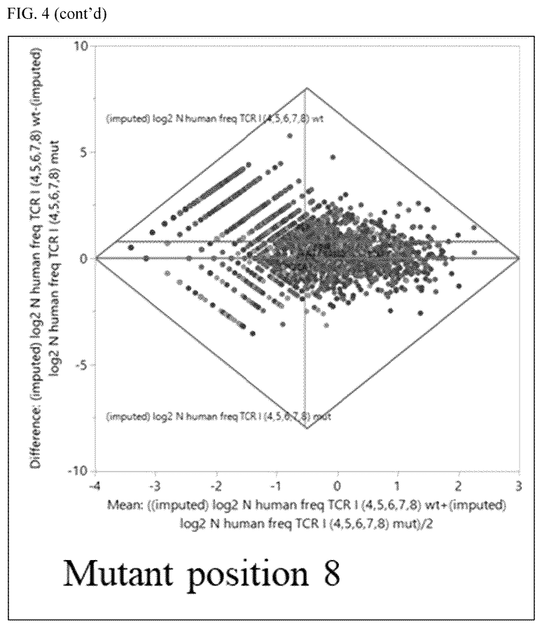

Considering 7 proteins which are commonly mutated in 32 common types of cancer (BRAF, EGFR, ERBB2, KIT, P53, PK3CA and PTEM), the T cell exposed motif frequency category is a standard deviation unit lower (less common) than the wildtype, regardless of the position in the T cell exposed motif at which the mutation occurs (FIG. 4). This Figure shows the stochastic mutation process inserts amino acids into protein sequences that are either much more rare, or in some cases (14% overall), completely absent in normal protein sequences in the human proteome.

It was also noted that when the frequency category of the T cell exposed motif comprising mutated acids in tumors are compared to the frequency of occurrence in the human immunoglobulinome, they correspond on average to the immunoglobulin frequency category FC20; indicating that on average the T cell exposed motif amino acid motifs would be found in 1 in 2²⁰ immunoglobulin variable regions (less than 1 in a million B cell clonal lines). This is 1000 fold below the mean frequency in immunoglobulin variable regions; another indicator that tumor T cell exposed motif are uncommon and that there may be a low frequency of cognate T cells.

### Cross Presentation of MHC I and II Binding Peptides

While the primary focus is on stimulating a cytotoxic T cell response, driven by CD8+ T cells, such a response is enhanced and helped by the simultaneous stimulation of a CD4+ T helper response. This may be particularly important to the development of a population of memory T cells which can ensure ongoing surveillance and elimination of cancer cells. In some instances, a naturally occurring T helper response may be driven from the native mutated protein. In the present invention we also describe how a tumor specific T helper response can be stimulated by peptides designed to have a high binding affinity to the patient's MHC II alleles and to target T cell exposed motifs which comprise the mutated amino acid. Therefore, in one embodiment the invention provides for designing 15mer peptides by maintaining the TCEM II and varying the flanking sequences.

### Maximizing Targeting of Mutations and Stimulation of Cytotoxic T Cell Responses

The combination of these factors: low binding affinity of mutated peptides and rare T cell exposed motif category reduces the chance of a strong natural cytotoxic response. Mutations detected in proteins in tumor biopsies are the “surviving mutations” which have escaped immune surveillance and have not been effectively eliminated after they occur, and so continue to be propagated in the tumor. In one embodiment, the present invention reverses this balance and provides strongly binding peptides which comprise the rare T cell exposed motif and are thus likely to elicit a strong cytotoxic response. Each of the peptides is designed to provide such conditions for a specific patient allele. If a patient is homozygous for any one of their MHC loci, this is detrimental as it limits the number of T cell clones which can be stimulated by the tumor mutations, likely reducing the chances of tumor elimination. Some cancer patients are further handicapped in stimulating the development of effective cytotoxic T cell responses to tumors due to low numbers of mutations.

In some embodiments, therefore, the present invention provides methods to maximize the utilization of available tumor specific antigens to generate effective cytotoxic T cell response that can bring about elimination of the tumor cells. This is achieved by identifying the T cell exposed motif containing the mutant amino acids and generating an array of peptides which combine these T cell exposed motifs with an array of different flanking amino acids of varying predicted binding affinity to enable selection of appropriate high binding peptides. In the case of TCEM I located in a 9-mer comprising 5 exposed amino acids flanked by 4 groove exposed amino acids, for each T cell exposed motif there is a maximum of 20⁴ or 160,000 possible variant amino acid combinations in the groove exposed position. In some embodiments, an array of 1000 peptides is created by random amino acid substitution in the groove exposed positions, in other embodiments an array of 10,000 peptides is likewise created, and in further embodiments a 50,000 peptide array is created. In the case of TCEM II to create peptides binding differentially to MHC II, we consider a 15 mer in which exposed positions 2,3,5,7,8 or −1,3,5,7,8 are kept constant, as all other amino acids in the peptide that are presumed to be involved in the binding affinity are changed by random substitution to create arrays of 1000, 5,000 or 10,000 peptides. In both cases the array sizes cited here are examples that are considered non limiting.

In each case, both MHC I and MHC II, the TCEM is maintained identical to the mutated peptides in the native mutated protein and all TCEM which comprise a mutated amino acid are selected as the basis for generation of binding variants.

In further steps embodied in this invention, the initial array of peptides generated by amino acid substitution is then filtered to remove any duplicate peptides, and in some preferred embodiments peptides predicted to be of low solubility are removed by assigning a score to the polarity of their constituent amino acids. The peptides are then selected to be suitable for the specific patient and his/her combination of MHC I and MHC II alleles. In preferred embodiments all alleles are typed, including MHC I A, MHC I B, MHC I C, and MHC II DRB, DP and DQ loci. In one embodiment, the predicted affinity of the peptides in the native mutant protein is reviewed to determine the probability that a particular peptide would be bound by one or more of the patient's MHC alleles, albeit with a low affinity, and hence presented for T cell recognition. As the goal is to stimulate or “train” T cells to target the specific mutated T cell exposed motifs (TCEM) in the tumor, these must be exposed to T cell recognition to enable targeting of tumor cells. In one embodiment we identify each of the TCEM-allele combinations in each native mutant protein which binds with an affinity greater than the mean for the comprising protein. Such TCEM are targetable by T cells which are also specific to that MHC allele histotope. TCEM-allele combinations which have a predicted binding affinity above the mean are set aside as unlikely to ever be presented. For this subset of “presentable” TCEM-allele combinations, we then assess the array of randomly generated peptides, filtered for binding and solubility, and identify a peptide for each TCEM-allele combination with a desired predicted binding affinity. In some embodiments, the peptide with maximum predicted binding affinity for each allele may be chosen. This may be a peptide that binds at 2.5 or 3 or more standard deviation units below the mean for peptides in the protein (ie higher affinity). Such a high binding peptide would be comparable to those detected as part of the presentome by mass spectroscopy and equivalent to approximately <20 nM to 100 nM, depending on the protein context. In preferred embodiments, peptides are chosen with high, but not excessive predicted binding affinity, keeping in mind the probability that this may be more likely to stimulate an effective cytotoxic response and memory and mitigate against T cell exhaustion. Such a binding affinity may be from 1-2 standard deviation units below the mean for peptides in the protein, typically equivalent to 100-500 nM. Overall, the invention embodies the ability to select for a desired binding affinity and can be considered “tunable” to that selected binding affinity for each patient allele.

Given that each mutated protein has 5 possible TCEM I and TCEM II which exposed the mutated amino acid, in a patient who, for example, has 6 known MHC I alleles and 4 known MHC II alleles, there is a maximum of 30 possible high binding peptides for CD8+ stimulation and 20 for CD4+ stimulation for every known mutated protein. This may be reduced, sometimes by half, due to filtering of non-presented TCEM but still offers a vastly greater number of ways to stimulate T cells which will target the TCEM of interest that depending on natural binding peptides. Simply put, if a binding peptide does not exist, we will create one and if a poor binder is found the affinity is improved by modification of the MHC groove exposed amino acids. The novel peptide thus created will stimulate T cells bearing TCR specific to the tumor.

In some embodiments the novel peptides are used in vitro to stimulate dendritic cells or T cells. In some embodiments such cells are of autologous source, in yet other embodiments they are obtained from allele-matched donors. Stimulated cells are then administered to the cancer patient to passively provide an active T cell population or to provide dendritic cells presenting the TCEM of interest which can stimulate T cells in the patient. In yet other embodiments the peptides are used as components of a peptide vaccine. In yet other embodiments the peptides are applied as a fusion with antibody sequences. In further embodiments the peptides may be encoded in RNA or DNA for administration.

In some embodiments, the frequency classification of the TCEM in the human proteome is noted. In further embodiments the frequency classification of the TCEM in the human immunoglobulinome is noted. In both cases this is achieved by reference to a precomputed reference database comprising over 88,000 human proteins including multiple isoforms and over 35 million unique human immunoglobulin variable regions. Based on this, in some embodiments peptides comprising rare TCEM are identified for priority use.

In desired embodiments, therefore, the process described above yields a unique array of peptides for a particular patient, enabling stimulation of T cells targeting the maximum possible TCEM specific to that patient's tumor-specific mutations and mutated proteins, by presentation of peptides of selected binding affinity in each of the known alleles the patient carries, and said peptides further selected to be soluble. This is a panel of peptides which can then be deployed to stimulate T cells in vivo and in vitro by application in a number of different formats.

TCEMs comprise 5 amino acids, or 20⁵=3.2 million possible configurations. T cell receptor polyspecificity is well recognized [33]. Any neoantigen carries with it the risk of generating an off-target T cell targeting of a self-protein with potential adverse consequences, which may be magnified by immunodulatory interventions such as checkpoint inhibitors. Prior developers of neoantigen vaccines have not addressed this aspect. In a further embodiment of the present invention therefore, TCEM are identified which comprise mutated amino acids and which are bound and presented in the patient's alleles, and are therefore identified as candidates for targeting with T cells stimulated by highly bound peptides. The stimulation of T cells targeting these peptides, when enhanced by high binding affinity neoantigens and potentially further boosted by a checkpoint inhibitor blockade could potentially give rise to self-protein targeting. In one embodiment, therefore, a “call list” of such TCEM is cross-correlated with the reference data set of the human proteome to identify all human proteins carrying said TCEM. These proteins are reviewed to determine the predicted binding affinity of the peptide in which the TCEM occurs for each of the patient's known alleles. If the human proteome carries that TCEM and the patient alleles would bind the contextual peptide at a moderate or high affinity (which may be considered to be an affinity at less than 1 standard deviation below the mean for the comprising protein, although this range is not considered limiting) then the protein carrying the TCEM is added to an advisory list. In preferred embodiments the protein is identified by its Uniprot identifier or identifiers linking it to other reference databases. In preferred embodiments the advisory list is reviewed to further identify proteins where deficiencies or blockades are associated with known pathologies, and to identify proteins which are of critical function and non-redundant. Such proteins may not be suitable for inclusion in a neoantigen vaccine and may be added to a caution list. However, the advisory and caution lists only identify potential sources of adverse reactions and must be weighed against the progression and severity of the cancer. Given the degree of inherent polyspecificity, the advisory list is typically quite extensive. Many proteins are shielded by anatomic or cellular location, some may be considered redundant, or may be considered an acceptable tradeoff to overcoming cancer. However, this embodiment allows an informed decision to be made regarding possible adverse effects in neoantigen selection.

As further illustrated in the Examples, this invention may be applied in two ways, to design and apply bespoke neoantigen vaccines for individual patients and to provide ready-to-go multi-cancer neoantigen arrays for neoantigens found commonly in many cancers.

### Bespoke Design of Neoantigen Vaccines

In a preferred embodiment the present invention allows the rapid design of a personalized immunotherapeutic intervention designed for each cancer patient based on their HLA alleles and particular set of mutations. In some applications of this embodiment the mutations are unique to one patient. This intervention becomes feasible as soon as sequencing of a tumor biopsy and HLA typing is available and can be rapidly computed. In some embodiments the process of sequencing a biopsy may be repeated several times in the course of treatment and the selection of peptides updated based on detection of new mutations. In some preferred embodiments the invention provides an immunotherapy solution for patients who have few proteins with known mutations, for example, but not limited to, glioblastoma patients, who would otherwise be limited to only one neoantigen per protein and possibly no neoantigens with appropriate HLA binding. The preferred embodiment of the present invention is to provide the maximum number of T cell stimulating peptides which will result in targeting of every possible TCEM in which the mutant amino acid occurs and by utilizing every possible HLA. In a further embodiment of the invention the peptides are down-selected to those which will target TCEM presented in vivo and those which are less likely to cause adverse targeting of other human proteins. In an extension of this preferred embodiment, the selected stimulatory peptides may be grouped to provide a series of vaccinations or treatments which allow the utilization of all available alleles the patient carries, while not causing competition for peptide presentation in any one group of peptides.

In some embodiments the selected peptides are applied to dendritic cells in vitro which are then administered to the patient to stimulate T cells. In yet other embodiments the selected peptides are applied in vitro to stimulate a population of T cells which are administered to the patient. In yet other embodiments the peptides, or nucleic acids encoding them are administered directly to the patient in one or more groups spaced over time.

### Neoantigen Array for Common Mutations in Multiple Cancers

Recognizing that many cancers share common mutations in certain proteins, an embodiment of the present invention provides an array of pre-computed and designed peptides which will provide high affinity binding peptides, or nucleic acids that encode them, for said common mutations in commonly mutated proteins shared by many cancers. In preferred embodiments, the proteins with common mutations which are pre-computed and have designed peptides include but are not limited to those shown in Table1 or isoforms thereof.

TABLE 1
Examples of proteins with mutations shared across cancer types

| Gene ID | Protein name |
|---|---|
| AKT1 | RAC-alpha serine/threonine-protein kinase |
| BRAF | Serine/threonine-protein kinase B-raf |
| CASP8 | Caspase-8 |
| CDH1 | CDH1 protein |
| CDKN2A | Cyclin-dependent kinase inhibitor 2A |
| CHEK2 | Serine/threonine-protein kinase Chk2 |
| CTNNB1 | Catenin beta-1 |
| DDX3X | ATP-dependent RNA helicase DDX3X |
| DICER1 | DICER variant 1 |
| EGFR | Epidermal growth factor receptor |
| EP300 | Histone acetyltransferase p300 |
| ERBB2 | Receptor tyrosine-protein kinase erbB-2 |
| ERBB3 | Receptor tyrosine-protein kinase erbB-3 |
| ERBB4 | Receptor tyrosine-protein kinase erbB-4 |
| FBXW7 | F-box/WD repeat-containing protein 7 |
| FGFR2 | Fibroblast growth factor receptor 2 |
| FGFR3 | Fibroblast growth factor receptor 3 |
| FLT3 | Receptor-type tyrosine-protein kinase FLT3 |
| GNA11 | Guanine nucleotide-binding protein subunit alpha-11 |
| GNAQ | Guanine nucleotide-binding protein G(q) subunit alpha |
| HRAS | GTPase HRas |
| IDH1 | Isocitrate dehydrogenase [NADP] 1 |
| IDH2 | Isocitrate dehydrogenase [NADP] 2 |
| KEAP1 | Kelch-like ECH-associated protein 1 |
| KIT | Mast/stem cell growth factor receptor Kit |
| KMT2C | Histone-lysine N-methyltransferase 2C |
| KRAS | GTPase KRas |
| MAP2K1 | MAP kinase |
| MET | Hepatocyte growth factor receptor |
| MTOR | Serine/threonine-protein kinase mTOR |
| NFE2L2 | Nuclear factor erythroid 2-related factor 2 |
| NOTCH1 | NOTCH1 protein |
| NRAS | GTPase NRas |
| PIK3CA | Phosphatidylinositol 4,5-bisphosphate 3-kinase catalytic |
| | subunit alpha isoform |
| PIK3R1 | Phosphatidylinositol 3-kinase regulatory subunit alph |
| PPP2R1A | Serine/threonine-protein phosphatase 2A 65 kDa regulatory |
| | subunit A alpha isoform |
| PTPN11 | Tyrosine-protein phosphatase non-receptor type 11 |
| RAC1 | Ras-related C3 botulinum toxin substrate 1 |
| RASA1 | Ras GTPase-activating protein 1 |
| RB1 | RB1 protein |
| RHEB | GTP-binding protein Rheb |
| RHOA | Transforming protein RhoA |
| RRAS2 | Ras-related protein R-Ras2 |
| RUNX1 | Runt-related transcription factor 1 |
| SETD2 | Histone-lysine N-methyltransferase SETD2 |
| SF3B1 | Splicing factor 3B subunit 1 |
| SMAD2 | Mothers against decapentaplegic homolog 2 |
| SMAD4 | Mothers against decapentaplegic homolog 4 |
| SPOP | Speckle-type POZ protein |
| TGFBR2 | TGF-beta receptor type-2 |
| TP53 | TP 53 |
| VHL | von Hippel-Lindau disease tumor suppressor |
| ZFP36L2 | mRNA decay activator protein ZFP36L2 |

In some proteins, and in the particular case of EGFR, in addition to the common amino acid substitution mutations, insertion-deletions are also common in many types of cancer. In a further embodiment of the invention, we therefore also provide a method of selecting an array of peptides which can serve as tumor specific T cell stimulating peptides for these common deletions. The is an approach which can be applied wherever a deletion creates a novel amino acid motif and thus the example for EGFR is not considered limiting.

In preferred embodiments one or more said pre-computed and designed high affinity peptide from common mutated proteins are applied in the treatment of cancers, including but not limited to adrenocortical carcinoma, bladder urothelial carcinoma, breast adenocarcinoma, cervical squamous cell carcinoma, cholangiocarcinoma, colon carcinoma, lymphoid neoplasm diffuse large b-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, chronic myelogenous leukemia, brain lower grade glioma, liver hepatocellular carcinoma, lung adenocarcinoma, lung squamous cell carcinoma, mesothelioma, ovarian serous carcinoma, pancreatic adenocarcinoma, pheochromocytoma and paraganglioma, prostate adenocarcinoma, rectal carcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma. In preferred embodiments said precomputed and designed peptides included in the array are designed to have high binding for any one of the following alleles A_0101, A_0201, A_0202, A_0203, A_0206, A_0211, A_0212, A_0216, A_0217, A_0219, A_0250, A_0301, A_0801, A_1101, A_2301, A_2402, A_2403, A_2501, A_2601, A_2602, A_2603, A_2902, A_3001, A_3002, A_3101, A_3201, A_3301, A_6801, A_6802, A_6901, A_8001, B_0702, B_0801, B_0802, B_0803, B_1501, B_1502, B_1503, B_1509, B_1517, B_1542, B_1801, B_2703, B_2705, B_3501, B_3801, B_3901, B_4001, B_4002, B_4402, B_4403, B_4501, B_4506, B_4601, B_4801, B_5101, B_5301, B_5401, B_5701, B_5801, B_7301, B_8301, C_0303, C_0401, C_0501, C_0602, C_0702, C_1203, C_1402, C_1502, DPA1_0103-DPB1_0201, DPA1_0201-DPB1_0101, DPA1_0201-DPB1_0501, DPA1_0301-DPB1_0401, DPA1_0301-DPB1_0402, DPB1_0101, DPB1_0201, DPB1_0301, DPB1_0401, DPB1_0402, DPB1_0501, DPB1_1401, DPB1_2001, DQA1_0101-DQB1_0501, DQA1_0102-DQB1_0501, DQA1_0102-DQB1_0502, DQA1_0102-DQB1_0602, DQA1_0103-DQB1_0603, DQA1_0104-DQB1_0503, DQA1_0201-DQB1_0202, DQA1_0201-DQB1_0301, DQA1_0201-DQB1_0303, DQA1_0201-DQB1_0402, DQA1_0301-DQB1_0302, DQA1_0303-DQB1_0402, DQA1_0401-DQB1_0402, DQA1_0501-DQB1_0201, DQA1_0501-DQB1_0301, DQA1_0501-DQB1_0302, DQA1_0501-DQB1_0303, DQA1_0501-DQB1_0402, DQA1_0601-DQB1_0402, DQB1_0201-, DQB1_0202-, DQB1_0301-, DQB1_0302-, DQB1_0402-, DQB1_0501-, DQB1_0502-, DQB1_0503-, DQB1_0602-, DRB1_0101, DRB1_0101_C30S mutant, DRB1_0301, DRB1_0401, DRB1_0404, DRB1_0405, DRB1_0701, DRB1_0801, DRB1_0802, DRB1_0901, DRB1_1001, DRB1_1101, DRB1_1201, DRB1_1301, DRB1_1302, DRB1_1454, DRB1_1501, DRB1_1602, DRB3_0101, DRB3_0202, DRB3_0301, DRB4_0101, DRB4_0103, DRB5_0101. Additional alleles may be added to this list as training sets become available and thus this allele list is not considered limiting. In preferred embodiments, as soon as a patient is identified as carrying a common mutation in a tumor, and his or her HLA typing is known, one or more peptides from the pre-computed ready-to-go array is selected and used in vitro to provide dendritic cells that stimulate T cells on administration to the patient, stimulate T cells which are administered to the patient, or is administered as a component of a peptide vaccination regimen or vaccination with nucleic acids encoding said peptides. In a further embodiment the TCEM matches which can give rise to off-target cytotoxic effects are also precomputed for all potential allele binding situations, enabling risk analysis of peptide use for each patient based on their allele combination.

Neoantigen Based Interventions Combined with Additional Immunotherapies

Application of the bespoke and multi-cancer designed peptides described in the prior sections may, in some embodiments, be combined with other cancer immunotherapies. In some embodiments the peptides or their encoding nucleic acids may be used in vitro to prime dendritic cells or stimulate T cells, or as vaccines in conjunction with drugs targeting upregulated cancer-expressed proteins, biopharmaceuticals binding to tumors, CAR T therapies, radiotherapy, chemotherapy and other clinical interventions. In preferred embodiments said combined chemotherapy should not lead to lymphodepletion. In one particular embodiment the application of the designed peptides or encoding nucleic acids to stimulate dendritic cells or T cells administered to the patient may be combined with a check point inhibitor blockade. In other preferred embodiments, the methods of the present invention comprise administering an immune checkpoint inhibitor to a subject following administration of a multi peptide vaccine or nucleic acid vaccine encoding said peptides. Checkpoint inhibitors act by blocking the inhibition of T cell responses or blocking the termination of a T cell response, thereby unleashing continuing T cell actions. The present invention is applied to ensure that the appropriate tumor targeting T cells are present prior to administration of such a check point blockade. In preferred embodiments, therefore, the peptides designed by the present invention are applied prior to a checkpoint blockade. Suitable checkpoint inhibitors include, but are not limited to, antigen binding proteins that inhibit immune checkpoints, for example by PD-1, PD-L1 or CTLA-4. Suitable checkpoint inhibitors include, but are not limited to, Pembrolizumab, Nivolumab, Ipilimumab, Atezolizumab, Durvalumab, REGN2810 (Anti-PD-1), BMS-936558 (Anti-PD-1), SHR1210 (Anti-PD-1), KN035 (Anti-PD-L1), IBI308 (Anti-PD-1), PDR001 (Anti-PD-1), BGB-A317 (Anti-PD-1), BCD-100 (Anti-PD-1), and JS001 (Anti-PD-1). Other immunomodulatory interventions having the effect of enhancing or extending cellular immune function include but are not limited to ALT-803 and N-803 (IL-15), and haNK, tank and other NK cells.

### Utilization of Designed Peptides

In some embodiments the present invention will yield an array of many peptides suitable for enhancing the CD8+ response of a particular patient to his/her mutated tumor proteins and a list of many peptides suitable for enhancing a CD4+ helper response to these proteins. In some particular embodiments the number of peptides designed to bind MHC and stimulate T cells in a particular patient may be up to 5, in others it is about 20, in yet others it is over 100 and in yet others over 200 peptides. In some embodiments said peptide array will include those which bind to 1 allele, 2 alleles or up to 6 MHC I alleles and others which bind 1, 2 or up to 6 MHC II alleles. In order to optimize the application of said peptides and maximize the use of binding alleles while minimizing competition for binding at any single administration, a further embodiment of the present invention is to prioritize and group the peptides for sequential administration. In a preferred embodiment the peptides may be grouped into subgroups of about 5, in other embodiments subgroups of about 10 are preferred, and in yet other embodiments subgroups of about 20 are preferred and in further embodiments larger groups are preferred. Said subgroups may combine both MHC I and MHC II binding peptides. Some peptides may be repeated in several subgroups. In some embodiments where vaccination regimens comprise sequential administration of a subset of selected peptides, each peptide administration may be followed by check point inhibitor treatment. In some embodiments, consideration is given to whether particular TCEM encompassed in the peptides in each group are rare or common TCEM in the human proteome or immunoglobulinome. In some preferred embodiments priority is given to inclusion of peptides that comprise rare TCEM. In each instance where a peptide is mentioned above, this may also refer to the application of a nucleic acid encoding said peptide. In preferred embodiments peptides that have TCEM matches in certain human proteins are excluded from consideration, where stimulating a T cell response which may target said human proteins may result in an adverse effect. In yet another embodiment, where transcription levels of the mutated proteins in a tumor are known, peptides may be prioritized based on their transcription level to increase the chance of successful targeting of tumor cells.

### Many Delivery Formulations

Many delivery formulations have been proposed for neoepitope vaccines, including but not limited to, peptide vaccines, antibody-antigen fusion proteins, DNA or RNA encoding antigens, particulate vaccines. Neoantigens have been administered directly to subjects or have served to prime dendritic cells or stimulate T cells in vitro for administration of such cells to the subject. The dendritic cells or T cells have included those of autologous or of donor origin. Any of these delivery formulations may be used for delivery of peptides designed by the present invention.

In some embodiments of the present invention the peptides, or their encoding nucleic acids, designed to bind to the patient alleles and stimulate T cells that are specific for tumor TCEM may be administered parenterally. In yet other embodiments the peptides or their encoding nucleic acids may be delivered intradermally or subcutaneously. In some embodiments intradermal administration may be achieved by needle injection. In preferred embodiments intradermal administration may be provided by micro needle patch or array. In yet further embodiments said microneedle patch or array may deliver multiple different peptides or encoding nucleic acids thereof.

In some embodiments the designed peptides or their encoding nucleic acids may be delivered with an adjuvant. Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, squalene, squalene emulsions, liposomes, imiquimod, keyhole limpet hemocyanins, dinitrophenol, and potentially useful human adjuvants such as BCG (Bacille Calmette-Guerin) and Corynebacterium parvum. In other embodiments a cytokine may be co-administered, including but not limited to interferon gamma or stimulators thereof, interleukin 12, or granulocyte stimulating factor. In other embodiments the peptides or their encoding nucleic acids may be co-administered with a local inflammatory agent, either chemical or physical. Examples include, but are not limited to, heat, infrared light, proinflammatory drugs, including but not limited to imiquimod.

In some embodiments the designed peptides may be administered as a fusion to a moiety which favors formation of nanoparticles. Examples of such moieties include but are not limited to leucine multimers (polyleucine), unnatural hydrophobic amino acids, or liposomes. The peptide of interest may be attached to its fusion partner by a linker. In some instances the linker is cleavable. Said cleavable linker may be one or more lysine or arginine residues, or a cathepsin cleavable linker.

Having knowledge of the patient's HLA alleles is a prerequisite to designing a bespoke peptide vaccine. Several approaches to HLA typing may be employed, including PCR, and such testing is widely available. As the patient tumor sequencing is often conducted in association with whole genome sequencing of normal and tumor tissue, the HLA can be derived from the whole genome sequence at the same time by analysis of chromosome 6 using the appropriate BAM slice of chromosome 6 derived from the whole exome sequence.

Bespoke vaccines, designed based on the mutations and HLA of an individual cancer patient are distinctly personal. The particular combination of peptides and the modifications to said peptides to ensure MHC binding and exposure of a particular T cell exposed motif are only suitable for that one individual. As such, the combination of peptides may be determined and selected in consultation with the patient's clinician and prescribed for that patient. In some embodiments, therefore, this may enable preparation of a bespoke vaccine by an entity functioning as a compounding pharmacy.

### Treatment of Other Immunopathologies

Modified epitopes can also play a role in modulation of other immunopathologies, outside the field of oncology. This includes, but is not limited to, applications in autoimmune diseases, allergies and inflammation where the problem is not an insufficient T cell stimulation, but rather an overexuberant response. Provision of a very high affinity binding peptide can serve to exhaust or diminish the T cell response to the particular T cell exposed motif in question and thereby diminish CD4 T cell help or a CD8 cytotoxic response and ameliorate the pathogenesis of the disease. In each case the peptides are customized to ensure binding appropriate the HLA alleles of the individual patient.

Autoimmune diseases in which such an approach may be useful include, but are not limited to rheumatoid arthritis, diabetes type I and type II, Ankylosing Spondylitis, Atopic allergy, Atopic Dermatitis, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome, Autoimmune progesterone dermatitis, Autoimmune thrombocytopenia purpura, Autoimmune uveitis, Bullous Pemphigoid, Castleman's disease, Celiac disease, Cogan syndrome, Cold agglutinin disease, Crohns Disease, Dermatomyositis, Eosinophilic fasciitis, Gastrointestinal pemphigoid, Goodpasture's syndrome, Graves' disease, Guillain-Barré syndrome, Anti-ganglioside Hashimoto's encephalitis, Hashimoto's thyroiditis, Systemic Lupus erythematosus, Miller-Fisher syndrome, Mixed Connective Tissue Disease, Myasthenia gravis, Narcolepsy, Pemphigus vulgaris, Polymyositis, Primary biliary cirrhosis, Psoriasis, Psoriatic Arthritis, Relapsing polychondritis, Sjögren's syndrome, Temporal arteritis, Ulcerative Colitis, Vasculitis, and Wegener's granulomatosis. Allergic responses which may benefit from immunomodulation by design of personal peptides of modified binding include but are not limited to allergies to plant, animal, insect, arachnoid materials and other environmental materials comprising allergen epitopes. Allergies may result form airborne or gastrointestinal exposure or from skin contact.

In some instances, an immunopathology can arise as the result of an adverse response to a therapeutic agent administered to a subject. In some cases said therapeutic is a biopharmaceutical protein.

In each case an individual subject afflicted by an autoimmune disease or allergen may be typed as to their HLA alleles and a peptide array designed specifically for that person to provide peptides that exhaust the T cell response. Examples of such customized peptides are shown in Example 12.

### EXAMPLES

### Example 1: Selection of Mutant Peptides and Generation of Better Binding Peptides

The development of vaccines and stimulants for dendritic cells and T cells in vitro to comprise multiple peptides with a selected desired affinity for the patient's alleles builds on methods previously described to precisely predict MHC binding, identify and analyze T cell exposed motifs and generate peptides with altered binding affinity (See PCT Appl. US14/41523, PCT Appl. US15/39969, and PCT Appl US17/21781, all of which are incorporated herein by reference in their entirety).

### Identification of Relevant Peptide Positions.

In order for a T cell to differentially target a tumor cell expressing a mutated protein, the mutated amino acid has to be located in a position “visible” or exposed to the T cell receptor and not hidden in the pocket or groove exposed positions that determine binding. A first step in designing a multi peptide vaccine or stimulant panel is therefore to identify those peptide positions which expose the mutated amino acid. For MHC I this means the mutant amino acid must be at positions 4,5,6,7 or 8 of a 9-mer peptide and for MHC II at positions 2,3,5,7,8 of the 9-mer core of a 15 mer. This identifies TCEM IIA; TCEM IIB positions are at −1,3,5,7,8. We first calculated the predicted binding affinity of all sequential peptide positions in the mutant protein and then selected those peptides with relevant TCEM comprising mutated amino acids.

A T cell is only able to target a TCEM if that motif is presented in the host from the naturally occurring mutant peptide. Mutant TCEM that lie in peptides that are extremely unlikely to ever be presented are thus poor targets. We therefore filtered the TCEM to identify those which have some likelihood of exposure in the host, limiting to those whose predicted binding affinity is greater than the mean for the protein. This is not an absolute requirement but maximizes the potential for a successful targeting.

For each of the selected peptides comprising a mutant TCEM, a bank of peptides was generated by randomly varying the flanking amino acids, and recalculating the new binding affinity for each allele of interest. For a 9-mer with a pentamer exposed TCEM, this implies up to 160,000 (20⁴) different peptides could be generated, each with a different binding affinity. For practical purposes a bank of 1000 or up to 10,000 peptides is usually sufficient to provide peptides within the range of binding affinity desired. For MHC II we opted to vary only those amino acids outside the core 9 mer peptide comprising the TCEM, as the intercalated amino acids which are in pocket (groove exposed) positions affect binding but may also influence the positioning of the exposed amino acids.

A further practical consideration is solubility of the peptide. A score was generated based on the polarity of the constituent amino acids and only peptides likely to be soluble were put forward as candidates. Sufficient peptides can be generated to prevent this from becoming a limitation.

For a group of 5 proteins each with one mutation and a patient with 4 known alleles therefore a maximum number of allele TCEM combinations is 5 TCEM x 5 proteins X 4 alleles or 100 possible ways to stimulate T cells which will uniquely target those proteins. This is reduced by the TCEM of low probability of natural presentation.

### Example 2: Selection of Personalized Simulated Peptides

The process described in Example 1 generates a selection of peptides of different binding affinity for each combination of mutant-containing-TCEM and patient allele. Peptides are then selected which have a desired predicted binding affinity. We have discussed the relevance of binding affinity on T cell phenotype in the Description above. As peptides of many different binding affinities are provided the desired affinity may be selected. In the subsequent examples we have opted to focus on peptides with predicted binding affinity at about 2 standard deviations below the mean of the protein, placing them at about the 95^{th} percentile; i.e. the top 5% binders, but not higher, because conceivably very high affinity peptide could lead to immunosuppression or exhaustion. We have shown the number of peptides available at this level and in some cases at 3 SD or greater (very high binders).

Utilization of the available peptides may depend on the intended use as a neoepitope vaccine or in vitro stimulant of dendritic cells and T cells to be administered to the patient.

Peptides may be selected to use in groups that target the maximum number of combinations of allele and TCEM in any one application. One desired aspect is to ensure not all peptides administered at any one time as a multi-neoepitope vaccine target the same allele, thus competing with each other for space in MHC and presentation. When dendritic cells and T cells are targeted in vitro it may be desirable to provide as many combinations as possible.

### Example 3: Reference to Human Proteome to Identify Potential Adverse Reactions

To identify potential off target effects of the T cells stimulated by the peptides designed to generate targeting of cancer mutations, we compare the TCEM with those in the human proteome to identify relevant matches. The entire human proteome, comprising over 88,000 proteins (including all known isoforms of each protein), was pre-analyzed to determine the binding affinity of each peptide in each protein for all MHC alleles. The TCEM comprised in the peptides selected for each cancer patient, selected as described in Example 1 are assembled into a “call list”. The human proteome reference database is searched for all TCEM on the patient call list; a subset of proteins with matching TCEM is assembled. The peptides in this subset which contain the TCEM on the call list are then examined to determine if the TCEM would be likely to be presented in the MHC corresponding to that patient's alleles. If the proteome peptide comprising the TCEM of interest is predicted to bind to any one of the patient's known alleles with an affinity <1 SD below the mean for the protein, the protein is included in an advisory list. The list is curated to remove duplicates and references to any protein fragments catalogued in UniProt (www.uniprot.org). Individual proteins may be reviewed in UniProt and elsewhere to determine if there is evidence of pathologies arising from deficiencies or mutations in the protein. Instances in which a protein of immediate concern is targeted are flagged with a “caution” and excluded from the proposed peptides encoded in a vaccine or in vitro cell stimulation. Examples include, but are not limited to, coagulation factors, neurotransmitters, complement, other proteins with known essential and non-redundant functions. Decision on off-targeting of proteins in the advisory list may be based on a risk-benefit analysis of the patient's condition but access to such a list allows the oncologist to make an informed decision. The most complete typing of a patient's alleles enables a more complete assessment of potential off-targets. Notably, as the relevance of each target will depend on its presentation as a result of the MHC binding of the peptide in which the TCEM occurs, identifying the potential off-target impacts is as personalized as the design of the peptide array for that cancer patient. Specific examples of such advisory and caution proteins are shown in Example 4 below.

### Example 4: Application of Personalized Multiplex Vaccine or In Vitro T Cell and Dendritic Cell Stimulants in a Glioblastoma Patient

In this Example and the two following Examples 5 and 6 we illustrate the design of a personalized array of peptides to stimulate cancer specific cytotoxic T cells for patients with three different types of cancer: glioblastoma, melanoma and small cell lung cancer. Such peptides may be used to stimulate dendritic cells or T cells in vitro for subsequent administration to the patient, or may form the basis for a personalized vaccine. Said vaccine may be administered by any one of many delivery vehicles. The peptides may be encoded as DNA or RNA for delivery. The peptides may be used alone or expressed as a fusion to an antibody or partial immunoglobulin molecule. Peptides or nucleic acids encoding them may be injected intradermally or parenterally or may be applied in a transdermal microneedle array. The peptides or nucleic acids may be delivered with an adjuvant, cytokine, chemokine or with a physical stimulus of inflammation. In addition, each peptide or nucleic acid administration to stimulate the tumor specific T cells may be accompanied with or followed by a check point inhibitor drug. In each case, to the extent possible based on allele typing, we identify potential off target effects.

### Glioblastoma Patient Personalized Peptide Neoepitope Array

Patient X, diagnosed with glioblastoma, has 10 proteins with identified mutations and is MHC typed as A0301, B3501, B 4402 and C0401 for MHC I, and DRB1_0401 and DRB 1_0701. The proteins and mutations are shown in Table 2. While mutations identified in a tumor biopsy were demonstrated by comparison with contemporaneous normal tissue (PBMCs), complete sequencing was not available from the normal patient tissue, so a reference sequence was used as the basis for whole protein peptide affinity predictions.

TABLE 2
Protein and mutations for Patient X

| | Reference | Amino acid |
|---|---|---|
| Protein | gi | Mutation |
| Angiomotin isoform 1 | 166064029 | P415L |
| ATP-dependent RNA helicase DDX3X | 301171467 | E481K |
| isoform 2 | | |
| Coiled-coil domain-containing protein | 41281911 | Q122P |
| 50 long isoform | | |
| Dipeptidyl peptidase 4 | 18765694 | K56M |
| Kelch-like ECH-associated protein 1 | 22027642 | R614W |
| Kinesin heavy chain isoform 5C | 4758650 | E492K |
| Nephrocystin-4 isoform a | 23510323 | S43P |
| Peroxisomal acyl-coenzyme A oxidase 1 | 30089972 | P126L |
| isoform a | | |
| Phosphatidylinositol 3 | 73765544 | K6E |
| Symplekin H | 124028529 | P1069S |

### Tumor Specific MHC I Binding Targets to Generate CD8 T Cells.

Table 3 summarizes for MHC I alleles that 200 TCEM allele combinations are available for potential targeting in this patient and shows the process of down selection to those TCEM likely to be accessible to T cells as a result of natural presentation and down-selected for other reasons. It determines that if binding affinity of ~2 SD is used, a panel of 1000 simulated peptides for each TCEM allele combination generates 88 distinct T cell targets for which T cell stimulating peptides have been identified. If a higher affinity of <3 SD is preferred this number is reduced to 56 peptides or their encoding nucleotides.

TABLE 3
Potential TCEM allele combinations and filtration to actual available

| | Patient X |
|---|---|
| Proteins with identified | 10 |
| mutations | |
| TCEM with mutations | 50 for 4 alleles = 200 MHC I and 100 MHC II |

| Patient MHC Alleles | A0301 | B3501 | B4402 | C0401 | DRB0401 | DRB0701 |
|---|---|---|---|---|---|---|
| TCEM naturally presented | 24 | 24 | 25 | 29 | 28 | 24 |
| for allele | | | | | | |
| Mutated proteins in which | 10 | 10 | 10 | 10 | 9 | 8 |
| natural presentation occurs | | | | | | |
| Proteins omitted as no | 0 | 0 | 0 | 0 | 1 | 2 |
| natural presentation | | | | | | |
| Unique peptides simulated | 20915 | 24731 | 21780 | 25752 | 39818 | 39344 |
| with any binding | | | | | | |
| Subset for which TCEM | 10738 | 13016 | 13255 | 16781 | 15904 | 15916 |
| is naturally presented | | | | | | |
| Filtered by polarity score <1 | 7765 | 9207 | 8232 | 13337 | 11986 | 11563 |
| indicating solubility | | | | | | |
| Peptides selected in binding | 948 | 545 | 667 | 294 | 1061 | 356 |
| window <−1.75 > −2.25 SD | | | | | | |
| Peptides selected in binding | 129 | 11 | 375 | 145 | 8 | 5 |
| window <3.0 SD | | | | | | |
| Represent TCEM allele | 24 | 21 | 25 | 26 | 24 | 22 |
| combos <−1.75 > −2.25 SD | | | | | | |
| Represent TCEM allele | 18 | 6 | 24 | 13 | 1 | 5 |
| combos <3.0 | | | | | | |
| Removed due to immediate | 1 | 3 | 2 | 2 | 0 | 0 |
| off target caution; or high | | | | | | |
| frequency Fc | | | | | | |
| Net TCEM allele combos | 23 | 18 | 23 | 24 | 24 | 22 |
| available <−1.75 > −2.25 SD | | | | | | |
| Net TCEM allele combos | 17 | 6 | 22 | 11 | 1 | 5 |
| available <3.0 SD | | | | | | |
| Potential vaccine peptides | MHC I | <−1.75 > | 88 | MHC II | <−1.75 > | 46 |
| per patient for all mutated | | −2.25 | | | −2.25 | |
| proteins | | <3.0 | 56 | | <3.0 | 6 |

Binding shown in standard deviation units

Table 4 shows example peptides and their predicted binding affinity for each of the MHC I TCEM allele combinations and shows those combinations for which presentation in the native mutant protein is not likely. Table 4 also shows TCEM removed from consideration due to an immediate caution of off target responses. These are further explained in Table 6. Table 5 shows how the peptides identified in Table 4 could be grouped into arrays of 10 for sequential application to maximize utilization of alleles and minimize competition for binding sites at any one time. Table 7 provides details of the concerns for potential adverse reactions arising from targeting for the immediate caution proteins with matching and presented TCEM I

For MHC II Table 8 shows example peptides and their predicted binding affinity for each of the MHC IIA TCEM allele combinations designed to stimulate CD4 stimulation and shows those combinations for which presentation in the native mutant protein is not likely. One protein, the ATP-dependent RNA helicase DDX3X, is not represented in the simulated peptide list as it would be expected to have very poor binding in the peptides overlapping the mutated amino acid. However, it would be expected to benefit from T cell help from a very close downstream set of peptides (index positions 481-493) which have high predicted binding for the alleles of interest and would be naturally presented in the mutated protein. Table 9 shows how the peptides identified in Table 7 could be grouped into arrays for sequential application to maximize utilization of alleles and minimize competition for binding sites at any one time. Table 10 shows the advisory list of potential off target binding for the selected TCEM and patient X MHC II alleles. A set of the peptides designed were administered intradermally to Patient X and subsequent Elispots detected responses to groups of peptides.

TABLE 4
TCEM Allele combinations and selected peptides for each designed to stimulate CD8 T cells in Patient X

| Protein | | | | | | | | | | | | | |
|---|---|---|---|---|---|---|---|---|---|---|---|---|---|
| curation and | | | | SEQ | | | | | | | | | |
| reference | aa | | | ID | | | | | | | C0401 | | |
| sequence | Mut | position | TCEM 1 | NO: | A0301 Simulated | A0301 | B3501 Simulated | B3501 | B4402 Simulated | B4402 | Simulated | C0401 | Caution |
| kelch-like | W | 607 | ~~~MEPCW~ | 285 | KTFMEPCWP | −1.80 | no | | no | | no | | |
| ECH- | | | | | (SEQ ID NO: 335) | | | | | | | | |
| associated | W | 608 | ~~~EPCWK~ | 286 | AGKEPCWKP | −1.91 | HGVEPCWKI | −1.95 | ASLEPCWKH | −1.93 | no | | |
| protein 1 | | | | | (SEQ ID NO: 336) | | (SEQ ID NO: 357) | | (SEQ ID NO: 377) | | | | |
| Homo sapiens | W | 609 | ~~~PCWKQ~ | 287 | no | | MKGPCWKQF | −2.04 | VESPCWKQS | −1.76 | LLRPCWKQ | −1.79 | |
| gi:22027642 | | | | | | | (SEQ ID NO: 358) | | (SEQ ID NO: 378) | | A (SEQ ID | | |
| | | | | | | | | | | | NO: 400) | | |
| | W | 610 | ~~~CWKQI~ | 288 | LAACWKQIK | −2.00 | No | | IERCWKQIE | −1.95 | no | | |
| | | | | | (SEQ ID NO: 337) | | | | (SEQ ID NO: 379) | | | | |
| | W | 611 | ~~~WKQID~ | 289 | no | | RPSWKQIDF | −1.88 | CERWKQIDD | −1.99 | PFDWKQIDP | −1.97 | |
| | | | | | | | (SEQ ID NO: 359) | | (SEQ ID NO: 380) | | (SEQ ID NO: | | |
| | | | | | | | | | | | 401) | | |
| dipeptidyl | M | 49 | ~~~TYRLM~ | 290 | no | | FKNTYRLML | −1.97 | TETTYRLMV | −2.00 | No | | |
| peptidase 4 | | | | | | | (SEQ ID NO: 360) | | (SEQ ID NO: 381) | | | | |
| Homo sapiens | M | 50 | ~~~YRLML~ | 291 | LDPYRLMLK | −2.19 | No | | no | | No | | |
| gi:18765694 | | | | | (SEQ ID NO: 338) | | | | | | | | |
| | M | 51 | ~~~RLMLY~ | 292 | no | | No | | no | | SKIRLMLYS | −1.82 | |
| | | | | | | | | | | | (SEQ ID NO: | | |
| | | | | | | | | | | | 402) | | |
| | M | 52 | ~~~LMLYS~ | 293 | no | | No | | REELMLYSQ | −2.04 | RRYLMLYS | −1.92 | |
| | | | | | | | | | (SEQ ID NO: 382) | | K (SEQ ID | | |
| | | | | | | | | | | | NO: 403) | | |
| | M | 53 | ~~~MLYSL~ | 294 | TQSMLYSLK | −1.96 | RPRMLYSLM | −1.55 | IERMLYSLR | −1.96 | KKLMLYSL | −1.35 | |
| | | | | | (SEQ ID NO: 339) | | (SEQ ID NO: 361) | | (SEQ ID NO: 383) | | K (SEQ ID | | |
| | | | | | | | | | | | NO: 404) | | |
| peroxisomal | L | 119 | ~~~RFFML~ | 295 | no | | No | | No | | SAERFFMLK | −1.82 | |
| acyl- | | | | | | | | | | | (SEQ ID NO: | | |
| coenzyme A | | | | | | | | | | | 405) | | |
| oxidase 1 | L | 120 | ~~~FFMLA~ | 296 | DDRFFMLAK | −2.05 | KSEFFMLAR | −1.15 | EEQFFMLAQ | −2.19 | DRRFFMLA | −1.83 | |
| isoform a | | | | | (SEQ ID NO: 340) | | (SEQ ID NO: 362) | | (SEQ ID NO: 384) | | K (SEQ ID | | |
| gi:30089972 | | | | | | | | | | | NO: 406) | | |
| | L | 121 | ~~~FMLAW~ | 297 | no | | No | | AEKFMLAWE | −1.91 | no | | |
| | | | | | | | | | (SEQ ID NO: 385) | | | | |
| | L | 122 | ~~~MLAWN~ | 298 | EPSMLAWNK | −1.88 | QARMLAWNY | −2.36 | SGPMLAWNR | −2.00 | PGSMLAWN | −1.63 | |
| | | | | | (SEQ ID NO: 341) | | (SEQ ID NO: 363) | | (SEQ ID NO: 386) | | K (SEQ ID | | |
| | | | | | | | | | | | NO: 407) | | |
| | L | 123 | ~~~LAWNL~ | 299 | SGRLAWNLP | −2.04 | RQELAWNLW | −1.64 | TEVLAWNLK | −1.98 | no | | |
| | | | | | (SEQ ID NO: 342) | | (SEQ ID NO: 364) | | (SEQ ID NO: 387) | | | | |
| | L | 408 | ~~~PRAQL~ | 300 | RFYPRAQLP | −2.06 | No | | EEVPRAQLP | −1.94 | KLKPRAQLL | −1.90 | |
| | | | | | (SEQ ID NO: 343) | | | | (SEQ ID NO: 388) | | (SEQ ID NO: | | |
| | | | | | | | | | | | 408) | | |
| angiomotin | L | 409 | ~~~RAQLS~ | 301 | no | | No | | DCSRAQLSA | −1.91 | LELRAQLSS | −1.98 | |
| isoform 1 | | | | | | | | | (SEQ ID NO: 389) | | (SEQ ID NO: | | |
| Homo sapiens | | | | | | | | | | | 409) | | |
| gi:166064029 | L | 410 | ~~~AQLSS~ | 302 | no | | Caution | | no | | caution | | #1 |
| | L | 411 | ~~~QLSSA~ | 303 | no | | No | | no | | SLRQLSSAL | −1.86 | |
| | | | | | | | | | | | (SEQ ID NO: | | |
| | | | | | | | | | | | 410) | | |
| | L | 412 | ~~~LSSAS~ | 304 | EGRLSSASK | −1.76 | KNVLSSASW | −2.02 | no | | no | | |
| | | | | | (SEQ ID NO: 344) | | (SEQ ID NO: 365) | | | | | | |
| coiled-coil | P | 115 | ~~~EKELP~ | 305 | KGSEKELPQ | −2.03 | DAEEKELPY | −2.14 | no | | APFEKELPR | −1.82 | |
| domain- | | | | | (SEQ ID NO: 345 | | (SEQ ID NO: 366) | | | | (SEQ ID NO: | | |
| containing | | | | | | | | | | | 411) | | |
| protein 50 | P | 116 | ~~~KELPE~ | 306 | no | | caution | | no | | no | | #2 |
| long isoform | P | 117 | ~~~ELPQQ~ | 307 | yes | | no | | GHFELPEEM | −2.02 | no | | |
| Homo sapiens | | | | | | | | | (SEQ ID NO: 390) | | | | |
| gi:41281911 | P | 118 | ~~~LPEEK~ | 308 | no | | no | | No | | WLQLPEEK | −1.95 | |
| | | | | | | | | | | | W (SEQ ID | | |
| | | | | | | | | | | | NO: 412) | | |
| | P | 119 | ~~~PEEKK~ | 309 | GGYPEEKKP | −1.82 | DLIPEEKKF | −1.96 | VELPEEKKS | | | | |
| | | | | | (SEQ ID NO: 346) | | (SEQ ID NO: 367) | | (SEQ ID NO: 391) | −2.01 | QCLPEEKKS | −1.80 | |
| | | | | | | | | | | | (SEQ ID | | |
| | | | | | | | | | | | NO: 413) | | |
| kinesin heavy | K | 485 | ~~~LQALK~ | 310 | caution | | No | | Caution | | caution | | #3 |
| shain isoform | K | 486 | ~~~QALKE~ | 311 | no | | VLRQALKEF | −1.99 | FESQALKEV | −2.02 | no | | |
| 5C | | | | | | | (SEQ ID NO: 368) | | (SEQ ID NO: 392) | | | | |
| gi:4758650 | K | 487 | ~~~ALKEL~ | 312 | no | | VGDALKELM | −1.90 | PEKALKELQ | −1.91 | no | | |
| | | | | | | | (SEQ ID NO: 369) | | (SEQ ID NO: 393) | | | | |
| | K | 488 | ~~~LKELA~ | 313 | VEFLKERLAR | −1.95 | KSQLKELAY | −1.99 | QCELKELAT | −1.90 | YQMLKELA | −1.97 | |
| | | | | | (SEQ ID NO: 347) | | (SEQ ID NO: 370) | | (SEQ ID NO: 394) | | (SEQ ID | | |
| | | | | | | | | | | | NO: 414) | | |
| | K | 489 | ~~~KELAV~ | 314 | YVRKELAVQ | −1.98 | WPSKELAVR | −1.94 | no | | no | | |
| | | | | | (SEQ ID NO: 348) | | (SEQ ID NO: 371 | | | | | | |
| symplekin | S | 1062 | ~~~FDKCS~ | 315 | KCFFDKCSD | −1.77 | No | | LCLFDKCSS | −1.80 | QWRFDKCS | −1.88 | |
| Homo sapiens | | | | | (SEQ ID NO: 349) | | | | (SEQ ID NO: 395) | | Q (SEQ ID | | |
| gi:124028529 | | | | | | | | | | | NO: 415) | | |
| | S | 1063 | ~~~DKCSE~ | 316 | VCFDKCSEQ | −1.87 | No | | no | | LQHDKCSE | −1.94 | |
| | | | | | (SEQ ID NO: 350) | | | | | | (SEQ ID | | |
| | | | | | | | | | | | NO: 416) | | |
| | S | 1064 | ~~~KCSL~ | 317 | DVMKCSELP | −2.14 | RSNKCSELY | −1.96 | no | | LKFKCSELA | −1.80 | |
| | | | | | (SEQ ID NO: 351) | | (SEQ ID NO: 372) | | | | (SEQ ID | | |
| | | | | | | | | | | | NO: 417) | | |
| | S | 1065 | ~~~CSELR~ | 318 | no | | No | | LEECSELRT | −1.84 | no | | |
| | | | | | | | | | (SEQ ID NO: 396) | | | | |
| | S | 1066 | ~~~SELRE~ | 319 | no | | Caution | | caution | | no | | #4 |
| ATP- | K | 474 | ~~~QRDRK~ | 320 | no | | No | | no | | VILQRDRKK | −1.46 | |
| dependent | | | | | | | | | | | (SEQ ID | | |
| RNA helicase | | | | | | | | | | | NO: 418) | | |
| | K | 475 | ~~~RDRKE~ | 321 | no | | No | | no | | no | | |
| DDX3X | K | 476 | ~~~DRKEA~ | 322 | no | | No | | no | | FDWDRKEA | −1.88 | |
| isoform 2 | | | | | | | | | | | G (SEQ ID | | |
| Homo sapiens | | | | | | | | | | | NO: 419) | | |
| Gi: | K | 477 | ~~~RKEAL~ | 323 | no | | No | | no | | no | | |
| 301171467 | K | 478 | ~~~KEALH~ | 324 | KPYKEALHP | −1.90 | No | | no | | AYDKEALH | −1.90 | |
| | | | | | (SEQ ID NO: 352) | | | | | | L (SEQ ID | | |
| | | | | | | | | | | | NO: 420) | | |
| phosphatidyli | E | 14 | ~~~TAIIE~ | 325 | no | | No | | REATAIIEE | −1.92 | AAVTAIIEK | −1.76 | |
| nositol 3 | | | | | | | | | (SEQ ID NO: 397) | | (SEQ ID NO: | | |
| gi:73765544 | | | | | | | | | | | 421) | | |
| | E | 15 | ~~~AIIEE~ | 326 | no | | No | | no | | no | | |
| | E | 16 | ~~~IIEEI~ | 327 | KCKEEIVSP | −2.00 | LAEIIEEIH (SEQ | −1.87 | EEHIIEEID (SEQ | −1.94 | no | | |
| | | | | | (SEQ ID NO: 353) | | ID NO: 373) | | ID NO: 398) | | | | |
| | E | 17 | ~~~IEEIV~ | 328 | no | | KGEIEEIVY | −2.07 | no | | PTCIEEIVK | −1.96 | |
| | | | | | | | (SEQ ID NO: 374) | | | | (SEQ ID NO: | | |
| | | | | | | | | | | | 422) | | |
| | E | 18 | ~~~EEIVS~ | 329 | DAWEEIVSY | −1.86 | LGSEEIVSR | −1.75 | no | | RRFEEIVSD | −1.93 | |
| | | | | | (SEQ ID NO: 354) | | (SEQ ID NO: 375) | | | | (SEQ ID NO: | | |
| | | | | | | | | | | | 423) | | |
| nephrocystin- | P | 36 | ~~~PWKEP~ | 330 | no | | No | | no | | no | | |
| 4 isoform a | P | 37 | ~~~WKEPT~ | 331 | no | | No | | no | | STCWKEPT | −1.97 | |
| gi:23510323 | | | | | | | | | | | K (SEQ ID | | |
| | | | | | | | | | | | NO: 424) | | |
| | P | 38 | ~~~KEPTA~ | 332 | MYLKEPTAK | −1.93 | LPSKEPTAA | −1.78 | no | | AMKKEPTA | −1.83 | |
| | | | | | (SEQ ID NO: 355) | | (SEQ ID NO: 376) | | | | L (SEQ ID | | |
| | | | | | | | | | | | NO: 425) | | |
| | P | 39 | ~~~EPTAF~ | 333 | no | | No | | no | | IYREPTAFS | −1.88 | |
| | | | | | | | | | | | (SEQ ID NO: | | |
| | | | | | | | | | | | 426) | | |
| | P | 40 | ~~~PTAFQ~ | 334 | IVRPTAFQQ | −2.20 | NSAPTAFQF | −1.92 | VERPTAFQG | −1.96 | no | | |
| | | | | | (SEQ ID NO: 356) | | (SEQ ID NO: 377) | | (SEQ ID NO: 399) | | | | |

#1 Cautions: 1: Complement factor H; 2: Choline O-acetyltransferase; 3: 5-hydroxytryptamine receptor 1D; 4: DNA mismatch repair protein Msh2
#2 Several peptides were included at the highest binding affinity possible; but fall below the desired/selected window: RPRNILYSLM (SEQ ID NO: 361), KKLMLYSLK (SEQ ID NO: 404), KSEFFMLAR (SEQ ID NO: 362), DRRFFMLAK (SEQ ID NO: 406), QARNILAWNY (SEQ ID NO: 363), PGSMLAWNK (SEQ ID NO: 407), RQELAWNLW (SEQ ID NO: 364), VILQRDRKK (SEQ ID NO: 418)
#3 No indicates
TCEM which would not be presented in vivo in the natural mutated protein and thus never exposed as targets
Binding shown in standard deviation
units

TABLE 5
Recommended grouping for application of MHC I peptides or nucleotides encoding
the same to Patient X

| | | SEQ | | | | | SEQ | | |
|---|---|---|---|---|---|---|---|---|---|
| | | ID | | | | | ID | | Human |
| Series | Peptide | NO: | Allele | Protein Target | position | TCEM | NO: | Fc | Freq# |
| 1 | PEKALKELQ | 393 | B4402 | kinesin heavy chain isoform 5C | 487 | ~~~ALKEL~ | 312 | 14 | 1.68 |
| | RFYPRAQLP | 343 | A0301 | angiomotin isoform 1 | 408 | ~~~PRAQL~ | 300 | 21 | 0.35 |
| | VILQRDRKK | 418 | C0401 | ATP-dependent RNA helicase DDX3X isoform 2 | 474 | ~~~QRDRK~ | 320 | 22 | −1.36 |
| | FKNTYRLML | 360 | B3501 | dipeptidyl peptidase 4 | 49 | ~~~TYRLM~ | 290 | 23 | −3.66 |
| | KTFMEPCWP | 335 | A0301 | kelch-like ECH-associated protein 1 | 607 | ~~~MEPCW~ | 285 | 23 | −3.66 |
| | STCWKEPTK | 424 | C0401 | nephrocystin-4 isoform a | 37 | ~~~WKEPT~ | 331 | 23 | −3.16 |
| | EPSMLAWNK | 341 | A0301 | peroxisomal acyl-coenzyme A oxidase 1 isoform a | 122 | ~~~MLAWN~ | 298 | 23 | −3.66 |
| | KGEIEEIVY | 374 | B3501 | phosphatidylinositol 3 | 17 | ~~~IEEIV~ | 328 | 23 | −0.47 |
| | KCFFDKCSD | 349 | A0301 | symplekin | 1062 | ~~~FDKCS~ | 315 | 24 | −3.16 |
| | GHFELPEEM | 390 | B4402 | coiled-coil domain-containing protein 50 long | 117 | ~~~ELPEE~ | 307 | 21 | 1.17 |
| | | | | isoform | | | | | |
| 2 | YVRKELAVQ | 348 | A0301 | kinesin heavy chain isoform 5C | 489 | ~~~KELAV~ | 314 | 16 | 0.15 |
| | EEVPRAQLP | 388 | B4402 | angiomotin isoform 1 | 408 | ~~~PRAQL~ | 300 | 21 | 0.35 |
| | KPYKEALHP | 352 | A0301 | ATP-dependent RNA helicase DDX3X isoform 2 | 478 | ~~~KEALH~ | 324 | 20 | −0.61 |
| | DAEEKELPY | 366 | B3501 | coiled-coil domain-containing protein 50 long | 115 | ~~~EKELP~ | 305 | 22 | 0.65 |
| | | | | isoform | | | | | |
| | TETTYRLMV | 381 | B4402 | dipeptidyl peptidase 4 | 49 | ~~~TYRLM~ | 290 | 23 | −3.66 |
| | RPSWKQIDF | 359 | B3501 | kelch-like ECH -associated protein 1 | 611 | ~~~WKQID~ | 289 | 24 | −3.66 |
| | IYREPTAFS | 426 | C0401 | nephrocystin-4 isoform a | 39 | ~~~EPTAF~ | 333 | 22 | −0.77 |
| | QARMLAWNY | 363 | B3501 | peroxisomal acyl-coenzyme A oxidase 1 isoform a | 122 | ~~~MLAWN~ | 298 | 23 | −3.66 |
| | PTCIEEIVK | 422 | C0401 | phosphatidylinositol 3 | 17 | ~~~IEEIV~ | 328 | 23 | −0.47 |
| | LCLFDKCSS | 395 | B4402 | symplekin | 1062 | ~~~FDKCS~ | 315 | 24 | −3.16 |
| 3 | HGVEPCWKI | 357 | B3501 | kelch-like ECH-associated protein 1 | 608 | ~~~EPCWK~ | 286 | 24 | −0.96 |
| | KLKPRAQLL | 408 | C0401 | angiomotin isoform 1 | 408 | ~~~PRAQL~ | 300 | 21 | 0.35 |
| | AYDKEALHL | 420 | C0401 | ATP-dependent RNA helicase DDX3X isoform 2 | 478 | ~~~KEALH~ | 324 | 20 | −0.61 |
| | REELMLYSQ | 382 | B4402 | dipeptidyl peptidase 4 | 52 | ~~~LMLYS~ | 293 | 18 | −2.45 |
| | MKGPCWKQF | 358 | B3501 | kelch-like ECH-associated protein 1 | 609 | ~~~PCWKQ~ | 287 | 24 | −3.66 |
| | VEFLKELAR | 347 | A0301 | kinesin heavy chain isoform 5C | 488 | ~~~LKELA~ | 313 | 21 | 1.13 |
| | IVRPTAFQQ | 356 | A0301 | nephrocystin-4 isoform a | 40 | ~~~PTAFQ~ | 334 | 22 | 0.29 |
| | SGPMLAWNR | 386 | B4402 | peroxisomal acyl-coenzyme A oxidase 1 isoform a | 122 | ~~~MLAWN~ | 298 | 23 | −3.66 |
| | REATAIIEE | 397 | B4402 | phosphatidylinositol 3 | 14 | ~~~TAIIE~ | 325 | 20 | −0.20 |
| | QWRFDKCSQ | 415 | C0401 | symplekin | 1062 | ~~~FDKCS~ | 315 | 24 | −3.16 |
| 4 | TEVLAWNLK | 387 | B4402 | peroxisomal acyl-coenzyme A oxidase 1 isoform a | 123 | ~~~LAWNL~ | 299 | 23 | −1.08 |
| | ASLEPCWKH | 427 | B4402 | kelch-like ECH-associated protein 1 | 608 | ~~~EPCWK~ | 286 | 24 | −0.96 |
| | KGSEKELPQ | 345 | A0301 | coiled-coil domain-containing protein 50 long | 115 | ~~~EKELP~ | 305 | 22 | 0.65 |
| | | | | isoform | | | | | |
| | LDPYRLMLK | 338 | A0301 | dipeptidyl peptidase 4 | 50 | ~~~YRLML~ | 291 | 23 | −0.61 |
| | SLRQLSSAL | 410 | C0401 | angiomotin isoform 1 | 411 | ~~~QLSSA~ | 303 | 18 | 1.43 |
| | FDWDRKEAG | 419 | C0401 | ATP-dependent RNA helicase DDX3X isoform 2 476 | 476 | ~~~DRKEA~ | 322 | 22 | −0.20 |
| | CERWKQIDD | 380 | B4402 | kelch-like ECH-associated protein 1 | 611 | ~~~WKQID~ | 289 | 24 | −3.66 |
| | KSQLKELAY | 370 | B3501 | kinesin heavy chain isoform 5C | 488 | ~~~LKELA~ | 313 | 21 | 1.13 |
| | NSAPTAFQF | 377 | B3501 | nephrocystin-4 isoform a | 40 | ~~~PTAFQ~ | 334 | 22 | 0.29 |
| | AAVTAIIEK | 421 | C0401 | phosphatidylinositol 3 | 14 | ~~~TAIIE~ | 325 | 20 | −0.20 |
| | VCFDKCSEQ | 350 | A0301 | symplekin | 1063 | ~~~DKCSE~ | 316 | 23 | −1.21 |
| 5 | PGSMLAWNK | 407 | C0401 | peroxisomal acyl-coenzyme A oxidase 1 isoform a | 122 | ~~~MLAWN~ | 298 | 23 | −3.66 |
| | RQELAWNLW | 364 | B3501 | peroxisomal acyl-coenzyme A oxidase 1 isoform a | 123 | ~~~LAWNL~ | 299 | 23 | −1.08 |
| | RRFEEIVSD | 423 | C0401 | phosphatidylinositol 3 | 18 | ~~~EEIVS~ | 329 | 21 | 0.47 |
| | DCSRAQLSA | 389 | B4402 | angiomotin isoform 1 | 409 | ~~~RAQLS~ | 301 | 17 | 0.15 |
| | TQSMLYSLK | 339 | A0301 | dipeptidyl peptidase 4 | 53 | ~~~MLYSL~ | 294 | 20 | −1.54 |
| | VESPCWKQS | 378 | B4402 | kelch-like ECH-associated protein 1 | 609 | ~~~PCWKQ~ | 287 | 24 | −3.66 |
| | QCELKELAT | 394 | B4402 | kinesin heavy chain isoform 5C | 488 | ~~~LKELA~ | 313 | 21 | 1.13 |
| | DDRFFMLAK | 340 | A0301 | peroxisomal acyl-coenzyme A oxidase 1 isoform | 120 | ~~~FFMLA~ | 296 | 24 | −3.16 |
| | | | | a | | | | | |
| | KCKEEIVSP | 353 | A0301 | phosphatidylinositol 3 | 16 | ~~~IIEET~ | 327 | 22 | 0.32 |
| | LQHDKCSEK | 416 | C0401 | symplekin | 1063 | ~~~DKCSE~ | 316 | 23 | −1.21 |
| | DLIPEEKKF | 367 | B3501 | coiled-coil domain-containing protein 50 long | 119 | ~~~PEEKK~ | 309 | 23 | 1.53 |
| | | | | isoform | | | | | |
| 6 | AEKFMLAWE | 385 | B4402 | peroxisomal acyl-coenzyme A oxidase 1 isoform | 121 | ~~~FMLAW~ | 297 | 24 | −0.86 |
| | | | | a | | | | | |
| | LELRAQLSS | 409 | C0401 | angiomotin isoform 1 | 409 | ~~~RAQLS~ | 301 | 17 | 0.15 |
| | GGYPEEKKP | 346 | A0301 | coiled-coil domain-containing protein 50 long | 119 | ~~~PEEKK~ | 309 | 23 | 1.53 |
| | | | | isoform | | | | | |
| | RPRMLYSLM | 361 | B3501 | dipeptidyl peptidase 4 | 53 | ~~~MLYSL~ | 294 | 20 | −1.54 |
| | PFDWKQIDP | 401 | C0401 | kelch-like ECH-associated protein 1 | 611 | ~~~WKQID~ | 289 | 24 | −3.66 |
| | YQMLKELAP | 414 | C0401 | kinesin heavy chain isoform 5C | 488 | ~~~LKELA~ | 313 | 21 | 1.13 |
| | MYLKEPTAK | 355 | A0301 | nephrocystin-4 isoform a | 38 | ~~~KEPTA~ | 332 | 19 | −0.69 |
| | KSEFFMLAR | 362 | B3501 | peroxisomal acyl-coenzyme A oxidase 1 isoform | 120 | ~~~FFMLA~ | 296 | 24 | −3.16 |
| | | | | a | | | | | |
| | LAEIIEEIH | 373 | B3501 | phosphatidylinositol 3 | 16 | ~~~IIEEI~ | 327 | 22 | 0.32 |
| | LEECSELRT | 396 | B4402 | symplekin | 1065 | ~~~CSELR~ | 318 | 16 | −0.25 |
| 7 | AGKEPCWKP | 336 | A0301 | kelch-like ECH-associated protein 1 | 608 | ~~~EPCWK~ | 286 | 24 | −0.96 |
| | RRYLMLYSK | 403 | C0401 | dipeptidyl peptidase 4 | 52 | ~~~LMLYS~ | 293 | 18 | −2.45 |
| | EGRLSSASK | 344 | A0301 | angiomotin isoform 1 | 412 | ~~~LSSAS~ | 304 | 13 | 2.11 |
| | IERMLYSLR | 383 | B4402 | dipeptidyl peptidase 4 | 53 | ~~~MLYSL~ | 294 | 20 | −1.54 |
| | LLRPCWKQA | 400 | C0401 | kelch-like ECH-associated protein 1 | 609 | ~~~PCWKQ~ | 287 | 24 | −3.66 |
| | VLRQALKEF | 368 | B3501 | kine sin heavy chain isoform 5C | 486 | ~~~QALKE~ | 311 | 21 | 1.61 |
| | LPSKEPTAA | 376 | B3501 | nephrocystin-4 isoform a | 38 | ~~~KEPTA~ | 332 | 19 | −0.69 |
| | EEQFFMLAQ | 384 | B4402 | peroxisomal acyl-coenzyme A oxidase 1 isoform | 120 | ~~~FFMLA~ | 296 | 24 | −3.16 |
| | | | | a | | | | | |
| | EEHIIEEID | 398 | B4402 | phosphatidylinositol 3 | 16 | ~~~IIEEI~ | 327 | 22 | 0.32 |
| | DVMKC SELP | 351 | A0301 | symplekin | 1064 | ~~~KCSEL~ | 317 | 16 | 0.08 |
| 8 | APFEKELPR | 411 | C0401 | coiled-coil domain-containing protein 50 long | 115 | ~~~EKELP~ | 305 | 22 | 0.65 |
| | | | | isoform | | | | | |
| | WLQLPEEKW | 428 | C0401 | coiled-coil domain-containing protein 50 long | 118 | ~~~LPEEK~ | 308 | 22 | 1.02 |
| | | | | isoform | | | | | |
| | KNVL SSASW | 365 | B3501 | angiomotin isoform 1 | 412 | ~~~LSSAS~ | 304 | 13 | 2.11 |
| | VELPEEKKS | 391 | B4402 | coiled-coil domain-containing protein 50 long | 119 | ~~~PEEKK~ | 309 | 23 | 1.53 |
| | | | | isoform | | | | | |
| | KKLMLYSLK | 404 | C0401 | dipeptidyl peptidase 4 | 53 | ~~~MLYSL~ | 294 | 20 | −1.54 |
| | LAACWKQIK | 337 | A0301 | kelch-like ECH-associated protein 1 | 610 | ~~~CWKQI~ | 288 | 24 | −2.04 |
| | FESQALKEV | 392 | B4402 | kinesin heavy chain isoform 5C | 486 | ~~~QALKE~ | 311 | 21 | 1.61 |
| | AMKKEPTAL | 425 | C0401 | nephrocystin-4 isoform a | 38 | ~~~KEPTA~ | 332 | 19 | −0.69 |
| | DRRFFMLAK | 406 | C0401 | peroxisomal acyl-coenzyme A oxidase 1 isoform | 120 | ~~~FFMLA~ | 296 | 24 | −3.16 |
| | | | | a | | | | | |
| | DAWEEIVSY | 354 | A0301 | phosphatidylinositol 3 | 18 | ~~~EEIVS~ | 329 | 21 | 0.47 |
| | RSNKCSELY | 372 | B3501 | symplekin | 1064 | ~~~KCSEL~ | 317 | 16 | 0.08 |
| 9 | WPSKELAVR | 371 | B3501 | kinesin heavy chain isoform 5C | 489 | ~~~KELAV~ | 314 | 16 | 0.15 |
| | SGRLAWNLP | 342 | A0301 | peroxisomal acyl-coenzyme A oxidase 1 isoform | 123 | ~~~LAWNL~ | 299 | 23 | −1.08 |
| | | | | a | | | | | |
| | VERPTAFQG | 399 | B4402 | nephrocystin-4 isoform a | 40 | ~~~PTAFQ~ | 334 | 22 | 0.29 |
| | QCLPEEKKS | 413 | C0401 | coiled-coil domain-containing protein 50 long | 119 | ~~~PEEKK~ | 309 | 23 | 1.53 |
| | | | | isoform | | | | | |
| | SKIRLMLYS | 402 | C0401 | dipeptidyl peptidase 4 | 51 | ~~~RLMLY~ | 292 | 22 | −1.21 |
| | IERCWKQIE | 379 | B4402 | kelch-like ECH-associated protein 1 | 610 | ~~~CWKQI~ | 288 | 24 | −2.04 |
| | VGDALKELM | 369 | B3501 | kinesin heavy chain isoform 5C | 487 | ~~~ALKEL~ | 312 | 14 | 1.68 |
| | SAERFFMLK | 405 | C0401 | peroxisomal acyl-coenzyme A oxidase 1 isoform | 119 | ~~~RFFML~ | 295 | 22 | −2.04 |
| | | | | a | | | | | |
| | LGSEEIVSR | 375 | B3501 | phosphatidylinositol 3 | 18 | ~~~EEIVS~ | 329 | 21 | 0.47 |
| | LKFKCSELA | 417 | C0401 | symplekin | 1064 | ~~~KCSEL~ | 317 | 16 | 0.08 |

#Human Frequency based on a zero mean unit variance transformation of the TCEM I frequencies in the human proteome

TABLE 6
Patient X Advisory list of potential off-target binding of T cells matched to
tumor specific sites for MHC I.

| | | | | Advisory |
|---|---|---|---|---|
| | SEQ | | | vs |
| TCEM | ID | | UniProt | Immediate |
| core | NO: | Human proteome target | identifier | caution |
| LQALK | 310 | 5-hydroxytryptamine receptor 1D | 5HT1D_HUMAN | Immediate |
| | | | | caution |
| SELRE | 319 | Acid-sensing ion channel 4 | ASIC4_HUMAN | |
| ELPEE | 307 | ADAMTS-like protein 3 | ATL3_HUMAN | |
| KCSEL | 317 | A-kinase anchor protein 9 | AKAP9_HUMAN | |
| TAIIE | 325 | Amyotrophic lateral sclerosis 2 chromosomal | AL2SB_HUMAN | |
| | | region candidate gene 12 protein | | |
| ALKEL | 312 | Ankyrin repeat | ASZ1_HUMAN | |
| ELPEE | 307 | Arsenite methyltransferase | AS3MT_HUMAN | |
| LQALK | 310 | Arylsulfatase A | ARSA_HUMAN | |
| RAQLS | 301 | ATP-binding cassette sub-family A member 2 | ABCA2_HUMAN | |
| ELPEE | 307 | ATP-dependent RNA helicase DDX39A | DX39A_HUMAN | |
| LSSAS | 304 | Bromodomain adjacent to zinc finger domain protein | BAZ1A_HUMAN | |
| | | 1A | | |
| ALKEL | 312 | Butyrophilin subfamily 2 member A1 | BT2A1_HUMAN | |
| EKELP | 305 | C2 calcium-dependent domain-containing protein 4C | C2C4C_HUMAN | |
| QLSSA | 303 | C6orf182 protein | Q6P2R3_HUMAN | |
| LQALK | 310 | Calcium-activated chloride channel regulator 2 | CLCA2_HUMAN | |
| QLSSA | 303 | Centrosomal protein CEP57L1 | CE57L_HUMAN | |
| ALKEL | 312 | Centrosomal protein of 120 kDa | CE120_HUMAN | |
| KELPE | 306 | Choline O-acetyltransferase | CLAT_HUMAN | Immediate |
| | | | | caution |
| QALKE | 311 | Cleavage and polyadenylation specific factor 3-like | C9IYS7_HUMAN | |
| KCSEL | 317 | Coiled-coil domain-containing protein 150 | CC150_HUMAN | |
| AQLSS | 302 | Complement factor H | CFAH_HUMAN | Immediate |
| | | | | caution |
| IEEIV | 328 | Conserved oligomeric Golgi complex subunit 5 | COGS_HUMAN | |
| QLSSA | 303 | Cyclic AMP-responsive element-binding protein 5 | CREB5_HUMAN | |
| QALKE | 311 | Cytochrome c oxidase subunit 4 isoform 2 | COX42_HUMAN | |
| QALKE | 311 | DEAD (Asp-Glu-Ala-Asp) box polypeptide 56 | G3V0G3_HUMAN | |
| KELPE | 306 | Decorin | PGS2_HUMAN | |
| ELPEE | 307 | Disks large-associated protein 3 | DLGP3_HUMAN | |
| SELRE | 319 | DNA mismatch repair protein Msh2 | MSH2_HUMAN | Immediate |
| | | | | caution |
| IIEEI | 327 | DNA nucleotidylexotransferase | TDT_HUMAN | |
| ALKEL | 312 | DNA-binding protein SATB2 | SATB2_HUMAN | |
| PRAQL | 300 | Down syndrome cell adhesion molecule | DSCAM_HUMAN | |
| KELAV | 314 | Dynein heavy chain 8 | DYH8_HUMAN | |
| QALKE | 311 | E1A-binding protein p400 | EP400_HUMAN | |
| QLSSA | 303 | E3 ubiquitin-protein ligase E3D | UBE3D_HUMAN | |
| EEIVS | 329 | E3 ubiquitin-protein ligase RFWD2 | RFWD2_HUMAN | |
| ALKEL | 312 | EH domain-containing protein 2 | EHD2_HUMAN | |
| QALKE | 311 | EP400 N-terminal-like protein | E400N_HUMAN | |
| EEIVS | 329 | Epidermal growth factor-like protein 6 | EGFL6_HUMAN | |
| ELPEE | 307 | Exonuclease 3′-5′ domain-containing protein 1 | EXD1_HUMAN | |
| SELRE | 319 | Fanconi anemia group D2 protein | FACD2_HUMAN | |
| ELPEE | 307 | F-box only protein 42 | FBX42_HUMAN | |
| ALKEL | 312 | Fer-l-like protein 6 | FR1L6_HUMAN | |
| QALKE | 311 | FERM domain-containing protein 4A | FRM4A_HUMAN | |
| QALKE | 311 | Filamin-A-interacting protein 1 | FLIP1_HUMAN | |
| RAQLS | 301 | G patch domain-containing protein 1 | GPTC1_HUMAN | |
| RAQLS | 301 | Golgin subfamily A member 2 | GOGA2_HUMAN | |
| QALKE | 311 | Heat shock 70 kDa protein 12A | HS12A_HUMAN | |
| ALKEL | 312 | Homeobox protein SIX4 | SIX4_HUMAN | |
| LKELA | 313 | Homeodomain-interacting protein kinase 4 | HIPK4_HUMAN | |
| ALKEL | 312 | Insulin receptor substrate 2 | IRS2_HUMAN | |
| KELAV | 314 | Insulin-like growth factor-binding protein 2 | IBP2_HUMAN | |
| QALKE | 311 | Integrator complex subunit 11 | INT11_HUMAN | |
| ELPEE | 307 | Integrin alpha-L | ITAL_HUMAN | |
| SELRE | 319 | JmjC domain-containing protein 7 | JMJD7_HUMAN | |
| LSSAS | 304 | Kinesin-like protein KIF7 | KIF7_HUMAN | |
| QALKE | 311 | Leucine-rich repeat and IQ domain-containing | LRIQ4_HUMAN | |
| | | protein 4 | | |
| ALKEL | 312 | Leucine-rich repeat-containing protein 18 | LRC18_HUMAN | |
| LKELA | 313 | Leucine-rich repeat-containing protein 7 | LRRC7_HUMAN | |
| EEIVS | 329 | Leucine-rich repeat-containing protein 8C | LRC8C_HUMAN | |
| IEEIV | 328 | Leucine-rich repeat-containing protein 8C | LRC8C_HUMAN | |
| ALKEL | 312 | Low-density lipoprotein receptor-related protein 8 | LRP8_HUMAN | |
| ALKEL | 312 | Meiosis-specific nuclear structural protein 1 | MNS1_HUMAN | |
| IIEEI | 327 | Metal transporter CNNM1 | CNNM1_HUMAN | |
| ALKEL | 312 | Mitogen-activated protein kinase kinase kinase 4 | M3K4_HUMAN | |
| SELRE | 319 | Mixed lineage kinase domain-like protein | MLKL_HUMAN | |
| LSSAS | 304 | Mucin-16 | MUC16_HUMAN | |
| EEIVS | 329 | NACHT | NALP9_HUMAN | |
| LSSAS | 304 | Neurobeachin-like protein 1 | NBEL1_HUMAN | |
| SELRE | 319 | Neuron navigator 3 | NAV3_HUMAN | |
| KELPE | 306 | Niban-like protein 2 | MOQZV9_HUMAN | |
| KELPE | 306 | Niban-like protein 2 | MOQZV9_HUMAN | |
| LMLYS | 293 | Nodal homolog | NODAL_HUMAN | |
| LSSAS | 304 | Olfactory receptor 14A16 | O14AG_HUMAN | |
| ALKEL | 312 | Origin recognition complex subunit 4 | ORC4_HUMAN | |
| QALKE | 311 | Peroxisomal 2 | H7C078_HUMAN | |
| LSSAS | 304 | PERQ amino acid-rich with GYF domain-containing | PERQ1_HUMAN | |
| | | protein 1 | | |
| LKELA | 313 | PH and SEC7 domain-containing protein 1 | PSD1_HUMAN | |
| LAWNL | 299 | Placenta-specific protein 9 | PLAC9_HUMAN | |
| AQLSS | 302 | Pleckstrin homology domain-containing family H | PKHH1_HUMAN | |
| | | member 1 | | |
| ALKEL | 312 | Polycystic kidney disease protein 1-like 3 | PK1L3_HUMAN | |
| QALKE | 311 | Probable ATP-dependent RNA helicase DDX56 | DDX56_HUMAN | |
| KELPE | 306 | Probable ATP-dependent RNA helicase DHX37 | DHX37_HUMAN | |
| SELRE | 319 | Probable C-mannosyltransfemse DPY19L3 | D19L3_HUMAN | |
| ALKEL | 312 | Probable phospholipid-transporting ATPase IK | AT8B3_HUMAN | |
| KELPE | 306 | Programmed cell death 6-interacting protein | PDC61_HUMAN | |
| SELRE | 319 | Pro-interleukin-16 | IL16_HUMAN | |
| PTAFQ | 334 | Protein bassoon | BSN_HUMAN | |
| AQLSS | 302 | Protein dopey-1 | DOP1_HUMAN | |
| ELPEE | 307 | Protein FAM178B | F178B_HUMAN | |
| RAQLS | 301 | Protein FAM189A1 | F1891_HUMAN | |
| EPTAF | 333 | Protein-tyrosine kinase 2-beta | FAK2_HUMAN | |
| KCSEL | 317 | P-selectin | LYAM3_HUMAN | |
| EKELP | 305 | Putative ankyrin repeat domain-containing | ANR31_HUMAN | |
| | | protein 31 | | |
| PRAQL | 300 | Putative uncharacterized protein CXorf49 | CX049_HUMAN | |
| ELPEE | 307 | Ras-associating and dilute domain-containing | RADIL_HUMAN | |
| | | protein | | |
| SELRE | 319 | Ras-related protein Rab-43 | RAB43_HUMAN | |
| ELPEE | 307 | Regulatory solute carrier protein family 1 member 1 | RSCA1_HUMAN | |
| ALKEL | 312 | Regulatory solute carrier protein family 1 member 1 | RSCA1_HUMAN | |
| AQLSS | 302 | Rho GTPase-activating protein 40 | RHG40_HUMAN | |
| IIEEI | 327 | RING finger protein 17 | RNF17_HUMAN | |
| LSSAS | 304 | Rotatin | RTTN_HUMAN | |
| ELPEE | 307 | Sarcalumenin | I3L4D6_HUMAN | |
| LKELA | 313 | Schlafen-like protein 1 | SLNL1_HUMAN | |
| ELPEE | 307 | Solute carrier family 26 member 10 | E9PIH7_HUMAN | |
| LSSAS | 304 | Sorting nexin-5 | U3KQP5_HUMAN | |
| ALKEL | 312 | Spectrin beta chain | SPTN4_HUMAN | |
| ALKEL | 312 | Spectrin beta chain | MOQZQ3_HUMAN | |
| LQALK | 310 | Spermatogenesis-associated protein 31E1 | S31E1_HUMAN | |
| PRAQL | 300 | Sulfide quinone oxidoreductase | SQRD_HUMAN | |
| ELPEE | 307 | Syndecan-3 | SDC3_HUMAN | |
| QALKE | 311 | Threonine--tRNA ligase | U3KQG0_HUMAN | |
| QALKE | 311 | Threonine--tRNA ligase | SYTM_HUMAN | |
| LSSAS | 304 | TNF receptor-associated factor 1 | TRAF1_HUMAN | |
| AQLSS | 302 | Transcription factor Spl | SP1_HUMAN | |
| SELRE | 319 | Tudor domain-containing protein 7 | TDRD7_HUMAN | |
| AQLSS | 302 | Uncharacterized protein C16orf59 | CP059_HUMAN | |
| KELPE | 306 | Vitamin D-binding protein | D6RBJ7_HUMAN | |
| EKELP | 305 | Zinc finger and SCAN domain-containing protein 25 | ZSC25_HUMAN | |
| PEEKK | 309 | Zinc finger C3H1 domain-containing protein | ZC3H1_HUMAN | |
| LSSAS | 304 | Zinc finger protein 184 | ZN184_HUMAN | |
| LSSAS | 304 | Zinc finger protein 469 | ZN469_HUMAN | |
| ELPEE | 307 | Zinc finger protein 541 | ZN541_HUMAN | |
| ELPEE | 307 | Zinc finger protein castor homolog 1 | CASZ1_HUMAN | |

TABLE 7
Caution list for Patient X MHC I TCEM

| | SEQ | Protein sharing | | |
|---|---|---|---|---|
| TCEM | ID | TCEM bound by | UniProt | Basis for caution: Potential adverse effects |
| core | NO: | patient alleles | Identifier | described in Uniprot (abbreviated) |
| LQALK | 310 | 5-hydroxytryptamine | 5HT1D_HUMAN | G-protein coupled receptor for |
| | | receptor 1D | | 5-hydroxytryptamine (serotonin). Also |
| | | | | functions as a receptor for ergot alkaloid |
| | | | | derivatives, various anxiolytic and |
| | | | | antidepressant drugs and other psychoactive |
| | | | | substances. Regulates the release of |
| | | | | 5-hydroxytryptamine in the brain, and |
| | | | | thereby affects neural activity. May also |
| | | | | play a role in regulating the release of |
| | | | | other neurotransmitters. May play a role |
| | | | | in vasoconstriction. No documented pathology |
| | | | | from deficiency. |
| KELPE | 306 | Choline O- | CLAT_HUMAN | Myasthenic syndrome, congenital, 6, |
| | | acetyltransferase | | presynaptic (CMS6) Catalyzes the reversible |
| | | | | synthesis of acetylcholine (ACh) from acetyl |
| | | | | CoA and choline at cholinergic synapses |
| | | | | The disease is caused by mutations affecting |
| | | | | this gene. A form of congenital myasthenic |
| | | | | syndrome, a group of disorders characterized |
| | | | | by failure of neuromuscular transmission, |
| | | | | including pre-synaptic, synaptic, and |
| | | | | post-synaptic disorders that are not of |
| | | | | autoimmune origin. |
| AQLSS | 302 | Complement factor H | CFAH_HUMAN | Basal laminar drusen (BLD): Glycoprotein |
| | | | | that plays an essential role in maintaining |
| | | | | a well-balanced immune response by modulating |
| | | | | complement activation. Acts as a soluble |
| | | | | inhibitor of complement. The gene represented |
| | | | | in this entry is involved in disease |
| | | | | pathogenesis. Drusen are extracellular |
| | | | | deposits that accumulate below the retinal |
| | | | | pigment epithelium on Bruch membrane. |
| | | | | Complement factor H deficiency (CFHD): A |
| | | | | disorder that can manifest as several |
| | | | | different phenotypes, including asymptomatic, |
| | | | | recurrent bacterial infections, and renal |
| | | | | failure. It is associated with a number of |
| | | | | renal diseases with variable clinical |
| | | | | presentation and progression, including |
| | | | | membranoproliferative glomerulonephritis |
| | | | | and atypical hemolytic uremic syndrome. |
| SELRE | 319 | DNA mismatch repair | MSH2_HUMAN | Hereditary non-polyposis colorectal cancer 1 |
| | | protein Msh2 | | (HNPCC1): Component of the post-replicative |
| | | | | DNA mismatch repair system (MMR) Associated |
| | | | | with an autosomal dominant disease associated |
| | | | | with marked increase in cancer susceptibility. |
| | | | | HNPCC is reported to be the most common form |
| | | | | of inherited colorectal cancer in the |
| | | | | Western world. |

TABLE 8
TCEM Allele combinations and selected peptides for each designed to stimulate
CD4 T cells in Patient X

| | | | | SEQ | | SEQ | DRB0401 | | SEQ | DRB0701 |
|---|---|---|---|---|---|---|---|---|---|---|
| Protein curation and | aa | | | ID | DRB0401 | ID | Predicted | DRB0701 | ID | Predicted |
| reference sequence | Mut | position | TCEM IIA | NO: | Simulated | NO: | Affinity | Simulated | NO: | Affinity |
| kelch-like ECH- | W | 609 | CW~Q~DQ | 429 | MLMWCWKQIDQNHQY | 465 | −1.85 | no | | |
| associated protein 1 | W | 610 | WK~I~QQ | 430 | no | | | ISRLWKQIDQQYIIA | 493 | −1.93 |
| gi:22027642 | | | | | | | | | | |
| dipeptidyl | M | 46 | KN~Y~LM | 431 | QIIYKNTYRLMLDGL | 466 | −1.49 | HRIFKNTYRLMVLLH | 494 | −1.95 |
| peptidase 4 | M | 47 | NT~R~ML | 432 | QIWSNTYRLMLTTVG | 467 | −1.51 | no | | |
| gi:18765694 | M | 49 | YR~M~YS | 433 | DSLGYRLMLYSDQGD | 468 | −2.03 | FAFRYRLMLYSKQEF | 495 | −1.95 |
| | M | 51 | LM~Y~LR | 434 | TLPHLMLYSLRSENG | 469 | −1.87 | MQPQLMLYSLRIDKV | 496 | −1.85 |
| | M | 52 | ML~S~RW | 435 | RKIHMLYSLRWGLAQ | 470 | −2.11 | RQRQMLYSLRWVDRF | 497 | −1.75 |
| peroxisomal acyl- | L | 116 | QE~F~ML | 436 | no | | | RQIRQERFFMLQRYF | 498 | −1.63 |
| coenzyme A | L | 117 | ER~F~LA | 437 | no | | | KMLEERFFMLAKLYP | 499 | −1.88 |
| oxidase 1 isoform a | L | 119 | FF~L~WN | 438 | RDIQFFMLAWNHQDL | 471 | −1.98 | RPQFFFMLAWNNRLR | 500 | −2.03 |
| gi:30089972 | L | 121 | ML~W~LE | 439 | SYTFMLAWNLESDTE | 472 | −1.92 | QVSHMLAWNLEFIQE | 501 | −1.99 |
| | L | 122 | LA~N~EI | 440 | SYMRLAWNLEISSEI | 473 | −1.85 | RRWLLAWNLEICDLD | 502 | −1.99 |
| angiomotin | L | 406 | MP~A~LS | 441 | LPLLMPRAQLSQSQD | 474 | −1.91 | no | | |
| isoform 1 | L | 408 | RA~L~SA | 442 | QRFQRAQLSSAATPL | 475 | −1.78 | GVYLRAQLSSAPIPA | 503 | −2.01 |
| gi:166064029 | L | 410 | QL~S~SY | 443 | DQMWQLSSASYLDTT | 476 | −1.91 | FEFLQLSSASYAHCR | 504 | −1.94 |
| | L | 411 | LS~A~YQ | 444 | PALILSSASYQNSLP | 477 | −1.97 | KFLHLSSASYQWRIM | 505 | −2.03 |
| coiled-coil domain- | P | 112 | LQ~K~LP | 445 | TLLILQEKELPALNT | 478 | −1.91 | no | | |
| containing protein | P | 113 | QE~E~PE | 446 | YSMWQEKELPELSYS | 479 | −1.19 | no | | |
| 50 long isoform | P | 115 | KE~P~EK | 447 | AYLFKELPEEKDDDK | 480 | −2.04 | no | | |
| gi:41281911 | P | 117 | LP~E~KR | 448 | PKLFLPEEKKRPQLP | 481 | −2.11 | no | | |
| | P | 118 | PE~K~RK | 449 | LFHYPEEKKRKNLRK | 482 | −1.09 | no | | |
| kinesin heavy chain | K | 482 | EV~Q~LK | 450 | SLNLEVLQALKGTGL | 483 | −1.93 | PTLPEVLQALKAMLE | 506 | −2.09 |
| isoform 5C | K | 483 | VL~A~KE | 451 | no | | | RMPLVLQALKEVRSI | 507 | −2.12 |
| gi:4758650 | K | 485 | QA~K~LA | 452 | FSRWQALKELALARP | 484 | −1.99 | KSLFQALKELALNPV | 508 | −2.02 |
| | K | 487 | LK~L~VN | 453 | FLNILKELAVNLTQD | 485 | −1.83 | HEKMLKELAVNPNFL | 509 | −2.07 |
| | K | 488 | KE~A~NY | 454 | SIQWKELAVNYYKKE | 486 | −1.90 | ELWYKELAVNYWRLP | 510 | −1.97 |
| symplekin gi: | S | 1062 | DK~S~LR | 455 | FILLDKCSELRADTP | 487 | −1.86 | no | | |
| 124028529 | S | 1065 | SE~R~PL | 456 | no | | | HELWSELREPLLLIS | 511 | −2.09 |
| phosphatidylinositol | E | 12 | MT~I~EE | 457 | ALKMMTAIIEELSPS | 488 | −2.01 | RYLTMTAIIEEYDVL | 512 | −1.81 |
| 3 | E | 14 | AI~E~IV | 458 | ARFFAIIEEIVQEAE | 489 | −1.79 | no | | |
| gi:73765544 | E | 16 | IE~I~SR | 459 | MWLRIEEIVSRNSDL | 490 | −1.81 | no | | |
| | E | 17 | EE~V~RN | 460 | PDLWEEIVSRNLQLA | 491 | −1.82 | no | | |
| nephrocystin-4 | P | 34 | QP~K~PT | 461 | no | | | SLNVQPWKEPTLVIM | 513 | −2.00 |
| isoform a | P | 36 | WK~P~AF | 462 | no | | | DKFRWKEPTAFFKVC | 514 | −1.91 |
| gi:23510323 | P | 38 | EP~A~QC | 463 | no | | | LKKQEPTAFQCLLII | 515 | −1.64 |
| | P | 39 | PT~F~CV | 464 | DFYVPTAFQCVPKTQ | 492 | −1.90 | KPLYPTAFQCVPYQM | 516 | −1.91 |

#“No” indicates TCEM which would not be presented in vivo in the natural mutated protein and thus never exposed as targets
No immediate cautions were detected in the MHC II call list

TABLE 9
Recommended grouping for application of MHC II peptides or nucleotides
encoding the same to Patient X

| | | SEQ | | | | | SEQ | | |
|---|---|---|---|---|---|---|---|---|---|
| Sub | | ID | | | | | ID | | Human |
| groups | Peptide | NO: | Allele | Protein Target | position | TCEM | NO: | Fc | Freq |
| 1 | QRFQRAQLSSAATPL | 475 | DRB0401 | angiomotin isoform 1 | 408 | RA~L~SA | 442 | 18 | 0.99 |
| | AYLFKELPEEKDDDK | 480 | DRB0401 | coiled-coil domain- | 115 | KE~P~EK | | 23 | −0.06 |
| | | | | containing protein | | | | | |
| | RQRQMLYSLRWVDRF | 497 | DRB0701 | dipeptidyl peptidase 4 | 52 | ML~S~RW | 447 | 24 | −1.54 |
| | ISRLWKQIDQQYIIA | 493 | DRB0701 | kelch-like ECH-associated | 610 | WK~I~QQ | 430 | 23 | −3.62 |
| | | | | protein 1 | | | | | |
| | FLNILKELAVNLTQD | 485 | DRB0401 | kinesin heavy chain isoform | 487 | LK~L~VN | 453 | 23 | 0.54 |
| | | | | 5C | | | | | |
| | KSLFQALKELALNPV | 508 | DRB0701 | kinesin heavy chain isoform | 485 | QA~K~LA | 452 | 21 | 1.11 |
| | | | | 5C | | | | | |
| | LKKQEPTAFQCLLII | 515 | DRB0701 | nephrocystin-4 isoform a | 38 | EP~A~QC | 463 | 21 | −3.12 |
| | RPQFFFMLAWNNRLR | 500 | DRB0701 | peroxisomal acyl-coenzyme | 119 | FF~L~WN | 438 | 23 | −3.12 |
| | | | | A oxidase 1 | | | | | |
| | ALKMMTAIIEELSPS | 488 | DRB0401 | phosphatidylinositol 3 | 12 | MT~I~EE | 457 | 23 | −3.62 |
| | FILLDKCSELRADTP | 487 | DRB0401 | symplekin | 1062 | DK~S~LR | 455 | 22 | 0.60 |
| 2 | GVYLRAQLSSAPIPA | 503 | DRB0701 | angiomotin isoform 1 | 408 | RA~L~SA | 442 | 18 | 0.99 |
| | LFHYPEEKKRKNLRK | 482 | DRB0401 | coiled-coil domain- | 118 | PE~K~RK | 449 | 22 | 0.19 |
| | | | | containing protein 50 | | | | | |
| | DSLGYRLMLYSDQGD | 468 | DRB0401 | dipeptidyl peptidase 4 | 49 | YR~M~YS | 433 | 11 | −3.62 |
| | FAFRYRLMLYSKQEF | 495 | DRB0701 | dipeptidyl peptidase 4 | 49 | YR~M~YS | 433 | 11 | −3.62 |
| | QIIYKNTYRLMLDGL | 466 | DRB0401 | dipeptidyl peptidase 4 | 46 | KN~Y~LM | 431 | 21 | −1.08 |
| | MLMWCWKQIDQNHQY | 465 | DRB0401 | kelch-like ECH-associated | 609 | CW~Q~DQ | 429 | 23 | −1.08 |
| | | | | protein 1 | | | | | |
| | HEKMLKELAVNPNFL | 509 | DRB0701 | kinesin heavy chain isoform | 487 | LK~L~VN | 453 | 23 | 0.54 |
| | | | | 5C | | | | | |
| | DFYVPTAFQCVPKTQ | 492 | DRB0401 | nephrocystin-4 isoform a | 39 | PT~F~CV | 464 | 23 | −2.04 |
| | RDIQFFMLAWNHQDL | 471 | DRB0401 | peroxisomal acyl-coenzyme | 119 | FF~L~WN | 438 | 23 | −3.12 |
| | | | | A oxidase 1 | | | | | |
| | RYLTMTAIIEEYDVL | 512 | DRB0701 | phosphatidylinositol 3 | 12 | MT~I~EE | 457 | 23 | −3.62 |
| | HELWSELREPLLLIS | 511 | DRB0701 | symplekin | 1065 | SE~R~PL | 456 | 21 | 1.16 |
| 3 | FEFLQLSSASYAHCR | 504 | DRB0701 | angiomotin isoform 1 | 410 | QL~S~SY | 443 | 16 | −0.24 |
| | PALILSSASYQNSLP | 477 | DRB0401 | angiomotin isoform 1 | 411 | LS~A~YQ | 444 | 14 | 0.13 |
| | YSMWQEKELPELSYS | 479 | DRB0401 | coiled-coil domain- | 113 | QE~E~PE | 446 | 22 | 0.82 |
| | | | | containing protein 50 | | | | | |
| | QIWSNTYRLMLTTVG | 467 | DRB0401 | dipeptidyl peptidase 4 | 47 | NT~R~ML | 432 | 23 | −2.44 |
| | HRIFKNTYRLMVLLH | 494 | DRB0701 | dipeptidyl peptidase 4 | 46 | KN~Y~LM | 431 | 21 | −1.08 |
| | RKIHMLYSLRWGLAQ | 470 | DRB0401 | dipeptidyl peptidase 4 | 52 | ML~S~RW | 435 | 24 | −1.54 |
| | SLNLEVLQALKGTGL | 483 | DRB0401 | kinesin heavy chain isoform | 482 | EV~Q~LK | 450 | 23 | 1.04 |
| | SLNVQPWKEPTLVIM | 513 | DRB0701 | nephrocystin-4 isoform a | 34 | QP~K~PT | 461 | 22 | −2.04 |
| | RQIRQERFFMLQRYF | 498 | DRB0701 | peroxisomal acyl-coenzyme | 116 | QE~F~ML | 436 | 22 | −2.04 |
| | | | | 5CA oxidase 1 | | | | | |
| | PDLWEEIVSRNLQLA | 491 | DRB0401 | phosphatidylinositol 3 | 17 | EE~V~RN | 460 | 20 | −0.19 |
| 4 | LPLLMPRAQLSQSQD | 474 | DRB0401 | angiomotin isoform 1 | 406 | MP~A~LS | 441 | 21 | −0.06 |
| | TLLILQEKELPALNT | 478 | DRB0401 | coiled-coil domain- | 112 | LQ~K~LP | 445 | 22 | |
| | | | | containing protein 50 | | | | | |
| | TLPHLMLYSLRSENG | 469 | DRB0401 | dipeptidyl peptidase 4 | 51 | LM~Y~LR | 434 | 19 | −2.04 |
| | PTLPEVLQALKAMLE | 506 | DRB0701 | kinesin heavy chain isoform | 482 | EV~Q~LK | 450 | 23 | 1.04 |
| | | | | 5C | | | | | |
| | ELWYKELAVNYWRLP | 510 | DRB0701 | kinesin heavy chain isoform | 488 | KE~A~NY | 454 | 16 | −0.41 |
| | | | | 5C | | | | | |
| | RMPLVLQALKEVRSI | 507 | DRB0701 | kinesin heavy chain isoform | 483 | VL~A~KE | 451 | 22 | 1.11 |
| | | | | 5C | | | | | |
| | KPLYPTAFQCVPYQM | 516 | DRB0701 | nephrocystin-4 isoform a | 39 | PT~F~CV | 464 | 23 | −2.04 |
| | SYTFMLAWNLESDTE | 472 | DRB0401 | peroxisomal acyl-coenzyme | 121 | ML~W~LE | 439 | 22 | −1.75 |
| | | | | A oxidase 1 | | | | | |
| | RRWLLAWNLEICDLD | 502 | DRB0701 | peroxisomal acyl-coenzyme | 122 | LA~N~EI | 440 | 22 | −0.02 |
| | | | | A oxidase 1 | | | | | |
| | SYMRLAWNLEISSEI | 473 | DRB0401 | peroxisomal acyl-coenzyme | 122 | LA~N~EI | 440 | 22 | −0.02 |
| | | | | A oxidase 1 | | | | | |
| | MWLRIEEIVSRNSDL | 490 | DRB0401 | phosphatidylinositol 3 | 16 | IE~I~SR | 459 | 22 | −0.02 |
| 5 | DQMWQLSSASYLDTT | 476 | DRB0401 | angiomotin isoform 1 | 410 | QL~S~SY | 443 | 16 | −0.24 |
| | KFLHLSSASYQWRIM | 505 | DRB0701 | angiomotin isoform 1 | 411 | LS~A~YQ | 444 | 14 | 0.13 |
| | PKLFLPEEKKRPQLP | 481 | DRB0401 | coiled-coil domain- | 117 | LP~E~KR | 448 | 21 | 1.05 |
| | | | | containing protein 50 | | | | | |
| | MQPQLMLYSLRIDKV | 496 | DRB0701 | dipeptidyl peptidase 4 | 51 | LM~Y~LR | 434 | 19 | −2.04 |
| | SIQWKELAVNYYKKE | 486 | DRB0401 | kinesin heavy chain isoform | 488 | KE~A~NY | 454 | 16 | −0.41 |
| | | | | 5C | | | | | |
| | FSRWQALKELALARP | 484 | DRB0401 | kinesin heavy chain isoform | 485 | QA~K~LA | 452 | 21 | 1.11 |
| | | | | 5C | | | | | |
| | DKFRWKEPTAFFKVC | 514 | DRB0701 | nephrocystin-4 isoform a | 36 | WK~P~AF | 462 | 19 | −1.75 |
| | QVSHMLAWNLEFIQE | 501 | DRB0701 | peroxisomal acyl-coenzyme | 121 | ML~W~LE | 439 | 22 | −1.75 |
| | | | | A oxidase 1 | | | | | |
| | KMLEERFFMLAKLYP | 499 | DRB0701 | peroxisomal acyl-coenzyme | 117 | ER~F~LA | 437 | 16 | 0.39 |
| | | | | A oxidase 1 | | | | | |
| | ARFFAIIEEIVQEAE | 489 | DRB0401 | phosphatidylinositol 3 | 14 | AI~E~IV | 458 | 18 | −0.24 |

TABLE 10
Patient X Advisory list for potential MHC II off target effects;
no immediate cautions were flagged

| TCEM II A | SEQ ID | | UniProt |
|---|---|---|---|
| Motif | NO: | Protein Annotation | Identifier |
| RD~K~AL | 517 | 5-hydroxytryptamine receptor 6 | 5HT6R_HUMAN |
| SE~R~PL | 456 | Actin-binding protein anillin | ANLN_HUMAN |
| LQ~K~LP | 445 | AF4 FMR2 family member 2 | AFF2_HUMAN |
| ER~F~LA | 437 | Alanine aminotransferase 1 | ALAT1_HUMAN |
| EV~Q~LK | 450 | Aldehyde oxidase | ADO_HUMAN |
| QL~S~SY | 443 | CDK5 regulatory subunit-associated protein 3 | J3KS63_HUMAN |
| LK~L~VN | 453 | Cellular retinoic acid-binding protein 1 | RABP1_HUMAN |
| LK~L~VN | 453 | Cellular retinoic acid-binding protein 2 | RABP2_HUMAN |
| VL~A~KE | 451 | Centrosomal protein of 152 kDa | CE152_HUMAN |
| RA~L~SA | 442 | Coiled-coil domain-containing protein 9 | CCDC9_HUMAN |
| EV~Q~LK | 450 | Copine-3 | CPNE3_HUMAN |
| QE~E~PE | 446 | Cullin-4A | CUL4A_HUMAN |
| AI~E~IV | 458 | DNA polymerase theta | DPOLQ_HUMAN |
| KE~L~QF | 518 | E3 ubiquitin-protein ligase UBR4 | UBR4_HUMAN |
| MP~A~LS | 441 | Endoplasmic reticulum-Golgi intermediate compartment | ERGI3_HUMAN |
| | | protein 3 | |
| LQ~K~LP | 445 | ERC protein 2 | ERC2_HUMAN |
| KE~P~EK | 447 | Ermin | ERMIN_HUMAN |
| MP~A~LS | 441 | Gamma-glutamyltranspeptidase 1 | GGT1_HUMAN |
| MP~A~LS | 441 | Gamma-glutamyltranspeptidase 2 | GGT2_HUMAN |
| QE~E~PE | 446 | General transcription factor IIF subunit 1 | T2FA_HUMAN |
| LK~L~VN | 453 | Glycerophosphocholine phosphodiesterase GPCPD1 | GPCPl_HUMAN |
| MP~A~LS | 441 | GPI mannosyltransferase 4 | PIGZ_HUMAN |
| AI~E~IV | 458 | HCF N-terminal chain 5 | A6NEM2_HUMAN |
| AI~E~IV | 458 | Host cell factor 1 | HCFC1_HUMAN |
| KE~L~QF | 518 | Inositol hexakisphosphate kinase 2 | IP6K2_HUMAN |
| RA~L~SA | 442 | Inverted formin-2 | INF2_HUMAN |
| ML~W~LE | 439 | Laminin subunit alpha-3 | LAMA3_HUMAN |
| RA~L~SA | 442 | MAP kinase-interacting serine_threonine-protein kinase 2 | MKNK2_HUMAN |
| LK~L~VN | 453 | Midasin | MDN1_HUMAN |
| CS~L~EP | 519 | N-acetyltransferase ESCO1 | ESCO1_HUMAN |
| LQ~K~LP | 445 | Oxysterols receptor LXR-beta | NR1H2_HUMAN |
| LP~E~KR | 448 | Probable G-protein coupled receptor 111 | GP111_HUMAN |
| MP~A~LS | 441 | Probable G-protein coupled receptor 61 | GPR61_HUMAN |
| LS~A~YQ | 444 | Prolyl 4-hydroxylase subunit alpha-1 | P4HA1_HUMAN |
| RS~R~RK | 520 | Protamine-2 | PRM2_HUMAN |
| AV~D~CS | 521 | Protein NOV homolog | NOV_HUMAN |
| QE~E~PE | 446 | Protein phosphatase 1 regulatory subunit 14B | F5H2U0_HUMAN |
| RS~R~RK | 520 | Receptor-binding cancer antigen expressed on SiSo cells | RCAS1_HUMAN |
| VL~A~KE | 451 | SH3 domain-binding protein 5 | 3BP5_HUMAN |
| LK~L~VN | 453 | Small nuclear ribonucleoprotein polypeptide A′ | H0YKK0_HUMAN |
| ML~W~LE | 439 | Spectrin beta chain | SPTN5_HUMAN |
| QA~K~LA | 452 | Talin-1 | TLN1_HUMAN |
| QL~S~SY | 443 | Taste receptor type 2 member 5 | TA2R5_HUMAN |
| LK~L~VN | 453 | Transcription factor BTF3 | BTF3_HUMAN |
| RD~K~AL | 517 | Transducin beta-like protein 3 | TBL3_HUMAN |
| RA~L~SA | 442 | Translation initiation factor eIF-2B subunit alpha | EI2BA_HUMAN |
| LK~L~VN | 453 | Tubulin-specific chaperone E | TBCE_HUMAN |
| LK~L~VN | 453 | U2 small nuclear ribonucleoprotein A′ | RU2A_HUMAN |
| VL~A~KE | 451 | Ubiquitin carboxyl-terminal hydrolase 19 | UBP19_HUMAN |
| DK~S~LR | 455 | Ubiquitin thioesterase ZRANB1 | ZRAN1_HUMAN |
| ML~W~LE | 439 | Zinc finger BED domain-containing protein 6 | ZBED6_HUMAN |
| QL~S~SY | 443 | Zinc finger protein PLAGL2 | PLAL2_HUMAN |

### Example 5: Increasing Personalized T Cell Targeting Options for Melanoma Patients

A recent report documented a group of patients with metastatic melanoma, whose biopsies were sequenced and mutations identified in several proteins [29]. Peptides encompassing the mutations were produced and used to identify T cells reactive to MHC tetramers carrying the peptides of interest, demonstrating that T cell populations reactive to epitopes in the cancer expressed proteins were generated at detectable levels. Tetramers were only available for a limited number of HLA. Peptides were selected based on the predicted MHC binding using publicly available algorithms. A limited number of peptides comprising the mutated amino acids were identified which bound to the A0201, A0101 and A1101 alleles carried by these patients. However, very few of the peptides had the mutated amino acid located in a position which would expose that amino acid to the TCR. Hence the T cell responses would not have differentiated tumor-mutated from normal protein. Using this patient data, we addressed the question of whether peptides could be generated which would potentially stimulate cytotoxic T cells targeting tumor cells, within the limited allele and mutation information available. The natural binding affinity for the mutant protein did not permit selection of peptides that would bind MHC and achieve this differential targeting by exposing T cell exposed motifs containing the mutant amino acids. We therefore sought to design peptides which could bind the available MHC with sufficient affinity and expose the mutated amino acids. Affinity predictions were generated for all sequential peptides in each protein. This allowed identification of which T cell exposed motifs comprising the mutated amino acid had any likelihood of being transiently bound in an MHC. For these TCEM we then generated 10,000 simulated peptides for each allele TCEM combination, by changing the four flanking amino acids which determine binding, and identified peptides with sufficient binding affinity which could be used as vaccine components to stimulate T cells cognate for the presented TCEM. We document below for each patient how the limited information does permit potential tumor targeting neoantigens to be created which would stimulate CTLs targeting the tumor, and enabling the preparation of a multi-peptide vaccine targeting the melanoma of these patients.

Patient A: Patient A is A0201; other alleles are unknown. This patient has mutations in SPRX (sushi repeat-containing protein SRPX isoform 1 precursor) and WDR46 (WD repeat-containing protein 46 isoform 1). The mutation in SPRX is a P to L at position 1275 in SPRX; T cells reactive to a peptide TLWCSPIKV were identified. The mutation in WDR46 is a T to I at position 300 and T cells reactive to peptide FLIYLDVSV were identified. In both cases the mutant amino acid is in a binding position not exposed to the TCR. We identified the TCEM comprising the mutant peptides and generated simulated peptides for A0201 designed to stimulate cytotoxic T cells to each of the 5 TCEM which have the mutant amino acid and are exposed to the TCR. Simulation of 10,000 peptides, after elimination of duplicates and non-binders, generated 2,417 unique peptides which correspond to TCEM that would be presented by A0201 naturally, and which are soluble. Of these, 88 are predicted to bind better than 1.5 SD below the mean for the protein and collectively these peptides encode the 6 different TCEM which would be naturally presented from these two proteins and which contain the mutated amino acids differentiating the tumor from the normal protein. One exemplar peptide simulated and its predicted binding for A0201 is shown below for each TCEM core. A vaccine comprising these 6 peptides would elicit CTL targeting Patient A's melanoma.

TABLE 11
Patient A peptides.

| | | | | SEQ | Affinity | | SEQ |
|---|---|---|---|---|---|---|---|
| | Posi- | | Proposed | ID | for | TCEM Core | ID |
| ID | tion | Protein curation | peptide | NO: | A0201# | pentamer | NO: |
| 256773176- | 294 | WDR46-WD repeat- | KDKGFLIYV | 522 | −1.53 | GFLIY | 528 |
| 63-mut | 296 | containing protein | KMKLIYLDA | 523 | −2.27 | LIYLD | 529 |
| | 297 | 46 isoform 1 | KLKIYLDVG | 524 | −1.51 | IYLDV | 530 |
| 5454086- | 48 | SRPX-sushi repeat- | KLLYKDTLV | 525 | −2.35 | YKDTL | 531 |
| 292- mut | 51 | containing protein | KMSTLWCSG | 526 | −2.01 | TLWCS | 532 |
| | 52 | SRPX isoform 1 | KLRLWCSPA | 527 | −1.53 | LWCSP | 533 |
| | | precursor | | | | | |

#Affinity predicted in standard deviation units below the mean for the respective protein

Patient B: Patient B carries alleles A0201 and A1101; other alleles are unknown. This patient has mutations in NSDHL (sterol-4-alpha-carboxylate 3-dehydrogenase). The mutation in NSDHL is a A to V at position 290 and T cells were identified that are cognate for a peptide ILTGLNYEV. The mutant amino acid is in a binding position not exposed to the TCR. We identified the TCEM comprising the mutant peptides and generated simulated peptides for A0201 and A1101 designed to stimulate cytotoxic T cells to each of the 5 TCEM which have the mutant amino acid and are exposed to the TCR. Simulation of 10,000 peptides for each allele, after elimination of duplicates and non-binders, generated 3,046 peptides which correspond to the only TCEM (~YEVPK~) (SEQ ID NO: 534) that would be presented by A0201 naturally, and which are soluble. Of these, 445 bind better than −1.5 SD below the mean. One peptide was selected as shown in Table 12 below. The same process generated 13,306 peptides which bind

A1101 across 4 TCEM (~~~LNYEV~ (SEQ ID NO: 535), ~~~NYEVP~ (SEQ ID NO: 536), ~~~YEVPK~ (SEQ ID NO: 534), ~~~EVPKY~ (SEQ ID NO: 537)) which would be naturally presented. Of these, 3,514 bind better than −1.5 SD below the mean. A vaccine comprising these 5 peptides would elicit CTL targeting Patient B's melanoma.

TABLE 12
Patient B peptides.

| | | | | | SEQ | Affinity | TCEM | SEQ |
|---|---|---|---|---|---|---|---|---|
| | | | | Proposed | ID | for | Core | ID |
| ID | Position | Protein curation | Allele | peptide | NO: | allele# | pentamer | NO: |
| 8393516- | 283 | NSDHL-sterol-4- | A1101 | LELLNYEVK | 538 | −2.00 | LNYEV | 535 |
| 151-mut | 284 | alpha-carboxylate | A1101 | KATNYEVPR | 539 | −2.05 | NYEVP | 536 |
| | 285 | 3-dehydrogenase | A1101 | SIVYEVPKP | 540 | −2.02 | YEVPK | 534 |
| | 285 | | A0201 | DMLYEVPKI | 541 | −2.10 | YEVPK | 534 |
| | 286 | | A1101 | TIAEVPKYR | 542 | −2.14 | EVPKY | 537 |

#Affinity predicted in standard deviation units below the mean for the respective protein

Patient C: Patient C is A0201; other alleles are unknown. This patient has mutations in ERBB2 (receptor tyrosine-protein kinase erbB-2 isoform a precursor), COL181A (collagen alpha-1 (XVIII) chain isoform 1 preproprotein), and TEAD1 (transcriptional enhancer factor TEF-). The mutation in ERBB2 is a H to Y at position 473 and T cells were identified with a peptide ALIHHNTYL (SEQ ID NO: 543). The mutation in TEAD1 is a L to F at position 388 and T cells were identified with peptides VLENFTIFLV (SEQ ID NO: 544) and SVLENFTIFL (SEQ ID NO: 545). COL181A is mutated S to F at position 306 and T cells were identified with VLLGVKLEGV (SEQ ID NO: 546). The mutant amino acid is only not exposed to the TCR in VLENFTIFLV (SEQ ID NO: 544) and VLLGVKLEGV (SEQ ID NO: 546), utilizing in each case only one of 5 potential peptides due to the limitations of natural binding. We identified the TCEM comprising the mutant peptides and generated simulated peptides for A0201 designed to stimulate cytotoxic T cells to each of the 5 TCEM in each protein which have the mutant amino acid and are exposed to the TCR. Simulation of 10,000 peptides for each protein, after elimination of duplicates and non-binders, generated 13,425 peptides which correspond to TCEM that would be presented by A0201 naturally, and which are soluble. Of these 1200 bind better than −1.5 SD below the mean and represent 14 different TCEM. Table 13 below shows 14 peptides simulated to bind at approximately 2 SD below the mean where possible. For two TCEM positions where there were no peptides generated that bind at this affinity; the two highest affinity peptides are shown. A vaccine comprising these 14 peptides would elicit CTL targeting Patient C's melanoma.

TABLE 13
Patient C peptides.

| | | | | SEQ | Affinity | TCEM | SEQ |
|---|---|---|---|---|---|---|---|
| | | | Proposed | ID | for | Core | ID |
| ID | Position | Protein curation | peptide | NO: | A0201 | pentamer | NO: |
| 110611235- | 299 | COL18A1-collagen | QMRGVKLFG | 547 | −2.03 | GVKLF | 561 |
| 186-mut | 300 | alpha-1(XVIII) chain | GMDVKLFGG | 548 | −1.96 | VKLFG | 562 |
| | 302 | isoform 1 | RMRLFGVQA | 549 | −1.97 | LFGVQ | 563 |
| | 303 | preproprotein | KLVFGVQDA | 550 | −2.01 | FGVQD | 564 |
| 54792096-5- | 466 | ERBB2-receptor | GMIHHNTYG | 551 | -2.01 | HHNTY | 565 |
| mut | 467 | tyrosine-protein | QTIHNTYLV | 552 | -2.00 | HNTYL | 566 |
| | 468 | kinase erbB-2 | KGVNTYLCV | 553 | −2.08 | NTYLC | 567 |
| | 469 | isoform a | KLRTYLCFS | 554 | −2.01 | TYLCF | 568 |
| | 470 | precursor | KQQYLCFVG | 555 | −1.04 | YLCFV | 569 |
| 296434319- | 378 | TEAD1-transcriptional | RLKVLENFV | 556 | -2.10 | VLENF | 570 |
| 303-mut | 379 | enhancer factor TEF-1 | ALPLENFTG | 557 | -1.98 | LENFT | 571 |
| | 380 | | YSAENFTIV | 558 | −2.02 | ENFTI | 572 |
| | 381 | | KTPNFTIFA | 559 | −2.06 | NFTIF | 573 |
| | 382 | | RQKFTIFLG | 560 | −0.87 | FTIFL | 574 |

Patient D: Patient D carries A0101 and A0201; other alleles are unknown. This patient has mutations in GANAB (neutral alpha-glucosidase AB isoform 2 precursor). The mutation in a S to F at position 298 and T cells were originally identified with a peptide ALYGEVPVL (SEQ ID NO: 575). In this instance, the mutant amino acid is exposed to the TCR. We identified all the TCEM comprising the mutant peptides and generated simulated peptides for A0101 and A0201 designed to stimulate cytotoxic T cells to each of the 5 TCEM for each protein which have the mutant amino acid and are exposed to the TCR. 10,000 peptides were simulated for each TCEM/allele combination, after elimination of duplicates and non-binders, generated 2713 peptides which correspond to TCEM that would be presented by A0101 or A0201 naturally, and which are soluble. Of those binding better than −1.0 SD below the mean 5 different TCEM are represented. Notably there were no very high binders generated for A0201 among the 50,000 original peptides simulated, with only 33 of −1 SD or better; This underscores the difficulty of finding naturally binding peptides which allow targeting of mutants and the value of simulation to maximize potential binding allele TCEM combinations which can stimulate appropriate T cells. Representative simulated peptides are shown in Table 14. A vaccine comprising these 7 peptides would elicit CTL targeting Patient D's melanoma.

TABLE 14
Patient D peptides

| | | | | | SEQ | Affinity | | SEQ |
|---|---|---|---|---|---|---|---|---|
| | | Protein | | Proposed | ID | for | TCEM Core | ID |
| ID | Position | curation | Allele | peptide | NO: | Allele | pentamer | NO: |
| 38202257- | 291 | GANAB- | A0201 | RTRALYGFV | 576 | −1.35 | ALYGF | 583 |
| 12-mut | 292 | neutral | A0101 | SSDLYGFVR | 577 | −2.03 | LYGFV | 584 |
| | 292 | alpha- | A0201 | KDELYGFVV | 578 | −1.13 | LYGFV | 585 |
| | 293 | glucosidase | A0101 | LADYGFVPD | 579 | −2.06 | YGFVP | 586 |
| | 293 | AB isoform | A0201 | KLRYGFVPA | 580 | −1.88 | YGFVP | 587 |
| | 294 | 2 precursor | A0201 | KVDGFVPVA | 581 | −1.55 | GFVPV | 588 |
| | 295 | | A0101 | RSDFVPVLN | 582 | −2.04 | FVPVL | 589 |

Patient E: Patient E is A0101; other alleles are unknown. This patient has mutations in TRIP12 (E3 ubiquitin-protein ligase TRIP12 isoform a). The mutation in a F to S at position 1592 and was originally targeted with a peptide PSDTRQMLFY (SEQ ID NO: 590). The mutant amino acid in this peptide is not exposed to the TCR. We identified the TCEM comprising the mutant peptides and generated simulated peptides for A0101 designed to stimulate cytotoxic T cells to each of the 5 TCEM in which have the mutant amino acid is exposed to the TCR. However, we found that only 1 of the TCEM would be naturally presented in the context of the mutant protein. This and the fact we have only one known allele only provides one combination which can generate relevant T cells. However, even this single peptide is an advantage over the naturally bound peptides which do not expose the mutated amino acid and which therefore would generate T cells which cannot differentiate the tumor from normal cells. Over 4000 unique peptides were simulated for this single TCEM which are soluble; 650 of these bind better than −1.5 SD below the mean for the protein. Three peptides were selected with different predicted affinities (Table 15). These have essentially the same function but illustrate that the desired binding can be selected from the bank of simulated peptides.

TABLE 15
Patient E peptides

| | | | | SEQ | Affinity | TCEM | SEQ |
|---|---|---|---|---|---|---|---|
| | | Protein | Proposed | ID | for | Core | ID |
| ID | Position | curation | peptide | NO: | A0101 | pentamer | NO: |
| 545746335- | 1586 | E3 ubiquitin- | EGDFFPSDP | 591 | −3.00 | FFPSD | 594 |
| 228-mut | 1586 | protein ligase | FEDFFPSDL | 592 | −2.02 | FFPSD | 594 |
| | 1586 | TRIP12 | WDDFFPSDE | 593 | −1.75 | FFPSD | 594 |

### Example 6: Personalized Neoepitope Peptides for Small Cell Lung Cancer

A recent report of a small cell lung cancer case, in which the patient alleles were well documented, identified mutations in five proteins (EGFR, STK11, NAV3, EPHB1 and PTCH2) [11]. Four of these were simple amino acid substitutions; STK11 was a frameshift. Peptides had been selected for use as neoantigens, but notably several of the peptides placed the mutated amino acid in a binding pocket position. This means that the T cell exposed motif in the mutant and the wildtype protein is unchanged. We therefore elected to explore whether additional peptides could be generated which provide high binding to the patient alleles and also place the mutant amino acid in a position exposed to the T cell to allow a differential response between mutant and wildtype proteins. Small cell lung cancers are often associated with highly mutated proteins, requiring a personalized approach to neoepitope vaccination.

We elected to design an array of vaccine or T cell stimulating peptides or encoding nucleic acids for this patient (Patient Y). For the four proteins with simple SNP mutations we assembled wildtype and mutant sequences and determined the predicted binding of all sequential peptides. Based on this we determined which TCEM comprising the mutant amino acid would be presented in vivo in this patient as the result of binding of the flanking region by the patient's alleles to expose that amino acid in the T cell exposed motif. We then identified the TCEM comprising the mutant amino acid and generated an array of 1000 peptides for each TCEM with randomly replaced flanking amino acids. Peptides were selected based on predicted affinity, solubility and likelihood of stimulating T cells which target naturally presented mutant TCEM. Peptides were selected to have a predicted affinity near 2 SD below the mean of the respective proteins; however other affinities may be selected and so this example is not considered limiting. Table 15 below summarizes the findings for the four proteins and this patient's MHC I and MHC II alleles.

Simulated binding peptides were then selected for each TCEM allele combination in the desired predicted affinity range. These are shown for MHC I in Table 16 and for MHC II in Table 17.

The TCEM for both MHC I and II were mapped onto the human protein reference database to review potential off target effects. A total of 348 unique proteins comprised TCEM which would be presented as the result of binding and presentation by one or more of Patient Ys alleles. This list would be provided to a clinician as the basis for a risk assessment of that patient. We do not include the complete advisory list here in the interests of space. Two proteins potentially targeted were flagged as being of immediate concern and for which T cell stimulating peptides would not be advised. These are shown in Table 18.

TABLE 15
Small cell lung cancer patient-summary of available TCEM targets and peptides available

| | Proteins with identified mutations | | | | | | |
|---|---|---|---|---|---|---|---|
| | 4 | | | | | | |
| | TCEM with mutations | | | | | | |
| | 20 | | | | | | |
| Patient Alleles | A1101 | A3101 | B3501 | C0303 | DRB0405 | DRB1501 | DQB0602 |
| TCEM naturally presented | 7 | 9 | 10 | 8 | 12 | 11 | 11 |
| for allele | | | | | | | |
| Mutated proteins in which | 3 | 4 | 3 | 3 | 3 | 4 | 4 |
| natural presentation occurs | | | | | | | |
| Proteins omitted as no | 1 | 0 | 1 | 1 | 1 | 1 | 0 |
| natural presentation | | | | | | | |
| Unique peptides simulated | 14543 | 16585 | 17952 | 17925 | 17954 | 17964 | 17816 |
| with any binding | | | | | | | |
| Subset for which TCEM | 3916 | 9872 | 11022 | 4718 | 7040 | 7175 | 7382 |
| is naturally presented | | | | | | | |
| Filtered by polarity score <1 | 2195 | 6320 | 7420 | 3920 | 4127 | 4252 | 6459 |
| indicating solubility | | | | | | | |
| Peptides selected in desired | 254 | 273 | 209 | 298 | 261 | 135 | 607 |
| binding window <−1.75> −2.25 | | | | | | | |
| Represent TCEM allele | 7 | 9 | 9 | 7 | 12 | 11 | 11 |
| combos <−1.75> −2.25 | | | | | | | |
| Removed due to immediate | 1 | 1 | 1 | 1 | 0 | 0 | 1 |
| off target caution; or high | | | | | | | |
| frequency Fc # | | | | | | | |
| Net TCEM allele combos | 7 | 8 | 8 | 6 | 12 | 11 | 11 |
| available <−1.75> −2.25 | | | | | | | |

| | | |
|---|---|---|
| Potential vaccine peptides | 29 | 33 |
| per patient for all mutated | | |
| proteins | | |

# One removed due to TCEM I match to Complement C4; One removed for DQB0602 match to Coagulation factor VIII

TABLE 16
Peptides available to stimulate CD8 T cells specific to
Patient Y mutations and alleles.

| | | | | Simulated | SEQ | | | SEQ |
|---|---|---|---|---|---|---|---|---|
| Protein | | | Allele | binding | ID | Predicted | TCEM I | ID |
| ID | Protein | Position | MHC I | peptide | NO: | Affinity | target | NO: |
| m-P00533 | EGFR_HUMAN | 852 | A_1101 | AVDDFGRAR | 595 | −2.15 | ~~~DFGRA~ | 629 |
| | Epidermal growth | 852 | A_3101 | SRDDFGRAR | 596 | −2.23 | ~~~DFGRA~ | 630 |
| | factor receptor | 853 | B-3501 | LSHFGRAKF | 597 | −1.92 | ~~~FGRAK~ | 631 |
| | | 854 | B-3501 | FAEGRAKLH | 598 | −2.07 | ~~~GRAKL~ | 632 |
| | | 855 | B-3501 | YGHRAKLLL | 599 | −2.17 | ~~~RAKLL~ | 633 |
| | | 851 | C0303 | LAITDFGRA | 600 | −2.17 | ~~~TDFGR~ | 634 |
| | | 852 | C0303 | VAADFGRAY | 601 | −1.98 | ~~~DFGRA~ | 635 |
| | | 853 | C0303 | WGIFGRAKA | 602 | −1.83 | ~~~FGRAK~ | 636 |
| | | 854 | C0303 | LEVGRAKLL | 603 | −2.16 | ~~~GRAKL~ | 637 |
| m-P54762 | EPHB1_HUMAN | 458 | A_3101 | PESSGIILR | 604 | −2.25 | ~~~SGIIL~ | 638 |
| | Ephrin type-B | 454 | B-3501 | QQMPEQPSF | 605 | −2.07 | ~~~PEQPS~ | 639 |
| | receptor 1 | 455 | B-3501 | GLCEQPSGF | 606 | −1.97 | ~~~EQPSG~ | 640 |
| | | 456 | B-3501 | HAPQPSGIF | 607 | −2.16 | ~~~QPSGI~ | 641 |
| | | 454 | C0303 | PAGPEQPST | 608 | −2.14 | ~~~PEQPS~ | 642 |
| | | 455 | C0303 | ASGEQPSGF | 609 | −2.14 | ~~~EQPSG~ | 643 |
| m-Q8IVL0 | NAV3_HUMAN Neuron | 2236 | A_1101 | SGCGPRLLR | 610 | −1.95 | ~~~GPRLL~ | 644 |
| | navigator 3 | 2237 | A_1101 | YQQPRLLLR | 611 | −1.92 | ~~~PRLLL~ | 645 |
| | | 2238 | A_1101 | NTGRLLLPP | 612 | −2.13 | ~~~RLLLP~ | 646 |
| | | 2239 | A_1101 | KTQLLLPCR | 613 | −2.01 | ~~~LLLPC~ | 647 |
| | | 2236 | A_3101 | RVTGPRLLD | 614 | −2.06 | ~~~GPRLL~ | 648 |
| | | 2237 | A_3101 | QGGPRLLLK | 615 | −2.23 | ~~~PRLLL~ | 649 |
| | | 2238 | A_3101 | SDWRLLLPK | 616 | −2.15 | ~~~RLLLP~ | 650 |
| | | 2239 | A_3101 | SHELLLPCR | 617 | −2.14 | ~~~LLLPC~ | 651 |
| | | 2236 | B-3501 | KACGPRLLY | 618 | −2.08 | ~~~GPRLL~ | 652 |
| | | 2237 | B-3501 | RGPPRLLLY | 619 | −2.17 | ~~~PRLLL~ | 653 |
| | | 2238 | B-3501 | DPTRLLLPY | 620 | −2.07 | ~~~RLLLP~ | 654 |
| | | 2236 | C0303 | PGSGPRLLS | 621 | −2.02 | ~~~GPRLL~ | 655 |
| | | 2239 | B-3501 | RSGLLLPCR | 622 | −1.30 | ~~~LLLPC~ | 656 |
| | | 2237 | C0303 | SSSPRLLLP | 623 | −1.59 | ~~~PRLLL~ | 657 |
| m-Q9Y6C5 | PTC2_HUMAN | 804 | A_1101 | RSLRHSYCR | 624 | −2.10 | ~~~RHSYC~ | 658 |
| | Protein patched | 805 | A_1101 | SKLHSYCNK | 625 | −2.06 | ~~~HSYCN~ | 659 |
| | homolog 2 | 804 | A_3101 | GARRHSYCR | 626 | −1.95 | ~~~RHSYC~ | 660 |
| | | 805 | A_3101 | PLGHSYCNR | 627 | −2.21 | ~~~HSYCN~ | 661 |
| | | 807 | A_3101 | GLTYCNGSR | 628 | −1.94 | ~~~YCNGS~ | 662 |

Predicted affinity in standard deviation units below the mean

TABLE 17
Peptides available to stimulate CD8 T cells specific to
Patient Y mutations and alleles.

| | | | | Simulated | SEQ | | | SEQ |
|---|---|---|---|---|---|---|---|---|
| | | | Allele | binding | ID | Predicted | TCEM IA | ID |
| Protein ID | Protein | Position | MHC I | peptide | NO: | Affinity | target | NO: |
| m-P00533 | EGFR_HUMAN | 848 | DQB0602 | LCMGKITDFGRANHE | 663 | −1.75 | KI~D~GR | 697 |
| | Epidermal | 848 | DRB0405 | RLVFKITDFGRENIM | 664 | −1.96 | KI~D~GR | 697 |
| | growth factor | 848 | DRB1501 | KLLLKITDFGRGQYL | 665 | −1.95 | KI~D~GR | 697 |
| | receptor | 849 | DQB0602 | LDFIITDFGRASTQT | 666 | −2.09 | IT~F~RA | 698 |
| | | 849 | DRB0405 | PGFWITDFGRAELMD | 667 | −2.00 | IT~F~RA | 698 |
| | | 849 | DRB1501 | QTLLITDFGRASMYT | 668 | −1.99 | IT~F~RA | 698 |
| | | 854 | DQB0602 | VMEKRAKLLGAPKYS | 669 | −2.00 | RA~L~GA | 699 |
| m-P54762 | EPHB1_HUMAN | 451 | DQB0602 | PHLLPQPEQPSLHYC | 670 | −1.94 | PQ~E~PS | 700 |
| | Ephrin type-B | 451 | DRB0405 | LKWFPQPEQPSIMSF | 671 | −1.59 | PQ~E~PS | 700 |
| | receptor 1 | 451 | DRB1501 | WNILPQPEQPSQILK | 672 | −1.74 | PQ~E~PS | 700 |
| | | 452 | DQB0602 | LGSAQPEQPSGSYAC | 673 | −1.69 | QP~Q~SG | 701 |
| | | 452 | DRB0405 | RLIFQPEQPSGLSIV | 674 | −1.53 | QP~Q~SG | 701 |
| | | 452 | DRB1501 | GSLMQPEQPSGLLFS | 675 | −1.27 | QP~Q~SG | 701 |
| | | 454 | DQB0602 | LSTLEQPSGIISRNS | 676 | −2.01 | EQ~S~II | 702 |
| | | 454 | DRB0405 | HPFWEQPSGIIQQID | 677 | −1.58 | EQ~S~II | 702 |
| | | 454 | DRB1501 | SYLFEQPSGIITINS | 678 | −1.75 | EQ~S~II | 702 |
| | | 456 | DRB0405 | FTWRPSGIILDNIRN | 679 | −2.20 | PS~I~LD | 703 |
| | | 457 | DQB0602 | SNPASGIILDYLKAV | 680 | −2.11 | SG~I~DY | 704 |
| | | 457 | DRB0405 | FSTYSGIILDYPRHM | 681 | −1.87 | SG~I~DY | 704 |
| | | 457 | DRB1501 | KLPISGIILDYHVDS | 682 | −1.78 | SG~I~DY | 704 |
| m-Q8IVL0 | NAV3_HUMAN | 2233 | DRB0405 | RTYMTIGPRLLIARQ | 683 | −1.97 | TI~P~LL | 705 |
| | Neuron | 2233 | DRB1501 | RLGETIGPRLLLVRQ | 684 | −2.01 | TI~P~LL | 705 |
| | navigator 3 | 2234 | DRB0405 | PKEYIGPRLLLTVQT | 685 | −1.99 | IG~R~LL | 706 |
| | | 2234 | DRB1501 | DSRFIGPRLLLPSAN | 686 | −2.04 | IG~R~LL | 706 |
| | | 2236 | DRB0405 | NILTPRLLLPCPECE | 687 | −2.10 | PR~L~PC | 707 |
| | | 2236 | DRB1501 | DEYLPRLLLPCAQYD | 688 | −2.09 | PR~L~PC | 707 |
| | | 2238 | DRB0405 | YEEYLLLPCPMPRTA | 689 | −2.15 | LL~P~PM | 708 |
| | | 2238 | DRB1501 | PRRPLLLPCPMQTAT | 690 | −2.00 | LL~P~PM | 708 |
| | | 2239 | DQB0602 | QQVSLLPCPMDPEFS | 691 | −2.12 | LL~C~MD | 709 |
| | | 2239 | DRB0405 | NGILLLPCPMDSSES | 692 | −1.98 | LL~C~MD | 709 |
| | | 2239 | DRB1501 | KHSLLLPCPMDKVLD | 693 | −2.01 | LL~C~MD | 709 |
| m-Q9Y6C5 | PTC2_HUMAN | 802 | DQB0602 | LCFRTRHSYCNTRTI | 694 | −1.94 | TR~S~CN | 710 |
| | Protein patched | 804 | DQB0602 | MIAGHSYCNGSVACG | 695 | −2.00 | HS~C~GS | 711 |
| | homolog 2 | 806 | DQB0602 | HEAMYCNGSEDAQIT | 696 | −2.04 | YC~G~ED | 712 |

TABLE 18
TCEMs identified as of immediate concern for Patient Y

| Protein | | TCEM | | | | | | Immediate |
|---|---|---|---|---|---|---|---|---|
| ID | pos | IIA | TCEM I | | | Protein annotation | UniProt ID | Caution |
| P0C0L5 | 18 | | ~~~PRLLL~ | I | | Complement C4-B | CO4B_HUMAN | Caution |
| | | | (SEQ ID | | | | | |
| | | | NO: 645) | | | | | |
| P0C0L4 | 18 | | ~~~PRLLL~ | I | | Complement C4-A | CO4A_HUMAN | Caution |
| | | | (SEQ ID | | | | | |
| | | | NO: 645) | | | | | |
| P00748 | 390 | HS~C~GS | | | IIA | Coagulation factor | FA12_HUMAN | Caution |
| | | (SEQ ID | | | | XII | | |
| | | NO: 711) | | | | | | |

After removal of peptides comprising these TCEM the peptides in Tables 16 and 17 provide an array of T cell stimulating peptides which could be used as a neoepitope vaccine or in vitro stimulant of autologous dendritic cells or T cells for Patient Y. This provides an example of how this approach could be used in a small cell lung cancer case. This provides many more options for stimulating T cells specifically targeting the unique tumor epitopes than reliance on naturally bound peptides.

### Example 7: Application to Common Mutations Found in Many Cancers

This example describes the generation of “ready to go” neoantigens which are applicable to patients of known alleles who share common mutations found in many cancers. While the description is provided for five proteins which have common mutations across over 30 cancers, the approach is equally applicable to other mutations shared between different cancers and thus the example should not be considered limiting. Similarly, the set of alleles selected and shown in the Example is not considered limiting and this process can be executed for other combinations of alleles.

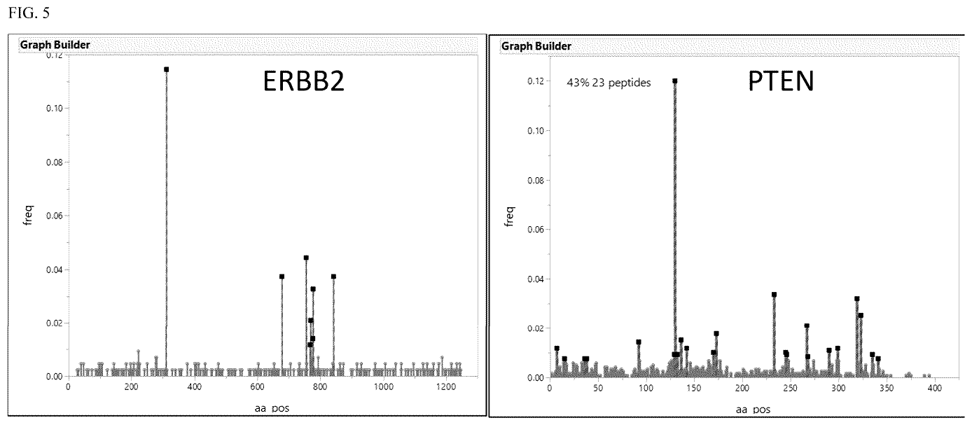

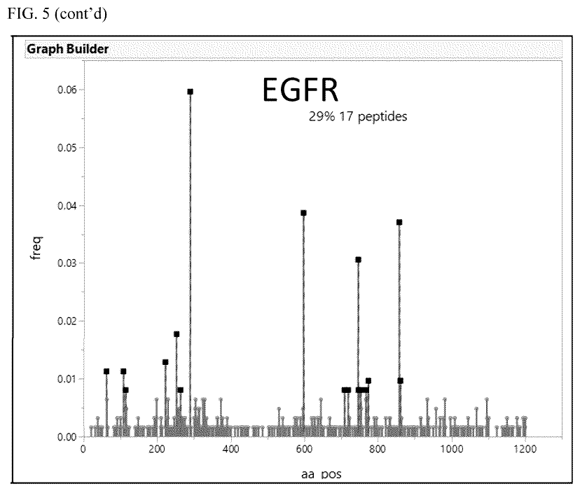

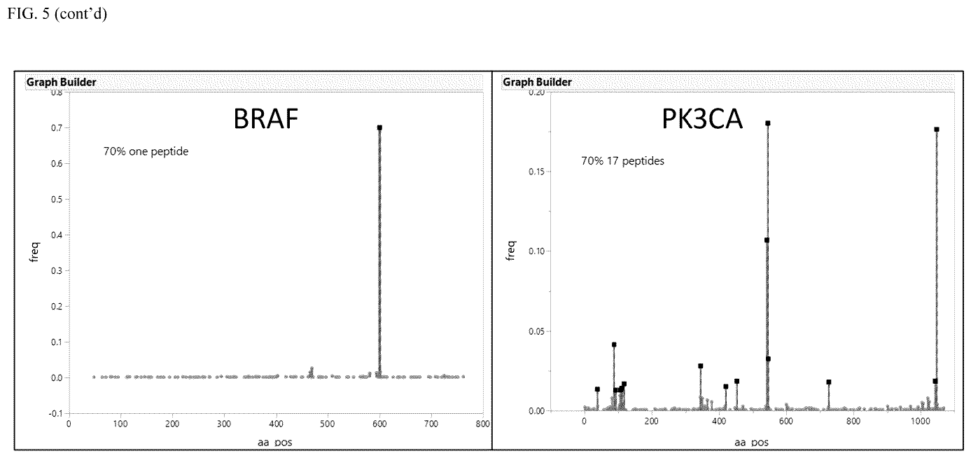

Table 19 identifies five proteins commonly mutated in many different cancers. The location of the dominant mutations is shown in FIG. 5. The preponderance of mutations at a few positions indicate it is possible to design peptides in anticipation of many different patient allele combinations, thereby providing a bank of peptides ready-to-use as soon as patient HLA typing and sequencing is available to identify mutational biomarkers.

TABLE 19
Frequency of mutations in selected proteins

| mutant type | N Rows | N, BRAF | N, EGFR | N, ERBB2 | N, PK3CA | N, PTEN |
|---|---|---|---|---|---|---|
| deletion | 116 | 7 | 29 | 1 | 19 | 7 |
| duplication | 13 | 0 | 5 | 3 | 0 | 0 |
| frameshift | 856 | 8 | 4 | 7 | 3 | 294 |
| SNP | 8604 | 898 | 583 | 417 | 1764 | 883 |
| splice | 414 | 7 | 4 | 7 | 2 | 80 |

Rows: Number of records of mutations in this protein in the TCGA at present date

In this Example we demonstrate the application of this approach to 10 common mutations in 5 proteins. These common mutations have been documented in the 32 common cancers shown in Table 20. The examples of mutated proteins, common mutation positions, and alleles we show below provide illustrations, but are not considered in any way limiting.

TABLE 20
Cancers in which mutations in the indicated proteins are documented

| Cancer type | CODE | BRAF | EGFR | ERBB | PK3CA | PTEN |
|---|---|---|---|---|---|---|
| Adrenocortical carcinoma | ACC | 1 | 4 | | 1 | 1 |
| Bladder urothelial | BLCA | 17 | 19 | 78 | 109 | 22 |
| carcinoma | | | | | | |
| Breast adenocarcinoma | BRCA | 10 | 25 | 38 | 407 | 67 |
| Cervical squamous cell | CESC | 5 | 16 | 22 | 101 | 45 |
| carcinoma | | | | | | |
| Cholangiocarcinoma | CHOL | 2 | 2 | 3 | 3 | 1 |
| Colon carcinoma | COAD | 66 | 30 | 27 | 171 | 43 |
| Diffuse Large B-cell | DLBC | 1 | | | | 2 |
| Lymphoma | | | | | | |
| Esophageal carcinoma | ESCA | 2 | 6 | 13 | 20 | 14 |
| Glioblastoma multiforme | GBM | 9 | 133 | 12 | 43 | 142 |
| Head and neck squamous | HNSC | 13 | 27 | 18 | 108 | 14 |
| cell carcinoma | | | | | | |
| Kidney Chromophobe | KICH | | 1 | | | 6 |
| kidney renal clear cell | KIRC | 1 | 2 | 4 | 5 | 15 |
| carcinoma | | | | | | |
| Kidney renal papillary | KIRP | 6 | 3 | 7 | 4 | 9 |
| cell carcinoma | | | | | | |
| Acute myeloid leukaemia | LAML | | | 1 | | |
| Brain Lower Grade Glioma | LGG | 5 | 46 | 2 | 50 | 26 |
| Liver hepatocellular | LIHC | | 9 | 3 | 13 | 10 |
| carcinoma | | | | | | |
| Lung adenocarcinoma | LUAD | 52 | 101 | 20 | 37 | 17 |
| Lung squamous cell | LUSC | 18 | 24 | 15 | 73 | 63 |
| carcinoma | | | | | | |
| Mesothelioma | MESO | 1 | | | 2 | 2 |
| Ovarian serous | OV | 2 | 13 | 6 | 11 | 7 |
| carcinoma | | | | | | |
| Pancreatic | PAAD | 2 | 2 | 6 | 5 | |
| adenocarcinoma | | | | | | |
| Pheochromocytoma and | PCPG | 1 | | | 1 | |
| Paraganglioma | | | | | | |
| Prostate adenocarcinoma | PRAD | 8 | 4 | 6 | 16 | 19 |
| Rectal carcinoma | READ | 7 | 8 | 8 | 26 | 13 |
| Sarcoma | SARC | 1 | 4 | 3 | 7 | 9 |
| Skin Cutaneous Melanoma | SKCM | 314 | 111 | 57 | 31 | 49 |
| Stomach adenocarcinoma | STAD | 25 | 35 | 41 | 102 | 53 |
| Testicular Germ Cell | TGCT | | 1 | 1 | 3 | |
| Tumors | | | | | | |
| Thyroid carcinoma | THCA | 314 | | | 5 | 3 |
| Thymoma | THYM | | | 2 | | |
| Uterine corpus | UCEC | 45 | 110 | 62 | 406 | 702 |
| endometrial carcinoma | | | | | | |
| Uterine Carcinosarcoma | UCS | 1 | 1 | 1 | 22 | 17 |

A cancer patient with one of the common mutations and a known set of alleles could benefit from the availability of a “ready to go” set of peptides designed and selected to allow stimulation of that patient's cytotoxic T cells and cross presented helper T cells. Thus once these mutations are identified, they provide a starting point for an immunotherapy approach to these cancers. The examples include both amino acid substitutions and amino acid duplications.

Predictions of binding affinity are currently made for 31 MHC I A alleles, 31 MHC IB alleles, 8 MHC I C alleles plus for MHC II predictions are currently made for 13 DP alleles or allele combinations, 28 DQ Alleles or allele combinations and 24 DRB alleles. This allows peptides to be designed for a very wide diversity of potential patients; indeed this combination of alleles represents over 85% of the world human population. Additional alleles may be added in future and the same process applied for them.

We applied predictions of MHC binding affinity and T cell exposed motif analysis methods previous developed (See e.g., PCT Appl. US 14/41523, incorporated by reference herein its entirety). From the selected commonly mutated proteins in Table 20, we chose 2 common mutation sites in each as examples. The T cell exposed motifs which comprise the mutant amino acid were identified for both CD8 and CD4 T cells. Peptides were designed to demonstrate application for 4 MHC I alleles (A0101, A2301, A3001, A8001) and 4 MHC II alleles (B2705, B3801, B4801, B5701. These are non-limiting examples, chosen to show how this approach can be generalized to all 122 alleles for which binding affinity predictions are currently performed. While CD8 T cells are critical to generating a cytotoxic response, CD4 cells may facilitate this as helper cells.

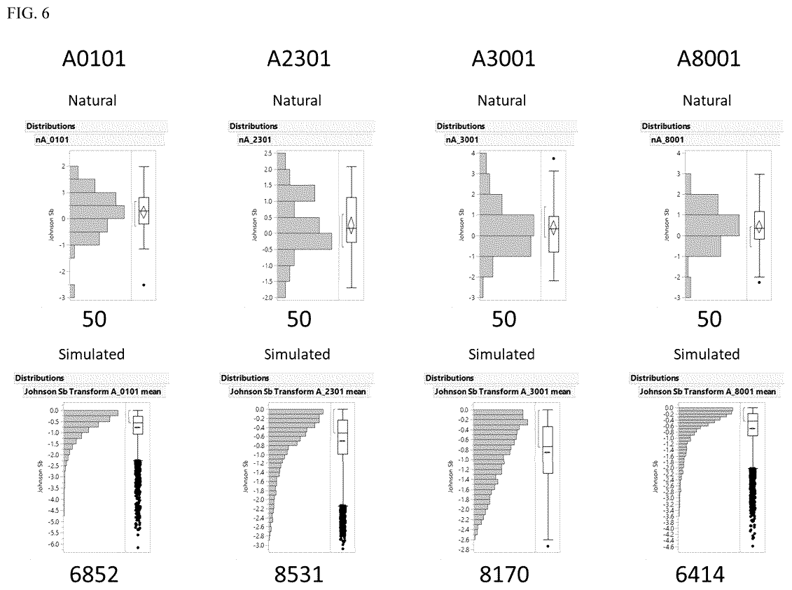

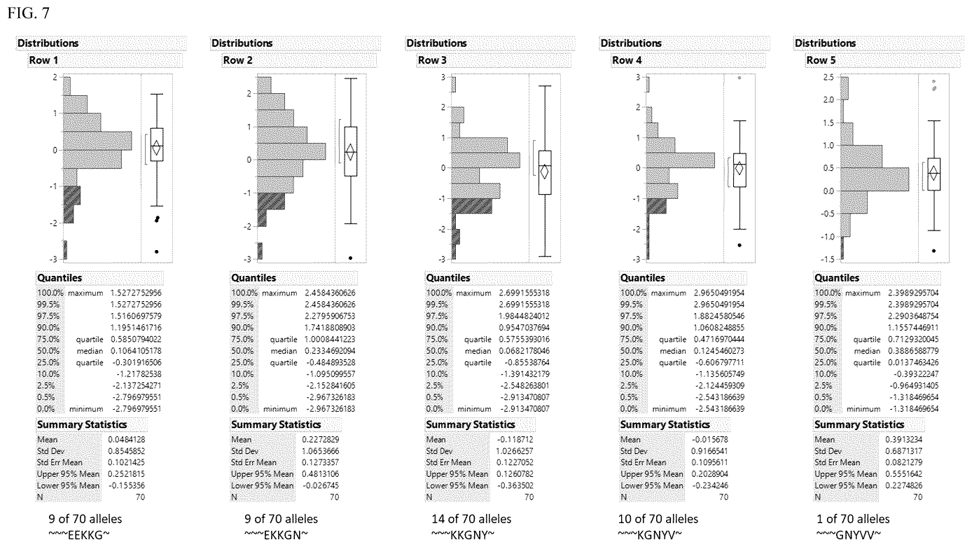

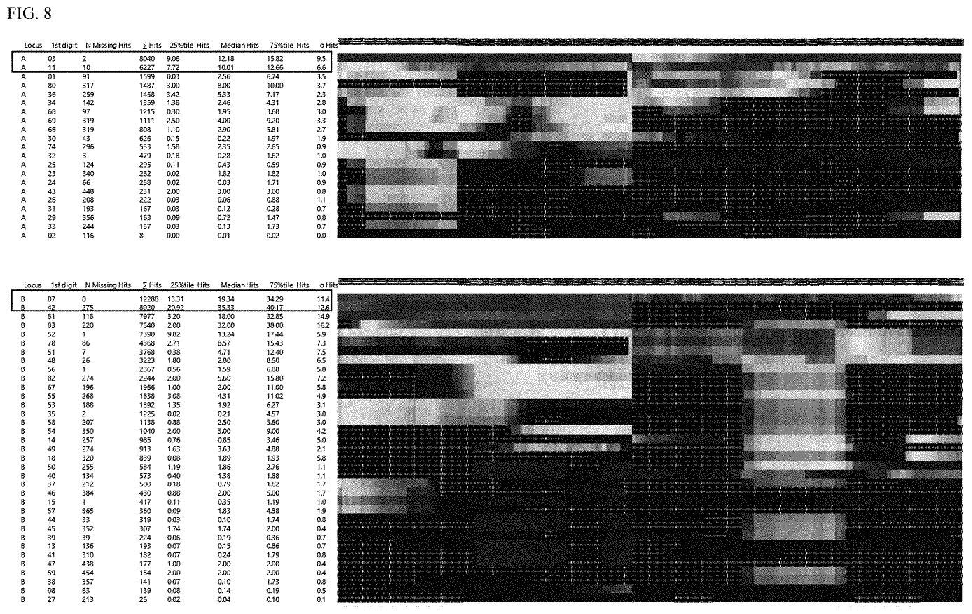

For each TCEM-selected allele combination, 1000 peptides were generated to provide a choice of high affinity peptides. Duplicates and non-binding peptides were eliminated as were potentially low solubility peptides. The choice of 1000 peptides is shown as a non-limiting example; this number could be 5000 or 10,000 or more and would result in a wider selection. Table 21 shows the number of such peptides generated for each selected allele. FIG. 6 shows (for the A alleles) how this simulation process generates peptides with a preponderance of high MHC binding relative to the native mutant peptide.

Binding affinity is measured in standard deviation units below the mean of all peptides in that protein. For example purposes, peptides with a predicted binding affinity of near 2 SD below the mean for each protein and allele-TCEM combination were selected, or as near to that as feasible. This places these peptides in the top ~5% of binding peptides relative to others in the protein. These peptides are show in in Table 22 for MHC I A example alleles, Table 23 for MHC I B example alleles, and Table 24 for MHC II example alleles. We have discussed elsewhere in the Description whether MHC binding affinities higher than this are beneficial or not; the approach we show here allows selection of peptides of whatever predicted binding affinity is desired. Hence, the criteria applied in this example are not considered limiting.

Not all proteins will have TCEM that will be accessible to T cells when the mutant protein is naturally presented, depending on the MHC alleles of that patient, but as every patient has multiple loci it is anticipated that it is possible to locate suitable peptides for every patient for one or more alleles. The peptides shown in Tables 23, 24 and 25 would elicit T cells specifically targeting the TCEM unique to the proteins with these mutations regardless of the type of cancer in which they occur. These peptides thus serve as examples of multi-cancer neoepitope peptides for use in vaccines, or as in vitro T cell stimulants. These peptides may be deployed singly or in groups together selected to stimulate T cells to target a maximum number of allele-TCEM combinations, or may be applied in groups at different time points. When used as a vaccine the peptides may be delivered intradermally, by injection or microneedle array, subcutaneously, parenterally or by any other route deemed appropriate by the clinician. The peptides may be applied in conjunction with an adjuvant or local inflammatory agent. Peptide application may be followed by a checkpoint inhibitor or other immunomodulatory intervention. The peptides may also be used in vitro to prime autologous dendritic cells or T cells that are then administered to the patient.

TABLE 21

| | Group of 5 proteins; overall 10 protein mutation combinations | | | | | | | | | | | |
|---|---|---|---|---|---|---|---|---|---|---|---|---|
| | Overall number of potential TCEM targets = 50 | | | | | | | | | | | |
| | | | | | | | | | DRB1 | DRB3 | DQB | DQB |
| Allele | A0101 | A2301 | A3001 | A8001 | B2705 | B3801 | B4801 | B5701 | 1201 | 0101 | 0302 | 0602 |
| TCEM naturally | 18 | 22 | 21 | 16 | 19 | 18 | 24 | 24 | 22 | 19 | 22 | 23 |
| presented for allele | | | | | | | | | | | | |
| # protein mutation | 9/10 | 6/10 | 9/10 | 10/10 | 10/10 | 10/10 | 10/10 | 9/10 | 8/10 | 7/10 | 7/10 | 10/10 |
| combo with TCEM | | | | | | | | | | | | |
| presented | | | | | | | | | | | | |
| Candidate peptides, | 6852 | 8531 | 8170 | 6414 | 7197 | 7608 | 9014 | 10197 | 10559 | 9784 | 13752 | 11376 |
| bound, presented | | | | | | | | | | | | |
| and soluble | | | | | | | | | | | | |
| Candidate peptides binding | 407 | 463 | 846 | 406 | 466 | 470 | 512 | 383 | 554 | 581 | 708 | 662 |
| at −1.75-2.25 SD | | | | | | | | | | | | |
| Candidate peptides | 132 | 1 | 0 | 85 | 185 | 9 | 148 | 359 | 10 | 34 | 40 | 3 |
| binding at <−3 SD | | | | | | | | | | | | |

TABLE 22
Exemplar peptides stimulating CD8 T cell responses to multi cancer
mutated proteins for the indicated MHC I A alleles

| | | | | | SEQ | | | | SEQ |
|---|---|---|---|---|---|---|---|---|---|
| | | Muta- | | Binding | ID | Predicted | | | ID |
| Protein ID | Protein Curation | tion | Allele | peptide | NO. | binding | Position | TCEM | NO. |
| P00533_A289V | EGFR_HUMAN | A289V | A_0101 | IGEYSFGVS | 713 | −2.03 | 282 | YSFGV | 1 |
| | Epidermal growth | A289V | A_0101 | KSESFGVAR | 714 | −2.02 | 283 | SFGVA | 2 |
| | factor receptor | A289V | A_0101 | FQSGVATCP | 715 | −2.02 | 285 | GVATC | 3 |
| | OS_Homo sapiens | A289V | A_0101 | DSSVATCVL | 716 | −2.01 | 286 | VATCV | 4 |
| | OX_9606 GN_EGFR | A289V | A_2301 | DKYSFGVAF | 717 | −1.74 | 283 | SFGVA | 5 |
| | PE_1 SV_2 | A289V | A_2301 | ERYFGVATL | 718 | −1.64 | 284 | FGVAT | 6 |
| | | A289V | A_2301 | HKWGVATCW | 719 | −2.11 | 285 | GVATC | 7 |
| | | A289V | A_3001 | IGRYSFGVQ | 720 | −2.00 | 282 | YSFGV | 8 |
| | | A289V | A_3001 | EQRSFGVAG | 721 | −2.00 | 283 | SFGVA | 9 |
| | | A289V | A_3001 | KGQVATCVP | 722 | −2.02 | 286 | VATCV | 10 |
| | | A289V | A_8001 | LNGFGVATR | 723 | −1.91 | 284 | FGVAT | 11 |
| P00533_L858R | EGFR_HUMAN | L858R | A_0101 | IKDFGRLAY | 724 | −2.00 | 853 | FGRLA | 12 |
| | Epidermal growth | L858R | A_2301 | EYHTDFGRL | 725 | −2.05 | 851 | TDFGR | 13 |
| | factor receptor | L858R | A_2301 | SLKDFGRLI | 726 | −2.01 | 852 | DFGRL | 14 |
| | OS_Homo sapiens | L858R | A_2301 | DVFGRLAKF | 727 | −2.02 | 854 | GRLAK | 15 |
| | OX_9606 GN_EGFR | L858R | A_2301 | RLRRLAKLL | 728 | −2.01 | 855 | RLAKL | 16 |
| | PE_1 SV_2 | L858R | A_3001 | TARDFGRLE | 729 | −2.01 | 852 | DFGRL | 17 |
| | | L858R | A_3001 | TNKFGRLAD | 730 | −2.05 | 853 | FGRLA | 18 |
| | | L858R | A_8001 | HGNDFGRLR | 731 | −2.00 | 852 | DFGRL | 19 |
| | | L858R | A_8001 | CPRFGRLAY | 732 | −2.00 | 853 | FGRLA | 20 |
| | | L858R | A_8001 | KCIRLAKLR | 733 | −2.05 | 855 | RLAKL | 21 |
| P04626_R678Q | ERBB2_HUMAN | R678Q | A_0101 | RSELIKRQL | 734 | −2.04 | 671 | LIKRQ | 22 |
| | Receptor tyrosine- | R678Q | A_2301 | SFAIKRQRL | 735 | −2.02 | 672 | IKRQR | 23 |
| | protein kinase | R678Q | A_2301 | PRLKRQRQI | 736 | −2.03 | 673 | KRQRQ | 24 |
| | erbB-2 | R678Q | A_2301 | VINRQRQQF | 737 | −2.02 | 674 | RQRQQ | 25 |
| | OS_Homo sapiens | R678Q | A_2301 | LSYQRQQKF | 738 | −2.03 | 675 | QRQQK | 26 |
| | OX_9606 | R678Q | A_3001 | SGKIKRQRN | 739 | −2.01 | 672 | IKRQR | 27 |
| | GN_ERBB2 PE_1 | R678Q | A_3001 | TMRRQRQQS | 740 | −1.99 | 674 | RQRQQ | 28 |
| | SV_1 | R678Q | A_8001 | AVELIKRQY | 741 | −2.02 | 671 | LIKRQ | 29 |
| P04626_S310F | ERBB2_HUMAN | S310F | A_0101 | RGNTDVGFL | 742 | −2.00 | 303 | TDVGF | 30 |
| | Receptor | S310F | A_0101 | YGQDVGFSQ | 743 | −2.09 | 304 | DVGFS | 31 |
| | tyrosine- | S310F | A_0101 | YTDVGFSCA | 744 | −2.02 | 305 | VGFSC | 32 |
| | protein kinase | S310F | A_2301 | HKLTDVGFF | 745 | −2.01 | 303 | TDVGF | 33 |
| | erbB-2 | S310F | A_2301 | QRTDVGFSF | 746 | −2.06 | 304 | DVGFS | 34 |
| | OS_Homo sapiens | S310F | A_2301 | PRYGFSCTF | 747 | −2.13 | 306 | GFSCT | 35 |
| | OX_9606 | S310F | A_3001 | VVKTDVGFA | 748 | −2.00 | 303 | TDVGF | 36 |
| | GN_ERBB2 PE_1 | S310F | A_3001 | SIKVGFSCS | 749 | −2.00 | 305 | VGFSC | 37 |
| | SV_1 | S310F | A_3001 | TIRFSCTLQ | 750 | −2.00 | 307 | FSCTL | 38 |
| | | S310F | A_8001 | ISTFSCTLR | 751 | −2.06 | 307 | FSCTL | 39 |
| P15056_V600E | BRAF_HUMAN | V600E | A_0101 | LLDATEVKP | 752 | −2.09 | 595 | AIEVK | 40 |
| | Serine_threonine- | V600E | A_0101 | FKCEVKSRP | 753 | −2.02 | 597 | EVKSR | 41 |
| | protein kinase | V600E | A_2301 | YLKGLAIEW | 754 | −2.02 | 593 | GLATE | 42 |
| | B-raf | V600E | A_2301 | KPPLAIEVF | 755 | −1.83 | 594 | LATEV | 43 |
| | OS_Homo sapiens | V600E | A_2301 | LFKAIEVKL | 756 | −2.01 | 595 | AIEVK | 44 |
| | OX_9606 GN_BRAF | V600E | A_2301 | LQFEVKSRL | 757 | −2.04 | 597 | EVKSR | 45 |
| | PE_1 SV_4 | V600E | A_3001 | AVKAIEVKA | 758 | −2.01 | 595 | AIEVK | 46 |
| | | V600E | A_8001 | VGLIEVKSY | 759 | −2.04 | 596 | IEVKS | 47 |
| | | V600E | A_8001 | IEIEVKSRY | 760 | −2.03 | 597 | EVKSR | 48 |
| P15056_V600M | BRAF_HUMAN | V600M | A_0101 | ASDLATMVE | 761 | −2.01 | 594 | LATMV | 49 |
| | Serine_threonine- | V600M | A_0101 | MSNATMVKL | 762 | −2.03 | 595 | ATMVK | 50 |
| | protein kinase | V600M | A_0101 | IAEMVKSRV | 763 | −1.99 | 597 | MVKSR | 51 |
| | B-raf | V600M | A_2301 | KWDGLATML | 764 | −1.98 | 593 | GLATM | 52 |
| | OS_Homo sapiens | V600M | A_2301 | RMSATMVKF | 765 | −2.00 | 595 | ATMVK | 53 |
| | OX_9606 GN_BRAF | V600M | A_2301 | IRRTMVKSI | 766 | −2.02 | 596 | TMVKS | 54 |
| | PE_1 SV_4 | V600M | A_2301 | VRTMVKSRF | 767 | −2.08 | 597 | MVKSR | 55 |
| | | V600M | A_3001 | NMKLATMVG | 768 | −2.01 | 594 | LATMV | 56 |
| | | V600M | A_3001 | YGKATMVKA | 769 | −2.09 | 595 | ATMVK | 57 |
| | | V600M | A_8001 | VELTMVKSY | 770 | −1.99 | 596 | TMVKS | 58 |
| | | V600M | A_8001 | TVIMVKSRR | 771 | −2.01 | 597 | MVKSR | 59 |
| P42336_E545K | PIK3CA_HUMAN | E545K | A_0101 | CPEITKEQY | 772 | −2.03 | 540 | ITKEQ | 60 |
| | Phos- | E545K | A_8001 | RGIEITKER | 773 | −2.02 | 539 | EITKE | 61 |
| | phatidylinositol | | | | | | | | |
| | 4 | | | | | | | | |
| P42336_H1047R | PIK3CA_HUMAN | H1047R | A_0101 | PSDMNDARL | 774 | −2.05 | 1040 | MNDAR | 62 |
| | Phos- | H1047R | A_0101 | YADARHHGC | 775 | −2.03 | 1043 | ARHHG | 63 |
| | phatidylinositol | H1047R | A_3001 | APRDARHHK | 776 | −2.00 | 1042 | DARHH | 64 |
| | 4 | H1047R | A_3001 | KARARHHGA | 777 | −2.06 | 1043 | ARHHG | 65 |
| | | H1047R | A_8001 | WKIRHHGGR | 778 | −2.04 | 1044 | RHHGG | 66 |
| P60484_R130G | PTEN_HUMAN | R130G | A_0101 | FGDKGGRTG | 779 | −2.01 | 125 | KGGRT | 67 |
| | Phos- | R130G | A_3001 | QGKAGKGGP | 780 | −2.08 | 123 | AGKGG | 68 |
| | phatidylinositol | R130G | A_3001 | DNRKGGRTK | 781 | −2.00 | 125 | KGGRT | 69 |
| | 3 | R130G | A_3001 | NNRGGRTGA | 782 | −2.00 | 126 | GGRTG | 70 |
| | | R130G | A 8001 | LITAGKGGY | 783 | −1.97 | 123 | AGKGG | 71 |
| | | R130G | A_8001 | EHFKGGRTY | 784 | −2.10 | 125 | KGGRT | 72 |
| | | R130Q | A_3001 | PAKAGKGQP | 785 | −2.07 | 123 | AGKGQ | 73 |
| | | R130Q | A_3001 | PDRKGQRTG | 786 | −2.03 | 125 | KGQRT | 74 |
| | | R130Q | A_3001 | RAWGQRTGP | 787 | −2.00 | 126 | GQRTG | 75 |
| | | R130Q | A_8001 | GVLAGKGQY | 788 | −2.00 | 123 | AGKGQ | 76 |
| | | R130Q | A_8001 | QECKGQRTY | 789 | −2.05 | 125 | KGQRT | 77 |

TABLE 23
Exemplar peptides eliciting CD8 T cell responses to multi cancer mutated
proteins for the indicated MHC I B alleles

| | | | | | SEQ | | Pre- | | SEQ |
|---|---|---|---|---|---|---|---|---|---|
| | | Mu- | | Binding | ID | Posi- | dicted | | ID |
| Protein ID | Protein Curation | tation | Allele | peptide | NO: | tion | binding | TCEM | NO. |
| P00533_A289V | EGFR_HUMAN | A289V | B_2705 | PDKSFGVAY | 790 | 283 | −2.04 | SFGVA | 162 |
| | Epidermal growth | A289V | B_2705 | WKTFGVATS | 791 | 284 | −2.03 | FGVAT | 163 |
| | factor receptor | A289V | B_2705 | PEFGVATCK | 792 | 285 | −2.00 | GVATC | 164 |
| | OS_Homo sapiens | A289V | B_3801 | FEHSFGVAS | 793 | 283 | −2.01 | SFGVA | 165 |
| | OX_9606 GN_EGFR | A289V | B_3801 | YRPGVATCV | 794 | 285 | −1.93 | GVATC | 166 |
| | PE_1 SV_2 | A289V | B_4801 | GKTSFGVAG | 795 | 283 | −2.00 | SFGVA | 167 |
| | | A289V | B_4801 | GKHFGVATL | 796 | 284 | −2.01 | FGVAT | 168 |
| | | A289V | B_4801 | REQGVATCL | 797 | 285 | −2.04 | GVATC | 169 |
| | | A289V | B_5701 | RGSYSFGVY | 798 | 282 | −2.08 | YSFGV | 170 |
| | | A289V | B_5701 | GGSGVATCY | 799 | 285 | −2.01 | GVATC | 171 |
| P00533_L858R | EGFR_HUMAN | L858R | B_2705 | DRLTDFGRE | 800 | 851 | −2.00 | TDFGR | 172 |
| | Epidermal growth | L858R | B_2705 | YEMDFGRLY | 801 | 852 | −2.00 | DFGRL | 173 |
| | factor receptor | L858R | B_2705 | VRGGRLAKR | 802 | 854 | −2.03 | GRLAK | 174 |
| | OS_Homo sapiens | L858R | B_3801 | FRFTDFGRT | 803 | 851 | −1.83 | TDFGR | 175 |
| | OX_9606 GN_EGFR | L858R | B_3801 | QELGRLAKP | 804 | 854 | −2.00 | GRLAK | 176 |
| | PE_1 SV_2 | L858R | B_4801 | KQYTDFGRL | 805 | 851 | −1.81 | TDFGR | 177 |
| | | L858R | B_4801 | RCYDFGRLW | 806 | 852 | −2.01 | DFGRL | 178 |
| | | L858R | B_4801 | YKLGRLAKI | 807 | 854 | −1.96 | GRLAK | 179 |
| | | L858R | B_4801 | ENPRLAKLI | 808 | 855 | −2.01 | RLAKL | 180 |
| | | L858R | B_5701 | ELNTDFGRW | 809 | 851 | −2.03 | TDFGR | 181 |
| | | L858R | B_5701 | CSNGRLAKF | 810 | 854 | −2.08 | GRLAK | 182 |
| P04626_R678Q | ERBB2_HUMAN | R678Q | B_2705 | IRSLIKRQL | 811 | 671 | −2.00 | LIKRQ | 183 |
| | Receptor | R678Q | B_3801 | THWQRQQKL | 812 | 675 | −1.97 | QRQQK | 184 |
| | tyrosine- | R678Q | B_4801 | RCLKRQRQL | 813 | 673 | −1.70 | KRQRQ | 185 |
| | protein kinase | R678Q | B_4801 | RDLQRQQKV | 814 | 675 | −2.03 | QRQQK | 186 |
| | erbB-2 | R678Q | B_5701 | SPWLIKRQI | 815 | 671 | −2.03 | LIKRQ | 187 |
| | OS_Homo sapiens | R678Q | B_5701 | TAAQRQQKY | 816 | 675 | −2.00 | QRQQK | 188 |
| | OX_9606 | | | | | | | | |
| | GN_ERBB2 PE_1 | | | | | | | | |
| | SV_1 | | | | | | | | |
| P04626_S310F | ERBB2_HUMAN | S310F | B_2705 | TKRGFSCTK | 817 | 306 | −2.00 | GFSCT | 189 |
| | Receptor | S310F | B_3801 | TRKDVGFSI | 818 | 304 | −2.00 | DVGFS | 190 |
| | tyrosine- | S310F | B_3801 | LRHGFSCTC | 819 | 306 | −2.01 | GFSCT | 191 |
| | protein kinase | S310F | B_4801 | QDEDVGFSM | 820 | 304 | −2.01 | DVGFS | 192 |
| | erbB-2 | S310F | B_4801 | QNEGFSCTA | 821 | 306 | −2.00 | GFSCT | 193 |
| | OS_Homo sapiens | S310F | B_4801 | PNQFSCTLS | 1093 | 307 | −2.00 | FSCTL | 194 |
| | OX_9606 | S310F | B_5701 | HSKDVGFSI | 1094 | 304 | −2.00 | DVGFS | 195 |
| | GN_ERBB2 PE_1 | S310F | B_5701 | GSKGFSCTM | 1095 | 306 | −2.00 | GFSCT | 196 |
| | SV_1 | | | | | | | | |
| P15056_V600E | BRAF_HUMAN | V600E | B_2705 | DQFGLATEK | 822 | 593 | −2.00 | GLATE | 197 |
| | Serine_threonine- | V600E | B_2705 | LRDEVKSRE | 823 | 597 | −2.03 | EVKSR | 198 |
| | protein kinase | V600E | B_3801 | IRKGLAIEY | 824 | 593 | −2.02 | GLAIE | 199 |
| | B-raf | V600E | B_4801 | PNVGLATEI | 825 | 593 | −2.02 | GLATE | 200 |
| | OS_Homo sapiens | V600E | B_4801 | AKAAIEVKL | 826 | 595 | −2.01 | AIEVK | 201 |
| | OX_9606 GN_BRAF | V600E | B_5701 | PSCGLATEM | 827 | 593 | −2.04 | GLATE | 202 |
| | PE_1 SV_4 | V600E | B_5701 | LSKATEVKL | 828 | 595 | −2.02 | AIEVK | 203 |
| | | V600E | B_5701 | SCITEVKSF | 829 | 596 | −2.00 | IEVKS | 204 |
| | | V600E | B_5701 | ESPEVKSRY | 830 | 597 | −2.01 | EVKSR | 205 |
| P15056_V600M | BRAF_HUMAN | V600M | B_2705 | DKVGLATMA | 831 | 593 | −2.03 | GLATM | 206 |
| | Serine_threonine- | V600M | B_2705 | IRGMVKSRN | 832 | 597 | −2.00 | MVKSR | 207 |
| | protein kinase | V600M | B_3801 | LRQGLATMQ | 833 | 593 | −2.01 | GLATM | 208 |
| | B-raf | V600M | B_4801 | ENPGLATMI | 834 | 593 | −2.06 | GLATM | 209 |
| | OS_Homo sapiens | V600M | B_4801 | SKGATMVKL | 835 | 595 | −1.98 | ATMVK | 210 |
| | OX_9606 GN_BRAF | V600M | B_4801 | GQVTMVKSI | 836 | 596 | −2.06 | TMVKS | 211 |
| | PE_1 SV_4 | V600M | B_5701 | KAKGLATMM | 837 | 593 | −2.04 | GLATM | 212 |
| | | V600M | B_5701 | RGDATMVKI | 838 | 595 | −2.02 | ATMVK | 213 |
| | | V600M | B_5701 | VGCTMVKSM | 839 | 596 | −2.03 | TMVKS | 214 |
| | | V600M | B_5701 | TITMVKSRW | 840 | 597 | −2.00 | MVKSR | 215 |
| P42336_E545K | PIK3CA_HUMAN | E545K | B_2705 | KKAEITKES | 841 | 539 | −2.00 | EITKE | 216 |
| | Phos- | E545K | B_2705 | MRPTKEQEQ | 842 | 541 | −2.02 | TKEQE | 217 |
| | phatidylinositol | E545K | B_4801 | ADLSEITKV | 843 | 538 | −2.07 | SEITK | 218 |
| | 4 | E545K | B_4801 | NKLITKEQL | 844 | 540 | −2.09 | ITKEQ | 219 |
| P42336_H1047R | PIK3CA_HUMAN | H1047R | B_2705 | LRVNDARHI | 845 | 1041 | −2.03 | NDARH | 220 |
| | Phos- | H1047R | B_2705 | LKPRHHGGN | 846 | 1044 | −2.03 | RHHGG | 221 |
| | phatidylinositol | H1047R | B_3801 | IRMDARHHV | 847 | 1042 | −1.60 | DARHH | 222 |
| | 4 | H1047R | B_3801 | PEWARHHGW | 848 | 1043 | −2.02 | ARHHG | 223 |
| | | H1047R | B_4801 | SQARHHGGC | 849 | 1044 | −2.02 | RHHGG | 224 |
| | | H1047R | B_5701 | IEQNDARHF | 850 | 1041 | −2.00 | NDARH | 225 |
| | | H1047R | B_5701 | FGHRHHGGR | 851 | 1044 | −2.01 | RHHGG | 226 |
| P60484_R130G | PTEN_HUMAN | R130G | B_2705 | KQLAGKGGP | 852 | 123 | −2.01 | AGKGG | 227 |
| | Phos- | R130G | B_2705 | REWGRTGVE | 853 | 127 | −2.02 | GRTGV | 228 |
| | phatidylinositol | R130G | B_3801 | VHCAGKGGL | 854 | 123 | −2.03 | AGKGG | 229 |
| | 3 | R130G | B_3801 | MCWGKGGRA | 855 | 124 | −1.62 | GKGGR | 230 |
| | | R130G | B_3801 | ARQGRTGVS | 856 | 127 | −2.00 | GRTGV | 231 |
| | | R130G | B_4801 | KTWKGGRTL | 857 | 125 | −2.02 | KGGRT | 232 |
| | | R130G | B_4801 | FKCGRTGVL | 858 | 127 | −2.02 | GRTGV | 233 |
| | | R130G | B_5701 | NITAGKGGW | 859 | 123 | −2.00 | AGKGG | 234 |
| | | R130G | B_5701 | LSHKGGRTR | 860 | 125 | −2.02 | KGGRT | 235 |
| | | R130G | B_5701 | GGPGGRTGM | 861 | 126 | −2.02 | GGRTG | 236 |
| | | R130Q | B_2705 | DRTAGKGQE | 862 | 123 | −2.00 | AGKGQ | 237 |
| P60484_R130Q | PTEN_HUMAN | R130Q | B_3801 | LRKAGKGQP | 863 | 123 | −2.03 | AGKGQ | 238 |
| | Phos- | R130Q | B_3801 | LRTQRTGVP | 864 | 127 | −2.02 | QRTGV | 239 |
| | phatidylinositol | R130Q | B_4801 | GMFKGQRTL | 865 | 125 | −2.04 | KGQRT | 240 |
| | 3 | R130Q | B_4801 | PDLQRTGVL | 866 | 127 | −1.98 | QRTGV | 241 |
| | | R130Q | B_5701 | GDFAGKGQF | 867 | 123 | −2.04 | AGKGQ | 242 |
| | | R130Q | B_5701 | QANKGQRTL | 868 | 125 | −2.00 | KGQRT | 243 |
| | | R130Q | B_5701 | EGMGQRTGL | 869 | 126 | −2.00 | GQRTG | 244 |

TABLE 24
Exemplar peptides elicting CD4 T cell responses to
multi cancer mutated proteins for the indicated MHC II alleles

| | | | | | SEQ | Pre- | | | SEQ |
|---|---|---|---|---|---|---|---|---|---|
| | | Mu- | | | ID | dicted | Posi- | | ID |
| Protein ID | Protein Curation | tation | Allele | Binding peptide | NO: | binding | tion | TCEM | NO. |
| P00533_A289V | EGFR_HUMAN | A289V | DQB0302 | SSDAGKYSFGVLRLM | 870 | −2.11 | 279 | GKySfGV | 78 |
| | Epidermal growth | A289V | DQB0302 | GEQQKYSFGVAQNWC | 871 | −2.01 | 280 | KYsFgVA | 79 |
| | factor receptor | A289V | DQB0302 | RAEPSFGVATCGHFN | 872 | −2.00 | 282 | SFgVaTC | 80 |
| | OS_Homo sapiens | A289V | DQB0302 | AISKGVATCVKGKFV | 873 | −2.00 | 284 | GVaTcVK | 81 |
| | OX_9606 GN_EGFR | A289V | DQB0302 | MNCNVATCVKKACVF | 874 | −2.00 | 285 | VAtCyKK | 82 |
| | PE_1 SV_2 | A289V | DQB0602 | LDLLGKYSFGVSAPG | 875 | −1.90 | 279 | GKySfGV | 83 |
| | | A289V | DQB0602 | VVNRGVATCVKAVNE | 876 | −2.00 | 284 | GVaTcVK | 84 |
| | | A289V | DQB0602 | LFQKVATCVKKAESS | 877 | −2.00 | 285 | VAtCyKK | 85 |
| | | A289V | DRB1201 | RCHFGVATCVKTMDF | 878 | −1.50 | 284 | GVaTcVK | 86 |
| | | A289V | DRB3_0101 | IRRHSFGVATCELVC | 879 | −1.88 | 282 | SFgVaTC | 87 |
| | | A289V | DRB3_0101 | HSDHGVATCVKPMYT | 880 | −1.92 | 284 | GVaTcVK | 88 |
| P00533_L858R | EGFR_HUMAN | L858R | DQB0602 | LSTPITDFGRLAGHA | 881 | −2.04 | 849 | ITdFgRL | 89 |
| | Epidermal growth | L858R | DQB0602 | FDQKGRLAKLLTWIC | 882 | −1.52 | 853 | GRlAkLL | 90 |
| | factor receptor | L858R | DRB1201 | FVIWKITDFGRVKYN | 883 | −2.00 | 848 | KItDfGR | 91 |
| | OS_Homo sapiens | L858R | DRB1201 | IVSWITDFGRLWKRN | 884 | −2.02 | 849 | ITdFgRL | 92 |
| | OX_9606 GN_EGFR | L858R | DRB1201 | TLLMDFGRLAKRTMK | 885 | −2.02 | 851 | DFgRlAK | 93 |
| | PE_1 SV_2 | L858R | DRB1201 | SEMFGRLAKLLEYAI | 886 | −2.00 | 853 | GRlAkLL | 94 |
| | | L858R | DRB1201 | SQEIRLAKLLGYRSR | 887 | −2.01 | 854 | RLaKlLG | 95 |
| | | L858R | DRB3_0101 | SAYEKITDFGRKIVI | 888 | −2.00 | 848 | KItDfGR | 96 |
| | | L858R | DRB3_0101 | LGYEITDFGRLRVGY | 889 | −2.07 | 849 | ITdFgRL | 97 |
| | | L858R | DRB3_0101 | NKIFDFGRLAKRLII | 890 | −2.00 | 851 | DFgRlAK | 98 |
| P04626_R678Q | ERBB2_HUMAN | R678Q | DQB0602 | LNLLIKRQRQQPPNC | 891 | −1.55 | 671 | IKrQrQQ | 99 |
| | Receptor | R678Q | DRB1201 | LGVHGILIKRQACHC | 892 | −2.00 | 668 | GIlIkRQ | 100 |
| | tyrosine-protein | R678Q | DRB1201 | YMCLILIKRQRLTNR | 893 | −2.01 | 669 | ILiKrQR | 101 |
| | kinase erbB-2 | R678Q | DRB1201 | FALFIKRQRQQSQCW | 894 | −1.83 | 671 | IKrQrQQ | 102 |
| | OS_Homo sapiens | R678Q | DRB1201 | LIVWQRQQKIRALTE | 895 | −1.90 | 674 | QRqQkIR | 103 |
| | OX_9606 GN_ERBB2 | R678Q | DRB3_0101 | DAWYIKRQRQQRLTC | 896 | −2.02 | 671 | IKrQrQQ | 104 |
| | PE_1 SV_1 | R678Q | DRB3_0101 | PGLDRQRQQKIIVQD | 897 | −2.01 | 673 | RQrQqKI | 105 |
| | | R678Q | DRB3_0101 | GFKTQRQQKIRVFLE | 898 | −2.00 | 674 | QRqQkIR | 106 |
| P04626_S310F | ERBB2_HUMAN | S310F | DQB0302 | GVDVLSTDVGFQDIC | 899 | −2.02 | 300 | LStDvGF | 107 |
| | Receptor | S310F | DQB0302 | PGTSSTDVGFSGEFH | 900 | −2.00 | 301 | STdVgFS | 108 |
| | tyrosine-protein | S310F | DQB0302 | SENYDVGFSCTYDLV | 901 | −2.00 | 303 | DVgFsCT | 109 |
| | kinase erbB-2 | S310F | DQB0302 | GFGNGFSCTLVQHDT | 902 | −2.01 | 305 | GFsCtLV | 110 |
| | OS_Homo sapiens | S310F | DQB0602 | LALQLSTDVGFSAPS | 903 | −2.05 | 300 | LStDvGF | 111 |
| | OX_9606 GN_ERBB2 | S310F | DQB0602 | SNISSTDVGFSPLAV | 904 | −2.02 | 301 | STdVgFS | 112 |
| | PE_1 SV_1 | S310F | DQB0602 | SATVDVGFSCTDHLT | 905 | −2.00 | 303 | DVgFsCT | 113 |
| | | S310F | DQB0602 | AEILGFSCTLVATRS | 906 | −1.95 | 305 | GFsCtLV | 114 |
| | | S310F | DRB1201 | DHFFLSTDVGFRIER | 907 | −2.05 | 300 | LStDvGF | 115 |
| | | S310F | DRB1201 | LMRISTDVGFSVKVC | 908 | −2.09 | 301 | STdVgFS | 116 |
| | | S310F | DRB1201 | LSRMFSCTLVCQSGH | 909 | −2.02 | 306 | FScTlVC | 117 |
| | | S310F | DRB3_0101 | SSWELSTDVGFYSEI | 910 | −2.03 | 300 | LStDvGF | 118 |
| | | S310F | DRB3_0101 | STLYSTDVGFSYITG | 911 | −1.96 | 301 | STdVgFS | 119 |
| | | S310F | DRB3_0101 | IERKGFSCTLVTMIQ | 912 | −2.00 | 305 | GFsCtLV | 120 |
| P15056_V600E | BRAF_HUMAN | V600E | DQB0302 | ANKKEVKSRWSAQLC | 913 | −2.00 | 596 | EVkSrWS | 121 |
| | Serine_threonine- | V600E | DQB0602 | LCKSLATEVKSPFKQ | 914 | −2.01 | 593 | LAtEvKS | 122 |
| | protein kinase | V600E | DQB0602 | FNLLTEVKSRWPYCD | 915 | −1.60 | 595 | TEvKsRW | 123 |
| | B-raf OS_Homo | V600E | DQB0602 | AQLPEVKSRWSTDWE | 916 | −1.99 | 596 | EVkSrWS | 124 |
| | sapiens OX_9606 | V600E | DRB1201 | SVLRDFGLAIELYKI | 917 | −2.04 | 590 | DFgLaTE | 125 |
| | GN_BRAF PE_1 | V600E | DRB1201 | GMRYFGLAIEVPASM | 918 | −2.08 | 591 | FGlAtEV | 126 |
| | SV_4 | V600E | DRB1201 | CPFCIEVKSRWFLLK | 919 | −2.02 | 595 | TEvKsRW | 127 |
| P15056_V600M | BRAF_HUMAN | V600M | DQB0302 | PRHRFGLATMVCCTG | 920 | −2.05 | 591 | FGlAtMV | 128 |
| | Serine_threonine- | V600M | DQB0302 | DCQDLATMVKSVCSS | 921 | −2.03 | 593 | LAtMvKS | 129 |
| | protein kinase | | | | | | | | |
| | B-raf OS_Homo | V600M | DQB0302 | FRKTMVKSRWSRCLC | 922 | −2.05 | 596 | MVkSrWS | 130 |
| | sapiens OX_9606 | V600M | DQB0602 | LNPTLATMVKSLEES | 923 | −2.01 | 593 | LAtMvKS | 131 |
| | GN_BRAF PE_1 SV_4 | V600M | DQB0602 | LALLMVKSRWSTGEV | 924 | −1.91 | 596 | MVkSrWS | 132 |
| | | V600M | DRB1201 | NYGVDFGLATMLTHH | 925 | −2.02 | 590 | DFgLaTM | 133 |
| | | V600M | DRB1201 | KYISFGLATMVKNVD | 926 | −2.01 | 591 | FGlAtMV | 134 |
| | | V600M | DRB1201 | VCEILATMVKSYRLD | 927 | −2.03 | 593 | LAtMvKS | 135 |
| | | V600M | DRB1201 | LNELTMVKSRWLPLK | 928 | −2.02 | 595 | TMvKsRW | 136 |
| P42336_E545K | PIK3CA_HUMAN | E545K | DQB0302 | DGENEITKEQEQCLE | 929 | −1.80 | 538 | EItKeQE | 137 |
| | Phos- | E545K | DQB0602 | LYFSLSEITKELGQC | 930 | −1.93 | 536 | LSeItKE | 138 |
| | phatidylinositol | E545K | DQB0602 | LCLGKEQEKDFVARA | 931 | −2.01 | 541 | KEqEkDF | 139 |
| | 4 | E545K | DRB3_0101 | FILLEITKEQERVYC | 932 | −2.01 | 538 | EItKeQE | 140 |
| | | E545K | DRB3_0101 | SYWQTKEQEKDRLVT | 933 | −2.02 | 540 | TKeQeKD | 141 |
| | | E545K | DRB3_0101 | KNLDKEQEKDFIIII | 934 | −2.00 | 541 | KEqEkDF | 142 |
| P42336_H1047R | PIK3CA_HUMAN | H1047R | DQB0602 | LDLTRHHGGWTASID | 935 | −1.98 | 1043 | RHhGgWT | 143 |
| | Phos- | H1047R | DRB3_0101 | DFNEKQMNDARYIIE | 936 | −2.00 | 1037 | KQmNdAR | 144 |
| | phatidylinositol | H1047R | DRB3_0101 | CPVVQMNDARHQLIV | 937 | −2.00 | 1038 | QMnDaRH | 145 |
| | 4 | H1047R | DRB3_0101 | KKYLNDARHHGIILV | 938 | −2.02 | 1040 | NDaRhHG | 146 |
| P60484_R130G | PTEN_HUMAN | R130G | DQB0302 | GQLRCKAGKGGYRPN | 939 | −2.00 | 120 | CKaGkGG | 147 |
| | Phos- | R130G | DQB0302 | LEENGKGGRTGPINC | 940 | −2.00 | 123 | GKgGrTG | 148 |
| | phatidylinositol | R130G | DQB0302 | NKEFGGRTGVMWCII | 941 | −2.04 | 125 | GGrTgVM | 149 |
| | 3 | R130G | DQB0302 | SNQDGRTGVMIMEID | 942 | −2.04 | 126 | GRtGyMI | 150 |
| | | R130G | DQB0602 | ICLLCKAGKGGSSES | 943 | −2.03 | 120 | CKaGkGG | 151 |
| | | R130G | DQB0602 | LVAQGKGGRTGLPIG | 944 | −2.06 | 123 | GKgGrTG | 152 |
| | | R130G | DQB0602 | LPAYGGRTGVMSYEG | 945 | −2.03 | 125 | GGrTgVM | 153 |
| | | R130Q | DQB0302 | TNNPCKAGKGQFEVW | 946 | −2.00 | 120 | CKaGkGQ | 154 |
| | | R130Q | DQB0302 | QFEKGKGQRTGGHVM | 947 | −2.01 | 123 | GKgQrTG | 155 |
| | | R130Q | DQB0302 | AELAGQRTGVMACYD | 948 | −1.76 | 125 | GQrTgVM | 156 |
| | | R130Q | DQB0302 | SQRLQRTGVMIPCFI | 949 | −2.00 | 126 | QRtGyMI | 157 |
| | | R130Q | DQB0602 | LVPTGKGQRTGAYYS | 950 | −1.97 | 123 | GKgQrTG | 158 |
| | | R130Q | DRB_1201 | SCIFKAGKGQRPHIT | 951 | −1.42 | 121 | KAgKgQR | 159 |
| | | R130Q | DRB3_0101 | FQRPGQRTGVMCMGM | 952 | −1.91 | 125 | GQrTgVM | 160 |
| | | R130Q | DRB3_0101 | LTQDQRTGVMIYDFC | 953 | −2.02 | 126 | QRtGyMI | 161 |

TCEM IIA motifs are shown with exposed amino acids in capital letters and hidden bound amino acids in lower case letters.

### Example 8: Bespoke Peptides Spanning the Oncogenic Deletion in Epidermal Growth Factor Receptor viii (EGFRviii)

EGFR is upregulated in 54% pf glioblastomas [34]. Various deletion mutants are recognized with EGFRviii being the most common, and like EGFRvii being oncogenic. In EGFRviii exons 2 and 7 are deleted leading to removal of amino acids 6-273 of the mature protein; a glycine is inserted in the bridge and the downstream sequence remains in frame. The adverse effects of EGFRviii are well documented [34] An effort was made to use a peptide spanning the deletion junction as a vaccine. This peptide, comprising 14 amino acids comprises a B cell epitope and was viewed as a way of inducing antibody dependent cytotoxicity. Despite initially promising results, a large phase III trial of the vaccine used in combination with temozolomide failed to show any benefit. Patients were HLA typed but no significant associations in benefit were reported [28].

Upon closer examination of the unique T cell exposed motifs spanning the deletion junction in EGFRviii we noted that relatively few MHC I alleles bound at least one of the five possible unique T cell exposed motifs. Overall 31 of 70 MHC I alleles bound at less than ~500 nM (1 SD), comprising 17 binding sites among the 31 B alleles, 9 of 31 A allele and 5 of 8 C alleles evaluated had binding less than 500 nM at any of the possible T cell exposed motifs. In particular, no binding of A0201 was predicted. In addition, A0101, B4001 and B 1542 had predicted binding in excess of 2.75 SD below the mean equivalent of approximately 20 nM which may be an affinity so high it could induce suppression or exhaustion.

Therefore, EGFRviii is a candidate for a personalized peptide vaccine approach in which peptides are selected specifically for to optimize binding to a patient's alleles. Among the 70 alleles for which predicted binding was evaluated in the natural mutated EGFRviii, 65 alleles have some probability of presentation of the native epitope based on at least a low level of binding of the natural peptide. These are candidates for using a synthetic bespoke peptide to stimulate T cells which are cognate for and can therefore target these T cell exposed motifs. Following the process laid out in the prior examples we generated a set of 10,000 peptides for each of the possible T cell exposed positions ~~~EEKKG~ (SEQ ID NO: 252), ~~~EKKGN~ (SEQ ID NO: 246), ~~~KKGNY~ (SEQ ID NO: 245), ~~~KGNYV~ (SEQ ID NO: 250), ~GNYVV~ (SEQ ID NO: 247).

Soluble peptides were selected, and those with binding affinity in two ranges of approximately −2.25 to −1.75 SD below the mean and −2.75 to −2.5 SD below the mean for all peptides in the protein, equivalent to approximately 25 nM and 50 nM selected. This binding affinity was selected from a range of affinities, other affinities could have been chosen for this example and thus this example is considered non limiting.

Table 25 shows the process of down selection of candidate peptides from the total simulated. Examples of peptides with selected predicted binding affinity are shown in Table 26 for a set of example alleles. These are assigned SEQ ID NOs.: 245-284.

TABLE 25

| | Simulated | | | | Predicted | Predicted |
|---|---|---|---|---|---|---|
| | peptides with | | | | binding | binding |
| | above median | | Available | | −2.5 to | −1.75- |
| | binding of | TCEM | presented | | −2.75 | −2.25 |
| Allele | protein | presented | peptides | Soluble | ~25 nM | ~50 nM |
| B0702 | 21507 | 2 | 12229 | 11685 | 112 | 793 |
| B3501 | 23863 | 2 | 9892 | 9892 | 69 | 524 |
| B4402 | 21851 | 2 | 8466 | 7930 | 273 | 397 |
| B5701 | 23521 | 2 | 7255 | 7255 | 65 | 165 |
| A0101 | 22473 | 3 | 15202 | 12359 | 125 | 636 |
| A0201 | 20727 | 1 | 3420 | 3420 | 37 | 153 |
| A2402 | 22574 | 2 | 11461 | 10828 | 58 | 850 |
| A6901 | 20524 | 1 | 4953 | 4410 | 91 | 324 |
| C0401 | 23755 | 2 | 10004 | 9683 | 47 | 488 |
| C0602 | 24416 | 3 | 16119 | 14895 | 164 | 969 |

TABLE 26

| | | | | | | TCEM | |
|---|---|---|---|---|---|---|---|
| | | | SEQ | Predicted | | core | SEQ |
| Binding | | | ID | binding in | Polarity/ | amino | ID |
| group | Allele | Peptide | NO: | SD units | solubility | acids | NO. |
| High | A0101 | LADKKGNYV | 954 | −2.59 | −1.09 | KKGNY | 245 |
| | A0101 | KASEKKGNY | 955 | −2.57 | −3.36 | EKKGN | 246 |
| | A0101 | DGDGNYVVS | 956 | −2.55 | −0.94 | GNYVV | 247 |
| | A0201 | KLAEKKGNV | 957 | −2.67 | −2.08 | EKKGN | 248 |
| | A2402 | QYTKKGNYF | 958 | −2.72 | −1.28 | KKGNY | 249 |
| | A2402 | KYTKGNYVW | 959 | −2.67 | −0.47 | KGNYV | 250 |
| | A6901 | ESDKGNYVC | 960 | −2.54 | −1.86 | KGNYV | 251 |
| | B0702 | APGEEKKGG | 961 | −2.66 | −2.93 | EEKKG | 252 |
| | B0702 | PPDKGNYVA | 962 | −2.64 | −1.09 | KGNYV | 253 |
| | B3501 | LLREEKKGF | 963 | −2.62 | −1.27 | EEKKG | 254 |
| | B3501 | FAMEKKGNY | 964 | −2.57 | −1.06 | EKKGN | 255 |
| | B4402 | ECRKGNYVE | 965 | −2.72 | −2.22 | KGNYV | 256 |
| | B4402 | PCQKKGNYV | 966 | −2.72 | −1.44 | KKGNY | 257 |
| | B5701 | LGDEKKGNF | 967 | −2.66 | −1.91 | EKKGN | 258 |
| | B5701 | PASEEKKGF | 968 | −2.65 | −2.25 | EEKKG | 259 |
| | C0401 | IRQKGNYVS | 969 | −2.65 | −1.19 | KGNYV | 260 |
| | C0401 | LWSEKKGNG | 970 | −2.64 | −1.70 | EKKGN | 261 |
| | C0602 | TKSKKGNYR | 971 | −2.74 | −3.66 | KKGNY | 262 |
| | C0602 | IRRGNYVVS | 972 | −2.66 | −0.17 | GNYVV | 263 |
| | C0602 | LKEEEKKGD | 973 | −2.23 | −4.15 | EEKKG | 264 |
| Medium | A0101 | RAEGNYVVR | 974 | −2.01 | −1.17 | GNYVV | 265 |
| | A0101 | MGEKKGNYD | 975 | −2.01 | −2.69 | KKGNY | 266 |
| | A0101 | TADEKKGNF | 976 | −2.01 | −2.64 | EKKGN | 267 |
| | A0201 | RLKEKKGNV | 977 | −1.99 | −2.86 | EKKGN | 268 |
| | A2402 | QLPKKGNYI | 978 | −2.00 | −0.74 | KKGNY | 269 |
| | A2402 | TKGKGNYVI | 979 | −2.00 | −0.74 | KGNYV | 270 |
| | A6901 | EVSKGNYVA | 980 | −2.00 | −0.81 | KGNYV | 271 |
| | B0702 | NVRKGNYVA | 981 | −1.99 | −1.06 | KGNYV | 272 |
| | B0702 | RTQEEKKGI | 982 | −1.99 | −3.40 | EEKKG | 273 |
| | B3501 | QSCEKKGNW | 983 | −2.00 | −2.55 | EKKGN | 274 |
| | B3501 | FPMEEKKGR | 984 | −1.99 | −2.28 | EEKKG | 275 |
| | B4402 | SEEKKGNYQ | 985 | −2.00 | −3.77 | KKGNY | 276 |
| | B4402 | LELKGNYVP | 986 | −2.00 | 0.34 | KGNYV | 277 |
| | B5701 | EGPEEKKGY | 987 | −2.00 | −3.27 | EEKKG | 278 |
| | B5701 | ISKEKKGNF | 988 | −1.99 | −2.18 | EKKGN | 279 |
| | C0401 | EHMKGNYVG | 989 | −2.01 | −1.03 | KGNYV | 280 |
| | C0401 | RELEKKGNA | 990 | −2.00 | −3.21 | EKKGN | 281 |
| | C0602 | AEHGNYVVT | 991 | −2.01 | −0.21 | GNYVV | 282 |
| | C0602 | TRVKKGNYS | 992 | −2.01 | −2.39 | KKGNY | 283 |
| | C0602 | WKEEEKKGR | 993 | −2.01 | −4.28 | EEKKG | 284 |

### Example 9: Determination of HLA Haplotypes Determined from Whole Exome Sequences

A ‘BAM slice’ of the exome file containing the HLA locus (GRch38=chr6: 29722700-33143300) was used. The principles outlined for the Optitype which focuses on the read matches to exons 2 and 3 of the MHC molecules was used in conjunction with the magicBLAST [36] aligner. magicBLAST has features that are particularly suited for this type of application. Optitype has been shown to be one of the most accurate methods but only has prediction capabilities for MHC I and thus teaches away from MHC II typing. This general approach was modified as follows to provide MHC II typing also.

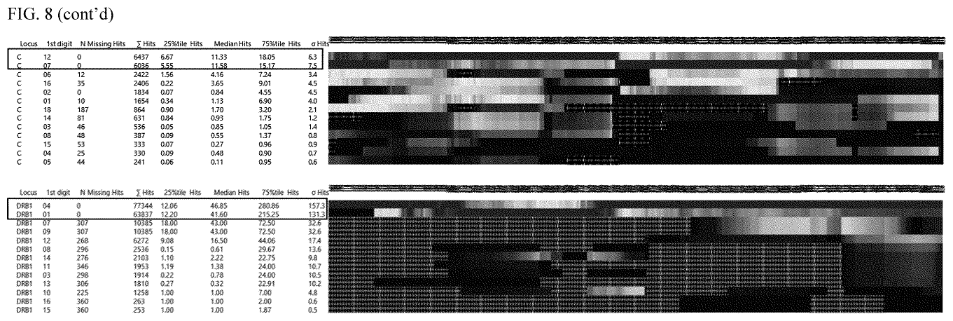

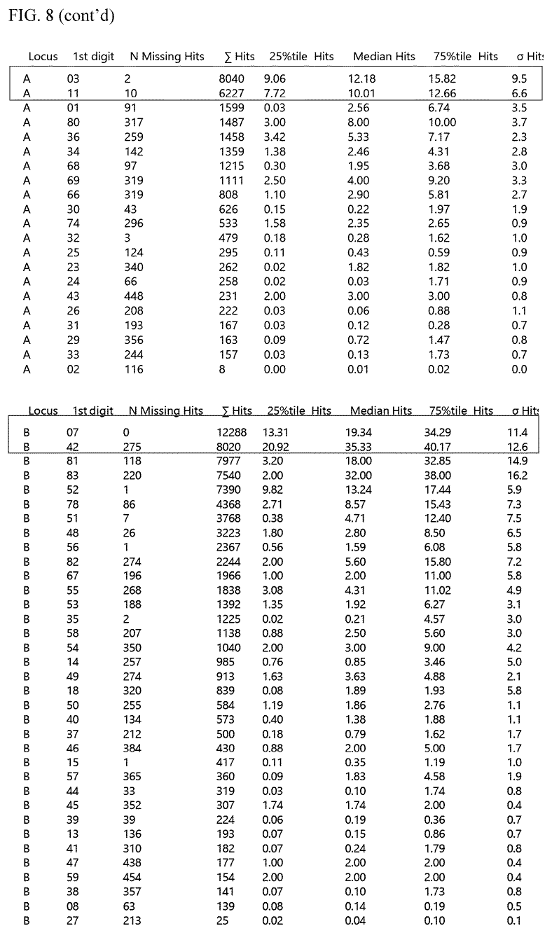

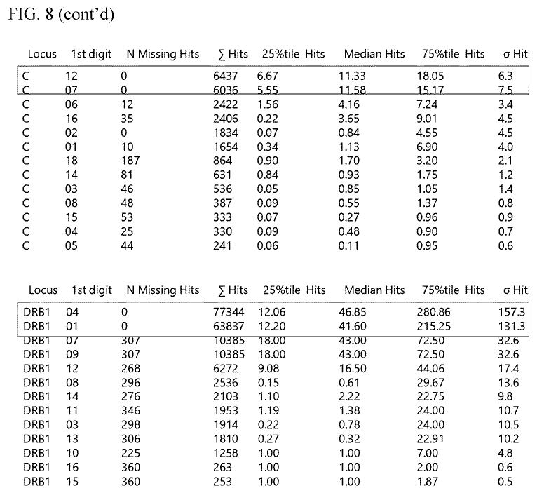

The BAM formatted ‘slice’ was converted to a fastq split read format required by magicBLAST using tools from GATK (Broad Institute). A special magicBLAST database for both MHC I and MHC II needed for the alignment process was created from the IMGT HLA sequence database (imgt.org). Exons 2 and 3 are each 270 nucleotides and code for the amino acid variations that form the basis of the different HLA haplotypes. A matrix 540×N (N=number of reads) was created and was used to tally the 100% read match at each nucleotide position produced by magicBLAST. The magicBLAST 100% alignment statistics in the matrix were then tallied across all reads and matched to the different MHC genotypes. Whereas Optitype uses a special integer linear programming approach with the hit matrix to assign the best fit HLA, we demonstrated that a simple tally of the hits in the matrix are adequate to clearly identify the haplotype of the exome data. FIG. 8 shows an example of the output.

### Example 10: Fusion Peptide Constructs

Peptides when delivered alone are usually poor immunogens. This can be overcome by delivery with an adjuvant, as described above. An alternative approach is to deliver selected peptides linked to a fusion partner which tends to facilitate nanoparticle formation, enhancing uptake by macrophages and dendritic cells. The design of such a peptide-linker-fusion partner combination must ensure that the selected peptide is excised precisely within the macrophage, dendritic cell or other antigen presenting cell to ensure that the intended binding register that exposes the desired T cell exposed motif. Several different linkers may be used, including but not limited to single amino acids, amino acid multimers, elastin, and cathepsin cleavable linkers. In one embodiment lysine and arginine residues are used which are readily cleaved by trypsin. An alternative, but more complex, approach is to design the selected peptide to terminate at a cathepsin cleavage site. Typically, an octomer must be considered that places the cathepsin scissile bond between amino acids 4 and 5 of that octomer. The fusion partner may be a polyhydrophobic amino acid peptide. In some embodiments a polyleucine may be used. Other hydrophobic amino acids may be used in place of leucine, including but not limited to phenylalanine, isoleucine or tryptophan. Alternatively, various hydrophobic unnatural amino acids may be linked to as the fusion partner. In some embodiments a lipid core peptide system comprising a lipoamino acid (LAA) moiety may be used to favor nanoparticle formation, facilitating uptake by antigen presenting cells. Other approaches to nanoparticle delivery may also be used in which the selected peptides are incorporated in liposomes or virosomes [38-41].

In another approach to enhancing uptake of neoepitope peptides of interest by antigen presenting cells the peptides, including the bespoke peptide antigens, may be linked to an immunoglobulin or to an immunoglobulin Fc region.

In preferred embodiments the selected peptide fusion constructs comprise one T cell stimulating peptide of interest. In yet other embodiments several T cell epitope peptides may be linked by linkers and attached to one fusion partner. In yet other embodiments one or more T cell stimulating peptides of interest may be linked to a B cell epitope peptide as a fusion partner.

### Example 11: Analysis of Glioblastoma and Lung Cancer Cases

Two sets of cancer cases were analyzed comprising 30 glioblastoma (GBM) and 30 squamous cell lung cancer cases (LUSC), for which all mutated protein sequences were downloaded from the Genome Data Commons which records the mutations in TCGA. As the mutations recorded in TCGA reflect the mutations detected in clinically presenting patients, they can be considered the “surviving mutations” which have not been previously eliminated by immune surveillance or by having rendered the cell apoptotic. Mutated proteins were designated as oncogenes, tumor suppressors, or passengers based on the application of that designation by Vogelstein et al and each was aligned to its normal counterpart sequences. Pairs of mutated and unmutated protein sequences were created and analyzed to determine predicted MHC binding, location of B cell linear epitopes, topology and predicted cathepsin cleavage sites as previously described [43-47]. Binding affinities were predicted for all peptide registers for each of 70 MHC class I alleles in loci A, B, and C and 70 class II alleles in loci DR, DQ and DP. Frequency of T cell exposed motifs was determined relative to both the human immunoglobulinome and the complete human proteome [44, 45]. This was done for both MHC I TCEM and MHC II TCEM. There was no significant difference observed in the patterns of TCEM frequency or topology between GBM and LUSC; tumor proteins of both sets of cases behaved similarly. Several salient observations were made upon further analysis.

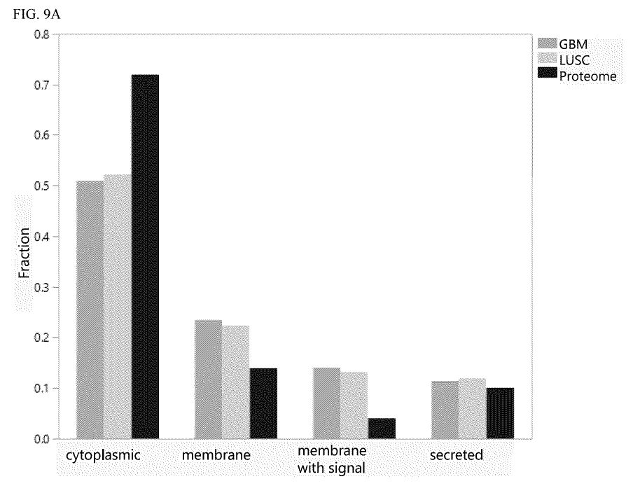

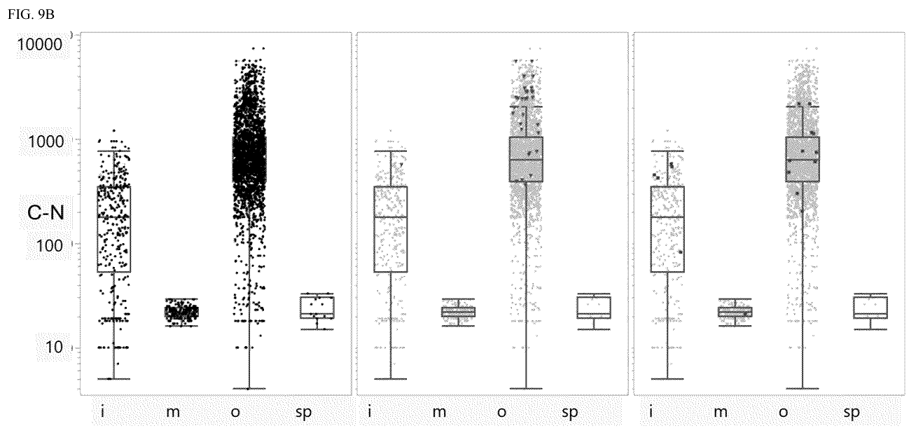

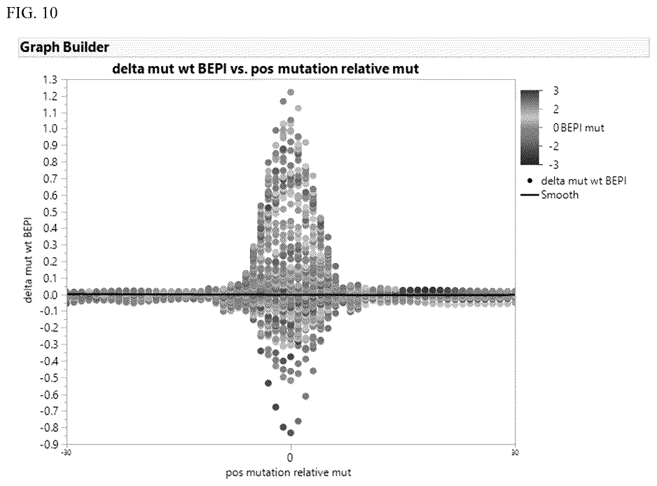

In the proteins with transmembrane domains, the mutations were more likely to be present in extracellular domains than in the portions of those proteins located in the cytoplasm. This is shown in FIG. 9, where it is seen that, among the mutated tumor proteins, the ratio of cytoplasmic to membrane or extracellular domain proteins is reversed as compared to the distribution in the proteome as a whole. As also shown in FIG. 1 in those proteins with extracellular domains and transmembrane domains, the mutations are more likely to be in the extracellular domain. This is the case for oncogenes, tumor suppressor proteins and proteins with passenger mutations alike of both GBM and LUSC cases. In addition, many of the mutated proteins have very extensive extracellular domain segments. As a result tumor proteins are more likely than the proteome as a whole to have exposed B cell epitopes, which in some cases comprised the mutated amino acid and in other instances were in close proximity to T cell epitopes with mutated amino acids, providing a unique immunologic signal. In some proteins the mutations generate de novo high probability B cell epitopes as shown in FIG. 10.

In 60% of cases, peptides which comprised mutant amino acids were not predicted to be in the top 15% of highest MHC binding affinity for either MHC I or MHC II alleles. Mutated amino acids only affected binding when they occurred in pocket position. In the case of MHC I this was particularly marked when the mutant amino acid was in pocket position 2 or 9, as shown in FIG. 11.

Mutations consistently generated motifs which were absent or less frequent in the total human proteome database than in their non mutated normal counterparts. This is shown in FIG. 12 where the residuals are all outside the 95% boundary of the regression.

These findings confirmed the observations in individual cancer patients cited in prior examples by demonstrating that mutations present in tumor proteins by the time of clinical diagnosis have developed several means of camouflage from immune surveillance and elimination and that strategies to overcome such camouflage must be employed to achieve effective immunotherapy. The present invention provides such strategies by devising means to expose and present the tumor specific peptides to T cell recognition on as many MHC alleles as possible, and by utilizing the B cell epitopes also exposed.

### Example 12: Immunopathologies

The ability to generate bespoke peptides to “tune” the T cell response of an individual subject of known HLA has applications outside the field of cancer immunology. Immunomodulation of excessive T cell responses can assist in the management of allergy and autoimmune diseases and other immunopathologies. To investigate this, we generated bespoke peptides for a commonly recognized peanut allergen Ara h6 and for two proteins recognized as drivers of rheumatoid arthritis. In both cases the goal was to design peptides which could down regulate CD4+ T helper cells.

Based on Genome Wide association studies there is evidence that peanut allergies may be linked to both DRB1 and DQB alleles [48, 49]. We modelled the design of novel peptides around the dominant T helper motif in ara6 h to create peptides with very high binding to DRB1_0101 and DQA1_0101_DQB1_0501. The choice of these alleles is not considered limiting as a similar approach could be used to generate peptides if a desired binding affinity for any of the DQA DQB combinations or any DRB allele.

### Peanut Ara h6

The ara h6 protein contains a number of T cell exposed motifs which are very rare in the human proteome and in the gastrointestinal microbiome. This is not unusual in proteins of allergens and it appears that the exposure of an individual to a sudden large pulse of such rare antigens has the effect of triggering an allergic reaction. This is in contradistinction to the previously cited situation in cancer where a single rare motif may be present but evade immune surveillance.

Table 27 provides non limiting examples of peptides with enhanced binding to various MHC II alleles (examples shown are nonlimiting), demonstrating and increase of approximately 2 standard deviations in predicted binding affinity over the natural peptide at that position. Such very high affinity binding peptides would be expected to induce exhaustion and anergy of the cognate T cell clonal population.

Proteins Associated with Rheumatoid Arthritis

The two proteins we examined are vimentin and Alpha enolase. In both cases peptides have been identified which are drivers of the autoimmune reaction when citrullinated at specific arginine residues [50-52]. Rheumatoid arthritis is predominantly found in individuals who carry the DRB1_0401 allele [53, 54]. Our goal was therefore to design peptides which would retain a T cell exposed motif that exposes the citrullinated residue to the T cell receptor, while modifying the flanking regions to create a very high binding peptide capable to leading to exhaustion and anergy of the T cell response. While the example shows design of high biding peptides for DRB1_0401 given that RA is the example of interest, for other autoimmune conditions other alleles may be relevant and thus the example is not considered limiting.

Table 28 shows the increased binding achieved by designing peptides to expose the citrullinated residues but alter amino acids in the flanking regions. An approximately two standard deviation unit increase in binding is achieved, making the bespoke peptides “super binders” likely to induce exhaustion and anergy of the corresponding Th clones

TABLE 27

| | Index | original | | SEQ | Enhanced | | SEQ | | SEQ |
|---|---|---|---|---|---|---|---|---|---|
| | amino | binding | | ID | binding | | ID | | ID |
| Allele | acid | SD units | original peptide | NO: | SD units | bespoke peptide | NO: | TCEM IIa | NO: |
| DRB1_0101 | 117 | −1.23 | FKRELMNLPQQCNFR | 994 | −3.81 | LNRLLMNLPQQATLI | 1013 | LM~L~QQ | 1032 |
| DRB1_0101 | 116 | −0.97 | QFKRELMNLPQQCNF | 995 | −3.31 | IRQLELMNLPQIYLN | 1014 | EL~N~PQ | 1033 |
| DRB1_0101 | 115 | −0.54 | QQFKRELMNLPQQCN | 996 | −2.88 | MIRLRELMNLPVARC | 1015 | RE~M~LP | 1034 |
| DRB1_0101 | 48 | −0.51 | EQHIMQRIMGEQEQY | 997 | −2.78 | VQAMMQRIMGELLLE | 1016 | MQ~I~GE | 1035 |
| DRB1_0101 | 116 | −0.97 | QFKRELMNLPQQCNF | 998 | −2.76 | RQMQELMNLPQLILI | 1017 | EL~N~PQ | 1036 |
| DRB1_0101 | 88 | −0.34 | NTQRCMCEALQQIME | 999 | −2.73 | MQFMCMCEALQALLV | 1018 | CM~E~LQ | 1037 |
| DRB1_0401 | 115 | −0.87 | QQFKRELMNLPQQCN | 1000 | −3.66 | PMLLRELMNLPRTRR | 1019 | RE~M~LP | 1038 |
| DRB1_0401 | 117 | −0.88 | FKRELMNLPQQCNFR | 1001 | −3.46 | LILLLMNLPQQNTVN | 1020 | LM~L~QQ | 1039 |
| DRB1_0401 | 116 | −0.78 | QFKRELMNLPQQCNF | 1002 | −3.16 | FLIFELMNLPQMRNI | 1021 | EL~N~PQ | 1040 |
| DRB1_0401 | 45 | −1.10 | KPCEQHIMQRIMGEQ | 1003 | −2.81 | IMFLQHIMQRIELQY | 1022 | QH~M~RI | 1041 |
| DRB1_0401 | 48 | −1.66 | EQHIMQRIMGEQEQY | 1004 | −2.75 | LRMLMQRIMGENQRV | 1023 | MQ~I~GE | 1042 |
| DQA1_0101- | 47 | −1.14 | CEQHIMQRIMGEQEQ | 1005 | −3.46 | RELQIMQRIMGAVLC | 1024 | IM~R~MG | 1043 |
| DQB1_0501 | | | | | | | | | |
| DQA1_0101- | 46 | −2.34 | PCEQHIMQRIMGEQE | 1006 | −3.33 | LHQRHIMQRIMAQVF | 1025 | HI~Q~IM | 1044 |
| DQB1_0501 | | | | | | | | | |
| DQA1_0101- | 45 | −1.23 | KPCEQHIMQRIMGEQ | 1007 | −3.08 | LQVDQHIMQRISCLM | 1026 | QH~M~RI | 1045 |
| DQB1_0501 | | | | | | | | | |
| DQA1_0101- | 113 | −1.01 | MVQQFKRELMNLPQQ | 1008 | −2.97 | NIILFKRELMNMHQC | 1027 | FK~E~MN | 1046 |
| DQB1_0501 | | | | | | | | | |
| DQA1_0101- | 116 | −0.65 | QFKRELMNLPQQCNF | 1009 | −2.44 | CCVQELMNLPQRCAA | 1028 | EL~N~PQ | 1047 |
| DQB1_0501 | | | | | | | | | |
| DQA1_0102- | 47 | −0.64 | CEQHIMQRIMGEQEQ | 1010 | −2.92 | MIMMIMQRIMGSVCG | 1029 | IM~R~MG | 1048 |
| DQB1_0602 | | | | | | | | | |
| DQA1_0102- | 45 | −0.40 | KPCEQHIMQRIMGEQ | 1011 | −2.64 | GCACQHIMQRIPCAR | 1030 | QH~M~RI | 1049 |
| DQB1_0602 | | | | | | | | | |
| DQA1_0102- | 46 | −0.62 | PCEQHIMQRIMGEQE | 1012 | −2.50 | CCSIHIMQRIMALAD | 1031 | HI~Q~IM | 1050 |
| DQB1_0602 | | | | | | | | | |

TABLE 28

| | | original | | | Enhanced | | | | |
|---|---|---|---|---|---|---|---|---|---|
| Index | | binding to | | SEQ | binding to | | SEQ | TCEM | SEQ |
| amino | | DRB1_0401 | original | ID | DRB1_0401 | | ID | IIA | ID |
| acid | curation | SD units | peptide | NO: | SD units | bespoke peptide | NO: | Core | NO: |
| 5 | alpha- | −1.19 | KIHAREIFDSXGNPT | 1051 | −3.02 | PLIFREIFDSXGVQI | 1065 | RE~F~SX | 1079 |
| 8 | enolase | −1.51 | AREIFDSXGNPTVEV | 1052 | −3.79 | KLIFFDSXGNPTADM | 1066 | FD~X~NP | 1080 |
| 8 | isoform 1 | −1.51 | AREIFDSXGNPTVEV | 1053 | −2.99 | DFNFFDSXGNPSASL | 1067 | FD~X~NP | 1081 |
| 25 | | −0.33 | FTSKGLFXAAVPSGA | 1054 | −3.05 | QLLFGLFXAAVLTKH | 1068 | GL~X~AV | 1082 |
| 27 | | −1.90 | SKGLFXAAVPSGAST | 1055 | −3.62 | ALQYFXAAVPSSGLM | 1069 | FX~A~PS | 1083 |
| 28 | | −1.98 | KGLFXAAVPSGASTG | 1056 | −2.67 | VIIFXAAVPSGGGLI | 1070 | XA~V~SG | 1084 |
| 57 | Vimentin | −0.96 | PGGVYATXSSAVXLX | 1057 | −3.09 | KQQYYATXSSAGSLF | 1071 | YA~X~SA | 1085 |
| 60 | | −0.10 | VYATXSSAVXLXSSV | 1058 | −2.88 | NAFFXSSAVXLGLST | 1072 | XS~A~XL | 1086 |
| 64 | | −0.03 | XSSAVXLXSSVPGVR | 1059 | −2.84 | RAILVXLXSSVKAQI | 1073 | VX~X~SV | 1087 |
| 65 | | −0.78 | SSAVXLXSSVPGVRL | 1060 | −2.91 | EMLWXLXSSVPGTQD | 1074 | XL~S~VP | 1088 |
| 66 | | −1.41 | SAVXLXSSVPGVRLL | 1061 | −2.91 | TLEWLXSSVPGSGLP | 1075 | LX~S~PG | 1089 |
| 414 | | −2.40 | PLPNFSSLNLXETNL | 1062 | −3.70 | PFYVFSSLNLXNNVA | 1076 | FS~L~LX | 1090 |
| 417 | | −1.27 | NFSSLNLXETNLDSL | 1063 | −3.86 | QLIWLNLXETNIQTA | 1077 | LN~X~TN | 1091 |
| 419 | | −1.64 | SSLNLXETNLDSLPL | 1064 | −2.99 | YQILLXETNLDDAPM | 1078 | LX~T~LD | 1092 |

Citrullinated amino acids represented by X

1. Lefranc M P, Giudicelli V, Ginestoux C, Jabado-Michaloud J, Folch G, Bellahcene F, et al. IMGT, the international ImMunoGeneTics information system. Nucleic acids research. 2009; 37 (Database issue): D1006-12. Epub 2008 Nov. 4. doi: 10.1093/nar/gkn838. PubMed PMID: 18978023; PubMed Central PMCID: PMC2686541.
2. Hanahan D, Weinberg R A. Hallmarks of cancer: the next generation. Cell. 2011; 144(5):646-74. Epub 2011 Mar. 8. doi: 10.1016/j.cell.2011.02.013. PubMed PMID: 21376230.
3. Chen D S, Mellman I. Elements of cancer immunity and the cancer-immune set point. Nature. 2017; 541(7637):321-30. Epub 2017 Jan. 20. doi: 10.1038/nature21349. PubMed PMID: 28102259.
4. Adusumilli P S, Cha E, Cornfeld M, Davis T, Diab A, Dubensky T W, Jr., et al. New Cancer Immunotherapy Agents in Development: a report from an associated program of the 31(st) Annual Meeting of the Society for Immunotherapy of Cancer, 2016. J Immunother Cancer. 2017; 5:50. Epub 2017 Jun. 27. doi: 10.1186/s40425-017-0253-2. PubMed PMID: 28649381; PubMed Central PMCID: PMCPMC5477277.
5. Ilyas S, Yang J C. Landscape of Tumor Antigens in T Cell Immunotherapy. J Immunol. 2015; 195(11):5117-22. Epub 2015 Nov. 22. doi: 10.4049/jimmunol. 1501657. PubMed PMID: 26589749; PubMed Central PMCID: PMCPMC4656134.
Aldous A R, Dong J Z. Personalized neoantigen vaccines: A new approach to cancer immunotherapy. Bioorg Med Chem. 2018; 26(10):2842-9. Epub 2017 Nov. 8. doi: 10.1016/j.bmc.2017.10.021. PubMed PMID: 29111369.
7. Ophir E, Bobisse S, Coukos G, Harari A, Kandalaft L E. Personalized approaches to active immunotherapy in cancer. Biochim Biophys Acta. 2016; 1865(1):72-82. Epub 2015 Aug. 5. doi: 10.1016/j.bbcan.2015.07.004. PubMed PMID: 26241169.
8. Fennemann F L, de Vries I J M, Figdor C G, Verdoes M. Attacking Tumors From All Sides: Personalized Multiplex Vaccines to Tackle Intratumor Heterogeneity. Frontiers in immunology. 2019; 10:824. Epub 2019 May 2. doi: 10.3389/fimmu.2019.00824. PubMed PMID: 31040852; PubMed Central PMCID: PMCPMC6476980.
9. Ott P A, Hu Z, Keskin D B, Shukla S A, Sun J, Bozym D J, et al. An immunogenic personal neoantigen vaccine for patients with melanoma. Nature. 2017; 547(7662):217-21. Epub 2017 Jul. 6. doi: 10.1038/nature22991. PubMed PMID: 28678778; PubMed Central PMCID: PMCPMC5577644.
10. Sahin U, Derhovanessian E, Miller M, Kloke B P, Simon P, Lower M, et al. Personalized RNA mutanome vaccines mobilize poly-specific therapeutic immunity against cancer. Nature. 2017; 547(7662):222-6. Epub 2017 Jul. 6. doi: 10.1038/nature23003. PubMed PMID: 28678784.
11. Li F, Chen C, Ju T, Gao J, Yan J, Wang P, et al. Rapid tumor regression in an Asian lung cancer patient following personalized neo-epitope peptide vaccination. Oncoimmunology. 2016; 5(12):e1238539. Epub 2017 Jan. 27. doi: 10.1080/2162402X.2016.1238539. PubMed PMID: 28123873; PubMed Central PMCID: PMCPMC5214696.
12. Hilf N, Kuttruff-Coqui S, Frenzel K, Bukur V, Stevanovic S, Gouttefangeas C, et al. Actively personalized vaccination trial for newly diagnosed glioblastoma. Nature. 2019; 565(7738):240-5. Epub 2018 Dec. 21. doi: 10.1038/s41586-018-0810-y. PubMed PMID: 30568303.
13. Keskin D B, Anandappa A J, Sun J, Tirosh I, Mathewson N D, Li S, et al. Neoantigen vaccine generates intratumoral T cell responses in phase Ib glioblastoma trial. Nature. 2019; 565(7738):234-9. Epub 2018 Dec. 21. doi: 10.1038/s41586-018-0792-9. PubMed PMID: 30568305.
14. Rabizadeh S, Garner C, Sanborn J Z, Benz S C, Reddy S, Soon-Shiong P. Comprehensive genomic transcriptomic tumor-normal gene panel analysis for enhanced precision in patients with lung cancer. Oncotarget. 2018; 9(27):19223-32. Epub 2018 May 4. doi: 10.18632/oncotarget.24973. PubMed PMID: 29721196; PubMed Central PMCID: PMCPMC5922390.
15. Yadav M, Jhunjhunwala S, Phung Q T, Lupardus P, Tanguay J, Bumbaca S, et al. Predicting immunogenic tumour mutations by combining mass spectrometry and exome sequencing. Nature. 2014; 515(7528):572-6. Epub 2014 Nov. 28. doi: 10.1038/nature14001. PubMed PMID: 25428506.
16. Abelin J G, Keskin D B, Sarkizova S, Hartigan C R, Zhang W, Sidney J, et al. Mass Spectrometry Profiling of HLA-Associated Peptidomes in Mono-allelic Cells Enables More Accurate Epitope Prediction. Immunity. 2017; 46(2):315-26. Epub 2017 Feb. 24. doi: 10.1016/j.immuni.2017.02.007. PubMed PMID: 28228285; PubMed Central PMCID: PMCPMC5405381.
17. Hoof I, Peters B, Sidney J, Pedersen L E, Sette A, Lund O, et al. NetMHCpan, a method for MHC class I binding prediction beyond humans. Immunogenetics. 2009; 61(1):1-13. doi: 10.1007/s00251-008-0341-z [doi].
18. Havel J J, Chowell D, Chan T A. The evolving landscape of biomarkers for checkpoint inhibitor immunotherapy. Nature reviews Cancer. 2019; 19 (3): 133-50. Epub 2019 Feb. 14. doi: 10.1038/s41568-019-0116-x. PubMed PMID: 30755690.
19. Mandal R, Samstein R M, Lee K W, Havel J J, Wang H, Krishna C, et al. Genetic diversity of tumors with mismatch repair deficiency influences anti-PD-1 immunotherapy response. Science. 2019; 364 (6439): 485-91. Epub 2019 May 3. doi: 10.1126/science.aau0447. PubMed PMID: 31048490.
20. Gibney G T, Weiner L M, Atkins M B. Predictive biomarkers for checkpoint inhibitor-based immunotherapy. The lancet oncology. 2016; 17(12):e542-e51. Epub 2016 Dec. 8. doi: 10.1016/S1470-2045(16)30406-5. PubMed PMID: 27924752; PubMed Central PMCID: PMCPMC5702534.
21. Bajwa R, Cheema A, Khan T, Amirpour A, Paul A, Chaughtai S, et al. Adverse Effects of Immune Checkpoint Inhibitors (Programmed Death-1 Inhibitors and Cytotoxic T-Lymphocyte-Associated Protein-4 Inhibitors): Results of a Retrospective Study. J Clin Med Res. 2019; 11(4):225-36. Epub 2019 Apr. 3. doi: 10.14740/jocmr3750. PubMed PMID: 30937112; PubMed Central PMCID: PMCPMC6436564.
22. Gubin M M, Zhang X, Schuster H, Caron E, Ward J P, Noguchi T, et al. Checkpoint blockade cancer immunotherapy targets tumour-specific mutant antigens. Nature. 2014; 515(7528):577-81. Epub 2014 Nov. 28. doi: 10.1038/nature13988. PubMed PMID: 25428507; PubMed Central PMCID: PMCPMC4279952.
23. Bailey M H, Tokheim C, Porta-Pardo E, Sengupta S, Bertrand D, Weerasinghe A, et al. Comprehensive Characterization of Cancer Driver Genes and Mutations. Cell. 2018; 173(2):371-85 e18. Epub 2018 Apr. 7. doi: 10.1016/j.cell.2018.02.060. PubMed PMID: 29625053; PubMed Central PMCID: PMCPMC6029450.
24. Chang M T, Asthana S, Gao S P, Lee B H, Chapman J S, Kandoth C, et al. Identifying recurrent mutations in cancer reveals widespread lineage diversity and mutational specificity. Nat Biotechnol. 2016; 34(2):155-63. Epub 2015 Dec. 1. doi: 10.1038/nbt.3391. PubMed PMID: 26619011; PubMed Central PMCID: PMCPMC4744099.
25. Kandoth C, McLellan M D, Vandin F, Ye K, Niu B, Lu C, et al. Mutational landscape and significance across 12 major cancer types. Nature. 2013; 502(7471):333-9. Epub 2013 Oct. 18. doi: 10.1038/nature12634. PubMed PMID: 24132290; PubMed Central PMCID: PMCPMC3927368.
26. Tamborero D, Gonzalez-Perez A, Perez-Llamas C, Deu-Pons J, Kandoth C, Reimand J, et al. Comprehensive identification of mutational cancer driver genes across 12 tumor types. Scientific reports. 2013; 3:2650. Epub 2013 Oct. 3. doi: 10.1038/srep02650. PubMed PMID: 24084849; PubMed Central PMCID: PMCPMC3788361.
27. Kim H, Kim Y M. Pan-cancer analysis of somatic mutations and transcriptomes reveals common functional gene clusters shared by multiple cancer types. Scientific reports. 2018; 8(1):6041. Epub 2018 Apr. 18. doi: 10.1038/s41598-018-24379-y. PubMed PMID: 29662161; PubMed Central PMCID: PMCPMC5902616.
28. Weller M, Butowski N, Tran D D, Recht L D, Lim M, Hirte H, et al. Rindopepimut with temozolomide for patients with newly diagnosed, EGFRvIII-expressing glioblastoma (ACT IV): a randomised, double-blind, international phase 3 trial. The lancet oncology. 2017; 18(10):1373-85. Epub 2017 Aug. 29. doi: 10.1016/S1470-2045 (17) 30517-X. PubMed PMID: 28844499.
29. Cohen C J, Gartner J J, Horovitz-Fried M, Shamalov K, Trebska-McGowan K, Bliskovsky V V, et al. Isolation of neoantigen-specific T cells from tumor and peripheral lymphocytes. J Clin Invest. 2015; 125(10):3981-91. Epub 2015 Sep. 22. doi: 10.1172/JCI82416. PubMed PMID: 26389673; PubMed Central PMCID: PMCPMC4607110.
30. Lauvau G, Soudja S M. Mechanisms of Memory T Cell Activation and Effective Immunity. Adv Exp Med Biol. 2015; 850:73-80. Epub 2015 Sep. 2. doi: 10.1007/978-3-319-15774-0_6. PubMed PMID: 26324347; PubMed Central PMCID: PMCPMC4836952.
31. Zehn D, Lee S Y, Bevan M J. Complete but curtailed T-cell response to very low-affinity antigen. Nature. 2009; 458(7235):211-4. Epub 2009 Feb. 3. doi: 10.1038/nature07657. PubMed PMID: 19182777; PubMed Central PMCID: PMCPMC2735344.
32. Soudja S M, Chandrabos C, Yakob E, Veenstra M, Palliser D, Lauvau G. Memory-T-cell-derived interferon-gamma instructs potent innate cell activation for protective immunity. Immunity. 2014; 40(6):974-88. Epub 2014 Jun. 17. doi: 10.1016/j.immuni.2014.05.005. PubMed PMID: 24931122; PubMed Central PMCID: PMCPMC4105986.
33. Wucherpfennig K W, Allen P M, Celada F, Cohen I R, De Boer R, Garcia K C, et al. Polyspecificity of T cell and B cell receptor recognition. Seminars in immunology. 2007; 19(4):216-24. Epub 2007 Apr. 3. doi: 10.1016/j.smim.2007.02.012. PubMed PMID: 17398114; PubMed Central PMCID: PMC2034306.
34. An Z, Aksoy O, Zheng T, Fan Q W, Weiss W A. Epidermal growth factor receptor and EGFRvIII in glioblastoma: signaling pathways and targeted therapies. Oncogene. 2018; 37(12):1561-75. Epub 2018 Jan. 13. doi: 10.1038/s41388-017-0045-7. PubMed PMID: 29321659; PubMed Central PMCID: PMCPMC5860944.
35. Szolek A, Schubert B, Mohr C, Sturm M, Feldhahn M, Kohlbacher O. OptiType: precision HLA typing from next-generation sequencing data. Bioinformatics. 2014; 30 (23): 3310-6. Epub 2014 Aug. 22. doi: 10.1093/bioinformatics/btu548. PubMed PMID: 25143287; PubMed Central PMCID: PMCPMC4441069.
36. Boratyn G M, Thierry-Mieg J, Thierry-Mieg D, Busby B, Madden T L. Magic-BLAST, an accurate RNA-seq aligner for long and short reads. BMC Bioinformatics. 2019; 20(1):405. Epub 2019 Jul. 28. doi: 10.1186/s12859-019-2996-x. PubMed PMID: 31345161; PubMed Central PMCID: PMCPMC6659269.
37. Larjo A, Eveleigh R, Kilpelainen E, Kwan T, Pastinen T, Koskela S, et al. Accuracy of Programs for the Determination of Human Leukocyte Antigen Alleles from Next-Generation Sequencing Data. Frontiers in immunology. 2017; 8:1815. Epub 2018 Jan. 13. doi: 10.3389/fimmu.2017.01815. PubMed PMID: 29326702; PubMed Central PMCID: PMCPMC5733459.
38. Skwarczynski M, Dougall A M, Khoshnejad M, Chandrudu S, Pearson M S, Loukas A, et al. Peptide-based subunit vaccine against hookworm infection. PloS one. 2012; 7(10):e46870. Epub 2012 Oct. 12. doi: 10.1371/journal.pone.0046870. PubMed PMID: 23056500; PubMed Central PMCID: PMCPMC3463534.
39. Skwarczynski M, Toth I. Non-invasive mucosal vaccine delivery: advantages, challenges and the future. Expert Opin Drug Deliv. 2020:1-3. Epub 2020 Feb. 16. doi: 10.1080/17425247.2020.1731468. PubMed PMID: 32059625.
40. Nandedkar T D. Nanovaccines: recent developments in vaccination. Journal of biosciences. 2009; 34(6):995-1003. Epub 2010 Jan. 23. doi: 10.1007/s12038-009-0114-3. PubMed PMID: 20093753.
41. Bartlett S, Eichenberger R M, Nevagi R J, Ghaffar K A, Marasini N, Dai Y, et al. Lipopeptide-based oral vaccine against hookworm infection. J Infect Dis. 2019. Epub 2019 Oct. 18. doi: 10.1093/infdis/jiz528. PubMed PMID: 31621864.
42. Vogelstein B, Papadopoulos N, Velculescu V E, Zhou S, Diaz L A, Jr., Kinzler K W. Cancer genome landscapes. Science. 2013; 339(6127):1546-58. Epub 2013 Mar. 30. doi: 10.1126/science. 1235122. PubMed PMID: 23539594; PubMed Central PMCID: PMCPMC3749880.
43. Bremel R D, Homan E J. An integrated approach to epitope analysis II: A system for proteomic-scale prediction of immunological characteristics. ImmunomeRes. 2010; 6(1):8. doi: 1745-7580-6-8 [pii]; 10.1186/1745-7580-6-8 [doi].
44. Bremel R D, Homan E J. Frequency Patterns of T-Cell Exposed Amino Acid Motifs in Immunoglobulin Heavy Chain Peptides Presented by MHCs. Frontiers in immunology. 2014; 5:541. doi: 10.3389/fimmu.2014.00541. PubMed PMID: 25389426; PubMed Central PMCID: PMC4211557.
45. Bremel R D, Homan J. Extensive T-cell epitope repertoire sharing among human proteome, gastrointestinal microbiome, and pathogenic bacteria: Implications for the definition of self. Frontiers in immunology. 2015; 6. doi: 10.3389/fimmu.2015.00538.
46. Homan E J, Bremel R D. Patterns of Predicted T-Cell Epitopes Associated with Antigenic Drift in Influenza H3N2 Hemagglutinin. PLOSOne. 2011; 6(10):e26711. doi: 10.1371/journal.pone.0026711 [doi]; PONE-D-11-07616 [pii].
47. Hoglund R A, Torsetnes S B, Lossius A, Bogen B, Homan E J, Bremel R, et al. Human Cysteine Cathepsins Degrade Immunoglobulin G In Vitro in a Predictable Manner. Int J Mol Sci. 2019; 20(19). Epub 2019 Oct. 2. doi: 10.3390/ijms20194843. PubMed PMID: 31569504; PubMed Central PMCID: PMCPMC6801702.
48. Asai Y, Eslami A, van Ginkel C D, Akhabir L, Wan M, Ellis G, et al. Genome-wide association study and meta-analysis in multiple populations identifies new loci for peanut allergy and establishes C11orf30/EMSY as a genetic risk factor for food allergy. J Allergy Clin Immunol. 2018; 141 (3): 991-1001. Epub 2017 Oct. 17. doi: 10.1016/j.jaci.2017.09.015. PubMed PMID: 29030101.
49. Asai Y, Eslami A, van Ginkel C D, Akhabir L, Wan M, Yin D, et al. A Canadian genome-wide association study and meta-analysis confirm HLA as a risk factor for peanut allergy independent of asthma. J Allergy Clin Immunol. 2018; 141(4):1513-6. Epub 2018 Jan. 13. doi: 10.1016/j.jaci.2017.10.047. PubMed PMID: 29325868.
50. Gerstner C, Dubnovitsky A, Sandin C, Kozhukh G, Uchtenhagen H, James E A, et al. Functional and Structural Characterization of a Novel HLA-DRB1*04:01-Restricted alpha-Enolase T Cell Epitope in Rheumatoid Arthritis. Frontiers in immunology. 2016; 7:494. Epub 2016 Nov. 30. doi: 10.3389/fimmu.2016.00494. PubMed PMID: 27895642; PubMed Central PMCID: PMCPMC5108039.
51. Gerstner C, Turcinov S, Hensvold A H, Chemin K, Uchtenhagen H, Ramwadhdoebe T H, et al. Multi-HLA class II tetramer analyses of citrulline-reactive T cells and early treatment response in rheumatoid arthritis. BMC Immunol. 2020; 21(1):27. Epub 2020 May 20. doi: 10.1186/s12865-020-00357-w. PubMed PMID: 32423478; PubMed Central PMCID: PMCPMC7236297.
52. James E A, Rieck M, Pieper J, Gebe J A, Yue B B, Tatum M, et al. Citrulline-specific Th1 cells are increased in rheumatoid arthritis and their frequency is influenced by disease duration and therapy. Arthritis Rheumatol. 2014; 66(7):1712-22. Epub 2014 Mar. 26. doi: 10.1002/art.38637. PubMed PMID: 24665079; PubMed Central PMCID: PMCPMC4248674.
53. Klareskog L, Padyukov L, Ronnelid J, Alfredsson L. Genes, environment and immunity in the development of rheumatoid arthritis. Curr Opin Immunol. 2006; 18(6):650-5. Epub 2006 Oct. 3. doi: 10.1016/j.coi.2006.06.004. PubMed PMID: 17010589.
54. Klareskog L, Stolt P, Lundberg K, Kallberg H, Bengtsson C, Grunewald J, et al. A new model for an etiology of rheumatoid arthritis: smoking may trigger HLA-DR (shared epitope)-restricted immune reactions to autoantigens modified by citrullination. Arthritis Rheum.
2006; 54(1):38-46. Epub 2005 Dec. 31. doi: 10.1002/art.21575. PubMed PMID: 16385494.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

What is claimed is:

1. A method for treating cancer in a subject comprising designing a group of one or more tumor-specific T-cell stimulating peptides, or nucleic acids encoding T cell stimulating peptides, which have a desired predicted binding affinity for the MHC alleles of the subject, comprising the following steps:
obtaining a biopsy of the subject's tumor;
obtaining sequences for proteins in said biopsy;
identifying proteins from the biopsy containing mutated amino acids and the peptide comprising each of said mutated amino acids;
determining T cell exposed motifs which comprise mutated amino acids in each of the proteins;
determining the predicted binding affinity to the subject's MHC alleles of peptides which comprises each of said T cell exposed motifs, or a subset thereof;
generating an array of alternative peptides not present in the tumor, wherein each peptide in the array comprises the amino acids of one of said T cell exposed motifs, and in which one or more of the amino acids not within the T cell exposed motif are substituted to change the predicted MHC binding affinity;
selecting a group of one or more selected peptides from said array of alternative peptides which have a desired predicted binding affinity for one or more of the subject's MHC alleles; and
synthesizing said group of one or more selected peptides, or nucleic acids encoding the selected peptides.

2. The method of claim 1 wherein said MHC alleles are MHC type I and said T cell response is a CD8+ response.

3. The method of claim 1 wherein said MHC alleles are MHC type II and said T cell response is a CD4+ response.

4. The method of claim 1 wherein said selected peptides are less than 20 amino acids long.

5. The method of claim 1, wherein said group of one or more selected peptides comprises at least 5 unique peptides not present in the proteins sequenced in the tumor.

6. The method of claim 1, wherein said desired predicted binding affinity is less than 100 nanomolar.

7. The method of claim 1, wherein the proteins in the subject's biopsy comprise mutations that are unique to that subject.

8. The method of claim 1, wherein the proteins in the subject's biopsy comprise mutations that are found in a multiplicity of cancers affecting a multiplicity of subjects.

9. The method of claim 1, wherein said group of one or more selected peptides, or nucleic acids encoding the peptides, are prescribed for an identified individual patient.

10. The method of claim 1, wherein said group of one or more selected peptides, or the nucleic acids encoding them, is administered to a subject as a vaccine.

11. The method of claim 1, wherein said group of one or more selected peptides, or the nucleic acids that encode them, is provided to contact an antigen presenting cell in vitro and said antigen presenting cells are subsequently administered to a subject.

12. The method of claim 1, wherein said group of one or more selected peptides is provided to stimulate T cells in vitro which are subsequently administered to a subject.

13. A vaccine for administration to a subject with cancer comprising a group of peptides, or nucleic acids encoding the same peptides, selected according to the method of claim 1.

14. The vaccine of claim 13, wherein said vaccination is accompanied by administration of an immunotherapy intervention

15. The vaccine of claim 13, wherein said group of peptides or nucleic acids encoding the same peptides, is selected to stimulate T cells that target mutations unique to the particular subject.

16. The vaccine of claim 13, wherein said group of peptides or nucleic acids encoding the same peptides, is selected to stimulate T cells that target mutations shared among a multiplicity of cancers.

17. The vaccine of claim 13, wherein said vaccine is administered to a subject parenterally.

18. The vaccine of claim 13, wherein said vaccine is administered to a subject intradermally.

19. The vaccine of claim 13, wherein said group of peptides or nucleic acids encoding the same peptides, spans the deletion of exons 2-7 in EGFRviii.

20. The vaccine of claim 13, wherein said peptides comprise the T cell exposed motifs from the group EEKKG, EKKGN, KKGNY, KGNYV, GNYVV.


SEQUENCE LISTING (1095 sequences)

SEQ ID NO: 1 - protein, 5 aa, Homo sapiens
YSFGV

SEQ ID NO: 2 - protein, 5 aa, Homo sapiens
SFGVA

SEQ ID NO: 3 - protein, 5 aa, Homo sapiens
GVATC

SEQ ID NO: 4 - protein, 5 aa, Homo sapiens
VATCV

SEQ ID NO: 5 - protein, 5 aa, Homo sapiens
SFGVA

SEQ ID NO: 6 - protein, 5 aa, Homo sapiens
FGVAT

SEQ ID NO: 7 - protein, 5 aa, Homo sapiens
GVATC

SEQ ID NO: 8 - protein, 5 aa, Homo sapiens
YSFGV

SEQ ID NO: 9 - protein, 5 aa, Homo sapiens
SFGVA

SEQ ID NO: 10 - protein, 5 aa, Homo sapiens
VATCV

SEQ ID NO: 11 - protein, 5 aa, Homo sapiens
FGVAT

SEQ ID NO: 12 - protein, 5 aa, Homo sapiens
FGRLA

SEQ ID NO: 13 - protein, 5 aa, Homo sapiens
TDFGR

SEQ ID NO: 14 - protein, 5 aa, Homo sapiens
DFGRL

SEQ ID NO: 15 - protein, 5 aa, Homo sapiens
GRLAK

SEQ ID NO: 16 - protein, 5 aa, Homo sapiens
RLAKL

SEQ ID NO: 17 - protein, 5 aa, Homo sapiens
DFGRL

SEQ ID NO: 18 - protein, 5 aa, Homo sapiens
FGRLA

SEQ ID NO: 19 - protein, 5 aa, Homo sapiens
DFGRL

SEQ ID NO: 20 - protein, 5 aa, Homo sapiens
FGRLA

SEQ ID NO: 21 - protein, 5 aa, Homo sapiens
RLAKL

SEQ ID NO: 22 - protein, 5 aa, Homo sapiens
LIKRQ

SEQ ID NO: 23 - protein, 5 aa, Homo sapiens
IKRQR

SEQ ID NO: 24 - protein, 5 aa, Homo sapiens
KRQRQ

SEQ ID NO: 25 - protein, 5 aa, Homo sapiens
RQRQQ

SEQ ID NO: 26 - protein, 5 aa, Homo sapiens
QRQQK

SEQ ID NO: 27 - protein, 5 aa, Homo sapiens
IKRQR

SEQ ID NO: 28 - protein, 5 aa, Homo sapiens
RQRQQ

SEQ ID NO: 29 - protein, 5 aa, Homo sapiens
LIKRQ

SEQ ID NO: 30 - protein, 5 aa, Homo sapiens
TDVGF

SEQ ID NO: 31 - protein, 5 aa, Homo sapiens
DVGFS

SEQ ID NO: 32 - protein, 5 aa, Homo sapiens
VGFSC

SEQ ID NO: 33 - protein, 5 aa, Homo sapiens
TDVGF

SEQ ID NO: 34 - protein, 5 aa, Homo sapiens
DVGFS

SEQ ID NO: 35 - protein, 5 aa, Homo sapiens
GFSCT

SEQ ID NO: 36 - protein, 5 aa, Homo sapiens
TDVGF

SEQ ID NO: 37 - protein, 5 aa, Homo sapiens
VGFSC

SEQ ID NO: 38 - protein, 5 aa, Homo sapiens
FSCTL

SEQ ID NO: 39 - protein, 5 aa, Homo sapiens
FSCTL

SEQ ID NO: 40 - protein, 5 aa, Homo sapiens
ATEVK

SEQ ID NO: 41 - protein, 5 aa, Homo sapiens
EVKSR

SEQ ID NO: 42 - protein, 5 aa, Homo sapiens
GLATE

SEQ ID NO: 43 - protein, 5 aa, Homo sapiens
LATEV

SEQ ID NO: 44 - protein, 5 aa, Homo sapiens
ATEVK

SEQ ID NO: 45 - protein, 5 aa, Homo sapiens
EVKSR

SEQ ID NO: 46 - protein, 5 aa, Homo sapiens
ATEVK

SEQ ID NO: 47 - protein, 5 aa, Homo sapiens
TEVKS

SEQ ID NO: 48 - protein, 5 aa, Homo sapiens
EVKSR

SEQ ID NO: 49 - protein, 5 aa, Homo sapiens
LATMV

SEQ ID NO: 50 - protein, 5 aa, Homo sapiens
ATMVK

SEQ ID NO: 51 - protein, 5 aa, Homo sapiens
MVKSR

SEQ ID NO: 52 - protein, 5 aa, Homo sapiens
GLATM

SEQ ID NO: 53 - protein, 5 aa, Homo sapiens
ATMVK

SEQ ID NO: 54 - protein, 5 aa, Homo sapiens
TMVKS

SEQ ID NO: 55 - protein, 5 aa, Homo sapiens
MVKSR

SEQ ID NO: 56 - protein, 5 aa, Homo sapiens
LATMV

SEQ ID NO: 57 - protein, 5 aa, Homo sapiens
ATMVK

SEQ ID NO: 58 - protein, 5 aa, Homo sapiens
TMVKS

SEQ ID NO: 59 - protein, 5 aa, Homo sapiens
MVKSR

SEQ ID NO: 60 - protein, 5 aa, Homo sapiens
ITKEQ

SEQ ID NO: 61 - protein, 5 aa, Homo sapiens
EITKE

SEQ ID NO: 62 - protein, 5 aa, Homo sapiens
MNDAR

SEQ ID NO: 63 - protein, 5 aa, Homo sapiens
ARHHG

SEQ ID NO: 64 - protein, 5 aa, Homo sapiens
DARHH

SEQ ID NO: 65 - protein, 5 aa, Homo sapiens
ARHHG

SEQ ID NO: 66 - protein, 5 aa, Homo sapiens
RHHGG

SEQ ID NO: 67 - protein, 5 aa, Homo sapiens
KGGRT

SEQ ID NO: 68 - protein, 5 aa, Homo sapiens
AGKGG

SEQ ID NO: 69 - protein, 5 aa, Homo sapiens
KGGRT

SEQ ID NO: 70 - protein, 5 aa, Homo sapiens
GGRTG

SEQ ID NO: 71 - protein, 5 aa, Homo sapiens
AGKGG

SEQ ID NO: 72 - protein, 5 aa, Homo sapiens
KGGRT

SEQ ID NO: 73 - protein, 5 aa, Homo sapiens
AGKGQ

SEQ ID NO: 74 - protein, 5 aa, Homo sapiens
KGQRT

SEQ ID NO: 75 - protein, 5 aa, Homo sapiens
GQRTG

SEQ ID NO: 76 - protein, 5 aa, Homo sapiens
AGKGQ

SEQ ID NO: 77 - protein, 5 aa, Homo sapiens
KGQRT

SEQ ID NO: 78 - protein, 7 aa, Homo sapiens
GKYSFGV

SEQ ID NO: 79 - protein, 7 aa, Homo sapiens
KYSFGVA

SEQ ID NO: 80 - protein, 7 aa, Homo sapiens
SFGVATC

SEQ ID NO: 81 - protein, 7 aa, Homo sapiens
GVATCVK

SEQ ID NO: 82 - protein, 7 aa, Homo sapiens
VATCVKK

SEQ ID NO: 83 - protein, 7 aa, Homo sapiens
GKYSFGV

SEQ ID NO: 84 - protein, 7 aa, Homo sapiens
GVATCVK

SEQ ID NO: 85 - protein, 7 aa, Homo sapiens
VATCVKK

SEQ ID NO: 86 - protein, 7 aa, Homo sapiens
GVATCVK

SEQ ID NO: 87 - protein, 7 aa, Homo sapiens
SFGVATC

SEQ ID NO: 88 - protein, 7 aa, Homo sapiens
GVATCVK

SEQ ID NO: 89 - protein, 7 aa, Homo sapiens
ITDFGRL

SEQ ID NO: 90 - protein, 7 aa, Homo sapiens
GRLAKLL

SEQ ID NO: 91 - protein, 7 aa, Homo sapiens
KITDFGR

SEQ ID NO: 92 - protein, 7 aa, Homo sapiens
ITDFGRL

SEQ ID NO: 93 - protein, 7 aa, Homo sapiens
DFGRLAK

SEQ ID NO: 94 - protein, 7 aa, Homo sapiens
GRLAKLL

SEQ ID NO: 95 - protein, 7 aa, Homo sapiens
RLAKLLG

SEQ ID NO: 96 - protein, 7 aa, Homo sapiens
KITDFGR

SEQ ID NO: 97 - protein, 7 aa, Homo sapiens
ITDFGRL

SEQ ID NO: 98 - protein, 7 aa, Homo sapiens
DFGRLAK

SEQ ID NO: 99 - protein, 7 aa, Homo sapiens
IKRQRQQ

SEQ ID NO: 100 - protein, 7 aa, Homo sapiens
GILIKRQ

SEQ ID NO: 101 - protein, 7 aa, Homo sapiens
ILIKRQR

SEQ ID NO: 102 - protein, 7 aa, Homo sapiens
IKRQRQQ

SEQ ID NO: 103 - protein, 7 aa, Homo sapiens
QRQQKIR

SEQ ID NO: 104 - protein, 7 aa, Homo sapiens
IKRQRQQ

SEQ ID NO: 105 - protein, 7 aa, Homo sapiens
RQRQQKI

SEQ ID NO: 106 - protein, 7 aa, Homo sapiens
QRQQKIR

SEQ ID NO: 107 - protein, 7 aa, Homo sapiens
LSTDVGF

SEQ ID NO: 108 - protein, 7 aa, Homo sapiens
STDVGFS

SEQ ID NO: 109 - protein, 7 aa, Homo sapiens
DVGFSCT

SEQ ID NO: 110 - protein, 7 aa, Homo sapiens
GFSCTLV

SEQ ID NO: 111 - protein, 7 aa, Homo sapiens
LSTDVGF

SEQ ID NO: 112 - protein, 7 aa, Homo sapiens
STDVGFS

SEQ ID NO: 113 - protein, 7 aa, Homo sapiens
DVGFSCT

SEQ ID NO: 114 - protein, 7 aa, Homo sapiens
GFSCTLV

SEQ ID NO: 115 - protein, 7 aa, Homo sapiens
LSTDVGF

SEQ ID NO: 116 - protein, 7 aa, Homo sapiens
STDVGFS

SEQ ID NO: 117 - protein, 7 aa, Homo sapiens
FSCTLVC

SEQ ID NO: 118 - protein, 7 aa, Homo sapiens
LSTDVGF

SEQ ID NO: 119 - protein, 7 aa, Homo sapiens
STDVGFS

SEQ ID NO: 120 - protein, 7 aa, Homo sapiens
GFSCTLV

SEQ ID NO: 121 - protein, 7 aa, Homo sapiens
EVKSRWS

SEQ ID NO: 122 - protein, 7 aa, Homo sapiens
LATEVKS

SEQ ID NO: 123 - protein, 7 aa, Homo sapiens
TEVKSRW

SEQ ID NO: 124 - protein, 7 aa, Homo sapiens
EVKSRWS

SEQ ID NO: 125 - protein, 7 aa, Homo sapiens
DFGLATE

SEQ ID NO: 126 - protein, 7 aa, Homo sapiens
FGLATEV

SEQ ID NO: 127 - protein, 7 aa, Homo sapiens
TEVKSRW

SEQ ID NO: 128 - protein, 7 aa, Homo sapiens
FGLATMV

SEQ ID NO: 129 - protein, 7 aa, Homo sapiens
LATMVKS

SEQ ID NO: 130 - protein, 7 aa, Homo sapiens
MVKSRWS

SEQ ID NO: 131 - protein, 7 aa, Homo sapiens
LATMVKS

SEQ ID NO: 132 - protein, 7 aa, Homo sapiens
MVKSRWS

SEQ ID NO: 133 - protein, 7 aa, Homo sapiens
DFGLATM

SEQ ID NO: 134 - protein, 7 aa, Homo sapiens
FGLATMV

SEQ ID NO: 135 - protein, 7 aa, Homo sapiens
LATMVKS

SEQ ID NO: 136 - protein, 7 aa, Homo sapiens
TMVKSRW

SEQ ID NO: 137 - protein, 7 aa, Homo sapiens
EITKEQE

SEQ ID NO: 138 - protein, 7 aa, Homo sapiens
LSEITKE

SEQ ID NO: 139 - protein, 7 aa, Homo sapiens
KEQEKDF

SEQ ID NO: 140 - protein, 7 aa, Homo sapiens
EITKEQE

SEQ ID NO: 141 - protein, 7 aa, Homo sapiens
TKEQEKD

SEQ ID NO: 142 - protein, 7 aa, Homo sapiens
KEQEKDF

SEQ ID NO: 143 - protein, 7 aa, Homo sapiens
RHHGGWT

SEQ ID NO: 144 - protein, 7 aa, Homo sapiens
KQMNDAR

SEQ ID NO: 145 - protein, 7 aa, Homo sapiens
QMNDARH

SEQ ID NO: 146 - protein, 7 aa, Homo sapiens
NDARHHG

SEQ ID NO: 147 - protein, 7 aa, Homo sapiens
CKAGKGG

SEQ ID NO: 148 - protein, 7 aa, Homo sapiens
GKGGRTG

SEQ ID NO: 149 - protein, 7 aa, Homo sapiens
GGRTGVM

SEQ ID NO: 150 - protein, 7 aa, Homo sapiens
GRTGVMI

SEQ ID NO: 151 - protein, 7 aa, Homo sapiens
CKAGKGG

SEQ ID NO: 152 - protein, 7 aa, Homo sapiens
GKGGRTG

SEQ ID NO: 153 - protein, 7 aa, Homo sapiens
GGRTGVM

SEQ ID NO: 154 - protein, 7 aa, Homo sapiens
CKAGKGQ

SEQ ID NO: 155 - protein, 7 aa, Homo sapiens
GKGQRTG

SEQ ID NO: 156 - protein, 7 aa, Homo sapiens
GQRTGVM

SEQ ID NO: 157 - protein, 7 aa, Homo sapiens
QRTGVMI

SEQ ID NO: 158 - protein, 7 aa, Homo sapiens
GKGQRTG

SEQ ID NO: 159 - protein, 7 aa, Homo sapiens
KAGKGQR

SEQ ID NO: 160 - protein, 7 aa, Homo sapiens
GQRTGVM

SEQ ID NO: 161 - protein, 7 aa, Homo sapiens
QRTGVMI

SEQ ID NO: 162 - protein, 5 aa, Homo sapiens
SFGVA

SEQ ID NO: 163 - protein, 5 aa, Homo sapiens
FGVAT

SEQ ID NO: 164 - protein, 5 aa, Homo sapiens
GVATC

SEQ ID NO: 165 - protein, 5 aa, Homo sapiens
SFGVA

SEQ ID NO: 166 - protein, 5 aa, Homo sapiens
GVATC

SEQ ID NO: 167 - protein, 5 aa, Homo sapiens
SFGVA

SEQ ID NO: 168 - protein, 5 aa, Homo sapiens
FGVAT

SEQ ID NO: 169 - protein, 5 aa, Homo sapiens
GVATC

SEQ ID NO: 170 - protein, 5 aa, Homo sapiens
YSFGV

SEQ ID NO: 171 - protein, 5 aa, Homo sapiens
GVATC

SEQ ID NO: 172 - protein, 5 aa, Homo sapiens
TDFGR

SEQ ID NO: 173 - protein, 5 aa, Homo sapiens
DFGRL

SEQ ID NO: 174 - protein, 5 aa, Homo sapiens
GRLAK

SEQ ID NO: 175 - protein, 5 aa, Homo sapiens
TDFGR

SEQ ID NO: 176 - protein, 5 aa, Homo sapiens
GRLAK

SEQ ID NO: 177 - protein, 5 aa, Homo sapiens
TDFGR

SEQ ID NO: 178 - protein, 5 aa, Homo sapiens
DFGRL

SEQ ID NO: 179 - protein, 5 aa, Homo sapiens
GRLAK

SEQ ID NO: 180 - protein, 5 aa, Homo sapiens
RLAKL

SEQ ID NO: 181 - protein, 5 aa, Homo sapiens
TDFGR

SEQ ID NO: 182 - protein, 5 aa, Homo sapiens
GRLAK

SEQ ID NO: 183 - protein, 5 aa, Homo sapiens
LIKRQ

SEQ ID NO: 184 - protein, 5 aa, Homo sapiens
QRQQK

SEQ ID NO: 185 - protein, 5 aa, Homo sapiens
KRQRQ

SEQ ID NO: 186 - protein, 5 aa, Homo sapiens
QRQQK

SEQ ID NO: 187 - protein, 5 aa, Homo sapiens
LIKRQ

SEQ ID NO: 188 - protein, 5 aa, Homo sapiens
QRQQK

SEQ ID NO: 189 - protein, 5 aa, Homo sapiens
GFSCT

SEQ ID NO: 190 - protein, 5 aa, Homo sapiens
DVGFS

SEQ ID NO: 191 - protein, 5 aa, Homo sapiens
GFSCT

SEQ ID NO: 192 - protein, 5 aa, Homo sapiens
DVGFS

SEQ ID NO: 193 - protein, 5 aa, Homo sapiens
GFSCT

SEQ ID NO: 194 - protein, 5 aa, Homo sapiens
FSCTL

SEQ ID NO: 195 - protein, 5 aa, Homo sapiens
DVGFS

SEQ ID NO: 196 - protein, 5 aa, Homo sapiens
GFSCT

SEQ ID NO: 197 - protein, 5 aa, Homo sapiens
GLATE

SEQ ID NO: 198 - protein, 5 aa, Homo sapiens
EVKSR

SEQ ID NO: 199 - protein, 5 aa, Homo sapiens
GLATE

SEQ ID NO: 200 - protein, 5 aa, Homo sapiens
GLATE

SEQ ID NO: 201 - protein, 5 aa, Homo sapiens
ATEVK

SEQ ID NO: 202 - protein, 5 aa, Homo sapiens
GLATE

SEQ ID NO: 203 - protein, 5 aa, Homo sapiens
ATEVK

SEQ ID NO: 204 - protein, 5 aa, Homo sapiens
TEVKS

SEQ ID NO: 205 - protein, 5 aa, Homo sapiens
EVKSR

SEQ ID NO: 206 - protein, 5 aa, Homo sapiens
GLATM

SEQ ID NO: 207 - protein, 5 aa, Homo sapiens
MVKSR

SEQ ID NO: 208 - protein, 5 aa, Homo sapiens
GLATM

SEQ ID NO: 209 - protein, 5 aa, Homo sapiens
GLATM

SEQ ID NO: 210 - protein, 5 aa, Homo sapiens
ATMVK

SEQ ID NO: 211 - protein, 5 aa, Homo sapiens
TMVKS

SEQ ID NO: 212 - protein, 5 aa, Homo sapiens
GLATM

SEQ ID NO: 213 - protein, 5 aa, Homo sapiens
ATMVK

SEQ ID NO: 214 - protein, 5 aa, Homo sapiens
TMVKS

SEQ ID NO: 215 - protein, 5 aa, Homo sapiens
MVKSR

SEQ ID NO: 216 - protein, 5 aa, Homo sapiens
EITKE

SEQ ID NO: 217 - protein, 5 aa, Homo sapiens
TKEQE

SEQ ID NO: 218 - protein, 5 aa, Homo sapiens
SEITK

SEQ ID NO: 219 - protein, 5 aa, Homo sapiens
ITKEQ

SEQ ID NO: 220 - protein, 5 aa, Homo sapiens
NDARH

SEQ ID NO: 221 - protein, 5 aa, Homo sapiens
RHHGG

SEQ ID NO: 222 - protein, 5 aa, Homo sapiens
DARHH

SEQ ID NO: 223 - protein, 5 aa, Homo sapiens
ARHHG

SEQ ID NO: 224 - protein, 5 aa, Homo sapiens
RHHGG

SEQ ID NO: 225 - protein, 5 aa, Homo sapiens
NDARH

SEQ ID NO: 226 - protein, 5 aa, Homo sapiens
RHHGG

SEQ ID NO: 227 - protein, 5 aa, Homo sapiens
AGKGG

SEQ ID NO: 228 - protein, 5 aa, Homo sapiens
GRTGV

SEQ ID NO: 229 - protein, 5 aa, Homo sapiens
AGKGG

SEQ ID NO: 230 - protein, 5 aa, Homo sapiens
GKGGR

SEQ ID NO: 231 - protein, 5 aa, Homo sapiens
GRTGV

SEQ ID NO: 232 - protein, 5 aa, Homo sapiens
KGGRT

SEQ ID NO: 233 - protein, 5 aa, Homo sapiens
GRTGV

SEQ ID NO: 234 - protein, 5 aa, Homo sapiens
AGKGG

SEQ ID NO: 235 - protein, 5 aa, Homo sapiens
KGGRT

SEQ ID NO: 236 - protein, 5 aa, Homo sapiens
GGRTG

SEQ ID NO: 237 - protein, 5 aa, Homo sapiens
AGKGQ

SEQ ID NO: 238 - protein, 5 aa, Homo sapiens
AGKGQ

SEQ ID NO: 239 - protein, 5 aa, Homo sapiens
QRTGV

SEQ ID NO: 240 - protein, 5 aa, Homo sapiens
KGQRT

SEQ ID NO: 241 - protein, 5 aa, Homo sapiens
QRTGV

SEQ ID NO: 242 - protein, 5 aa, Homo sapiens
AGKGQ

SEQ ID NO: 243 - protein, 5 aa, Homo sapiens
KGQRT

SEQ ID NO: 244 - protein, 5 aa, Homo sapiens
GQRTG

SEQ ID NO: 245 - protein, 5 aa, Homo sapiens
KKGNY

SEQ ID NO: 246 - protein, 5 aa, Homo sapiens
EKKGN

SEQ ID NO: 247 - protein, 5 aa, Homo sapiens
GNYVV

SEQ ID NO: 248 - protein, 5 aa, Homo sapiens
EKKGN

SEQ ID NO: 249 - protein, 5 aa, Homo sapiens
KKGNY

SEQ ID NO: 250 - protein, 5 aa, Homo sapiens
KGNYV

SEQ ID NO: 251 - protein, 5 aa, Homo sapiens
KGNYV

SEQ ID NO: 252 - protein, 5 aa, Homo sapiens
EEKKG

SEQ ID NO: 253 - protein, 5 aa, Homo sapiens
KGNYV

SEQ ID NO: 254 - protein, 5 aa, Homo sapiens
EEKKG

SEQ ID NO: 255 - protein, 5 aa, Homo sapiens
EKKGN

SEQ ID NO: 256 - protein, 5 aa, Homo sapiens
KGNYV

SEQ ID NO: 257 - protein, 5 aa, Homo sapiens
KKGNY

SEQ ID NO: 258 - protein, 5 aa, Homo sapiens
EKKGN

SEQ ID NO: 259 - protein, 5 aa, Homo sapiens
EEKKG

SEQ ID NO: 260 - protein, 5 aa, Homo sapiens
KGNYV

SEQ ID NO: 261 - protein, 5 aa, Homo sapiens
EKKGN

SEQ ID NO: 262 - protein, 5 aa, Homo sapiens
KKGNY

SEQ ID NO: 263 - protein, 5 aa, Homo sapiens
GNYVV

SEQ ID NO: 264 - protein, 5 aa, Homo sapiens
EEKKG

SEQ ID NO: 265 - protein, 5 aa, Homo sapiens
GNYVV

SEQ ID NO: 266 - protein, 5 aa, Homo sapiens
KKGNY

SEQ ID NO: 267 - protein, 5 aa, Homo sapiens
EKKGN

SEQ ID NO: 268 - protein, 5 aa, Homo sapiens
EKKGN

SEQ ID NO: 269 - protein, 5 aa, Homo sapiens
KKGNY

SEQ ID NO: 270 - protein, 5 aa, Homo sapiens
KGNYV

SEQ ID NO: 271 - protein, 5 aa, Homo sapiens
KGNYV

SEQ ID NO: 272 - protein, 5 aa, Homo sapiens
KGNYV

SEQ ID NO: 273 - protein, 5 aa, Homo sapiens
EEKKG

SEQ ID NO: 274 - protein, 5 aa, Homo sapiens
EKKGN

SEQ ID NO: 275 - protein, 5 aa, Homo sapiens
EEKKG

SEQ ID NO: 276 - protein, 5 aa, Homo sapiens
KKGNY

SEQ ID NO: 277 - protein, 5 aa, Homo sapiens
KGNYV

SEQ ID NO: 278 - protein, 5 aa, Homo sapiens
EEKKG

SEQ ID NO: 279 - protein, 5 aa, Homo sapiens
EKKGN

SEQ ID NO: 280 - protein, 5 aa, Homo sapiens
KGNYV

SEQ ID NO: 281 - protein, 5 aa, Homo sapiens
EKKGN

SEQ ID NO: 282 - protein, 5 aa, Homo sapiens
GNYVV

SEQ ID NO: 283 - protein, 5 aa, Homo sapiens
KKGNY

SEQ ID NO: 284 - protein, 5 aa, Homo sapiens
EEKKG

SEQ ID NO: 285 - protein, 5 aa, Homo sapiens
MEPCW

SEQ ID NO: 286 - protein, 5 aa, Homo sapiens
EPCWK

SEQ ID NO: 287 - protein, 5 aa, Homo sapiens
PCWKQ

SEQ ID NO: 288 - protein, 5 aa, Homo sapiens
CWKQI

SEQ ID NO: 289 - protein, 5 aa, Homo sapiens
WKQID

SEQ ID NO: 290 - protein, 5 aa, Homo sapiens
TYRLM

SEQ ID NO: 291 - protein, 5 aa, Homo sapiens
YRLML

SEQ ID NO: 292 - protein, 5 aa, Homo sapiens
RLMLY

SEQ ID NO: 293 - protein, 5 aa, Homo sapiens
LMLYS

SEQ ID NO: 294 - protein, 5 aa, Homo sapiens
MLYSL

SEQ ID NO: 295 - protein, 5 aa, Homo sapiens
RFFML

SEQ ID NO: 296 - protein, 5 aa, Homo sapiens
FFMLA

SEQ ID NO: 297 - protein, 5 aa, Homo sapiens
FMLAW

SEQ ID NO: 298 - protein, 5 aa, Homo sapiens
MLAWN

SEQ ID NO: 299 - protein, 5 aa, Homo sapiens
LAWNL

SEQ ID NO: 300 - protein, 5 aa, Homo sapiens
PRAQL

SEQ ID NO: 301 - protein, 5 aa, Homo sapiens
RAQLS

SEQ ID NO: 302 - protein, 5 aa, Homo sapiens
AQLSS

SEQ ID NO: 303 - protein, 5 aa, Homo sapiens
QLSSA

SEQ ID NO: 304 - protein, 5 aa, Homo sapiens
LSSAS

SEQ ID NO: 305 - protein, 5 aa, Homo sapiens
EKELP

SEQ ID NO: 306 - protein, 5 aa, Homo sapiens
KELPE

SEQ ID NO: 307 - protein, 5 aa, Homo sapiens
ELPEE

SEQ ID NO: 308 - protein, 5 aa, Homo sapiens
LPEEK

SEQ ID NO: 309 - protein, 5 aa, Homo sapiens
PEEKK

SEQ ID NO: 310 - protein, 5 aa, Homo sapiens
LQALK

SEQ ID NO: 311 - protein, 5 aa, Homo sapiens
QALKE

SEQ ID NO: 312 - protein, 5 aa, Homo sapiens
ALKEL

SEQ ID NO: 313 - protein, 5 aa, Homo sapiens
LKELA

SEQ ID NO: 314 - protein, 5 aa, Homo sapiens
KELAV

SEQ ID NO: 315 - protein, 5 aa, Homo sapiens
FDKCS

SEQ ID NO: 316 - protein, 5 aa, Homo sapiens
DKCSE

SEQ ID NO: 317 - protein, 5 aa, Homo sapiens
KCSEL

SEQ ID NO: 318 - protein, 5 aa, Homo sapiens
CSELR

SEQ ID NO: 319 - protein, 5 aa, Homo sapiens
SELRE

SEQ ID NO: 320 - protein, 5 aa, Homo sapiens
QRDRK

SEQ ID NO: 321 - protein, 5 aa, Homo sapiens
RDRKE

SEQ ID NO: 322 - protein, 5 aa, Homo sapiens
DRKEA

SEQ ID NO: 323 - protein, 5 aa, Homo sapiens
RKEAL

SEQ ID NO: 324 - protein, 5 aa, Homo sapiens
KEALH

SEQ ID NO: 325 - protein, 5 aa, Homo sapiens
TAIIE

SEQ ID NO: 326 - protein, 5 aa, Homo sapiens
AIIEE

SEQ ID NO: 327 - protein, 5 aa, Homo sapiens
IIEEI

SEQ ID NO: 328 - protein, 5 aa, Homo sapiens
IEEIV

SEQ ID NO: 329 - protein, 5 aa, Homo sapiens
EEIVS

SEQ ID NO: 330 - protein, 5 aa, Homo sapiens
PWKEP

SEQ ID NO: 331 - protein, 5 aa, Homo sapiens
WKEPT

SEQ ID NO: 332 - protein, 5 aa, Homo sapiens
KEPTA

SEQ ID NO: 333 - protein, 5 aa, Homo sapiens
EPTAF

SEQ ID NO: 334 - protein, 5 aa, Homo sapiens
PTAFQ

SEQ ID NO: 335 - protein, 9 aa, Homo sapiens
KTFMEPCWP

SEQ ID NO: 336 - protein, 9 aa, Homo sapiens
AGKEPCWKP

SEQ ID NO: 337 - protein, 9 aa, Homo sapiens
LAACWKQIK

SEQ ID NO: 338 - protein, 9 aa, Homo sapiens
LDPYRLMLK

SEQ ID NO: 339 - protein, 9 aa, Homo sapiens
TQSMLYSLK

SEQ ID NO: 340 - protein, 9 aa, Homo sapiens
DDRFFMLAK

SEQ ID NO: 341 - protein, 9 aa, Homo sapiens
EPSMLAWNK

SEQ ID NO: 342 - protein, 9 aa, Homo sapiens
SGRLAWNLP

SEQ ID NO: 343 - protein, 9 aa, Homo sapiens
RFYPRAQLP

SEQ ID NO: 344 - protein, 9 aa, Homo sapiens
EGRLSSASK

SEQ ID NO: 345 - protein, 9 aa, Homo sapiens
KGSEKELPQ

SEQ ID NO: 346 - protein, 9 aa, Homo sapiens
GGYPEEKKP

SEQ ID NO: 347 - protein, 9 aa, Homo sapiens
VEFLKELAR

SEQ ID NO: 348 - protein, 9 aa, Homo sapiens
YVRKELAVQ

SEQ ID NO: 349 - protein, 9 aa, Homo sapiens
KCFFDKCSD

SEQ ID NO: 350 - protein, 9 aa, Homo sapiens
VCFDKCSEQ

SEQ ID NO: 351 - protein, 9 aa, Homo sapiens
DVMKCSELP

SEQ ID NO: 352 - protein, 9 aa, Homo sapiens
KPYKEALHP

SEQ ID NO: 353 - protein, 9 aa, Homo sapiens
KCKEEIVSP

SEQ ID NO: 354 - protein, 9 aa, Homo sapiens
DAWEEIVSY

SEQ ID NO: 355 - protein, 9 aa, Homo sapiens
MYLKEPTAK

SEQ ID NO: 356 - protein, 9 aa, Homo sapiens
IVRPTAFQQ

SEQ ID NO: 357 - protein, 9 aa, Homo sapiens
HGVEPCWKI

SEQ ID NO: 358 - protein, 9 aa, Homo sapiens
MKGPCWKQF

SEQ ID NO: 359 - protein, 9 aa, Homo sapiens
RPSWKQIDF

SEQ ID NO: 360 - protein, 9 aa, Homo sapiens
FKNTYRLML

SEQ ID NO: 361 - protein, 9 aa, Homo sapiens
RPRMLYSLM

SEQ ID NO: 362 - protein, 9 aa, Homo sapiens
KSEFFMLAR

SEQ ID NO: 363 - protein, 9 aa, Homo sapiens
QARMLAWNY

SEQ ID NO: 364 - protein, 9 aa, Homo sapiens
RQELAWNLW

SEQ ID NO: 365 - protein, 9 aa, Homo sapiens
KNVLSSASW

SEQ ID NO: 366 - protein, 9 aa, Homo sapiens
DAEEKELPY

SEQ ID NO: 367 - protein, 9 aa, Homo sapiens
DLIPEEKKF

SEQ ID NO: 368 - protein, 9 aa, Homo sapiens
VLRQALKEF

SEQ ID NO: 369 - protein, 9 aa, Homo sapiens
VGDALKELM

SEQ ID NO: 370 - protein, 9 aa, Homo sapiens
KSQLKELAY

SEQ ID NO: 371 - protein, 9 aa, Homo sapiens
WPSKELAVR

SEQ ID NO: 372 - protein, 9 aa, Homo sapiens
RSNKCSELY

SEQ ID NO: 373 - protein, 9 aa, Homo sapiens
LAEIIEEIH

SEQ ID NO: 374 - protein, 9 aa, Homo sapiens
KGEIEEIVY

SEQ ID NO: 375 - protein, 9 aa, Homo sapiens
LGSEEIVSR

SEQ ID NO: 376 - protein, 9 aa, Homo sapiens
LPSKEPTAA

SEQ ID NO: 377 - protein, 9 aa, Homo sapiens
NSAPTAFQF

SEQ ID NO: 378 - protein, 9 aa, Homo sapiens
VESPCWKQS

SEQ ID NO: 379 - protein, 9 aa, Homo sapiens
IERCWKQIE

SEQ ID NO: 380 - protein, 9 aa, Homo sapiens
CERWKQIDD

SEQ ID NO: 381 - protein, 9 aa, Homo sapiens
TETTYRLMV

SEQ ID NO: 382 - protein, 9 aa, Homo sapiens
REELMLYSQ

SEQ ID NO: 383 - protein, 9 aa, Homo sapiens
IERMLYSLR

SEQ ID NO: 384 - protein, 9 aa, Homo sapiens
EEQFFMLAQ

SEQ ID NO: 385 - protein, 9 aa, Homo sapiens
AEKFMLAWE

SEQ ID NO: 386 - protein, 9 aa, Homo sapiens
SGPMLAWNR

SEQ ID NO: 387 - protein, 9 aa, Homo sapiens
TEVLAWNLK

SEQ ID NO: 388 - protein, 9 aa, Homo sapiens
EEVPRAQLP

SEQ ID NO: 389 - protein, 9 aa, Homo sapiens
DCSRAQLSA

SEQ ID NO: 390 - protein, 9 aa, Homo sapiens
GHFELPEEM

SEQ ID NO: 391 - protein, 9 aa, Homo sapiens
VELPEEKKS

SEQ ID NO: 392 - protein, 9 aa, Homo sapiens
FESQALKEV

SEQ ID NO: 393 - protein, 9 aa, Homo sapiens
PEKALKELQ

SEQ ID NO: 394 - protein, 9 aa, Homo sapiens
QCELKELAT

SEQ ID NO: 395 - protein, 9 aa, Homo sapiens
LCLFDKCSS

SEQ ID NO: 396 - protein, 9 aa, Homo sapiens
LEECSELRT

SEQ ID NO: 397 - protein, 9 aa, Homo sapiens
REATAIIEE

SEQ ID NO: 398 - protein, 9 aa, Homo sapiens
EEHIIEEID

SEQ ID NO: 399 - protein, 9 aa, Homo sapiens
VERPTAFQG

SEQ ID NO: 400 - protein, 9 aa, Homo sapiens
LLRPCWKQA

SEQ ID NO: 401 - protein, 9 aa, Homo sapiens
PFDWKQIDP

SEQ ID NO: 402 - protein, 9 aa, Homo sapiens
SKIRLMLYS

SEQ ID NO: 403 - protein, 9 aa, Homo sapiens
RRYLMLYSK

SEQ ID NO: 404 - protein, 9 aa, Homo sapiens
KKLMLYSLK

SEQ ID NO: 405 - protein, 9 aa, Homo sapiens
SAERFFMLK

SEQ ID NO: 406 - protein, 9 aa, Homo sapiens
DRRFFMLAK

SEQ ID NO: 407 - protein, 9 aa, Homo sapiens
PGSMLAWNK

SEQ ID NO: 408 - protein, 9 aa, Homo sapiens
KLKPRAQLL

SEQ ID NO: 409 - protein, 9 aa, Homo sapiens
LELRAQLSS

SEQ ID NO: 410 - protein, 9 aa, Homo sapiens
SLRQLSSAL

SEQ ID NO: 411 - protein, 9 aa, Homo sapiens
APFEKELPR

SEQ ID NO: 412 - protein, 8 aa, Homo sapiens
WLQLPEEK

SEQ ID NO: 413 - protein, 9 aa, Homo sapiens
QCLPEEKKS

SEQ ID NO: 414 - protein, 9 aa, Homo sapiens
YQMLKELAP

SEQ ID NO: 415 - protein, 9 aa, Homo sapiens
QWRFDKCSQ

SEQ ID NO: 416 - protein, 9 aa, Homo sapiens
LQHDKCSEK

SEQ ID NO: 417 - protein, 9 aa, Homo sapiens
LKFKCSELA

SEQ ID NO: 418 - protein, 9 aa, Homo sapiens
VILQRDRKK

SEQ ID NO: 419 - protein, 9 aa, Homo sapiens
FDWDRKEAG

SEQ ID NO: 420 - protein, 9 aa, Homo sapiens
AYDKEALHL

SEQ ID NO: 421 - protein, 9 aa, Homo sapiens
AAVTAIIEK

SEQ ID NO: 422 - protein, 9 aa, Homo sapiens
PTCIEEIVK

SEQ ID NO: 423 - protein, 9 aa, Homo sapiens
RRFEEIVSD

SEQ ID NO: 424 - protein, 9 aa, Homo sapiens
STCWKEPTK

SEQ ID NO: 425 - protein, 9 aa, Homo sapiens
AMKKEPTAL

SEQ ID NO: 426 - protein, 9 aa, Homo sapiens
IYREPTAFS

SEQ ID NO: 427 - protein, 9 aa, Homo sapiens
ASLEPCWKH

SEQ ID NO: 428 - protein, 9 aa, Homo sapiens
WLQLPEEKW

SEQ ID NO: 429 - protein, 5 aa, Homo sapiens
CWQDQ

SEQ ID NO: 430 - protein, 5 aa, Homo sapiens
WKIQQ

SEQ ID NO: 431 - protein, 5 aa, Homo sapiens
KNYLM

SEQ ID NO: 432 - protein, 5 aa, Homo sapiens
NTRML

SEQ ID NO: 433 - protein, 5 aa, Homo sapiens
YRMYS

SEQ ID NO: 434 - protein, 5 aa, Homo sapiens
LMYLR

SEQ ID NO: 435 - protein, 5 aa, Homo sapiens
MLSRW

SEQ ID NO: 436 - protein, 5 aa, Homo sapiens
QEFML

SEQ ID NO: 437 - protein, 5 aa, Homo sapiens
ERFLA

SEQ ID NO: 438 - protein, 5 aa, Homo sapiens
FFLWN

SEQ ID NO: 439 - protein, 5 aa, Homo sapiens
MLWLE

SEQ ID NO: 440 - protein, 5 aa, Homo sapiens
LANEI

SEQ ID NO: 441 - protein, 5 aa, Homo sapiens
MPALS

SEQ ID NO: 442 - protein, 5 aa, Homo sapiens
RALSA

SEQ ID NO: 443 - protein, 5 aa, Homo sapiens
QLSSY

SEQ ID NO: 444 - protein, 5 aa, Homo sapiens
LSAYQ

SEQ ID NO: 445 - protein, 5 aa, Homo sapiens
LQKLP

SEQ ID NO: 446 - protein, 5 aa, Homo sapiens
QEEPE

SEQ ID NO: 447 - protein, 5 aa, Homo sapiens
KEPEK

SEQ ID NO: 448 - protein, 5 aa, Homo sapiens
LPEKR

SEQ ID NO: 449 - protein, 5 aa, Homo sapiens
PEKRK

SEQ ID NO: 450 - protein, 5 aa, Homo sapiens
EVQLK

SEQ ID NO: 451 - protein, 5 aa, Homo sapiens
VLAKE

SEQ ID NO: 452 - protein, 5 aa, Homo sapiens
QAKLA

SEQ ID NO: 453 - protein, 5 aa, Homo sapiens
LKLVN

SEQ ID NO: 454 - protein, 5 aa, Homo sapiens
KEANY

SEQ ID NO: 455 - protein, 5 aa, Homo sapiens
DKSLR

SEQ ID NO: 456 - protein, 5 aa, Homo sapiens
SERPL

SEQ ID NO: 457 - protein, 5 aa, Homo sapiens
MTIEE

SEQ ID NO: 458 - protein, 5 aa, Homo sapiens
AIEIV

SEQ ID NO: 459 - protein, 5 aa, Homo sapiens
IEISR

SEQ ID NO: 460 - protein, 5 aa, Homo sapiens
EEVRN

SEQ ID NO: 461 - protein, 5 aa, Homo sapiens
QPKPT

SEQ ID NO: 462 - protein, 5 aa, Homo sapiens
WKPAF

SEQ ID NO: 463 - protein, 5 aa, Homo sapiens
EPAQC

SEQ ID NO: 464 - protein, 5 aa, Homo sapiens
PTFCV

SEQ ID NO: 465 - protein, 15 aa, Homo sapiens
MLMWCWKQIDQNHQY

SEQ ID NO: 466 - protein, 15 aa, Homo sapiens
QIIYKNTYRLMLDGL

SEQ ID NO: 467 - protein, 15 aa, Homo sapiens
QIWSNTYRLMLTTVG

SEQ ID NO: 468 - protein, 15 aa, Homo sapiens
DSLGYRLMLYSDQGD

SEQ ID NO: 469 - protein, 15 aa, Homo sapiens
TLPHLMLYSLRSENG

SEQ ID NO: 470 - protein, 15 aa, Homo sapiens
RKIHMLYSLRWGLAQ

SEQ ID NO: 471 - protein, 15 aa, Homo sapiens
RDIQFFMLAWNHQDL

SEQ ID NO: 472 - protein, 15 aa, Homo sapiens
SYTFMLAWNLESDTE

SEQ ID NO: 473 - protein, 15 aa, Homo sapiens
SYMRLAWNLEISSEI

SEQ ID NO: 474 - protein, 15 aa, Homo sapiens
LPLLMPRAQLSQSQD

SEQ ID NO: 475 - protein, 15 aa, Homo sapiens
QRFQRAQLSSAATPL

SEQ ID NO: 476 - protein, 15 aa, Homo sapiens
DQMWQLSSASYLDTT

SEQ ID NO: 477 - protein, 15 aa, Homo sapiens
PALILSSASYQNSLP

SEQ ID NO: 478 - protein, 15 aa, Homo sapiens
TLLILQEKELPALNT

SEQ ID NO: 479 - protein, 15 aa, Homo sapiens
YSMWQEKELPELSYS

SEQ ID NO: 480 - protein, 15 aa, Homo sapiens
AYLFKELPEEKDDDK

SEQ ID NO: 481 - protein, 15 aa, Homo sapiens
PKLFLPEEKKRPQLP

SEQ ID NO: 482 - protein, 15 aa, Homo sapiens
LFHYPEEKKRKNLRK

SEQ ID NO: 483 - protein, 15 aa, Homo sapiens
SLNLEVLQALKGTGL

SEQ ID NO: 484 - protein, 15 aa, Homo sapiens
FSRWQALKELALARP

SEQ ID NO: 485 - protein, 15 aa, Homo sapiens
FLNILKELAVNLTQD

SEQ ID NO: 486 - protein, 15 aa, Homo sapiens
SIQWKELAVNYYKKE

SEQ ID NO: 487 - protein, 15 aa, Homo sapiens
FILLDKCSELRADTP

SEQ ID NO: 488 - protein, 15 aa, Homo sapiens
ALKMMTAIIEELSPS

SEQ ID NO: 489 - protein, 15 aa, Homo sapiens
ARFFAIIEEIVQEAE

SEQ ID NO: 490 - protein, 15 aa, Homo sapiens
MWLRIEEIVSRNSDL

SEQ ID NO: 491 - protein, 15 aa, Homo sapiens
PDLWEEIVSRNLQLA

SEQ ID NO: 492 - protein, 15 aa, Homo sapiens
DFYVPTAFQCVPKTQ

SEQ ID NO: 493 - protein, 15 aa, Homo sapiens
ISRLWKQIDQQYIIA

SEQ ID NO: 494 - protein, 15 aa, Homo sapiens
HRIFKNTYRLMVLLH

SEQ ID NO: 495 - protein, 15 aa, Homo sapiens
FAFRYRLMLYSKQEF

SEQ ID NO: 496 - protein, 15 aa, Homo sapiens
MQPQLMLYSLRIDKV

SEQ ID NO: 497 - protein, 15 aa, Homo sapiens
RQRQMLYSLRWVDRF

SEQ ID NO: 498 - protein, 15 aa, Homo sapiens
RQIRQERFFMLQRYF

SEQ ID NO: 499 - protein, 15 aa, Homo sapiens
KMLEERFFMLAKLYP

SEQ ID NO: 500 - protein, 15 aa, Homo sapiens
RPQFFFMLAWNNRLR

SEQ ID NO: 501 - protein, 15 aa, Homo sapiens
QVSHMLAWNLEFIQE

SEQ ID NO: 502 - protein, 15 aa, Homo sapiens
RRWLLAWNLEICDLD

SEQ ID NO: 503 - protein, 15 aa, Homo sapiens
GVYLRAQLSSAPIPA

SEQ ID NO: 504 - protein, 15 aa, Homo sapiens
FEFLQLSSASYAHCR

SEQ ID NO: 505 - protein, 15 aa, Homo sapiens
KFLHLSSASYQWRIM

SEQ ID NO: 506 - protein, 15 aa, Homo sapiens
PTLPEVLQALKAMLE

SEQ ID NO: 507 - protein, 15 aa, Homo sapiens
RMPLVLQALKEVRSI

SEQ ID NO: 508 - protein, 15 aa, Homo sapiens
KSLFQALKELALNPV

SEQ ID NO: 509 - protein, 15 aa, Homo sapiens
HEKMLKELAVNPNFL

SEQ ID NO: 510 - protein, 15 aa, Homo sapiens
ELWYKELAVNYWRLP

SEQ ID NO: 511 - protein, 15 aa, Homo sapiens
HELWSELREPLLLIS

SEQ ID NO: 512 - protein, 15 aa, Homo sapiens
RYLTMTAIIEEYDVL

SEQ ID NO: 513 - protein, 15 aa, Homo sapiens
SLNVQPWKEPTLVIM

SEQ ID NO: 514 - protein, 15 aa, Homo sapiens
DKFRWKEPTAFFKVC

SEQ ID NO: 515 - protein, 15 aa, Homo sapiens
LKKQEPTAFQCLLII

SEQ ID NO: 516 - protein, 15 aa, Homo sapiens
KPLYPTAFQCVPYQM

SEQ ID NO: 517 - protein, 5 aa, Homo sapiens
RDKAL

SEQ ID NO: 518 - protein, 5 aa, Homo sapiens
KELQF

SEQ ID NO: 519 - protein, 5 aa, Homo sapiens
CSLEP

SEQ ID NO: 520 - protein, 5 aa, Homo sapiens
RSRRK

SEQ ID NO: 521 - protein, 5 aa, Homo sapiens
AVDCS

SEQ ID NO: 522 - protein, 9 aa, Homo sapiens
KDKGFLIYV

SEQ ID NO: 523 - protein, 9 aa, Homo sapiens
KMKLIYLDA

SEQ ID NO: 524 - protein, 9 aa, Homo sapiens
KLKIYLDVG

SEQ ID NO: 525 - protein, 9 aa, Homo sapiens
KLLYKDTLV

SEQ ID NO: 526 - protein, 9 aa, Homo sapiens
KMSTLWCSG

SEQ ID NO: 527 - protein, 9 aa, Homo sapiens
KLRLWCSPA

SEQ ID NO: 528 - protein, 5 aa, Homo sapiens
GFLIY

SEQ ID NO: 529 - protein, 5 aa, Homo sapiens
LIYLD

SEQ ID NO: 530 - protein, 5 aa, Homo sapiens
IYLDV

SEQ ID NO: 531 - protein, 5 aa, Homo sapiens
YKDTL

SEQ ID NO: 532 - protein, 5 aa, Homo sapiens
TLWCS

SEQ ID NO: 533 - protein, 5 aa, Homo sapiens
LWCSP

SEQ ID NO: 534 - protein, 5 aa, Homo sapiens
YEVPK

SEQ ID NO: 535 - protein, 5 aa, Homo sapiens
LNYEV

SEQ ID NO: 536 - protein, 5 aa, Homo sapiens
NYEVP

SEQ ID NO: 537 - protein, 5 aa, Homo sapiens
EVPKY

SEQ ID NO: 538 - protein, 9 aa, Homo sapiens
LELLNYEVK

SEQ ID NO: 539 - protein, 9 aa, Homo sapiens
KATNYEVPR

SEQ ID NO: 540 - protein, 9 aa, Homo sapiens
SIVYEVPKP

SEQ ID NO: 541 - protein, 9 aa, Homo sapiens
DMLYEVPKI

SEQ ID NO: 542 - protein, 9 aa, Homo sapiens
TIAEVPKYR

SEQ ID NO: 543 - protein, 9 aa, Homo sapiens
ALIHHNTYL

SEQ ID NO: 544 - protein, 10 aa, Homo sapiens
VLENFTIFLV

SEQ ID NO: 545 - protein, 10 aa, Homo sapiens
SVLENFTIFL

SEQ ID NO: 546 - protein, 10 aa, Homo sapiens
VLLGVKLFGV

SEQ ID NO: 547 - protein, 9 aa, Homo sapiens
QMRGVKLFG

SEQ ID NO: 548 - protein, 9 aa, Homo sapiens
GMDVKLFGG

SEQ ID NO: 549 - protein, 9 aa, Homo sapiens
RMRLFGVQA

SEQ ID NO: 550 - protein, 9 aa, Homo sapiens
KLVFGVQDA

SEQ ID NO: 551 - protein, 9 aa, Homo sapiens
GMIHHNTYG

SEQ ID NO: 552 - protein, 9 aa, Homo sapiens
QTIHNTYLV

SEQ ID NO: 553 - protein, 9 aa, Homo sapiens
KGVNTYLCV

SEQ ID NO: 554 - protein, 9 aa, Homo sapiens
KLRTYLCFS

SEQ ID NO: 555 - protein, 9 aa, Homo sapiens
KQQYLCFVG

SEQ ID NO: 556 - protein, 9 aa, Homo sapiens
RLKVLENFV

SEQ ID NO: 557 - protein, 9 aa, Homo sapiens
ALPLENFTG

SEQ ID NO: 558 - protein, 9 aa, Homo sapiens
YSAENFTIV

SEQ ID NO: 559 - protein, 9 aa, Homo sapiens
KTPNFTIFA

SEQ ID NO: 560 - protein, 9 aa, Homo sapiens
RQKFTIFLG

SEQ ID NO: 561 - protein, 5 aa, Homo sapiens
GVKLF

SEQ ID NO: 562 - protein, 5 aa, Homo sapiens
VKLFG

SEQ ID NO: 563 - protein, 5 aa, Homo sapiens
LFGVQ

SEQ ID NO: 564 - protein, 5 aa, Homo sapiens
FGVQD

SEQ ID NO: 565 - protein, 5 aa, Homo sapiens
HHNTY

SEQ ID NO: 566 - protein, 5 aa, Homo sapiens
HNTYL

SEQ ID NO: 567 - protein, 5 aa, Homo sapiens
NTYLC

SEQ ID NO: 568 - protein, 5 aa, Homo sapiens
TYLCF

SEQ ID NO: 569 - protein, 5 aa, Homo sapiens
YLCFV

SEQ ID NO: 570 - protein, 5 aa, Homo sapiens
VLENF

SEQ ID NO: 571 - protein, 5 aa, Homo sapiens
LENFT

SEQ ID NO: 572 - protein, 5 aa, Homo sapiens
ENFTI

SEQ ID NO: 573 - protein, 5 aa, Homo sapiens
NFTIF

SEQ ID NO: 574 - protein, 5 aa, Homo sapiens
FTIFL

SEQ ID NO: 575 - protein, 9 aa, Homo sapiens
ALYGFVPVL

SEQ ID NO: 576 - protein, 9 aa, Homo sapiens
RTRALYGFV

SEQ ID NO: 577 - protein, 9 aa, Homo sapiens
SSDLYGFVR

SEQ ID NO: 578 - protein, 9 aa, Homo sapiens
KDELYGFVV

SEQ ID NO: 579 - protein, 9 aa, Homo sapiens
LADYGFVPD

SEQ ID NO: 580 - protein, 9 aa, Homo sapiens
KLRYGFVPA

SEQ ID NO: 581 - protein, 9 aa, Homo sapiens
KVDGFVPVA

SEQ ID NO: 582 - protein, 9 aa, Homo sapiens
RSDFVPVLN

SEQ ID NO: 583 - protein, 5 aa, Homo sapiens
ALYGF

SEQ ID NO: 584 - protein, 5 aa, Homo sapiens
LYGFV

SEQ ID NO: 585 - protein, 5 aa, Homo sapiens
LYGFV

SEQ ID NO: 586 - protein, 5 aa, Homo sapiens
YGFVP

SEQ ID NO: 587 - protein, 5 aa, Homo sapiens
YGFVP

SEQ ID NO: 588 - protein, 5 aa, Homo sapiens
GFVPV

SEQ ID NO: 589 - protein, 5 aa, Homo sapiens
FVPVL

SEQ ID NO: 590 - protein, 10 aa, Homo sapiens
PSDTRQMLFY

SEQ ID NO: 591 - protein, 9 aa, Homo sapiens
EGDFFPSDP

SEQ ID NO: 592 - protein, 9 aa, Homo sapiens
FEDFFPSDL

SEQ ID NO: 593 - protein, 9 aa, Homo sapiens
WDDFFPSDE

SEQ ID NO: 594 - protein, 5 aa, Homo sapiens
FFPSD

SEQ ID NO: 595 - protein, 9 aa, Homo sapiens
AVDDFGRAR

SEQ ID NO: 596 - protein, 9 aa, Homo sapiens
SRDDFGRAR

SEQ ID NO: 597 - protein, 9 aa, Homo sapiens
LSHFGRAKF

SEQ ID NO: 598 - protein, 9 aa, Homo sapiens
FAEGRAKLH

SEQ ID NO: 599 - protein, 9 aa, Homo sapiens
YGHRAKLLL

SEQ ID NO: 600 - protein, 9 aa, Homo sapiens
LAITDFGRA

SEQ ID NO: 601 - protein, 9 aa, Homo sapiens
VAADFGRAY

SEQ ID NO: 602 - protein, 9 aa, Homo sapiens
WGIFGRAKA

SEQ ID NO: 603 - protein, 9 aa, Homo sapiens
LEVGRAKLL

SEQ ID NO: 604 - protein, 9 aa, Homo sapiens
PESSGIILR

SEQ ID NO: 605 - protein, 9 aa, Homo sapiens
QQMPEQPSF

SEQ ID NO: 606 - protein, 9 aa, Homo sapiens
GLCEQPSGF

SEQ ID NO: 607 - protein, 9 aa, Homo sapiens
HAPQPSGIF

SEQ ID NO: 608 - protein, 9 aa, Homo sapiens
PAGPEQPST

SEQ ID NO: 609 - protein, 9 aa, Homo sapiens
ASGEQPSGF

SEQ ID NO: 610 - protein, 9 aa, Homo sapiens
SGCGPRLLR

SEQ ID NO: 611 - protein, 9 aa, Homo sapiens
YQQPRLLLR

SEQ ID NO: 612 - protein, 9 aa, Homo sapiens
NTGRLLLPP

SEQ ID NO: 613 - protein, 9 aa, Homo sapiens
KTQLLLPCR

SEQ ID NO: 614 - protein, 9 aa, Homo sapiens
RVTGPRLLD

SEQ ID NO: 615 - protein, 9 aa, Homo sapiens
QGGPRLLLK

SEQ ID NO: 616 - protein, 9 aa, Homo sapiens
SDWRLLLPK

SEQ ID NO: 617 - protein, 9 aa, Homo sapiens
SHELLLPCR

SEQ ID NO: 618 - protein, 9 aa, Homo sapiens
KACGPRLLY

SEQ ID NO: 619 - protein, 9 aa, Homo sapiens
RGPPRLLLY

SEQ ID NO: 620 - protein, 9 aa, Homo sapiens
DPTRLLLPY

SEQ ID NO: 621 - protein, 9 aa, Homo sapiens
PGSGPRLLS

SEQ ID NO: 622 - protein, 9 aa, Homo sapiens
RSGLLLPCR

SEQ ID NO: 623 - protein, 9 aa, Homo sapiens
SSSPRLLLP

SEQ ID NO: 624 - protein, 9 aa, Homo sapiens
RSLRHSYCR

SEQ ID NO: 625 - protein, 9 aa, Homo sapiens
SKLHSYCNK

SEQ ID NO: 626 - protein, 9 aa, Homo sapiens
GARRHSYCR

SEQ ID NO: 627 - protein, 9 aa, Homo sapiens
PLGHSYCNR

SEQ ID NO: 628 - protein, 9 aa, Homo sapiens
GLTYCNGSR

SEQ ID NO: 629 - protein, 5 aa, Homo sapiens
DFGRA

SEQ ID NO: 630 - protein, 5 aa, Homo sapiens
DFGRA

SEQ ID NO: 631 - protein, 5 aa, Homo sapiens
FGRAK

SEQ ID NO: 632 - protein, 5 aa, Homo sapiens
GRAKL

SEQ ID NO: 633 - protein, 5 aa, Homo sapiens
RAKLL

SEQ ID NO: 634 - protein, 5 aa, Homo sapiens
TDFGR

SEQ ID NO: 635 - protein, 5 aa, Homo sapiens
DFGRA

SEQ ID NO: 636 - protein, 5 aa, Homo sapiens
FGRAK

SEQ ID NO: 637 - protein, 5 aa, Homo sapiens
GRAKL

SEQ ID NO: 638 - protein, 5 aa, Homo sapiens
SGIIL

SEQ ID NO: 639 - protein, 5 aa, Homo sapiens
PEQPS

SEQ ID NO: 640 - protein, 5 aa, Homo sapiens
EQPSG

SEQ ID NO: 641 - protein, 5 aa, Homo sapiens
QPSGI

SEQ ID NO: 642 - protein, 5 aa, Homo sapiens
PEQPS

SEQ ID NO: 643 - protein, 5 aa, Homo sapiens
EQPSG

SEQ ID NO: 644 - protein, 5 aa, Homo sapiens
GPRLL

SEQ ID NO: 645 - protein, 5 aa, Homo sapiens
PRLLL

SEQ ID NO: 646 - protein, 5 aa, Homo sapiens
RLLLP

SEQ ID NO: 647 - protein, 5 aa, Homo sapiens
LLLPC

SEQ ID NO: 648 - protein, 5 aa, Homo sapiens
GPRLL

SEQ ID NO: 649 - protein, 5 aa, Homo sapiens
PRLLL

SEQ ID NO: 650 - protein, 5 aa, Homo sapiens
RLLLP

SEQ ID NO: 651 - protein, 5 aa, Homo sapiens
LLLPC

SEQ ID NO: 652 - protein, 5 aa, Homo sapiens
GPRLL

SEQ ID NO: 653 - protein, 5 aa, Homo sapiens
PRLLL

SEQ ID NO: 654 - protein, 5 aa, Homo sapiens
RLLLP

SEQ ID NO: 655 - protein, 5 aa, Homo sapiens
GPRLL

SEQ ID NO: 656 - protein, 5 aa, Homo sapiens
LLLPC

SEQ ID NO: 657 - protein, 5 aa, Homo sapiens
PRLLL

SEQ ID NO: 658 - protein, 5 aa, Homo sapiens
RHSYC

SEQ ID NO: 659 - protein, 5 aa, Homo sapiens
HSYCN

SEQ ID NO: 660 - protein, 5 aa, Homo sapiens
RHSYC

SEQ ID NO: 661 - protein, 5 aa, Homo sapiens
HSYCN

SEQ ID NO: 662 - protein, 5 aa, Homo sapiens
YCNGS

SEQ ID NO: 663 - protein, 15 aa, Homo sapiens
LCMGKITDFGRANHE

SEQ ID NO: 664 - protein, 15 aa, Homo sapiens
RLVFKITDFGRENIM

SEQ ID NO: 665 - protein, 15 aa, Homo sapiens
KLLLKITDFGRGQYL

SEQ ID NO: 666 - protein, 15 aa, Homo sapiens
LDFIITDFGRASTQT

SEQ ID NO: 667 - protein, 15 aa, Homo sapiens
PGFWITDFGRAELMD

SEQ ID NO: 668 - protein, 15 aa, Homo sapiens
QTLLITDFGRASMYT

SEQ ID NO: 669 - protein, 15 aa, Homo sapiens
VMEKRAKLLGAPKYS

SEQ ID NO: 670 - protein, 15 aa, Homo sapiens
PHLLPQPEQPSLHYC

SEQ ID NO: 671 - protein, 15 aa, Homo sapiens
LKWFPQPEQPSIMSF

SEQ ID NO: 672 - protein, 15 aa, Homo sapiens
WNILPQPEQPSQILK

SEQ ID NO: 673 - protein, 15 aa, Homo sapiens
LGSAQPEQPSGSYAC

SEQ ID NO: 674 - protein, 15 aa, Homo sapiens
RLIFQPEQPSGLSIV

SEQ ID NO: 675 - protein, 15 aa, Homo sapiens
GSLMQPEQPSGLLFS

SEQ ID NO: 676 - protein, 15 aa, Homo sapiens
LSTLEQPSGIISRNS

SEQ ID NO: 677 - protein, 15 aa, Homo sapiens
HPFWEQPSGIIQQID

SEQ ID NO: 678 - protein, 15 aa, Homo sapiens
SYLFEQPSGIITINS

SEQ ID NO: 679 - protein, 15 aa, Homo sapiens
FTWRPSGIILDNIRN

SEQ ID NO: 680 - protein, 15 aa, Homo sapiens
SNPASGIILDYLKAV

SEQ ID NO: 681 - protein, 15 aa, Homo sapiens
FSTYSGIILDYPRHM

SEQ ID NO: 682 - protein, 15 aa, Homo sapiens
KLPISGIILDYHVDS

SEQ ID NO: 683 - protein, 15 aa, Homo sapiens
RTYMTIGPRLLIARQ

SEQ ID NO: 684 - protein, 15 aa, Homo sapiens
RLGETIGPRLLLVRQ

SEQ ID NO: 685 - protein, 15 aa, Homo sapiens
PKEYIGPRLLLTVQT

SEQ ID NO: 686 - protein, 15 aa, Homo sapiens
DSRFIGPRLLLPSAN

SEQ ID NO: 687 - protein, 15 aa, Homo sapiens
NILTPRLLLPCPECE

SEQ ID NO: 688 - protein, 15 aa, Homo sapiens
DEYLPRLLLPCAQYD

SEQ ID NO: 689 - protein, 15 aa, Homo sapiens
YEEYLLLPCPMPRTA

SEQ ID NO: 690 - protein, 15 aa, Homo sapiens
PRRPLLLPCPMQTAT

SEQ ID NO: 691 - protein, 15 aa, Homo sapiens
QQVSLLPCPMDPEFS

SEQ ID NO: 692 - protein, 15 aa, Homo sapiens
NGILLLPCPMDSSES

SEQ ID NO: 693 - protein, 15 aa, Homo sapiens
KHSLLLPCPMDKVLD

SEQ ID NO: 694 - protein, 15 aa, Homo sapiens
LCFRTRHSYCNTRTI

SEQ ID NO: 695 - protein, 15 aa, Homo sapiens
MIAGHSYCNGSVACG

SEQ ID NO: 696 - protein, 15 aa, Homo sapiens
HEAMYCNGSEDAQIT

SEQ ID NO: 697 - protein, 5 aa, Homo sapiens
KIDGR

SEQ ID NO: 698 - protein, 5 aa, Homo sapiens
ITFRA

SEQ ID NO: 699 - protein, 5 aa, Homo sapiens
RALGA

SEQ ID NO: 700 - protein, 5 aa, Homo sapiens
PQEPS

SEQ ID NO: 701 - protein, 5 aa, Homo sapiens
QPQSG

SEQ ID NO: 702 - protein, 5 aa, Homo sapiens
EQSII

SEQ ID NO: 703 - protein, 5 aa, Homo sapiens
PSILD

SEQ ID NO: 704 - protein, 5 aa, Homo sapiens
SGIDY

SEQ ID NO: 705 - protein, 5 aa, Homo sapiens
TIPLL

SEQ ID NO: 706 - protein, 5 aa, Homo sapiens
IGRLL

SEQ ID NO: 707 - protein, 5 aa, Homo sapiens
PRLPC

SEQ ID NO: 708 - protein, 5 aa, Homo sapiens
LLPPM

SEQ ID NO: 709 - protein, 5 aa, Homo sapiens
LLCMD

SEQ ID NO: 710 - protein, 5 aa, Homo sapiens
TRSCN

SEQ ID NO: 711 - protein, 5 aa, Homo sapiens
HSCGS

SEQ ID NO: 712 - protein, 5 aa, Homo sapiens
YCGED

SEQ ID NO: 713 - protein, 9 aa, Homo sapiens
IGEYSFGVS

SEQ ID NO: 714 - protein, 9 aa, Homo sapiens
KSESFGVAR

SEQ ID NO: 715 - protein, 9 aa, Homo sapiens
FQSGVATCP

SEQ ID NO: 716 - protein, 9 aa, Homo sapiens
DSSVATCVL

SEQ ID NO: 717 - protein, 9 aa, Homo sapiens
DKYSFGVAF

SEQ ID NO: 718 - protein, 9 aa, Homo sapiens
ERYFGVATL

SEQ ID NO: 719 - protein, 9 aa, Homo sapiens
HKWGVATCW

SEQ ID NO: 720 - protein, 9 aa, Homo sapiens
IGRYSFGVQ

SEQ ID NO: 721 - protein, 9 aa, Homo sapiens
EQRSFGVAG

SEQ ID NO: 722 - protein, 9 aa, Homo sapiens
KGQVATCVP

SEQ ID NO: 723 - protein, 9 aa, Homo sapiens
LNGFGVATR

SEQ ID NO: 724 - protein, 9 aa, Homo sapiens
IKDFGRLAY

SEQ ID NO: 725 - protein, 9 aa, Homo sapiens
EYHTDFGRL

SEQ ID NO: 726 - protein, 9 aa, Homo sapiens
SLKDFGRLI

SEQ ID NO: 727 - protein, 9 aa, Homo sapiens
DVFGRLAKF

SEQ ID NO: 728 - protein, 9 aa, Homo sapiens
RLRRLAKLL

SEQ ID NO: 729 - protein, 9 aa, Homo sapiens
TARDFGRLE

SEQ ID NO: 730 - protein, 9 aa, Homo sapiens
TNKFGRLAD

SEQ ID NO: 731 - protein, 9 aa, Homo sapiens
HGNDFGRLR

SEQ ID NO: 732 - protein, 9 aa, Homo sapiens
CPRFGRLAY

SEQ ID NO: 733 - protein, 9 aa, Homo sapiens
KCIRLAKLR

SEQ ID NO: 734 - protein, 9 aa, Homo sapiens
RSELIKRQL

SEQ ID NO: 735 - protein, 9 aa, Homo sapiens
SFAIKRQRL

SEQ ID NO: 736 - protein, 9 aa, Homo sapiens
PRLKRQRQI

SEQ ID NO: 737 - protein, 9 aa, Homo sapiens
VINRQRQQF

SEQ ID NO: 738 - protein, 9 aa, Homo sapiens
LSYQRQQKF

SEQ ID NO: 739 - protein, 9 aa, Homo sapiens
SGKIKRQRN

SEQ ID NO: 740 - protein, 9 aa, Homo sapiens
TMRRQRQQS

SEQ ID NO: 741 - protein, 9 aa, Homo sapiens
AVELIKRQY

SEQ ID NO: 742 - protein, 9 aa, Homo sapiens
RGNTDVGFL

SEQ ID NO: 743 - protein, 9 aa, Homo sapiens
YGQDVGFSQ

SEQ ID NO: 744 - protein, 9 aa, Homo sapiens
YTDVGFSCA

SEQ ID NO: 745 - protein, 9 aa, Homo sapiens
HKLTDVGFF

SEQ ID NO: 746 - protein, 9 aa, Homo sapiens
QRTDVGFSF

SEQ ID NO: 747 - protein, 9 aa, Homo sapiens
PRYGFSCTF

SEQ ID NO: 748 - protein, 9 aa, Homo sapiens
VVKTDVGFA

SEQ ID NO: 749 - protein, 9 aa, Homo sapiens
SIKVGFSCS

SEQ ID NO: 750 - protein, 9 aa, Homo sapiens
TIRFSCTLQ

SEQ ID NO: 751 - protein, 9 aa, Homo sapiens
ISTFSCTLR

SEQ ID NO: 752 - protein, 9 aa, Homo sapiens
LLDATEVKP

SEQ ID NO: 753 - protein, 9 aa, Homo sapiens
FKCEVKSRP

SEQ ID NO: 754 - protein, 9 aa, Homo sapiens
YLKGLATEW

SEQ ID NO: 755 - protein, 9 aa, Homo sapiens
KPPLATEVF

SEQ ID NO: 756 - protein, 9 aa, Homo sapiens
LFKATEVKL

SEQ ID NO: 757 - protein, 9 aa, Homo sapiens
LQFEVKSRL

SEQ ID NO: 758 - protein, 9 aa, Homo sapiens
AVKATEVKA

SEQ ID NO: 759 - protein, 9 aa, Homo sapiens
VGLTEVKSY

SEQ ID NO: 760 - protein, 9 aa, Homo sapiens
IEIEVKSRY

SEQ ID NO: 761 - protein, 9 aa, Homo sapiens
ASDLATMVE

SEQ ID NO: 762 - protein, 9 aa, Homo sapiens
MSNATMVKL

SEQ ID NO: 763 - protein, 9 aa, Homo sapiens
IAEMVKSRV

SEQ ID NO: 764 - protein, 9 aa, Homo sapiens
KWDGLATML

SEQ ID NO: 765 - protein, 9 aa, Homo sapiens
RMSATMVKF

SEQ ID NO: 766 - protein, 9 aa, Homo sapiens
IRRTMVKSI

SEQ ID NO: 767 - protein, 9 aa, Homo sapiens
VRTMVKSRF

SEQ ID NO: 768 - protein, 9 aa, Homo sapiens
NMKLATMVG

SEQ ID NO: 769 - protein, 9 aa, Homo sapiens
YGKATMVKA

SEQ ID NO: 770 - protein, 9 aa, Homo sapiens
VELTMVKSY

SEQ ID NO: 771 - protein, 9 aa, Homo sapiens
TVIMVKSRR

SEQ ID NO: 772 - protein, 9 aa, Homo sapiens
CPEITKEQY

SEQ ID NO: 773 - protein, 9 aa, Homo sapiens
RGIEITKER

SEQ ID NO: 774 - protein, 9 aa, Homo sapiens
PSDMNDARL

SEQ ID NO: 775 - protein, 9 aa, Homo sapiens
YADARHHGC

SEQ ID NO: 776 - protein, 9 aa, Homo sapiens
APRDARHHK

SEQ ID NO: 777 - protein, 9 aa, Homo sapiens
KARARHHGA

SEQ ID NO: 778 - protein, 9 aa, Homo sapiens
WKIRHHGGR

SEQ ID NO: 779 - protein, 9 aa, Homo sapiens
FGDKGGRTG

SEQ ID NO: 780 - protein, 9 aa, Homo sapiens
QGKAGKGGP

SEQ ID NO: 781 - protein, 9 aa, Homo sapiens
DNRKGGRTK

SEQ ID NO: 782 - protein, 9 aa, Homo sapiens
NNRGGRTGA

SEQ ID NO: 783 - protein, 9 aa, Homo sapiens
LITAGKGGY

SEQ ID NO: 784 - protein, 9 aa, Homo sapiens
EHFKGGRTY

SEQ ID NO: 785 - protein, 9 aa, Homo sapiens
PAKAGKGQP

SEQ ID NO: 786 - protein, 9 aa, Homo sapiens
PDRKGQRTG

SEQ ID NO: 787 - protein, 9 aa, Homo sapiens
RAWGQRTGP

SEQ ID NO: 788 - protein, 9 aa, Homo sapiens
GVLAGKGQY

SEQ ID NO: 789 - protein, 9 aa, Homo sapiens
QECKGQRTY

SEQ ID NO: 790 - protein, 9 aa, Homo sapiens
PDKSFGVAY

SEQ ID NO: 791 - protein, 9 aa, Homo sapiens
WKTFGVATS

SEQ ID NO: 792 - protein, 9 aa, Homo sapiens
PEFGVATCK

SEQ ID NO: 793 - protein, 9 aa, Homo sapiens
FEHSFGVAS

SEQ ID NO: 794 - protein, 9 aa, Homo sapiens
YRPGVATCV

SEQ ID NO: 795 - protein, 9 aa, Homo sapiens
GKTSFGVAG

SEQ ID NO: 796 - protein, 9 aa, Homo sapiens
GKHFGVATL

SEQ ID NO: 797 - protein, 9 aa, Homo sapiens
REQGVATCL

SEQ ID NO: 798 - protein, 9 aa, Homo sapiens
RGSYSFGVY

SEQ ID NO: 799 - protein, 9 aa, Homo sapiens
GGSGVATCY

SEQ ID NO: 800 - protein, 9 aa, Homo sapiens
DRLTDFGRE

SEQ ID NO: 801 - protein, 9 aa, Homo sapiens
YEMDFGRLY

SEQ ID NO: 802 - protein, 9 aa, Homo sapiens
VRGGRLAKR

SEQ ID NO: 803 - protein, 9 aa, Homo sapiens
FRFTDFGRT

SEQ ID NO: 804 - protein, 9 aa, Homo sapiens
QELGRLAKP

SEQ ID NO: 805 - protein, 9 aa, Homo sapiens
KQYTDFGRL

SEQ ID NO: 806 - protein, 9 aa, Homo sapiens
RCYDFGRLW

SEQ ID NO: 807 - protein, 9 aa, Homo sapiens
YKLGRLAKI

SEQ ID NO: 808 - protein, 9 aa, Homo sapiens
ENPRLAKLI

SEQ ID NO: 809 - protein, 9 aa, Homo sapiens
ELNTDFGRW

SEQ ID NO: 810 - protein, 9 aa, Homo sapiens
CSNGRLAKF

SEQ ID NO: 811 - protein, 9 aa, Homo sapiens
IRSLIKRQL

SEQ ID NO: 812 - protein, 9 aa, Homo sapiens
THWQRQQKL

SEQ ID NO: 813 - protein, 9 aa, Homo sapiens
RCLKRQRQL

SEQ ID NO: 814 - protein, 9 aa, Homo sapiens
RDLQRQQKV

SEQ ID NO: 815 - protein, 9 aa, Homo sapiens
SPWLIKRQI

SEQ ID NO: 816 - protein, 9 aa, Homo sapiens
TAAQRQQKY

SEQ ID NO: 817 - protein, 9 aa, Homo sapiens
TKRGFSCTK

SEQ ID NO: 818 - protein, 9 aa, Homo sapiens
TRKDVGFSI

SEQ ID NO: 819 - protein, 9 aa, Homo sapiens
LRHGFSCTC

SEQ ID NO: 820 - protein, 9 aa, Homo sapiens
QDEDVGFSM

SEQ ID NO: 821 - protein, 9 aa, Homo sapiens
QNEGFSCTA

SEQ ID NO: 822 - protein, 9 aa, Homo sapiens
PNQFSCTLS

SEQ ID NO: 823 - protein, 9 aa, Homo sapiens
HSKDVGFSI

SEQ ID NO: 824 - protein, 9 aa, Homo sapiens
GSKGFSCTM

SEQ ID NO: 825 - protein, 9 aa, Homo sapiens
DQFGLATEK

SEQ ID NO: 826 - protein, 9 aa, Homo sapiens
LRDEVKSRE

SEQ ID NO: 827 - protein, 9 aa, Homo sapiens
IRKGLATEY

SEQ ID NO: 828 - protein, 9 aa, Homo sapiens
PNVGLATEI

SEQ ID NO: 829 - protein, 9 aa, Homo sapiens
AKAATEVKL

SEQ ID NO: 830 - protein, 9 aa, Homo sapiens
PSCGLATEM

SEQ ID NO: 831 - protein, 9 aa, Homo sapiens
LSKATEVKL

SEQ ID NO: 832 - protein, 9 aa, Homo sapiens
SCITEVKSF

SEQ ID NO: 833 - protein, 9 aa, Homo sapiens
ESPEVKSRY

SEQ ID NO: 834 - protein, 9 aa, Homo sapiens
DKVGLATMA

SEQ ID NO: 835 - protein, 9 aa, Homo sapiens
IRGMVKSRN

SEQ ID NO: 836 - protein, 9 aa, Homo sapiens
LRQGLATMQ

SEQ ID NO: 837 - protein, 9 aa, Homo sapiens
ENPGLATMI

SEQ ID NO: 838 - protein, 9 aa, Homo sapiens
SKGATMVKL

SEQ ID NO: 839 - protein, 9 aa, Homo sapiens
GQVTMVKSI

SEQ ID NO: 840 - protein, 9 aa, Homo sapiens
KAKGLATMM

SEQ ID NO: 841 - protein, 9 aa, Homo sapiens
RGDATMVKI

SEQ ID NO: 842 - protein, 9 aa, Homo sapiens
VGCTMVKSM

SEQ ID NO: 843 - protein, 9 aa, Homo sapiens
TITMVKSRW

SEQ ID NO: 844 - protein, 9 aa, Homo sapiens
KKAEITKES

SEQ ID NO: 845 - protein, 9 aa, Homo sapiens
MRPTKEQEQ

SEQ ID NO: 846 - protein, 9 aa, Homo sapiens
ADLSEITKV

SEQ ID NO: 847 - protein, 9 aa, Homo sapiens
NKLITKEQL

SEQ ID NO: 848 - protein, 9 aa, Homo sapiens
LRVNDARHI

SEQ ID NO: 849 - protein, 9 aa, Homo sapiens
LKPRHHGGN

SEQ ID NO: 850 - protein, 9 aa, Homo sapiens
IRMDARHHV

SEQ ID NO: 851 - protein, 9 aa, Homo sapiens
PEWARHHGW

SEQ ID NO: 852 - protein, 9 aa, Homo sapiens
SQARHHGGC

SEQ ID NO: 853 - protein, 9 aa, Homo sapiens
IEQNDARHF

SEQ ID NO: 854 - protein, 9 aa, Homo sapiens
FGHRHHGGR

SEQ ID NO: 855 - protein, 9 aa, Homo sapiens
KQLAGKGGP

SEQ ID NO: 856 - protein, 9 aa, Homo sapiens
REWGRTGVE

SEQ ID NO: 857 - protein, 9 aa, Homo sapiens
VHCAGKGGL

SEQ ID NO: 858 - protein, 9 aa, Homo sapiens
MCWGKGGRA

SEQ ID NO: 859 - protein, 9 aa, Homo sapiens
ARQGRTGVS

SEQ ID NO: 860 - protein, 9 aa, Homo sapiens
KTWKGGRTL

SEQ ID NO: 861 - protein, 9 aa, Homo sapiens
FKCGRTGVL

SEQ ID NO: 862 - protein, 9 aa, Homo sapiens
NITAGKGGW

SEQ ID NO: 863 - protein, 9 aa, Homo sapiens
LSHKGGRTR

SEQ ID NO: 864 - protein, 9 aa, Homo sapiens
GGPGGRTGM

SEQ ID NO: 865 - protein, 9 aa, Homo sapiens
DRTAGKGQE

SEQ ID NO: 866 - protein, 9 aa, Homo sapiens
LRKAGKGQP

SEQ ID NO: 867 - protein, 9 aa, Homo sapiens
LRTQRTGVP

SEQ ID NO: 868 - protein, 9 aa, Homo sapiens
GMFKGQRTL

SEQ ID NO: 869 - protein, 9 aa, Homo sapiens
PDLQRTGVL

SEQ ID NO: 870 - protein, 9 aa, Homo sapiens
GDFAGKGQF

SEQ ID NO: 871 - protein, 9 aa, Homo sapiens
QANKGQRTL

SEQ ID NO: 872 - protein, 9 aa, Homo sapiens
EGMGQRTGL

SEQ ID NO: 873 - protein, 15 aa, Homo sapiens
SSDAGKYSFGVLRLM

SEQ ID NO: 874 - protein, 15 aa, Homo sapiens
GEQQKYSFGVAQNWC

SEQ ID NO: 875 - protein, 15 aa, Homo sapiens
RAEPSFGVATCGHFN

SEQ ID NO: 876 - protein, 15 aa, Homo sapiens
AISKGVATCVKGKFV

SEQ ID NO: 877 - protein, 15 aa, Homo sapiens
MNCNVATCVKKACVF

SEQ ID NO: 878 - protein, 15 aa, Homo sapiens
LDLLGKYSFGVSAPG

SEQ ID NO: 879 - protein, 15 aa, Homo sapiens
VVNRGVATCVKAVNE

SEQ ID NO: 880 - protein, 15 aa, Homo sapiens
LFQKVATCVKKAESS

SEQ ID NO: 881 - protein, 15 aa, Homo sapiens
RCHFGVATCVKTMDF

SEQ ID NO: 882 - protein, 15 aa, Homo sapiens
IRRHSFGVATCELVC

SEQ ID NO: 883 - protein, 15 aa, Homo sapiens
HSDHGVATCVKPMYT

SEQ ID NO: 884 - protein, 15 aa, Homo sapiens
LSTPITDFGRLAGHA

SEQ ID NO: 885 - protein, 15 aa, Homo sapiens
FDQKGRLAKLLTWIC

SEQ ID NO: 886 - protein, 15 aa, Homo sapiens
FVIWKITDFGRVKYN

SEQ ID NO: 887 - protein, 15 aa, Homo sapiens
IVSWITDFGRLWKRN

SEQ ID NO: 888 - protein, 15 aa, Homo sapiens
TLLMDFGRLAKRTMK

SEQ ID NO: 889 - protein, 15 aa, Homo sapiens
SEMFGRLAKLLEYAI

SEQ ID NO: 890 - protein, 15 aa, Homo sapiens
SQEIRLAKLLGYRSR

SEQ ID NO: 891 - protein, 15 aa, Homo sapiens
SAYEKITDFGRKIVI

SEQ ID NO: 892 - protein, 15 aa, Homo sapiens
LGYEITDFGRLRVGY

SEQ ID NO: 893 - protein, 15 aa, Homo sapiens
NKIFDFGRLAKRLII

SEQ ID NO: 894 - protein, 15 aa, Homo sapiens
LNLLIKRQRQQPPNC

SEQ ID NO: 895 - protein, 15 aa, Homo sapiens
LGVHGILIKRQACHC

SEQ ID NO: 896 - protein, 15 aa, Homo sapiens
YMCLILIKRQRLTNR

SEQ ID NO: 897 - protein, 15 aa, Homo sapiens
FALFIKRQRQQSQCW

SEQ ID NO: 898 - protein, 15 aa, Homo sapiens
LIVWQRQQKIRALTE

SEQ ID NO: 899 - protein, 15 aa, Homo sapiens
DAWYIKRQRQQRLTC

SEQ ID NO: 900 - protein, 15 aa, Homo sapiens
PGLDRQRQQKIIVQD

SEQ ID NO: 901 - protein, 15 aa, Homo sapiens
GFKTQRQQKIRVFLE

SEQ ID NO: 902 - protein, 15 aa, Homo sapiens
GVDVLSTDVGFQDIC

SEQ ID NO: 903 - protein, 15 aa, Homo sapiens
PGTSSTDVGFSGEFH

SEQ ID NO: 904 - protein, 15 aa, Homo sapiens
SENYDVGFSCTYDLV

SEQ ID NO: 905 - protein, 15 aa, Homo sapiens
GFGNGFSCTLVQHDT

SEQ ID NO: 906 - protein, 15 aa, Homo sapiens
LALQLSTDVGFSAPS

SEQ ID NO: 907 - protein, 15 aa, Homo sapiens
SNISSTDVGFSPLAV

SEQ ID NO: 908 - protein, 15 aa, Homo sapiens
SATVDVGFSCTDHLT

SEQ ID NO: 909 - protein, 15 aa, Homo sapiens
AEILGFSCTLVATRS

SEQ ID NO: 910 - protein, 15 aa, Homo sapiens
DHFFLSTDVGFRIER

SEQ ID NO: 911 - protein, 15 aa, Homo sapiens
LMRISTDVGFSVKVC

SEQ ID NO: 912 - protein, 15 aa, Homo sapiens
LSRMFSCTLVCQSGH

SEQ ID NO: 913 - protein, 15 aa, Homo sapiens
SSWELSTDVGFYSEI

SEQ ID NO: 914 - protein, 15 aa, Homo sapiens
STLYSTDVGFSYITG

SEQ ID NO: 915 - protein, 15 aa, Homo sapiens
IERKGFSCTLVTMIQ

SEQ ID NO: 916 - protein, 15 aa, Homo sapiens
ANKKEVKSRWSAQLC

SEQ ID NO: 917 - protein, 15 aa, Homo sapiens
LCKSLATEVKSPFKQ

SEQ ID NO: 918 - protein, 15 aa, Homo sapiens
FNLLTEVKSRWPYCD

SEQ ID NO: 919 - protein, 15 aa, Homo sapiens
AQLPEVKSRWSTDWE

SEQ ID NO: 920 - protein, 15 aa, Homo sapiens
SVLRDFGLATELYKI

SEQ ID NO: 921 - protein, 15 aa, Homo sapiens
GMRYFGLATEVPASM

SEQ ID NO: 922 - protein, 15 aa, Homo sapiens
CPFCTEVKSRWFLLK

SEQ ID NO: 923 - protein, 15 aa, Homo sapiens
PRHRFGLATMVCCTG

SEQ ID NO: 924 - protein, 15 aa, Homo sapiens
DCQDLATMVKSVCSS

SEQ ID NO: 925 - protein, 15 aa, Homo sapiens
FRKTMVKSRWSRCLC

SEQ ID NO: 926 - protein, 15 aa, Homo sapiens
LNPTLATMVKSLEES

SEQ ID NO: 927 - protein, 15 aa, Homo sapiens
LALLMVKSRWSTGEV

SEQ ID NO: 928 - protein, 15 aa, Homo sapiens
NYGVDFGLATMLTHH

SEQ ID NO: 929 - protein, 15 aa, Homo sapiens
KYISFGLATMVKNVD

SEQ ID NO: 930 - protein, 15 aa, Homo sapiens
VCEILATMVKSYRLD

SEQ ID NO: 931 - protein, 15 aa, Homo sapiens
LNELTMVKSRWLPLK

SEQ ID NO: 932 - protein, 15 aa, Homo sapiens
DGENEITKEQEQCLE

SEQ ID NO: 933 - protein, 15 aa, Homo sapiens
LYFSLSEITKELGQC

SEQ ID NO: 934 - protein, 15 aa, Homo sapiens
LCLGKEQEKDFVARA

SEQ ID NO: 935 - protein, 15 aa, Homo sapiens
FILLEITKEQERVYC

SEQ ID NO: 936 - protein, 15 aa, Homo sapiens
SYWQTKEQEKDRLVT

SEQ ID NO: 937 - protein, 15 aa, Homo sapiens
KNLDKEQEKDFIIII

SEQ ID NO: 938 - protein, 15 aa, Homo sapiens
LDLTRHHGGWTASID

SEQ ID NO: 939 - protein, 15 aa, Homo sapiens
DFNEKQMNDARYIIE

SEQ ID NO: 940 - protein, 15 aa, Homo sapiens
CPVVQMNDARHQLIV

SEQ ID NO: 941 - protein, 15 aa, Homo sapiens
KKYLNDARHHGIILV

SEQ ID NO: 942 - protein, 15 aa, Homo sapiens
GQLRCKAGKGGYRPN

SEQ ID NO: 943 - protein, 15 aa, Homo sapiens
LEENGKGGRTGPINC

SEQ ID NO: 944 - protein, 15 aa, Homo sapiens
NKEFGGRTGVMWCII

SEQ ID NO: 945 - protein, 15 aa, Homo sapiens
SNQDGRTGVMIMEID

SEQ ID NO: 946 - protein, 15 aa, Homo sapiens
ICLLCKAGKGGSSES

SEQ ID NO: 947 - protein, 15 aa, Homo sapiens
LVAQGKGGRTGLPIG

SEQ ID NO: 948 - protein, 15 aa, Homo sapiens
LPAYGGRTGVMSYEG

SEQ ID NO: 949 - protein, 15 aa, Homo sapiens
TNNPCKAGKGQFEVW

SEQ ID NO: 950 - protein, 15 aa, Homo sapiens
QFEKGKGQRTGGHVM

SEQ ID NO: 951 - protein, 15 aa, Homo sapiens
AELAGQRTGVMACYD

SEQ ID NO: 952 - protein, 15 aa, Homo sapiens
SQRLQRTGVMIPCFI

SEQ ID NO: 953 - protein, 15 aa, Homo sapiens
LVPTGKGQRTGAYYS

SEQ ID NO: 954 - protein, 15 aa, Homo sapiens
SCIFKAGKGQRPHIT

SEQ ID NO: 955 - protein, 15 aa, Homo sapiens
FQRPGQRTGVMCMGM

SEQ ID NO: 956 - protein, 15 aa, Homo sapiens
LTQDQRTGVMIYDFC

SEQ ID NO: 957 - protein, 9 aa, Homo sapiens
LADKKGNYV

SEQ ID NO: 958 - protein, 9 aa, Homo sapiens
KASEKKGNY

SEQ ID NO: 959 - protein, 9 aa, Homo sapiens
DGDGNYVVS

SEQ ID NO: 960 - protein, 9 aa, Homo sapiens
KLAEKKGNV

SEQ ID NO: 961 - protein, 9 aa, Homo sapiens
QYTKKGNYF

SEQ ID NO: 962 - protein, 9 aa, Homo sapiens
KYTKGNYVW

SEQ ID NO: 963 - protein, 9 aa, Homo sapiens
ESDKGNYVC

SEQ ID NO: 964 - protein, 9 aa, Homo sapiens
APGEEKKGG

SEQ ID NO: 965 - protein, 9 aa, Homo sapiens
PPDKGNYVA

SEQ ID NO: 966 - protein, 9 aa, Homo sapiens
LLREEKKGF

SEQ ID NO: 967 - protein, 9 aa, Homo sapiens
FAMEKKGNY

SEQ ID NO: 968 - protein, 9 aa, Homo sapiens
ECRKGNYVE

SEQ ID NO: 969 - protein, 9 aa, Homo sapiens
PCQKKGNYV

SEQ ID NO: 970 - protein, 9 aa, Homo sapiens
LGDEKKGNF

SEQ ID NO: 971 - protein, 9 aa, Homo sapiens
PASEEKKGF

SEQ ID NO: 972 - protein, 9 aa, Homo sapiens
IRQKGNYVS

SEQ ID NO: 973 - protein, 9 aa, Homo sapiens
LWSEKKGNG

SEQ ID NO: 974 - protein, 9 aa, Homo sapiens
TKSKKGNYR

SEQ ID NO: 975 - protein, 9 aa, Homo sapiens
IRRGNYVVS

SEQ ID NO: 976 - protein, 9 aa, Homo sapiens
LKEEEKKGD

SEQ ID NO: 977 - protein, 9 aa, Homo sapiens
RAEGNYVVR

SEQ ID NO: 978 - protein, 9 aa, Homo sapiens
MGEKKGNYD

SEQ ID NO: 979 - protein, 9 aa, Homo sapiens
TADEKKGNF

SEQ ID NO: 980 - protein, 9 aa, Homo sapiens
RLKEKKGNV

SEQ ID NO: 981 - protein, 9 aa, Homo sapiens
QLPKKGNYI

SEQ ID NO: 982 - protein, 9 aa, Homo sapiens
TKGKGNYVI

SEQ ID NO: 983 - protein, 9 aa, Homo sapiens
EVSKGNYVA

SEQ ID NO: 984 - protein, 9 aa, Homo sapiens
NVRKGNYVA

SEQ ID NO: 985 - protein, 9 aa, Homo sapiens
RTQEEKKGI

SEQ ID NO: 986 - protein, 9 aa, Homo sapiens
QSCEKKGNW

SEQ ID NO: 987 - protein, 9 aa, Homo sapiens
FPMEEKKGR

SEQ ID NO: 988 - protein, 9 aa, Homo sapiens
SEEKKGNYQ

SEQ ID NO: 989 - protein, 9 aa, Homo sapiens
LELKGNYVP

SEQ ID NO: 990 - protein, 9 aa, Homo sapiens
EGPEEKKGY

SEQ ID NO: 991 - protein, 9 aa, Homo sapiens
ISKEKKGNF

SEQ ID NO: 992 - protein, 9 aa, Homo sapiens
EHMKGNYVG

SEQ ID NO: 993 - protein, 9 aa, Homo sapiens
RELEKKGNA

SEQ ID NO: 994 - protein, 9 aa, Homo sapiens
AEHGNYVVT

SEQ ID NO: 995 - protein, 9 aa, Homo sapiens
TRVKKGNYS

SEQ ID NO: 996 - protein, 9 aa, Homo sapiens
WKEEEKKGR

SEQ ID NO: 997 - protein, 15 aa, Homo sapiens
FKRELMNLPQQCNFR

SEQ ID NO: 998 - protein, 15 aa, Homo sapiens
QFKRELMNLPQQCNF

SEQ ID NO: 999 - protein, 15 aa, Homo sapiens
QQFKRELMNLPQQCN

SEQ ID NO: 1000 - protein, 15 aa, Homo sapiens
EQHIMQRIMGEQEQY

SEQ ID NO: 1001 - protein, 15 aa, Homo sapiens
QFKRELMNLPQQCNF

SEQ ID NO: 1002 - protein, 15 aa, Homo sapiens
NTQRCMCEALQQIME

SEQ ID NO: 1003 - protein, 15 aa, Homo sapiens
QQFKRELMNLPQQCN

SEQ ID NO: 1004 - protein, 15 aa, Homo sapiens
FKRELMNLPQQCNFR

SEQ ID NO: 1005 - protein, 15 aa, Homo sapiens
QFKRELMNLPQQCNF

SEQ ID NO: 1006 - protein, 15 aa, Homo sapiens
KPCEQHIMQRIMGEQ

SEQ ID NO: 1007 - protein, 15 aa, Homo sapiens
EQHIMQRIMGEQEQY

SEQ ID NO: 1008 - protein, 15 aa, Homo sapiens
CEQHIMQRIMGEQEQ

SEQ ID NO: 1009 - protein, 15 aa, Homo sapiens
PCEQHIMQRIMGEQE

SEQ ID NO: 1010 - protein, 15 aa, Homo sapiens
KPCEQHIMQRIMGEQ

SEQ ID NO: 1011 - protein, 15 aa, Homo sapiens
MVQQFKRELMNLPQQ

SEQ ID NO: 1012 - protein, 15 aa, Homo sapiens
QFKRELMNLPQQCNF

SEQ ID NO: 1013 - protein, 15 aa, Homo sapiens
CEQHIMQRIMGEQEQ

SEQ ID NO: 1014 - protein, 15 aa, Homo sapiens
KPCEQHIMQRIMGEQ

SEQ ID NO: 1015 - protein, 15 aa, Homo sapiens
PCEQHIMQRIMGEQE

SEQ ID NO: 1016 - protein, 15 aa, Homo sapiens
LNRLLMNLPQQATLI

SEQ ID NO: 1017 - protein, 15 aa, Homo sapiens
IRQLELMNLPQIYLN

SEQ ID NO: 1018 - protein, 15 aa, Homo sapiens
MIRLRELMNLPVARC

SEQ ID NO: 1019 - protein, 15 aa, Homo sapiens
VQAMMQRIMGELLLE

SEQ ID NO: 1020 - protein, 15 aa, Homo sapiens
RQMQELMNLPQLILI

SEQ ID NO: 1021 - protein, 15 aa, Homo sapiens
MQFMCMCEALQALLV

SEQ ID NO: 1022 - protein, 15 aa, Homo sapiens
PMLLRELMNLPRTRR

SEQ ID NO: 1023 - protein, 15 aa, Homo sapiens
LILLLMNLPQQNTVN

SEQ ID NO: 1024 - protein, 15 aa, Homo sapiens
FLIFELMNLPQMRNI

SEQ ID NO: 1025 - protein, 15 aa, Homo sapiens
IMFLQHIMQRIELQY

SEQ ID NO: 1026 - protein, 15 aa, Homo sapiens
LRMLMQRIMGENQRV

SEQ ID NO: 1027 - protein, 15 aa, Homo sapiens
RELQIMQRIMGAVLC

SEQ ID NO: 1028 - protein, 15 aa, Homo sapiens
LHQRHIMQRIMAQVF

SEQ ID NO: 1029 - protein, 15 aa, Homo sapiens
LQVDQHIMQRISCLM

SEQ ID NO: 1030 - protein, 15 aa, Homo sapiens
NIILFKRELMNMHQC

SEQ ID NO: 1031 - protein, 15 aa, Homo sapiens
CCVQELMNLPQRCAA

SEQ ID NO: 1032 - protein, 15 aa, Homo sapiens
MIMMIMQRIMGSVCG

SEQ ID NO: 1033 - protein, 15 aa, Homo sapiens
GCACQHIMQRIPCAR

SEQ ID NO: 1034 - protein, 15 aa, Homo sapiens
CCSIHIMQRIMALAD

SEQ ID NO: 1035 - protein, 5 aa, Homo sapiens
LMLQQ

SEQ ID NO: 1036 - protein, 5 aa, Homo sapiens
ELNPQ

SEQ ID NO: 1037 - protein, 5 aa, Homo sapiens
REMLP

SEQ ID NO: 1038 - protein, 5 aa, Homo sapiens
MQIGE

SEQ ID NO: 1039 - protein, 5 aa, Homo sapiens
ELNPQ

SEQ ID NO: 1040 - protein, 5 aa, Homo sapiens
CMELQ

SEQ ID NO: 1041 - protein, 5 aa, Homo sapiens
REMLP

SEQ ID NO: 1042 - protein, 5 aa, Homo sapiens
LMLQQ

SEQ ID NO: 1043 - protein, 5 aa, Homo sapiens
ELNPQ

SEQ ID NO: 1044 - protein, 5 aa, Homo sapiens
QHMRI

SEQ ID NO: 1045 - protein, 5 aa, Homo sapiens
MQIGE

SEQ ID NO: 1046 - protein, 5 aa, Homo sapiens
IMRMG

SEQ ID NO: 1047 - protein, 5 aa, Homo sapiens
HIQIM

SEQ ID NO: 1048 - protein, 5 aa, Homo sapiens
QHMRI

SEQ ID NO: 1049 - protein, 5 aa, Homo sapiens
FKEMN

SEQ ID NO: 1050 - protein, 5 aa, Homo sapiens
ELNPQ

SEQ ID NO: 1051 - protein, 5 aa, Homo sapiens
IMRMG

SEQ ID NO: 1052 - protein, 5 aa, Homo sapiens
QHMRI

SEQ ID NO: 1053 - protein, 5 aa, Homo sapiens
HIQIM

SEQ ID NO: 1054 - protein, 15 aa, Homo sapiens
KIHAREIFDSXGNPT

SEQ ID NO: 1055 - protein, 15 aa, Homo sapiens
AREIFDSXGNPTVEV

SEQ ID NO: 1056 - protein, 15 aa, Homo sapiens
AREIFDSXGNPTVEV

SEQ ID NO: 1057 - protein, 15 aa, Homo sapiens
FTSKGLFXAAVPSGA

SEQ ID NO: 1058 - protein, 15 aa, Homo sapiens
SKGLFXAAVPSGAST

SEQ ID NO: 1059 - protein, 15 aa, Homo sapiens
KGLFXAAVPSGASTG

SEQ ID NO: 1060 - protein, 15 aa, Homo sapiens
PGGVYATXSSAVXLX

SEQ ID NO: 1061 - protein, 15 aa, Homo sapiens
VYATXSSAVXLXSSV

SEQ ID NO: 1062 - protein, 15 aa, Homo sapiens
XSSAVXLXSSVPGVR

SEQ ID NO: 1063 - protein, 15 aa, Homo sapiens
SSAVXLXSSVPGVRL

SEQ ID NO: 1064 - protein, 15 aa, Homo sapiens
SAVXLXSSVPGVRLL

SEQ ID NO: 1065 - protein, 15 aa, Homo sapiens
PLPNFSSLNLXETNL

SEQ ID NO: 1066 - protein, 15 aa, Homo sapiens
NFSSLNLXETNLDSL

SEQ ID NO: 1067 - protein, 15 aa, Homo sapiens
SSLNLXETNLDSLPL

SEQ ID NO: 1068 - protein, 15 aa, Homo sapiens
PLIFREIFDSXGVQI

SEQ ID NO: 1069 - protein, 15 aa, Homo sapiens
KLIFFDSXGNPTADM

SEQ ID NO: 1070 - protein, 15 aa, Homo sapiens
DFNFFDSXGNPSASL

SEQ ID NO: 1071 - protein, 15 aa, Homo sapiens
QLLFGLFXAAVLTKH

SEQ ID NO: 1072 - protein, 15 aa, Homo sapiens
ALQYFXAAVPSSGLM

SEQ ID NO: 1073 - protein, 15 aa, Homo sapiens
VIIFXAAVPSGGGLI

SEQ ID NO: 1074 - protein, 15 aa, Homo sapiens
KQQYYATXSSAGSLF

SEQ ID NO: 1075 - protein, 15 aa, Homo sapiens
NAFFXSSAVXLGLST

SEQ ID NO: 1076 - protein, 15 aa, Homo sapiens
RAILVXLXSSVKAQI

SEQ ID NO: 1077 - protein, 15 aa, Homo sapiens
EMLWXLXSSVPGTQD

SEQ ID NO: 1078 - protein, 15 aa, Homo sapiens
TLEWLXSSVPGSGLP

SEQ ID NO: 1079 - protein, 15 aa, Homo sapiens
PFYVFSSLNLXNNVA

SEQ ID NO: 1080 - protein, 15 aa, Homo sapiens
QLIWLNLXETNIQTA

SEQ ID NO: 1081 - protein, 15 aa, Homo sapiens
YQILLXETNLDDAPM

SEQ ID NO: 1082 - protein, 5 aa, Homo sapiens
REFSX

SEQ ID NO: 1083 - protein, 5 aa, Homo sapiens
FDXNP

SEQ ID NO: 1084 - protein, 5 aa, Homo sapiens
FDXNP

SEQ ID NO: 1085 - protein, 5 aa, Homo sapiens
GLXAV

SEQ ID NO: 1086 - protein, 5 aa, Homo sapiens
FXAPS

SEQ ID NO: 1087 - protein, 5 aa, Homo sapiens
XAVSG

SEQ ID NO: 1088 - protein, 5 aa, Homo sapiens
YAXSA

SEQ ID NO: 1089-1090 - skipped (2 entries)

SEQ ID NO: 1091 - protein, 5 aa, Homo sapiens
XLSVP

SEQ ID NO: 1092 - protein, 5 aa, Homo sapiens
LXSPG

SEQ ID NO: 1093 - protein, 5 aa, Homo sapiens
FSLLX

SEQ ID NO: 1094 - protein, 5 aa, Homo sapiens
LNXTN

SEQ ID NO: 1095 - protein, 5 aa, Homo sapiens
LXTLD